(19) United States
(12) Patent Application Publication
(10) Pub. No.: US 2026/0285846 A1
(43) Pub. Date: Sep. 24, 2026

(54) REV-ERB AGONISTS FOR TH17-MEDIATED INFLAMMATORY DISORDERS

(71) Applicants: UNIVERSITY OF FLORID RESEARCH FOUNDATION, iNCORPORATED, Gainesville, FL (US); SHANGPHARMA INNOVATION, South San Francisco, CA (US)

(72) Inventors: Theodore Mark Kamenecka, Gainesville, FL (US); Laura A, Solt, Gainesville, FL (US); Yuanjun He, Gainesville, FL (US); Mike Lizarzburu, Pacifica, CA (US); Brett C. Bookser, Kensington, CA (US)

(73) Assignees: UNIVERSITY OF FLORID RESEARCH FOUNDATION, iNCORPORATED, Gainesville, FL (US); SHANGPHARMA INNOVATION, South San Francisco, CA (US)

(21) Appl. No.: 18/721,334

(22) PCT Filed: Dec. 20, 2022
(86) PCT No.: PCT/US2022/053512

§ 371 Date: Jun. 18, 2024

Related U.S. Application Data
(60) Provisional application No. 63/291,771, filed on Dec. 20, 2021.

(51) Int. Cl. C07D 403/12; A61K 31/4045; A61K 31/4155; A61K 31/437; A61K 31/4439; A61K 31/444; A61K 31/497; A61K 31/506; C07B 59/00; C07D 209/14; C07D 401/04; C07D 403/04; C07D 405/04; C07D 405/14; C07D 471/04

(52) U.S. Cl. CPC C07D 403/12; A61K 31/4045; A61K 31/4155; A61K 31/437; A61K 31/4439; A61K 31/444; A61K 31/497; A61K 31/506; C07B 59/002; C07D 209/14; C07D 401/04; C07D 403/04; C07D 405/04; C07D 405/14; C07D 471/04; C07B 2200/05

(57) ABSTRACT

The present disclosure provides compounds and their pharmaceutical compositions as selective agonists of REV-ERBα. The compounds are useful in various methods and uses, such as in the treatment of diseases including hyperglycemia, dyslipidemia, atherosclerosis, and autoimmune and inflammatory disorders or diseases, and as cancer therapeutics, such as for the treatment of glioblastoma, hepatocellular carcinoma, and colorectal cancer, and for immune-oncology purposes.

### CROSS-REFERENCE TO RELATED APPLICATIONS

This application claims priority under 35 U.S.C. § 119 (e) to U.S. Provisional Application Ser. No. 63/291,771, filed Dec. 20, 2021, the entire content of which is incorporated herein by reference.

### BACKGROUND

T_{H}17 cells are a subset of CD4⁺ T helper cells that preferentially secrete IL-17A, IL-17F, IL-21, and IL-22, and are important during tissue inflammation and anti-microbial/anti-fungal immunity (McGeachy, M. J. et al. (2008) Immunity 28, 445-453). Under homeostatic conditions, T_{H}17 cells have essential roles in protective immunity against extracellular pathogens at mucosal barriers (McGeachy, 2008). However, T_{H}17 cells also are implicated in the pathogenesis of several autoimmune diseases, including multiple sclerosis and psoriasis (Cho, J. H. (2008) Nat Rev Immunol 8, 458-466; Lees, C. W. et al. (2011) Gut 60, 1739-1753; Nair, R. P. et al. (2009) Nat Genet 41, 199-204), indicating that failure of T_{H}17 cell homeostasis can give rise to disease.

A number of studies have identified factors that drive T_{H}17 cell development and pathogenicity, including both the nuclear receptors retinoic-acid-receptor related orphan receptor α and γt (Ivanov, I I et al. (2006) Cell 126, 1121-1133; Yang, X. O. et al. (2008) Immunity 28, 29-39). RORγt is considered the lineage defining transcription factor regulating T_{H}17 cell development and a considerable amount of research has elucidated RORγt's genomic functions. Two other members of the nuclear receptor superfamily, REV-ERBα (NR1D1) and REV-ERBβ (NR1D2), are often co-expressed in the same tissues as the RORs and bind the same DNA response elements resulting in mutual cross talk and co-regulation of their shared target genes (Kojetin, D. J. et al. (2014) Nat Rev Drug Discov 13, 197-216).

Outside of the immune system, the RORs and the REV-ERBs modulate a number of physiological processes, but they are best known for their roles in the regulation of the circadian rhythm, lipid, and glucose metabolic processes. The REV-ERBs are unique within the nuclear receptor superfamily in that they lack the carboxy-terminal tail of their ligand binding domain (LBD) called the activation function 2 region (AF-2, helix 12), which is required for coactivator recognition. Thus, in contrast to the RORs, which are constitutive activators of transcription, the REV-ERBs are transcriptional repressors (Kojetin, 2014). Collectively, the balance of expression of the RORs and REV-ERBs is critical for dynamic regulation of their target genes (Kojetin, 2014).

Most members of the nuclear receptor superfamily are ligand-regulated transcription factors and represent attractive therapeutic targets, including RORγt. After the initial identification of several synthetic RORY modulators, including the compounds SR1001 and digoxin (Huh, J. R. et al. (2011) Nature 472, 486-490; Solt, L. A. et al. (2011) Nature 472, 491-494), additional RORγ ligands were identified, demonstrating the tractability of RORγt-targeted treatment of T_{H}17-mediated autoimmunity (Bronner, S. M. et al. (2017) Expert Opin Ther Pat 27, 101-112).

The REV-ERBs are also ligand-regulated transcription factors: the porphyrin heme was identified as the endogenous ligand for both REV-ERBα and REV-ERBβ (Raghuram, S. et al. (2007) Nat Struct Mol Biol 14, 1207-1213; Yin, L. et al. (2007) Science 318, 1786-1789). Synthetic ligands also can modulate the activity of the REV-ERBs' both in vitro and in vivo (Banerjee, S. et al. (2014) Nat Commun 5, 5759; Kojetin, D. et al. (2011) ACS Chem Biol 6, 131-134; Solt, L. A. et al. (2012) Nature 485, 62-68). For example, use of the compounds SR9009 and SR9011, which selectively target the REV-ERBs, demonstrated that in vivo pharmacological modulation of REV-ERB activity affected REV-ERB-mediated processes, including regulation of the circadian rhythm, glucose, and lipid metabolic processes (Solt et al., 2012). Despite the well-documented overlap in genetic programs between the RORs and REV-ERBs in tissues outside of the immune system (Kojetin, 2014), the role for the REV-ERBs in T_{H}17 cell development is still poorly understood.

REV-ERBα was previously demonstrated to diurnally regulate T_{H}17 cell frequencies in vivo (Yu, W. et al. (2002) Biochem Biophys Res Commun 290, 933-941), and its function in the context of pro-inflammatory settings and autoimmunity is becoming more defined. REV-ERBα is expressed during T_{H}17 cell development and its presence is required for dampening T_{H}17-mediated pro-inflammatory cytokine expression (M. Amir et al., Cell Reports 25 (2018) 3733-3749). Overexpression of REV-ERBα suppressed T_{H}17 cell development whereas genetic deletion of REV-ERBα resulted in enhanced T_{H}17 cell development in vitro and exacerbated autoimmune responses in vivo (id). While REV-ERBα directly repressed Nfil3 (Yu, X. et al. (2013) Science 342, 727-730), it also competed with RORγt for binding at the Il17a promoter and CNS-5 enhancer region. Further, REV-ERBα binds within the Rorc promoter region, indicating potential cross-talk and autoregulation amongst these receptors for controlling T_{H}17 cytokine expression (Amir et al., 2018). REV-ERB-specific small molecules suppressed T_{H}17 cell development in vitro and the development of T_{H}17-mediated autoimmunity in vivo. They were effective when used in a “treatment mode” in several models of autoimmunity and chronic inflammation, demonstrating that REV-ERBα can function outside of its classical role as a core member of the circadian clock under pro-inflammatory conditions and is a cell-intrinsic negative regulator of T_{H}17 cell pro-inflammatory immune responses (Amir et al., 2018).

Pharmacological agonism of REV-ERBα has been shown to suppress hepatic fibrosis and inflammatory response in a non-alcoholic steatohepatitis (NASH) mouse model, indicating broader efficacy in treating non-alcoholic fatty liver disease (NAFLD), slowing its progression to NASH, and treating its component obesity, insulin resistance, and cardiovascular diseases (Griffett K. et al. (2020) PLOS ONE 15 (10): e0236000), such as atherosclerosis (S. Sitaula et al. (2015) Biochemical and Biophysical Research Communications 460(3) 566-571). Activation of REV-ERBα is operative in treatment of hyperglycemia (Griffett, 2020) and in treating dyslipidemia and regulating inflammation to improve survival following acute myocardial infarction (Stujanna E N et al. (2017) PLOS ONE 12 (12): e0189330). The anti-inflammatory properties of REV-ERBα agonism also indicate a pharmacological approach for treating allergic inflammation and asthma (E. Sturm et al. (2020) European Respiratory Journal (56): Suppl. 64, 2905).

Activation of REV-ERBα is an effective adversary to oncogenic processes, establishing pharmacological intervention for the treatment of cancers (Wagner P M et al. (2019) ASN Neuro.). REV-ERBα agonists can be effective as chemotherapeutics against tumor cell types including brain, leukemia, breast, colon, and melanoma (Sulli, G. et al. (2018) Nature 553:351-355), and in treating small-cell lung cancer (W. Shen et al. (2020) Theranostics 10 (1): 4466-4480).

### SUMMARY

Addressing these needs and others, the present disclosure provides in various embodiments a compound as shown in Table 1 hereinbelow, or a pharmaceutically acceptable salt thereof.

TABLE 1
Compounds of the Present Disclosure.

| Com- | |
|---|---|
| pound | Structure |
| 1 | 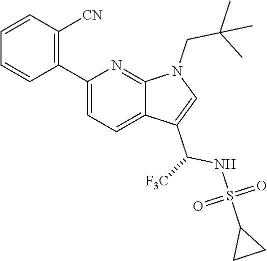 [SMILES: CC(C)(C)Cn1cc([C@H](NS(=O)(=O)C2CC2)C(F)(F)F)c2ccc(-c3ccccc3C#N)nc21] |
| 2 | 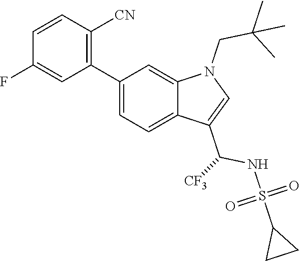 [SMILES: CC(C)(C)Cn1cc([C@H](NS(=O)(=O)C2CC2)C(F)(F)F)c2ccc(-c3cc(F)ccc3C#N)cc21] |
| 3 | 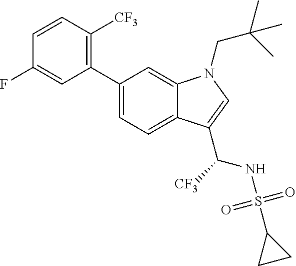 [SMILES: CC(C)(C)Cn1cc([C@H](NS(=O)(=O)C2CC2)C(F)(F)F)c2ccc(-c3cc(F)ccc3C(F)(F)F)cc21] |
| 4 | 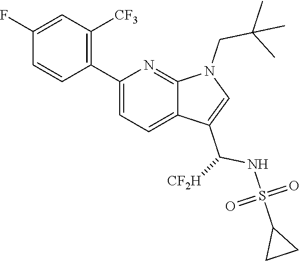 [SMILES: CC(C)(C)Cn1cc([C@H](NS(=O)(=O)C2CC2)C(F)F)c2ccc(-c3ccc(F)cc3C(F)(F)F)nc21] |
| 5 | 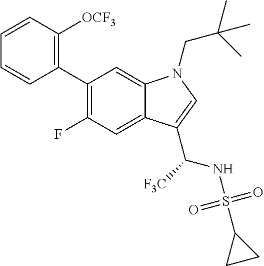 [SMILES: CC(C)(C)Cn1cc([C@H](NS(=O)(=O)C2CC2)C(F)(F)F)c2cc(F)c(-c3ccccc3OC(F)(F)F)cc21] |
| 6 | 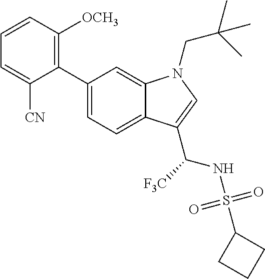 [SMILES: COc1cccc(C#N)c1-c1ccc2c([C@H](NS(=O)(=O)C3CCC3)C(F)(F)F)cn(CC(C)(C)C)c2c1] |
| 7 | 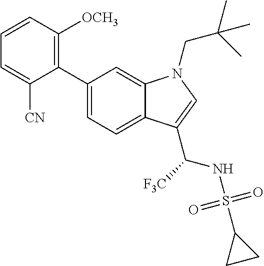 [SMILES: COc1cccc(C#N)c1-c1ccc2c([C@H](NS(=O)(=O)C3CC3)C(F)(F)F)cn(CC(C)(C)C)c2c1] |
| 8 | 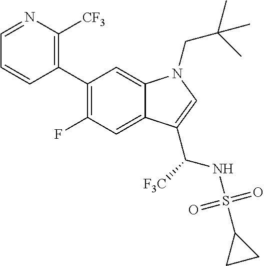 [SMILES: CC(C)(C)Cn1cc([C@H](NS(=O)(=O)C2CC2)C(F)(F)F)c2cc(F)c(-c3cccnc3C(F)(F)F)cc21] |
| 9 | 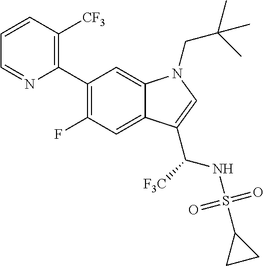 [SMILES: CC(C)(C)Cn1cc([C@H](NS(=O)(=O)C2CC2)C(F)(F)F)c2cc(F)c(-c3ncccc3C(F)(F)F)cc21] |
| 10 | 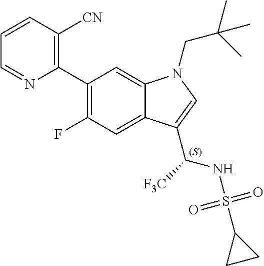 |
| 11 | 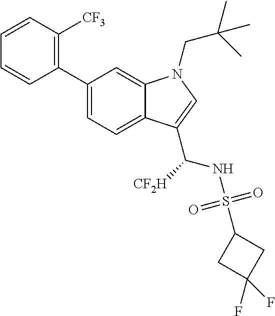 [SMILES: CC(C)(C)Cn1cc([C@H](NS(=O)(=O)C2CC(F)(F)C2)C(F)F)c2ccc(-c3ccccc3C(F)(F)F)cc21] |
| 12 | 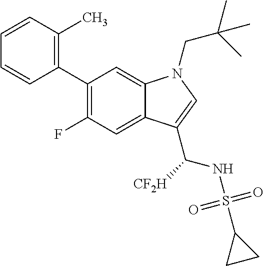 [SMILES: Cc1ccccc1-c1cc2c(cc1F)c([C@H](NS(=O)(=O)C1CC1)C(F)F)cn2CC(C)(C)C] |
| 13 | 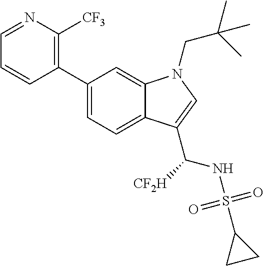 [SMILES: CC(C)(C)Cn1cc([C@H](NS(=O)(=O)C2CC2)C(F)F)c2ccc(-c3cccnc3C(F)(F)F)cc21] |
| 14 | 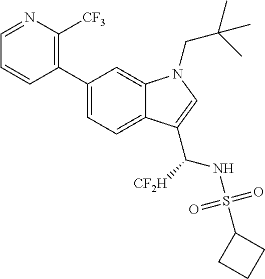 [SMILES: CC(C)(C)Cn1cc([C@H](NS(=O)(=O)C2CCC2)C(F)F)c2ccc(-c3cccnc3C(F)(F)F)cc21] |
| 15 | 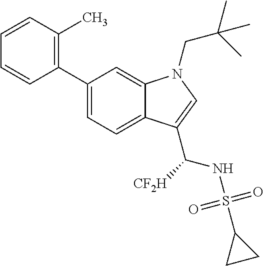 [SMILES: Cc1ccccc1-c1ccc2c([C@H](NS(=O)(=O)C3CC3)C(F)F)cn(CC(C)(C)C)c2c1] |
| 16 | 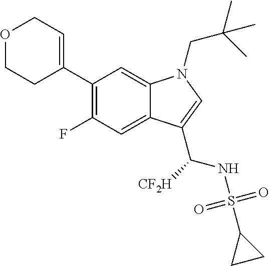 [SMILES: CC(C)(C)Cn1cc([C@H](NS(=O)(=O)C2CC2)C(F)F)c2cc(F)c(C3=CCOCC3)cc21] |
| 17 | 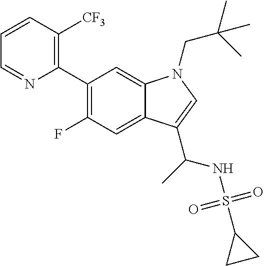 [SMILES: CC(NS(=O)(=O)C1CC1)c1cn(CC(C)(C)C)c2cc(-c3ncccc3C(F)(F)F)c(F)cc12] |
| 18 | 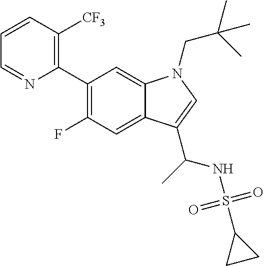 [SMILES: CC(NS(=O)(=O)C1CC1)c1cn(CC(C)(C)C)c2cc(-c3ncccc3C(F)(F)F)c(F)cc12] |
| 19 | 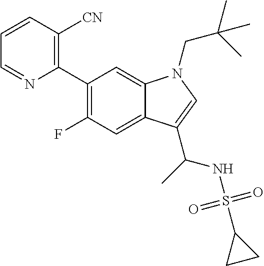 [SMILES: CC(NS(=O)(=O)C1CC1)c1cn(CC(C)(C)C)c2cc(-c3ncccc3C#N)c(F)cc12] |
| 20 | 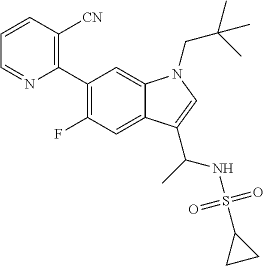 [SMILES: CC(NS(=O)(=O)C1CC1)c1cn(CC(C)(C)C)c2cc(-c3ncccc3C#N)c(F)cc12] |
| 21 | 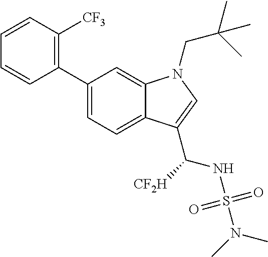 [SMILES: CN(C)S(=O)(=O)N[C@@H](c1cn(CC(C)(C)C)c2cc(-c3ccccc3C(F)(F)F)ccc12)C(F)F] |
| 22 | 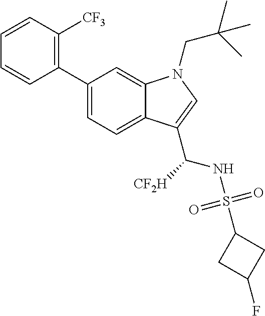 [SMILES: CC(C)(C)Cn1cc([C@H](NS(=O)(=O)C2CC(F)C2)C(F)F)c2ccc(-c3ccccc3C(F)(F)F)cc21] |
| 23 | 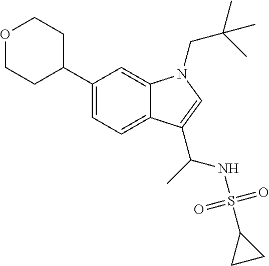 [SMILES: CC(NS(=O)(=O)C1CC1)c1cn(CC(C)(C)C)c2cc(C3CCOCC3)ccc12] |
| 24 | 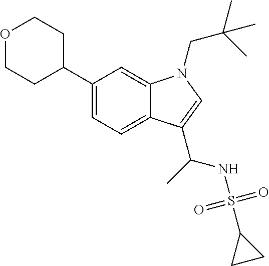 [SMILES: CC(NS(=O)(=O)C1CC1)c1cn(CC(C)(C)C)c2cc(C3CCOCC3)ccc12] |
| 26 | 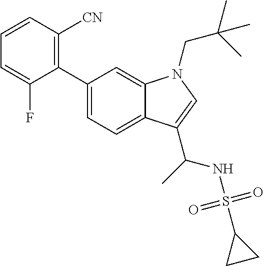 [SMILES: CC(NS(=O)(=O)C1CC1)c1cn(CC(C)(C)C)c2cc(-c3c(F)cccc3C#N)ccc12] |
| 27 | 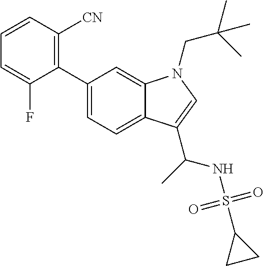 [SMILES: CC(NS(=O)(=O)C1CC1)c1cn(CC(C)(C)C)c2cc(-c3c(F)cccc3C#N)ccc12] |
| 28 | 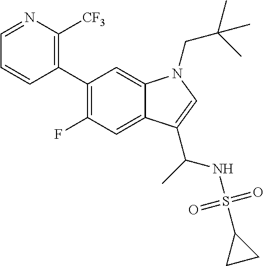 [SMILES: CC(NS(=O)(=O)C1CC1)c1cn(CC(C)(C)C)c2cc(-c3cccnc3C(F)(F)F)c(F)cc12] |
| 29 | 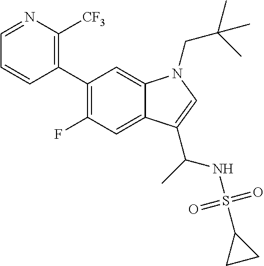 [SMILES: CC(NS(=O)(=O)C1CC1)c1cn(CC(C)(C)C)c2cc(-c3cccnc3C(F)(F)F)c(F)cc12] |
| 30 | 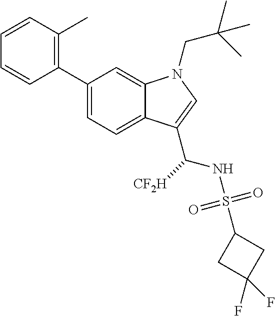 [SMILES: Cc1ccccc1-c1ccc2c([C@H](NS(=O)(=O)C3CC(F)(F)C3)C(F)F)cn(CC(C)(C)C)c2c1] |
| 31 | 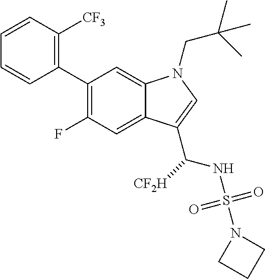 [SMILES: CC(C)(C)Cn1cc([C@H](NS(=O)(=O)N2CCC2)C(F)F)c2cc(F)c(-c3ccccc3C(F)(F)F)cc21] |
| 32 | 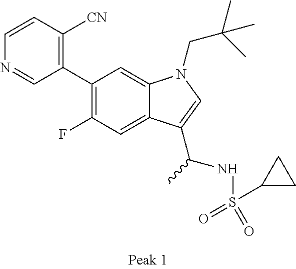 [SMILES: CC(NS(=O)(=O)C1CC1)c1cn(CC(C)(C)C)c2cc(-c3cnccc3C#N)c(F)cc12] |
| 33 | 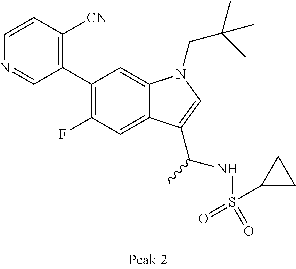 [SMILES: CC(NS(=O)(=O)C1CC1)c1cn(CC(C)(C)C)c2cc(-c3cnccc3C#N)c(F)cc12] |
| 34 | 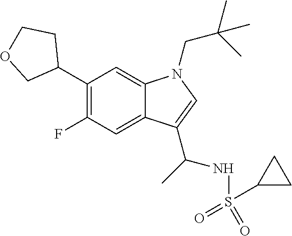 [SMILES: CC(NS(=O)(=O)C1CC1)c1cn(CC(C)(C)C)c2cc(C3CCOC3)c(F)cc12] |
| 35 | 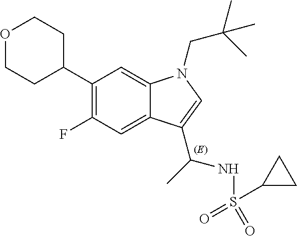 [SMILES: CC(NS(=O)(=O)C1CC1)c1cn(CC(C)(C)C)c2cc(C3CCOCC3)c(F)cc12] |
| 36 | 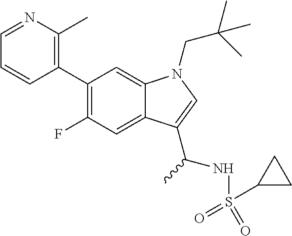 [SMILES: Cc1ncccc1-c1cc2c(cc1F)c(C(C)NS(=O)(=O)C1CC1)cn2CC(C)(C)C] |
| 37 | 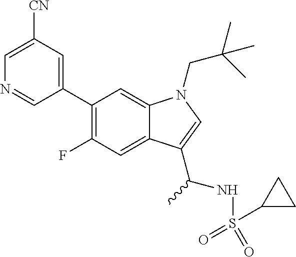 [SMILES: CC(NS(=O)(=O)C1CC1)c1cn(CC(C)(C)C)c2cc(-c3cncc(C#N)c3)c(F)cc12] |
| 38 | 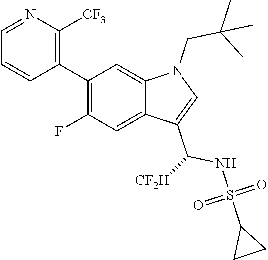 [SMILES: CC(C)(C)Cn1cc([C@H](NS(=O)(=O)C2CC2)C(F)F)c2cc(F)c(-c3cccnc3C(F)(F)F)cc21] |
| 39 | 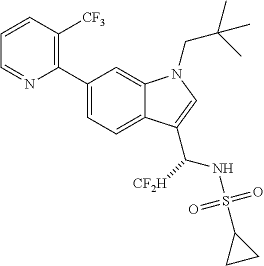 [SMILES: CC(C)(C)Cn1cc([C@H](NS(=O)(=O)C2CC2)C(F)F)c2ccc(-c3ncccc3C(F)(F)F)cc21] |
| 40 | 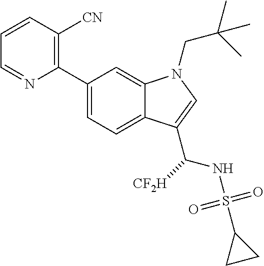 [SMILES: CC(C)(C)Cn1cc([C@H](NS(=O)(=O)C2CC2)C(F)F)c2ccc(-c3ncccc3C#N)cc21] |
| 41 | 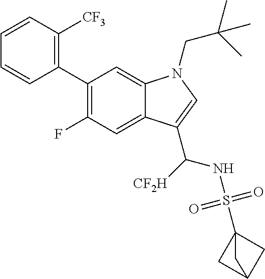 [SMILES: CC(C)(C)Cn1cc(C(NS(=O)(=O)C23CC(C2)C3)C(F)F)c2cc(F)c(-c3ccccc3C(F)(F)F)cc21] |
| 42 | 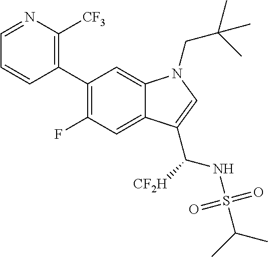 [SMILES: CC(C)S(=O)(=O)N[C@@H](c1cn(CC(C)(C)C)c2cc(-c3cccnc3C(F)(F)F)c(F)cc12)C(F)F] |
| 43 | 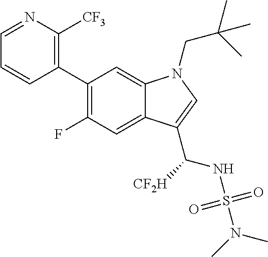 [SMILES: CN(C)S(=O)(=O)N[C@@H](c1cn(CC(C)(C)C)c2cc(-c3cccnc3C(F)(F)F)c(F)cc12)C(F)F] |
| 44 | 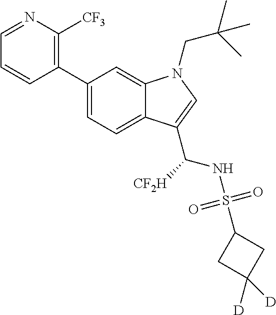 [SMILES: [2H]C1([2H])CC(S(=O)(=O)N[C@@H](c2cn(CC(C)(C)C)c3cc(-c4cccnc4C(F)(F)F)ccc23)C(F)F)C1] |
| 45 | 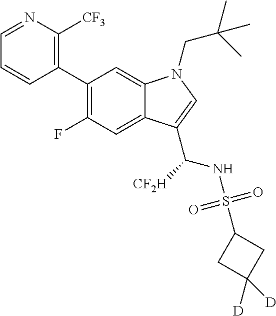 [SMILES: [2H]C1([2H])CC(S(=O)(=O)N[C@@H](c2cn(CC(C)(C)C)c3cc(-c4cccnc4C(F)(F)F)c(F)cc23)C(F)F)C1] |
| 46 | 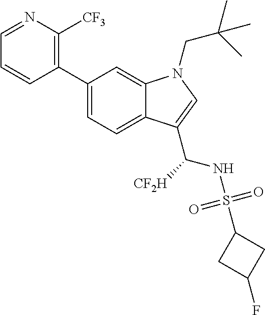 [SMILES: CC(C)(C)Cn1cc([C@H](NS(=O)(=O)C2CC(F)C2)C(F)F)c2ccc(-c3cccnc3C(F)(F)F)cc21] |
| 47 | 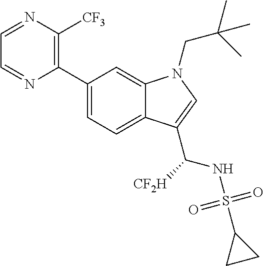 [SMILES: CC(C)(C)Cn1cc([C@H](NS(=O)(=O)C2CC2)C(F)F)c2ccc(-c3nccnc3C(F)(F)F)cc21] |
| 48 | 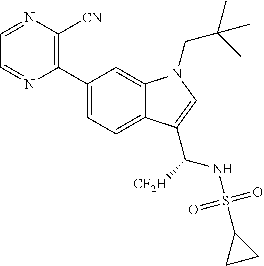 [SMILES: CC(C)(C)Cn1cc([C@H](NS(=O)(=O)C2CC2)C(F)F)c2ccc(-c3nccnc3C#N)cc21] |
| 49 | 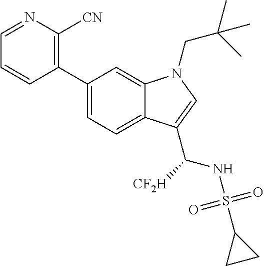 [SMILES: CC(C)(C)Cn1cc([C@H](NS(=O)(=O)C2CC2)C(F)F)c2ccc(-c3cccnc3C#N)cc21] |
| 51 | 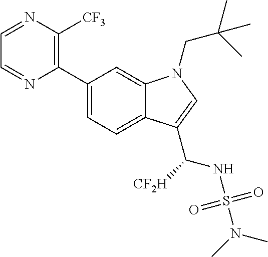 [SMILES: CN(C)S(=O)(=O)N[C@@H](c1cn(CC(C)(C)C)c2cc(-c3nccnc3C(F)(F)F)ccc12)C(F)F] |
| 52 | 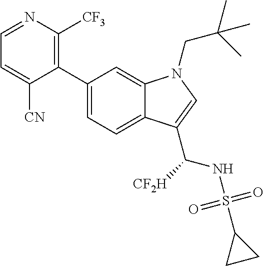 [SMILES: CC(C)(C)Cn1cc([C@H](NS(=O)(=O)C2CC2)C(F)F)c2ccc(-c3c(C#N)ccnc3C(F)(F)F)cc21] |
| 53 | 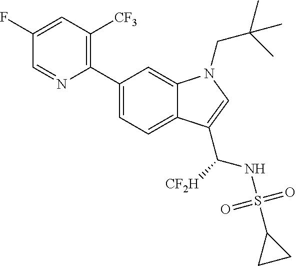 [SMILES: CC(C)(C)Cn1cc([C@H](NS(=O)(=O)C2CC2)C(F)F)c2ccc(-c3ncc(F)cc3C(F)(F)F)cc21] |
| 54 | 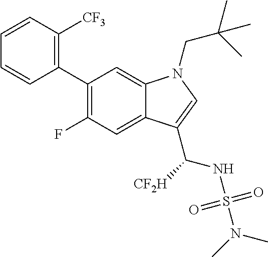 [SMILES: CN(C)S(=O)(=O)N[C@@H](c1cn(CC(C)(C)C)c2cc(-c3ccccc3C(F)(F)F)c(F)cc12)C(F)F] |
| 55 | 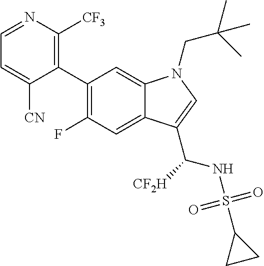 [SMILES: CC(C)(C)Cn1cc([C@H](NS(=O)(=O)C2CC2)C(F)F)c2cc(F)c(-c3c(C#N)ccnc3C(F)(F)F)cc21] |
| 56 | 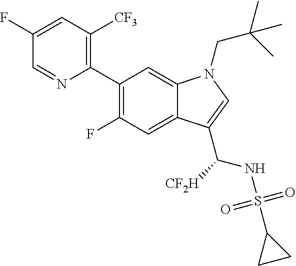 [SMILES: CC(C)(C)Cn1cc([C@H](NS(=O)(=O)C2CC2)C(F)F)c2cc(F)c(-c3ncc(F)cc3C(F)(F)F)cc21] |
| 57 | 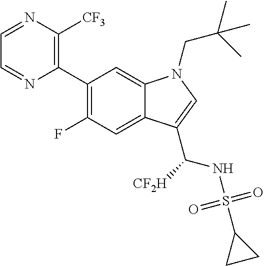 [SMILES: CC(C)(C)Cn1cc([C@H](NS(=O)(=O)C2CC2)C(F)F)c2cc(F)c(-c3nccnc3C(F)(F)F)cc21] |
| 58 | 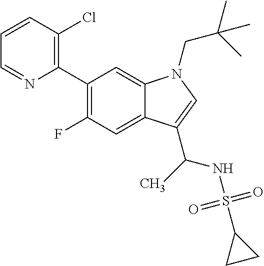 [SMILES: CC(NS(=O)(=O)C1CC1)c1cn(CC(C)(C)C)c2cc(-c3ncccc3Cl)c(F)cc12] |
| 59 | 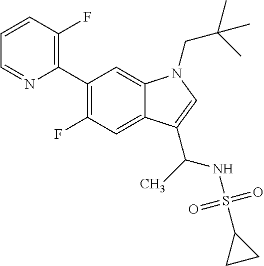 [SMILES: CC(NS(=O)(=O)C1CC1)c1cn(CC(C)(C)C)c2cc(-c3ncccc3F)c(F)cc12] |
| 60 | 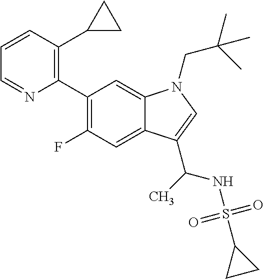 [SMILES: CC(NS(=O)(=O)C1CC1)c1cn(CC(C)(C)C)c2cc(-c3ncccc3C3CC3)c(F)cc12] |
| 61 | 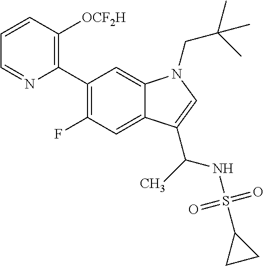 |
| 62 | 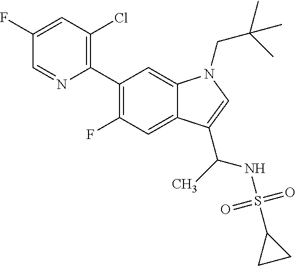 [SMILES: CC(NS(=O)(=O)C1CC1)c1cn(CC(C)(C)C)c2cc(-c3ncc(F)cc3Cl)c(F)cc12] |
| 63 | 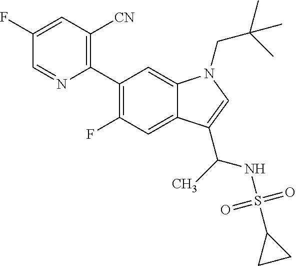 [SMILES: CC(NS(=O)(=O)C1CC1)c1cn(CC(C)(C)C)c2cc(-c3ncc(F)cc3C#N)c(F)cc12] |
| 64 | 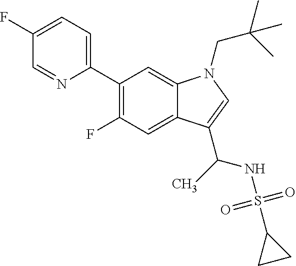 [SMILES: CC(NS(=O)(=O)C1CC1)c1cn(CC(C)(C)C)c2cc(-c3ccc(F)cn3)c(F)cc12] |
| 65 | 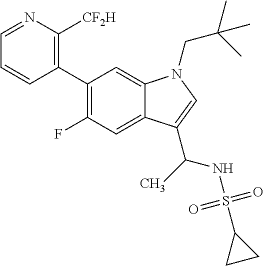 [SMILES: CC(NS(=O)(=O)C1CC1)c1cn(CC(C)(C)C)c2cc(-c3cccnc3C(F)F)c(F)cc12] |
| 66 | 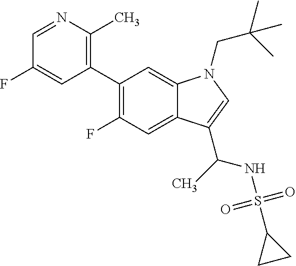 [SMILES: Cc1ncc(F)cc1-c1cc2c(cc1F)c(C(C)NS(=O)(=O)C1CC1)cn2CC(C)(C)C] |
| 67 | 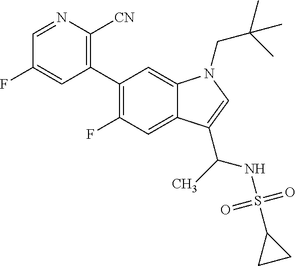 [SMILES: CC(NS(=O)(=O)C1CC1)c1cn(CC(C)(C)C)c2cc(-c3cc(F)cnc3C#N)c(F)cc12] |
| 68 | 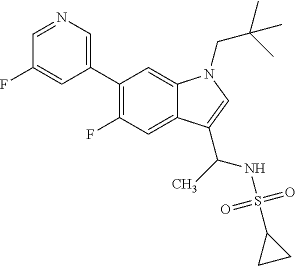 [SMILES: CC(NS(=O)(=O)C1CC1)c1cn(CC(C)(C)C)c2cc(-c3cncc(F)c3)c(F)cc12] |
| 69 | 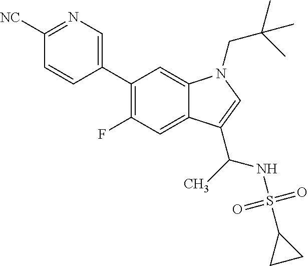 [SMILES: CC(NS(=O)(=O)C1CC1)c1cn(CC(C)(C)C)c2cc(-c3ccc(C#N)nc3)c(F)cc12] |
| 70 | 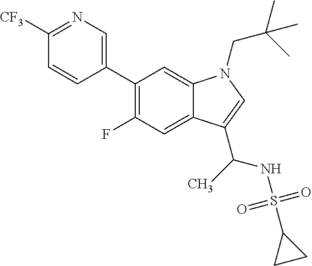 [SMILES: CC(NS(=O)(=O)C1CC1)c1cn(CC(C)(C)C)c2cc(-c3ccc(C(F)(F)F)nc3)c(F)cc12] |
| 71 | 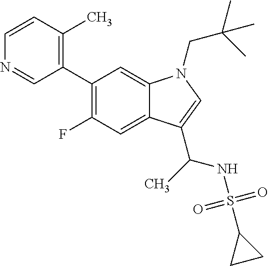 [SMILES: Cc1ccncc1-c1cc2c(cc1F)c(C(C)NS(=O)(=O)C1CC1)cn2CC(C)(C)C] |
| 72 | 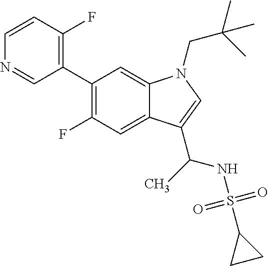 [SMILES: CC(NS(=O)(=O)C1CC1)c1cn(CC(C)(C)C)c2cc(-c3cnccc3F)c(F)cc12] |
| 73 | 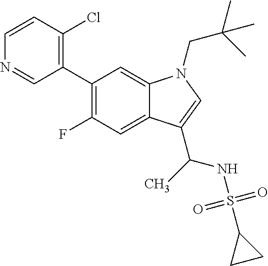 [SMILES: CC(NS(=O)(=O)C1CC1)c1cn(CC(C)(C)C)c2cc(-c3cnccc3Cl)c(F)cc12] |
| 74 | 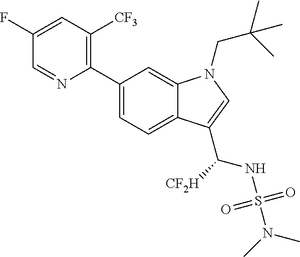 [SMILES: CN(C)S(=O)(=O)N[C@@H](c1cn(CC(C)(C)C)c2cc(-c3ncc(F)cc3C(F)(F)F)ccc12)C(F)F] |
| 75 | 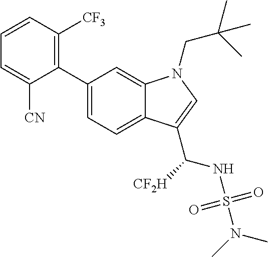 [SMILES: CN(C)S(=O)(=O)N[C@@H](c1cn(CC(C)(C)C)c2cc(-c3c(C#N)cccc3C(F)(F)F)ccc12)C(F)F] |
| 76 | 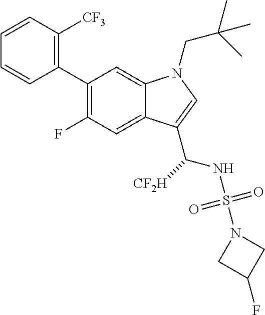 [SMILES: CC(C)(C)Cn1cc([C@H](NS(=O)(=O)N2CC(F)C2)C(F)F)c2cc(F)c(-c3ccccc3C(F)(F)F)cc21] |
| 77 | 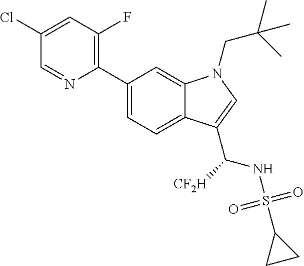 [SMILES: CC(C)(C)Cn1cc([C@H](NS(=O)(=O)C2CC2)C(F)F)c2ccc(-c3ncc(Cl)cc3F)cc21] |
| 78 | 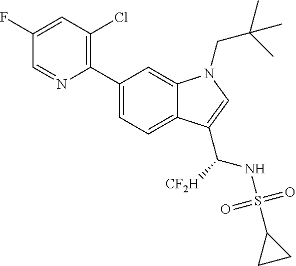 [SMILES: CC(C)(C)Cn1cc([C@H](NS(=O)(=O)C2CC2)C(F)F)c2ccc(-c3ncc(F)cc3Cl)cc21] |
| 79 | 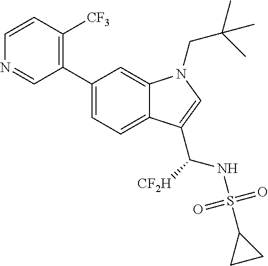 [SMILES: CC(C)(C)Cn1cc([C@H](NS(=O)(=O)C2CC2)C(F)F)c2ccc(-c3cnccc3C(F)(F)F)cc21] |
| 80 | 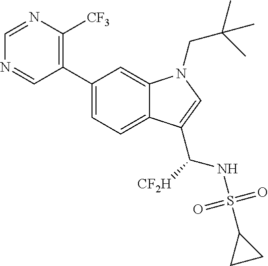 [SMILES: CC(C)(C)Cn1cc([C@H](NS(=O)(=O)C2CC2)C(F)F)c2ccc(-c3cncnc3C(F)(F)F)cc21] |
| 81 | 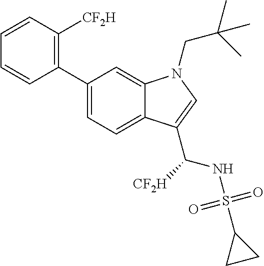 [SMILES: CC(C)(C)Cn1cc([C@H](NS(=O)(=O)C2CC2)C(F)F)c2ccc(-c3ccccc3C(F)F)cc21] |
| 82 | 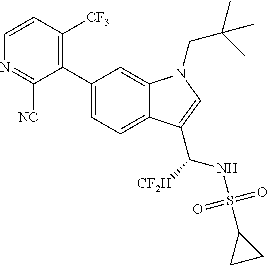 [SMILES: CC(C)(C)Cn1cc([C@H](NS(=O)(=O)C2CC2)C(F)F)c2ccc(-c3c(C(F)(F)F)ccnc3C#N)cc21] |
| 83 | 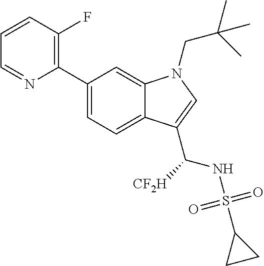 [SMILES: CC(C)(C)Cn1cc([C@H](NS(=O)(=O)C2CC2)C(F)F)c2ccc(-c3ncccc3F)cc21] |
| 84 | 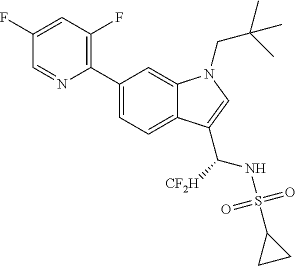 [SMILES: CC(C)(C)Cn1cc([C@H](NS(=O)(=O)C2CC2)C(F)F)c2ccc(-c3ncc(F)cc3F)cc21] |
| 85 | 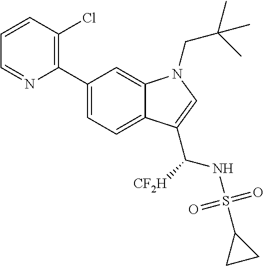 [SMILES: CC(C)(C)Cn1cc([C@H](NS(=O)(=O)C2CC2)C(F)F)c2ccc(-c3ncccc3Cl)cc21] |
| 86 | 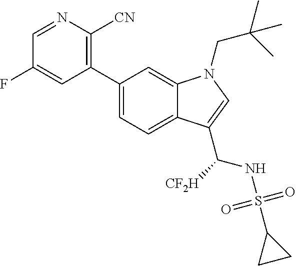 [SMILES: CC(C)(C)Cn1cc([C@H](NS(=O)(=O)C2CC2)C(F)F)c2ccc(-c3cc(F)cnc3C#N)cc21] |
| 87 | 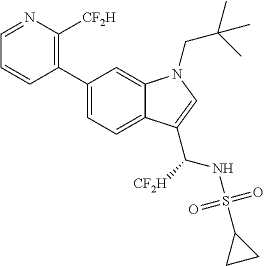 [SMILES: CC(C)(C)Cn1cc([C@H](NS(=O)(=O)C2CC2)C(F)F)c2ccc(-c3cccnc3C(F)F)cc21] |
| 88 | 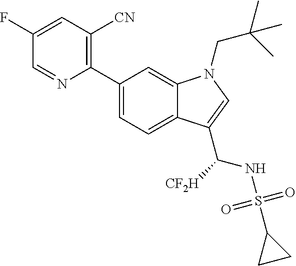 [SMILES: CC(C)(C)Cn1cc([C@H](NS(=O)(=O)C2CC2)C(F)F)c2ccc(-c3ncc(F)cc3C#N)cc21] |
| 89 | 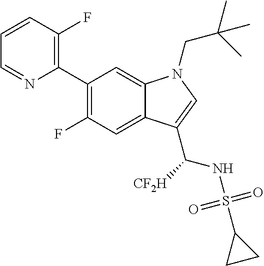 [SMILES: CC(C)(C)Cn1cc([C@H](NS(=O)(=O)C2CC2)C(F)F)c2cc(F)c(-c3ncccc3F)cc21] |
| 90 | 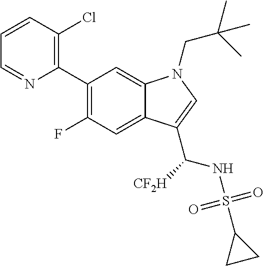 [SMILES: CC(C)(C)Cn1cc([C@H](NS(=O)(=O)C2CC2)C(F)F)c2cc(F)c(-c3ncccc3Cl)cc21] |
| 91 | 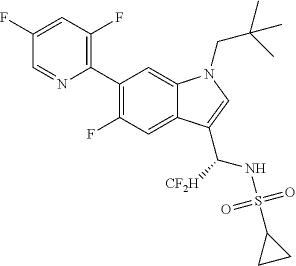 [SMILES: CC(C)(C)Cn1cc([C@H](NS(=O)(=O)C2CC2)C(F)F)c2cc(F)c(-c3ncc(F)cc3F)cc21] |
| 92 | 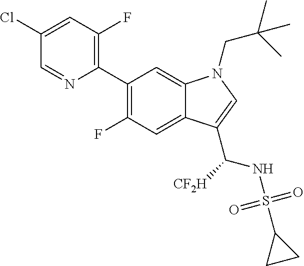 [SMILES: CC(C)(C)Cn1cc([C@H](NS(=O)(=O)C2CC2)C(F)F)c2cc(F)c(-c3ncc(Cl)cc3F)cc21] |
| 93 | 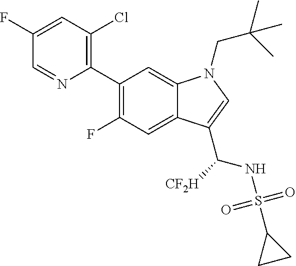 [SMILES: CC(C)(C)Cn1cc([C@H](NS(=O)(=O)C2CC2)C(F)F)c2cc(F)c(-c3ncc(F)cc3Cl)cc21] |
| 94 | 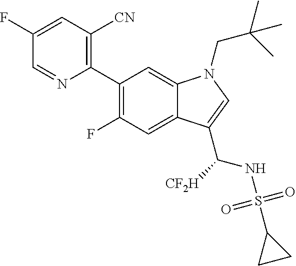 [SMILES: CC(C)(C)Cn1cc([C@H](NS(=O)(=O)C2CC2)C(F)F)c2cc(F)c(-c3ncc(F)cc3C#N)cc21] |
| 95 | 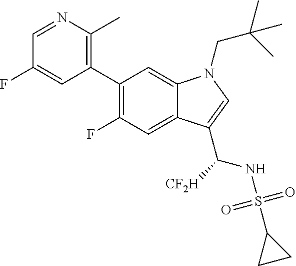 [SMILES: Cc1ncc(F)cc1-c1cc2c(cc1F)c([C@H](NS(=O)(=O)C1CC1)C(F)F)cn2CC(C)(C)C] |
| 96 | 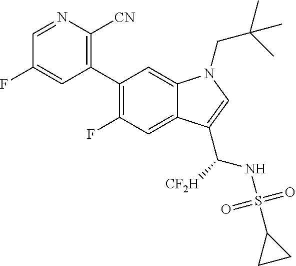 [SMILES: CC(C)(C)Cn1cc([C@H](NS(=O)(=O)C2CC2)C(F)F)c2cc(F)c(-c3cc(F)cnc3C#N)cc21] |
| 97 | 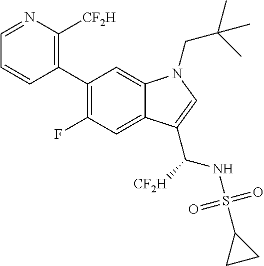 [SMILES: CC(C)(C)Cn1cc([C@H](NS(=O)(=O)C2CC2)C(F)F)c2cc(F)c(-c3cccnc3C(F)F)cc21] |
| 98 | 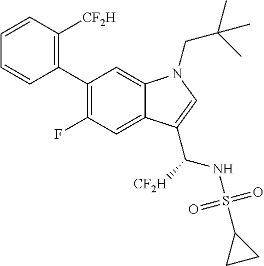 [SMILES: CC(C)(C)Cn1cc([C@H](NS(=O)(=O)C2CC2)C(F)F)c2cc(F)c(-c3ccccc3C(F)F)cc21] |
| 99 | 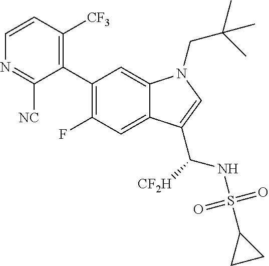 [SMILES: CC(C)(C)Cn1cc([C@H](NS(=O)(=O)C2CC2)C(F)F)c2cc(F)c(-c3c(C(F)(F)F)ccnc3C#N)cc21] |
| 100 | 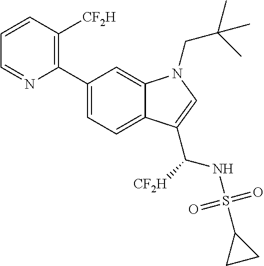 [SMILES: CC(C)(C)Cn1cc([C@H](NS(=O)(=O)C2CC2)C(F)F)c2ccc(-c3ncccc3C(F)F)cc21] |
| 101 | 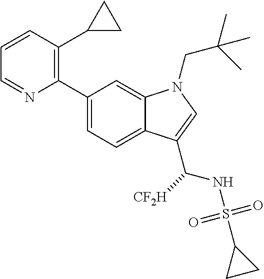 [SMILES: CC(C)(C)Cn1cc([C@H](NS(=O)(=O)C2CC2)C(F)F)c2ccc(-c3ncccc3C3CC3)cc21] |
| 102 | 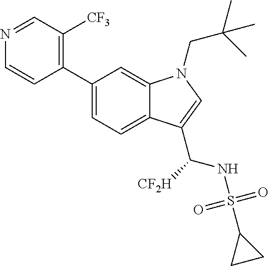 [SMILES: CC(C)(C)Cn1cc([C@H](NS(=O)(=O)C2CC2)C(F)F)c2ccc(-c3ccncc3C(F)(F)F)cc21] |
| 103 | 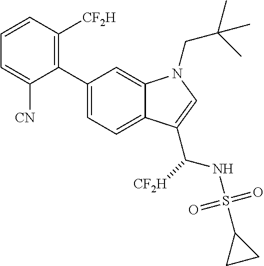 |
| 104 | 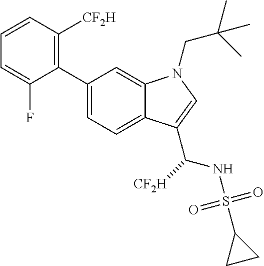 [SMILES: CC(C)(C)Cn1cc([C@H](NS(=O)(=O)C2CC2)C(F)F)c2ccc(-c3c(F)cccc3C(F)F)cc21] |
| 105 | 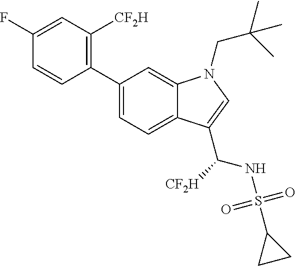 [SMILES: CC(C)(C)Cn1cc([C@H](NS(=O)(=O)C2CC2)C(F)F)c2ccc(-c3ccc(F)cc3C(F)F)cc21] |
| 106 | 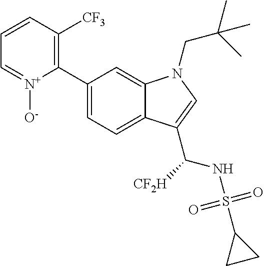 [SMILES: CC(C)(C)Cn1cc([C@H](NS(=O)(=O)C2CC2)C(F)F)c2ccc(-c3c(C(F)(F)F)ccc[n+]3[O-])cc21] |
| 107 | 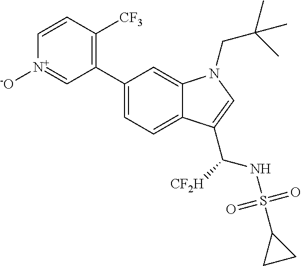 |
| 108 | 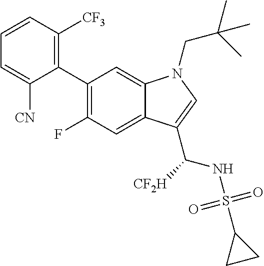 |
| 109 | 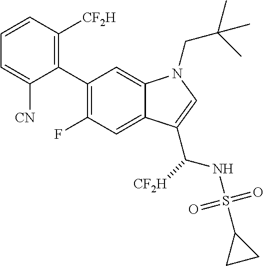 |
| 110 | 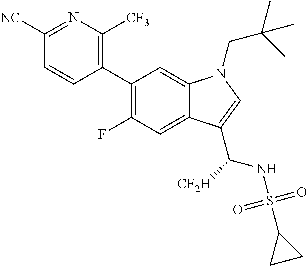 [SMILES: CC(C)(C)Cn1cc([C@H](NS(=O)(=O)C2CC2)C(F)F)c2cc(F)c(-c3ccc(C#N)nc3C(F)(F)F)cc21] |
| 111 | 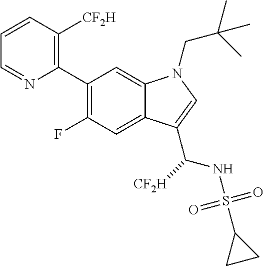 [SMILES: CC(C)(C)Cn1cc([C@H](NS(=O)(=O)C2CC2)C(F)F)c2cc(F)c(-c3ncccc3C(F)F)cc21] |
| 112 | 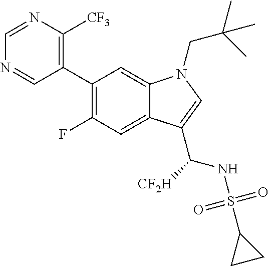 [SMILES: CC(C)(C)Cn1cc([C@H](NS(=O)(=O)C2CC2)C(F)F)c2cc(F)c(-c3cncnc3C(F)(F)F)cc21] |
| 113 | 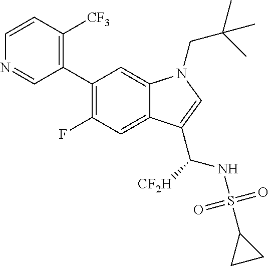 [SMILES: CC(C)(C)Cn1cc([C@H](NS(=O)(=O)C2CC2)C(F)F)c2cc(F)c(-c3cnccc3C(F)(F)F)cc21] |
| 114 | 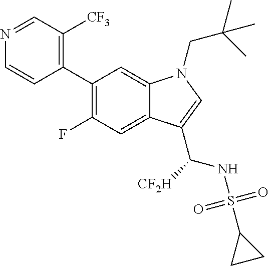 [SMILES: CC(C)(C)Cn1cc([C@H](NS(=O)(=O)C2CC2)C(F)F)c2cc(F)c(-c3ccncc3C(F)(F)F)cc21] |
| 115 | 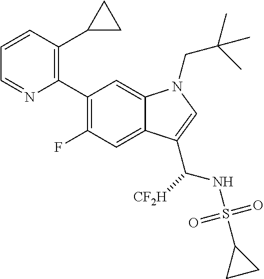 [SMILES: CC(C)(C)Cn1cc([C@H](NS(=O)(=O)C2CC2)C(F)F)c2cc(F)c(-c3ncccc3C3CC3)cc21] |
| 116 | 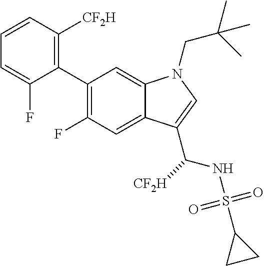 [SMILES: CC(C)(C)Cn1cc([C@H](NS(=O)(=O)C2CC2)C(F)F)c2cc(F)c(-c3c(F)cccc3C(F)F)cc21] |
| 117 | 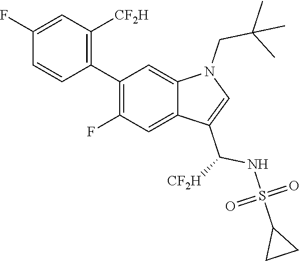 [SMILES: CC(C)(C)Cn1cc([C@H](NS(=O)(=O)C2CC2)C(F)F)c2cc(F)c(-c3ccc(F)cc3C(F)F)cc21] |
| 118 | 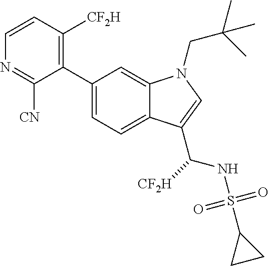 |
| 119 | 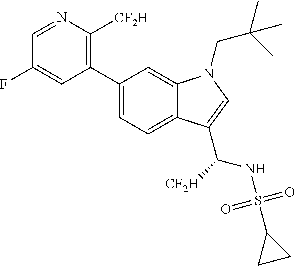 [SMILES: CC(C)(C)Cn1cc([C@H](NS(=O)(=O)C2CC2)C(F)F)c2ccc(-c3cc(F)cnc3C(F)F)cc21] |
| 120 | 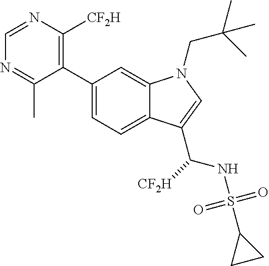 [SMILES: Cc1ncnc(C(F)F)c1-c1ccc2c([C@H](NS(=O)(=O)C3CC3)C(F)F)cn(CC(C)(C)C)c2c1] |
| 121 | 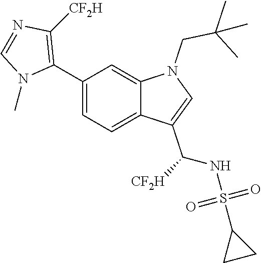 [SMILES: Cn1cnc(C(F)F)c1-c1ccc2c([C@H](NS(=O)(=O)C3CC3)C(F)F)cn(CC(C)(C)C)c2c1] |
| 122 | 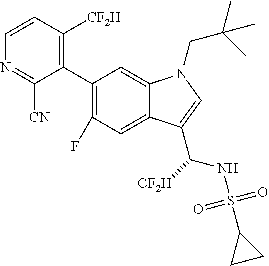 [SMILES: CC(C)(C)Cn1cc([C@H](NS(=O)(=O)C2CC2)C(F)F)c2cc(F)c(-c3c(C(F)F)ccnc3C#N)cc21] |
| 123 | 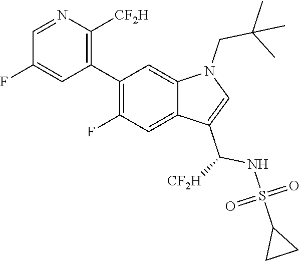 [SMILES: CC(C)(C)Cn1cc([C@H](NS(=O)(=O)C2CC2)C(F)F)c2cc(F)c(-c3cc(F)cnc3C(F)F)cc21] |
| 124 | 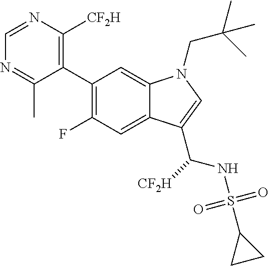 [SMILES: Cc1ncnc(C(F)F)c1-c1cc2c(cc1F)c([C@H](NS(=O)(=O)C1CC1)C(F)F)cn2CC(C)(C)C] |
| 125 | 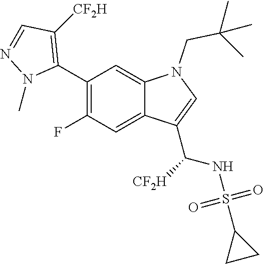 [SMILES: Cn1ncc(C(F)F)c1-c1cc2c(cc1F)c([C@H](NS(=O)(=O)C1CC1)C(F)F)cn2CC(C)(C)C] |
| 126 | 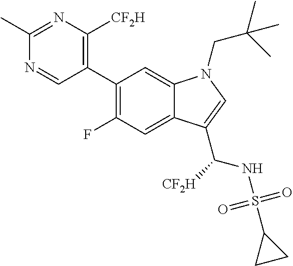 [SMILES: Cc1ncc(-c2cc3c(cc2F)c([C@H](NS(=O)(=O)C2CC2)C(F)F)cn3CC(C)(C)C)c(C(F)F)n1] |
| 127 | 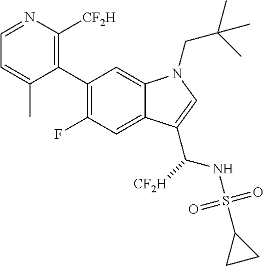 [SMILES: Cc1ccnc(C(F)F)c1-c1cc2c(cc1F)c([C@H](NS(=O)(=O)C1CC1)C(F)F)cn2CC(C)(C)C] |
| 128 | 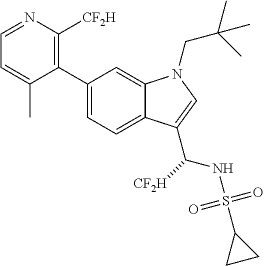 [SMILES: Cc1ccnc(C(F)F)c1-c1ccc2c([C@H](NS(=O)(=O)C3CC3)C(F)F)cn(CC(C)(C)C)c2c1] |
| 129 | 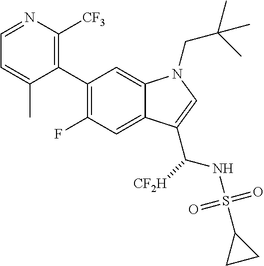 [SMILES: Cc1ccnc(C(F)(F)F)c1-c1cc2c(cc1F)c([C@H](NS(=O)(=O)C1CC1)C(F)F)cn2CC(C)(C)C] |
| 130 | 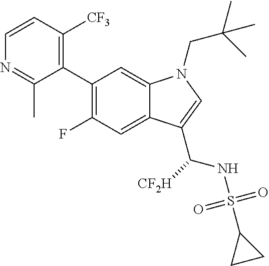 [SMILES: Cc1nccc(C(F)(F)F)c1-c1cc2c(cc1F)c([C@H](NS(=O)(=O)C1CC1)C(F)F)cn2CC(C)(C)C] |
| 131 | 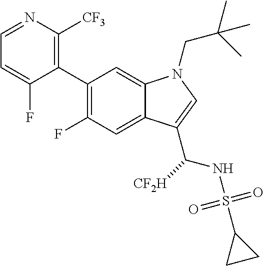 [SMILES: CC(C)(C)Cn1cc([C@H](NS(=O)(=O)C2CC2)C(F)F)c2cc(F)c(-c3c(F)ccnc3C(F)(F)F)cc21] |
| 132 | 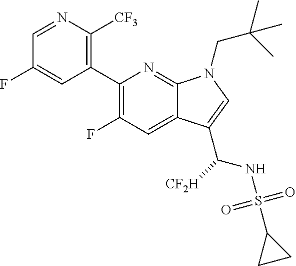 [SMILES: CC(C)(C)Cn1cc([C@H](NS(=O)(=O)C2CC2)C(F)F)c2cc(F)c(-c3cc(F)cnc3C(F)(F)F)nc21] |
| 133 | 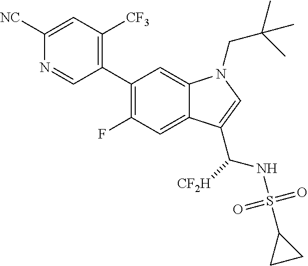 [SMILES: CC(C)(C)Cn1cc([C@H](NS(=O)(=O)C2CC2)C(F)F)c2cc(F)c(-c3cnc(C#N)cc3C(F)(F)F)cc21] |
| 134 | 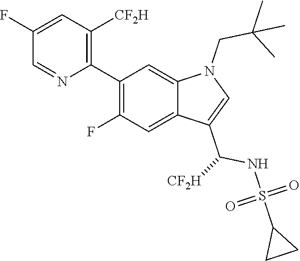 [SMILES: CC(C)(C)Cn1cc([C@H](NS(=O)(=O)C2CC2)C(F)F)c2cc(F)c(-c3ncc(F)cc3C(F)F)cc21] |
| 135 | 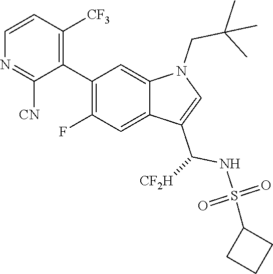 |
| 136 | 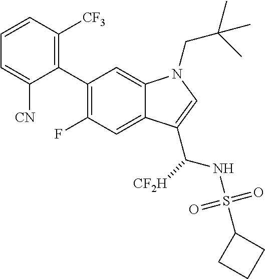 |
| 137 | 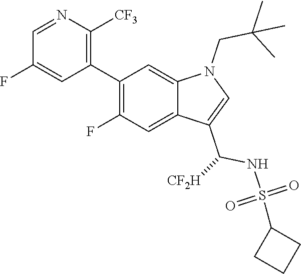 [SMILES: CC(C)(C)Cn1cc([C@H](NS(=O)(=O)C2CCC2)C(F)F)c2cc(F)c(-c3cc(F)cnc3C(F)(F)F)cc21] |
| 138 | 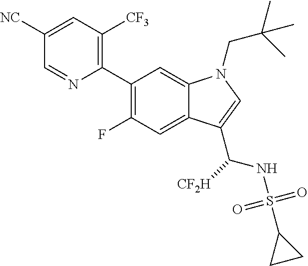 [SMILES: CC(C)(C)Cn1cc([C@H](NS(=O)(=O)C2CC2)C(F)F)c2cc(F)c(-c3ncc(C#N)cc3C(F)(F)F)cc21] |
| 139 | 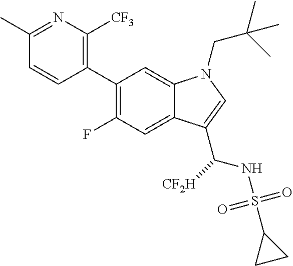 [SMILES: Cc1ccc(-c2cc3c(cc2F)c([C@H](NS(=O)(=O)C2CC2)C(F)F)cn3CC(C)(C)C)c(C(F)(F)F)n1] |
| 140 | 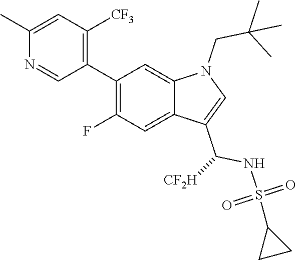 [SMILES: Cc1cc(C(F)(F)F)c(-c2cc3c(cc2F)c([C@H](NS(=O)(=O)C2CC2)C(F)F)cn3CC(C)(C)C)cn1] |
| 141 | 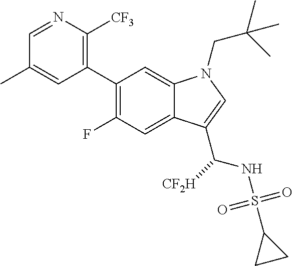 [SMILES: Cc1cnc(C(F)(F)F)c(-c2cc3c(cc2F)c([C@H](NS(=O)(=O)C2CC2)C(F)F)cn3CC(C)(C)C)c1] |
| 142 | 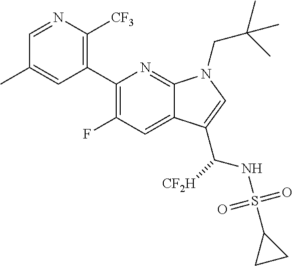 [SMILES: Cc1cnc(C(F)(F)F)c(-c2nc3c(cc2F)c([C@H](NS(=O)(=O)C2CC2)C(F)F)cn3CC(C)(C)C)c1] |
| 143 | 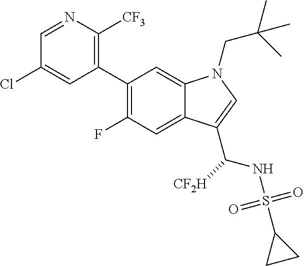 [SMILES: CC(C)(C)Cn1cc([C@H](NS(=O)(=O)C2CC2)C(F)F)c2cc(F)c(-c3cc(Cl)cnc3C(F)(F)F)cc21] |
| 144 | 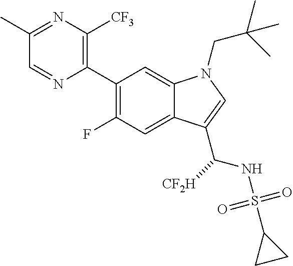 [SMILES: Cc1cnc(-c2cc3c(cc2F)c([C@H](NS(=O)(=O)C2CC2)C(F)F)cn3CC(C)(C)C)c(C(F)(F)F)n1] |
| 145 | 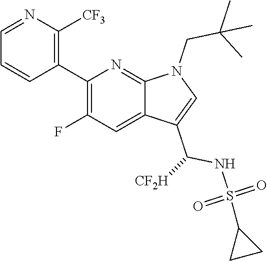 [SMILES: CC(C)(C)Cn1cc([C@H](NS(=O)(=O)C2CC2)C(F)F)c2cc(F)c(-c3cccnc3C(F)(F)F)nc21] |
| 146 | 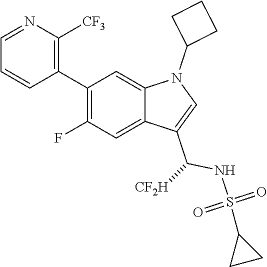 [SMILES: O=S(=O)(N[C@@H](c1cn(C2CCC2)c2cc(-c3cccnc3C(F)(F)F)c(F)cc12)C(F)F)C1CC1] |
| 147 | 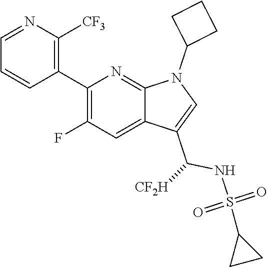 [SMILES: O=S(=O)(N[C@@H](c1cn(C2CCC2)c2nc(-c3cccnc3C(F)(F)F)c(F)cc12)C(F)F)C1CC1] |
| 148 | 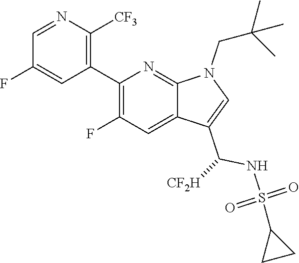 [SMILES: CC(C)(C)Cn1cc([C@H](NS(=O)(=O)C2CC2)C(F)F)c2cc(F)c(-c3cc(F)cnc3C(F)(F)F)nc21] |
| 149 | 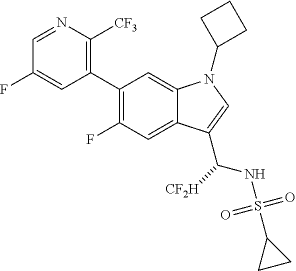 [SMILES: O=S(=O)(N[C@@H](c1cn(C2CCC2)c2cc(-c3cc(F)cnc3C(F)(F)F)c(F)cc12)C(F)F)C1CC1] |
| 150 | 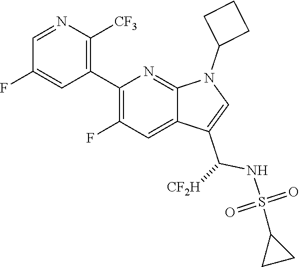 [SMILES: O=S(=O)(N[C@@H](c1cn(C2CCC2)c2nc(-c3cc(F)cnc3C(F)(F)F)c(F)cc12)C(F)F)C1CC1] |
| 151 | 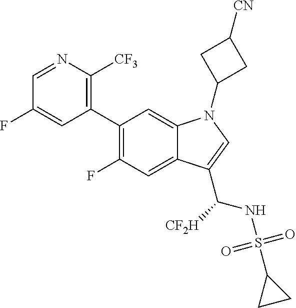 [SMILES: N#CC1CC(n2cc([C@H](NS(=O)(=O)C3CC3)C(F)F)c3cc(F)c(-c4cc(F)cnc4C(F)(F)F)cc32)C1] |
| 152 | 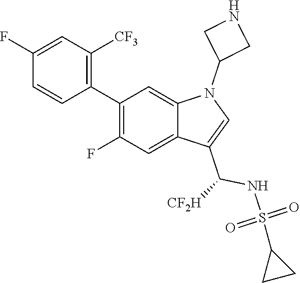 [SMILES: O=S(=O)(N[C@@H](c1cn(C2CNC2)c2cc(-c3ccc(F)cc3C(F)(F)F)c(F)cc12)C(F)F)C1CC1] |
| 153 | 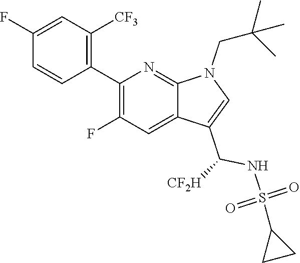 [SMILES: CC(C)(C)Cn1cc([C@H](NS(=O)(=O)C2CC2)C(F)F)c2cc(F)c(-c3ccc(F)cc3C(F)(F)F)nc21] |
| 154 | 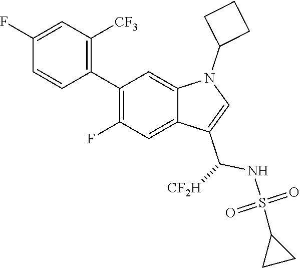 [SMILES: O=S(=O)(N[C@@H](c1cn(C2CCC2)c2cc(-c3ccc(F)cc3C(F)(F)F)c(F)cc12)C(F)F)C1CC1] |
| 155 | 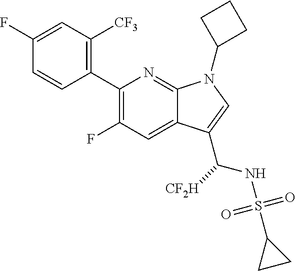 [SMILES: O=S(=O)(N[C@@H](c1cn(C2CCC2)c2nc(-c3ccc(F)cc3C(F)(F)F)c(F)cc12)C(F)F)C1CC1] |
| 156 | 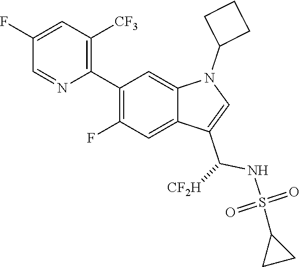 [SMILES: O=S(=O)(N[C@@H](c1cn(C2CCC2)c2cc(-c3ncc(F)cc3C(F)(F)F)c(F)cc12)C(F)F)C1CC1] |
| 157 | 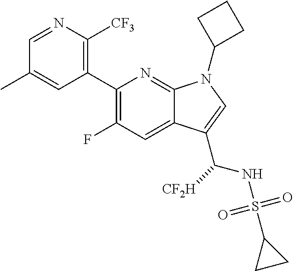 [SMILES: Cc1cnc(C(F)(F)F)c(-c2nc3c(cc2F)c([C@H](NS(=O)(=O)C2CC2)C(F)F)cn3C2CCC2)c1] |
| 158 | 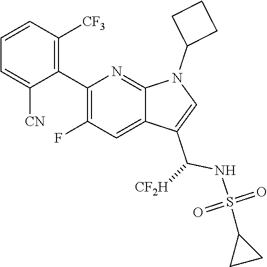 [SMILES: N#Cc1cccc(C(F)(F)F)c1-c1nc2c(cc1F)c([C@H](NS(=O)(=O)C1CC1)C(F)F)cn2C1CCC1] |
| 159 | 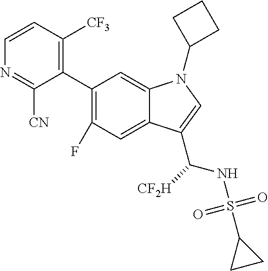 [SMILES: N#Cc1nccc(C(F)(F)F)c1-c1cc2c(cc1F)c([C@H](NS(=O)(=O)C1CC1)C(F)F)cn2C1CCC1] |
| 160 | 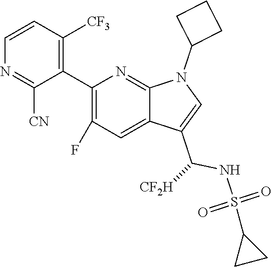 [SMILES: N#Cc1nccc(C(F)(F)F)c1-c1nc2c(cc1F)c([C@H](NS(=O)(=O)C1CC1)C(F)F)cn2C1CCC1] |
| 161 | 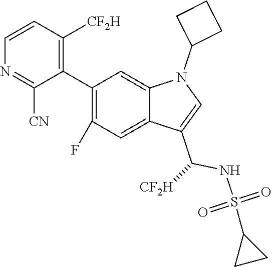 [SMILES: N#Cc1nccc(C(F)F)c1-c1cc2c(cc1F)c([C@H](NS(=O)(=O)C1CC1)C(F)F)cn2C1CCC1] |
| 162 | 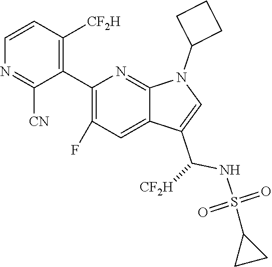 [SMILES: N#Cc1nccc(C(F)F)c1-c1nc2c(cc1F)c([C@H](NS(=O)(=O)C1CC1)C(F)F)cn2C1CCC1] |
| 163 | 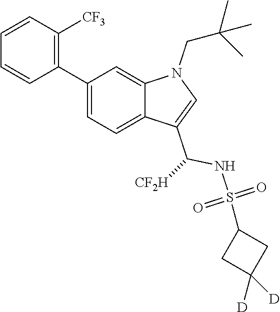 [SMILES: [2H]C1([2H])CC(S(=O)(=O)N[C@@H](c2cn(CC(C)(C)C)c3cc(-c4ccccc4C(F)(F)F)ccc23)C(F)F)C1] |
| 164 | 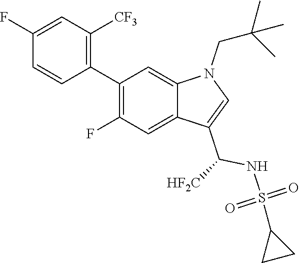 |
| 165 | 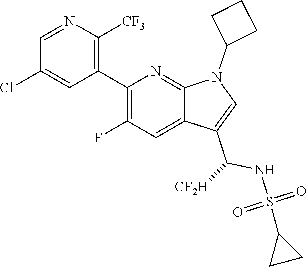 [SMILES: O=S(=O)(N[C@@H](c1cn(C2CCC2)c2nc(-c3cc(Cl)cnc3C(F)(F)F)c(F)cc12)C(F)F)C1CC1] |
| 166 | 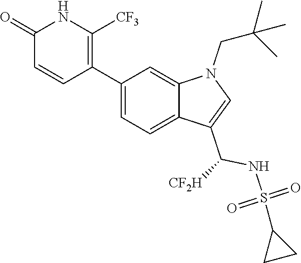 [SMILES: CC(C)(C)Cn1cc([C@H](NS(=O)(=O)C2CC2)C(F)F)c2ccc(-c3ccc(=O)[nH]c3C(F)(F)F)cc21] |
| 167 | 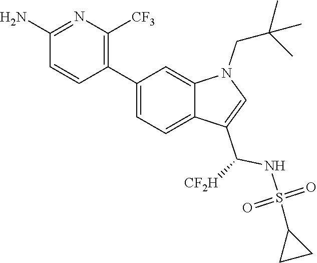 [SMILES: CC(C)(C)Cn1cc([C@H](NS(=O)(=O)C2CC2)C(F)F)c2ccc(-c3ccc(N)nc3C(F)(F)F)cc21] |
| 168 | 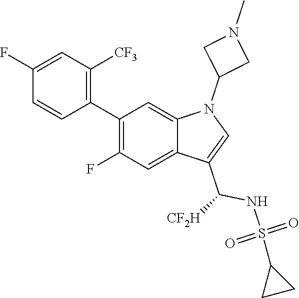 [SMILES: CN1CC(n2cc([C@H](NS(=O)(=O)C3CC3)C(F)F)c3cc(F)c(-c4ccc(F)cc4C(F)(F)F)cc32)C1] |
| 169 | 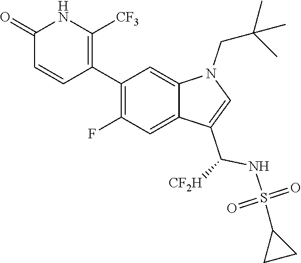 [SMILES: CC(C)(C)Cn1cc([C@H](NS(=O)(=O)C2CC2)C(F)F)c2cc(F)c(-c3ccc(=O)[nH]c3C(F)(F)F)cc21] |
| 170 | 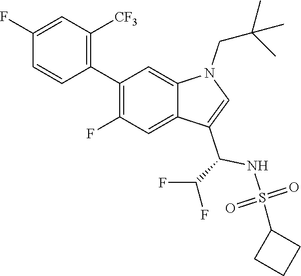 [SMILES: CC(C)(C)Cn1cc([C@H](NS(=O)(=O)C2CCC2)C(F)F)c2cc(F)c(-c3ccc(F)cc3C(F)(F)F)cc21] |
| 171 | 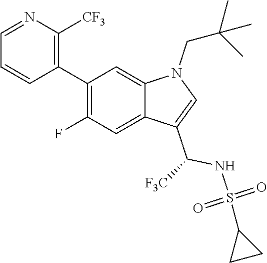 [SMILES: CC(C)(C)Cn1cc([C@H](NS(=O)(=O)C2CC2)C(F)(F)F)c2cc(F)c(-c3cccnc3C(F)(F)F)cc21] |
| 172 | 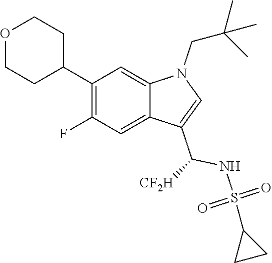 [SMILES: CC(C)(C)Cn1cc([C@H](NS(=O)(=O)C2CC2)C(F)F)c2cc(F)c(C3CCOCC3)cc21] |
| 173 | 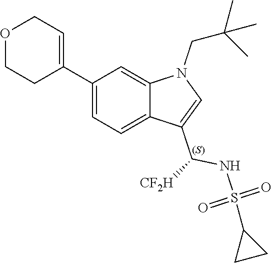 |
| 174 | 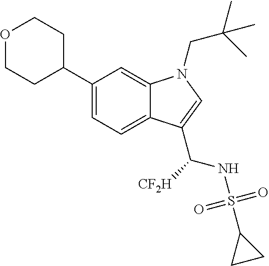 [SMILES: CC(C)(C)Cn1cc([C@H](NS(=O)(=O)C2CC2)C(F)F)c2ccc(C3CCOCC3)cc21] |
| 175 | 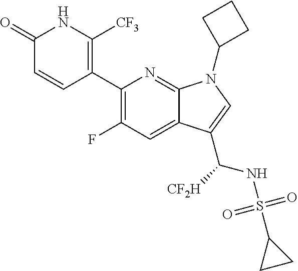 [SMILES: O=c1ccc(-c2nc3c(cc2F)c([C@H](NS(=O)(=O)C2CC2)C(F)F)cn3C2CCC2)c(C(F)(F)F)[nH]1] |
| 176 | 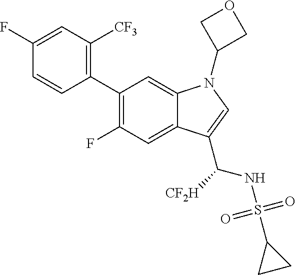 [SMILES: O=S(=O)(N[C@@H](c1cn(C2COC2)c2cc(-c3ccc(F)cc3C(F)(F)F)c(F)cc12)C(F)F)C1CC1] |
| 177 | 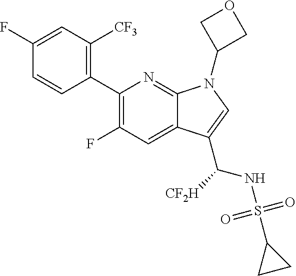 [SMILES: O=S(=O)(N[C@@H](c1cn(C2COC2)c2nc(-c3ccc(F)cc3C(F)(F)F)c(F)cc12)C(F)F)C1CC1] |
| 178 | 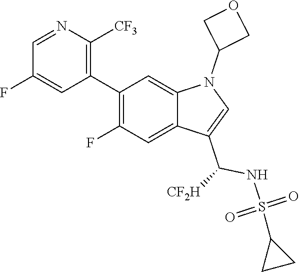 [SMILES: O=S(=O)(N[C@@H](c1cn(C2COC2)c2cc(-c3cc(F)cnc3C(F)(F)F)c(F)cc12)C(F)F)C1CC1] |
| 179 | 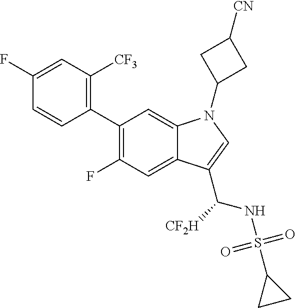 [SMILES: N#CC1CC(n2cc([C@H](NS(=O)(=O)C3CC3)C(F)F)c3cc(F)c(-c4ccc(F)cc4C(F)(F)F)cc32)C1] |
| 180 | 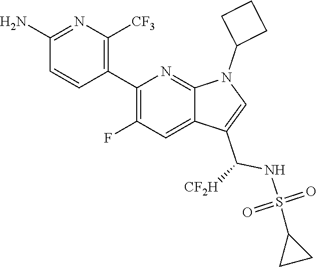 [SMILES: Nc1ccc(-c2nc3c(cc2F)c([C@H](NS(=O)(=O)C2CC2)C(F)F)cn3C2CCC2)c(C(F)(F)F)n1] |
| 181 | 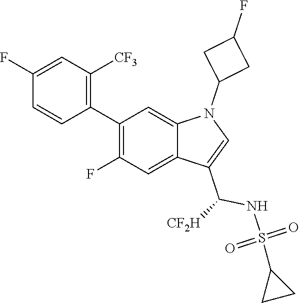 [SMILES: O=S(=O)(N[C@@H](c1cn(C2CC(F)C2)c2cc(-c3ccc(F)cc3C(F)(F)F)c(F)cc12)C(F)F)C1CC1] |
| 182 | 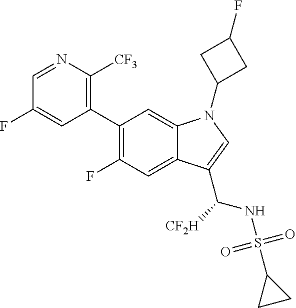 [SMILES: O=S(=O)(N[C@@H](c1cn(C2CC(F)C2)c2cc(-c3cc(F)cnc3C(F)(F)F)c(F)cc12)C(F)F)C1CC1] |
| 184 | 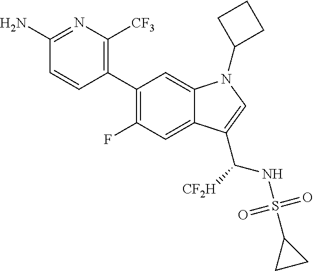 [SMILES: Nc1ccc(-c2cc3c(cc2F)c([C@H](NS(=O)(=O)C2CC2)C(F)F)cn3C2CCC2)c(C(F)(F)F)n1] |
| 185 | 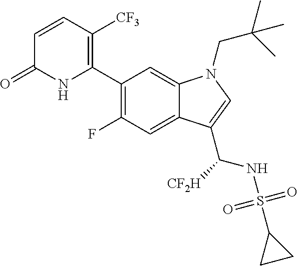 [SMILES: CC(C)(C)Cn1cc([C@H](NS(=O)(=O)C2CC2)C(F)F)c2cc(F)c(-c3[nH]c(=O)ccc3C(F)(F)F)cc21] |
| 186 | 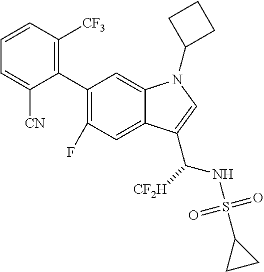 [SMILES: N#Cc1cccc(C(F)(F)F)c1-c1cc2c(cc1F)c([C@H](NS(=O)(=O)C1CC1)C(F)F)cn2C1CCC1] |
| 187 | 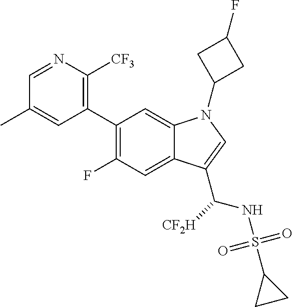 [SMILES: Cc1cnc(C(F)(F)F)c(-c2cc3c(cc2F)c([C@H](NS(=O)(=O)C2CC2)C(F)F)cn3C2CC(F)C2)c1] |
| 188 | 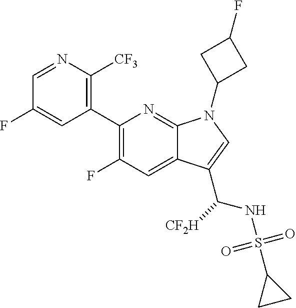 [SMILES: O=S(=O)(N[C@@H](c1cn(C2CC(F)C2)c2nc(-c3cc(F)cnc3C(F)(F)F)c(F)cc12)C(F)F)C1CC1] |
| 189 | 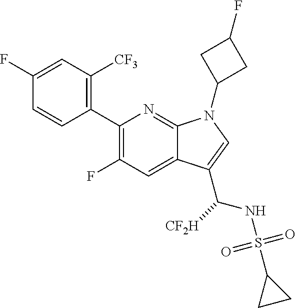 [SMILES: O=S(=O)(N[C@@H](c1cn(C2CC(F)C2)c2nc(-c3ccc(F)cc3C(F)(F)F)c(F)cc12)C(F)F)C1CC1] |
| 190 | 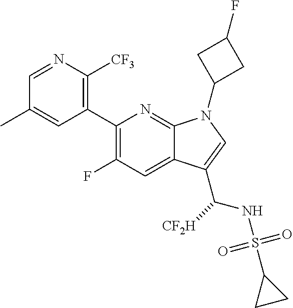 [SMILES: Cc1cnc(C(F)(F)F)c(-c2nc3c(cc2F)c([C@H](NS(=O)(=O)C2CC2)C(F)F)cn3C2CC(F)C2)c1] |
| 191 | 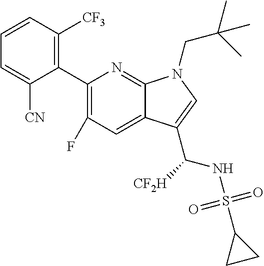 [SMILES: CC(C)(C)Cn1cc([C@H](NS(=O)(=O)C2CC2)C(F)F)c2cc(F)c(-c3c(C#N)cccc3C(F)(F)F)nc21] |
| 192 | 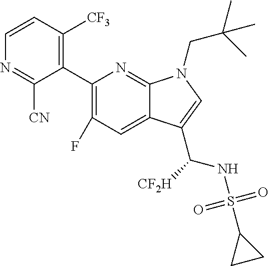 [SMILES: CC(C)(C)Cn1cc([C@H](NS(=O)(=O)C2CC2)C(F)F)c2cc(F)c(-c3c(C(F)(F)F)ccnc3C#N)nc21] |
| 193 | 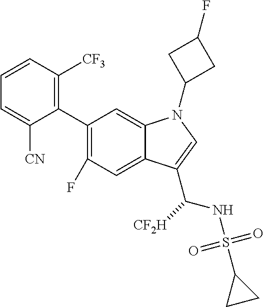 [SMILES: N#Cc1cccc(C(F)(F)F)c1-c1cc2c(cc1F)c([C@H](NS(=O)(=O)C1CC1)C(F)F)cn2C1CC(F)C1] |
| 194 | 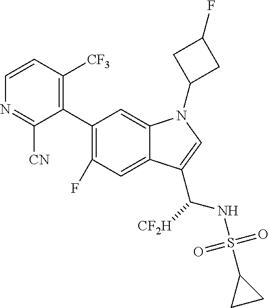 [SMILES: N#Cc1nccc(C(F)(F)F)c1-c1cc2c(cc1F)c([C@H](NS(=O)(=O)C1CC1)C(F)F)cn2C1CC(F)C1] |
| 195 | 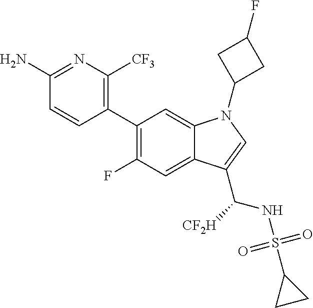 [SMILES: Nc1ccc(-c2cc3c(cc2F)c([C@H](NS(=O)(=O)C2CC2)C(F)F)cn3C2CC(F)C2)c(C(F)(F)F)n1] |
| 196 | 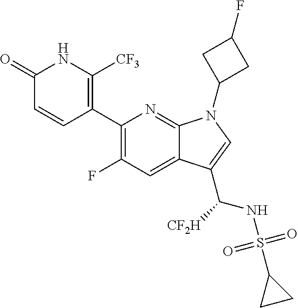 [SMILES: O=c1ccc(-c2nc3c(cc2F)c([C@H](NS(=O)(=O)C2CC2)C(F)F)cn3C2CC(F)C2)c(C(F)(F)F)[nH]1] |
| 197 | 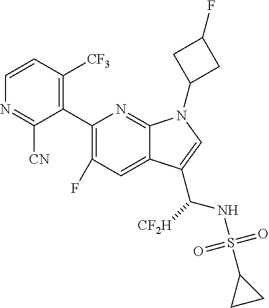 [SMILES: N#Cc1nccc(C(F)(F)F)c1-c1nc2c(cc1F)c([C@H](NS(=O)(=O)C1CC1)C(F)F)cn2C1CC(F)C1] |
| 198 | 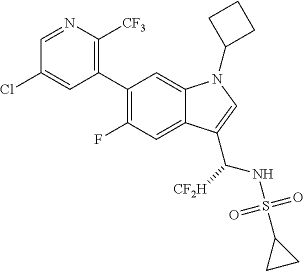 [SMILES: O=S(=O)(N[C@@H](c1cn(C2CCC2)c2cc(-c3cc(Cl)cnc3C(F)(F)F)c(F)cc12)C(F)F)C1CC1] |
| 199 | 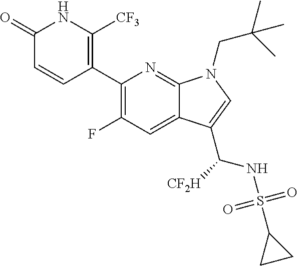 [SMILES: CC(C)(C)Cn1cc([C@H](NS(=O)(=O)C2CC2)C(F)F)c2cc(F)c(-c3ccc(=O)[nH]c3C(F)(F)F)nc21] |
| 200 | 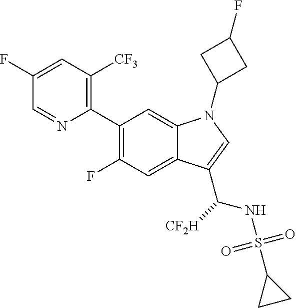 [SMILES: O=S(=O)(N[C@@H](c1cn(C2CC(F)C2)c2cc(-c3ncc(F)cc3C(F)(F)F)c(F)cc12)C(F)F)C1CC1] |
| 201 | 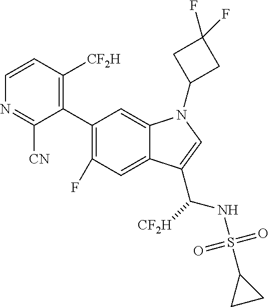 [SMILES: N#Cc1nccc(C(F)F)c1-c1cc2c(cc1F)c([C@H](NS(=O)(=O)C1CC1)C(F)F)cn2C1CC(F)(F)C1] |
| 202 | 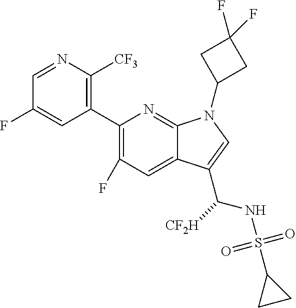 [SMILES: O=S(=O)(N[C@@H](c1cn(C2CC(F)(F)C2)c2nc(-c3cc(F)cnc3C(F)(F)F)c(F)cc12)C(F)F)C1CC1] |
| 203 | 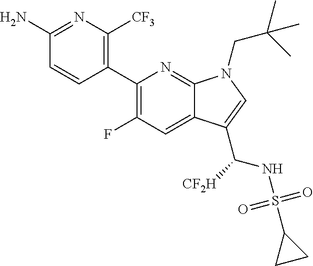 [SMILES: CC(C)(C)Cn1cc([C@H](NS(=O)(=O)C2CC2)C(F)F)c2cc(F)c(-c3ccc(N)nc3C(F)(F)F)nc21] |
| 204 | 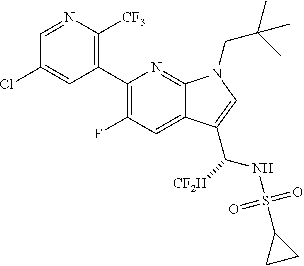 [SMILES: CC(C)(C)Cn1cc([C@H](NS(=O)(=O)C2CC2)C(F)F)c2cc(F)c(-c3cc(Cl)cnc3C(F)(F)F)nc21] |
| 205 | 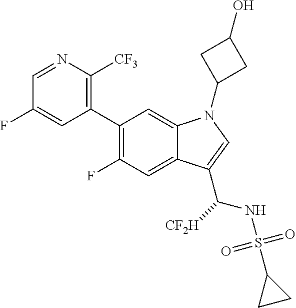 [SMILES: O=S(=O)(N[C@@H](c1cn(C2CC(O)C2)c2cc(-c3cc(F)cnc3C(F)(F)F)c(F)cc12)C(F)F)C1CC1] |
| 206 | 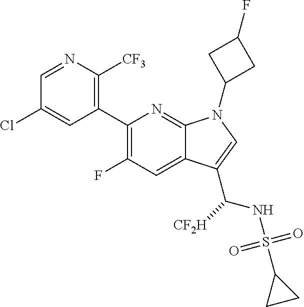 [SMILES: O=S(=O)(N[C@@H](c1cn(C2CC(F)C2)c2nc(-c3cc(Cl)cnc3C(F)(F)F)c(F)cc12)C(F)F)C1CC1] |
| 207 | 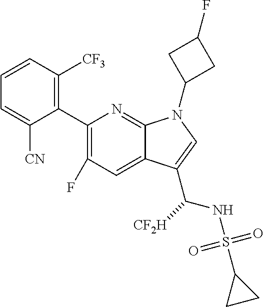 [SMILES: N#Cc1cccc(C(F)(F)F)c1-c1nc2c(cc1F)c([C@H](NS(=O)(=O)C1CC1)C(F)F)cn2C1CC(F)C1] |
| 208 | 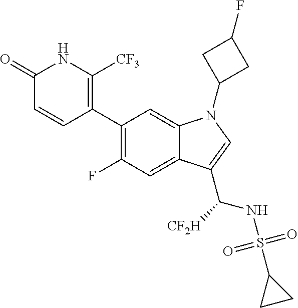 [SMILES: O=c1ccc(-c2cc3c(cc2F)c([C@H](NS(=O)(=O)C2CC2)C(F)F)cn3C2CC(F)C2)c(C(F)(F)F)[nH]1] |
| 209 | 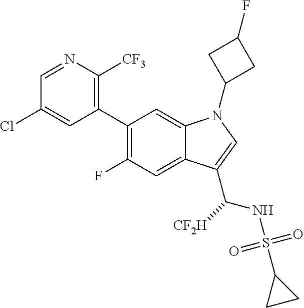 [SMILES: O=S(=O)(N[C@@H](c1cn(C2CC(F)C2)c2cc(-c3cc(Cl)cnc3C(F)(F)F)c(F)cc12)C(F)F)C1CC1] |
| 210 | 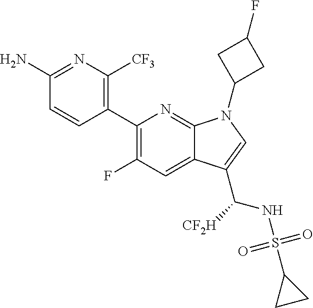 [SMILES: Nc1ccc(-c2nc3c(cc2F)c([C@H](NS(=O)(=O)C2CC2)C(F)F)cn3C2CC(F)C2)c(C(F)(F)F)n1] |
| 211 | 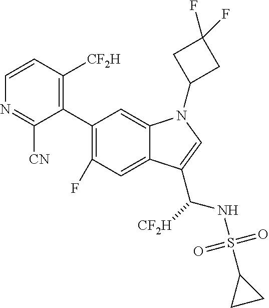 [SMILES: N#Cc1nccc(C(F)F)c1-c1cc2c(cc1F)c([C@H](NS(=O)(=O)C1CC1)C(F)F)cn2C1CC(F)(F)C1] |
| 212 | 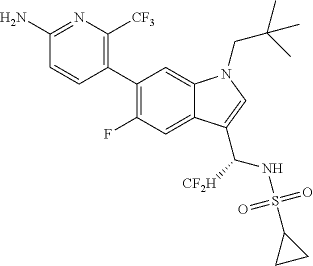 [SMILES: CC(C)(C)Cn1cc([C@H](NS(=O)(=O)C2CC2)C(F)F)c2cc(F)c(-c3ccc(N)nc3C(F)(F)F)cc21] |

The present disclosure also provides in embodiments a pharmaceutical composition comprising a compound or a pharmaceutically acceptable salt or isotopologue thereof as described herein and a pharmaceutically acceptable carrier.

In an embodiment, the present disclosure provides a method for treating a subject suffering from hyperglycemia, dyslipidemia, atherosclerosis, autoimmune or inflammatory disorders or diseases, and cancers, such as glioblastoma, hepatocellular carcinoma, and colorectal cancer. The method comprises administering to the subject a therapeutically effective amount of a compound as described herein or a pharmaceutically acceptable salt thereof.

Still another embodiment of the present disclosure is a method for repressing T_{H}17 cell development in a subject. The method comprises administering to the subject a therapeutically effective amount of a compound as described herein or a pharmaceutically acceptable salt thereof.

The present disclosure, in an embodiment, provides a method for selectively agonizing REV-ERBα over REV-ERBβ in a subject. The method comprises administering to the subject a therapeutically effective amount of a compound as described herein or a pharmaceutically acceptable salt thereof.

The present disclosure also provides, in an embodiment, the use of a compound or pharmaceutically acceptable salt thereof as described herein for treating a subject suffering from hyperglycemia, dyslipidemia, atherosclerosis, an autoimmune or inflammatory disorder or disease, or a cancer, such as glioblastoma, hepatocellular carcinoma, and colorectal cancer.

The present disclosure also provides, in a further embodiment, the use of a compound or pharmaceutically acceptable salt thereof as described herein for repressing T_{H}17 cell development in a subject.

In an embodiment, the present disclosure provides a use of a compound or pharmaceutically acceptable salt thereof as described herein for selectively agonizing REV-ERBα over REV-ERBβ in a subject.

### DETAILED DESCRIPTION

The present disclosure provides in various embodiments small-molecule modulators of REV-ERB activity that are useful for treating T_{H}17-mediated autoimmune diseases or disorders. In various embodiments, the small-molecule modulators are compounds that selectively agonize REV-ERBα relative to REV-ERBB.

### Definitions

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, the term “isotopologue” is an isotopically enriched compound. As used herein, and unless otherwise indicated, the term “isotopically enriched” refers to an atom having an isotopic composition other than the naturally abundant isotopic composition of that atom. “Isotopically enriched” can also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

Thus, as used herein, and unless otherwise indicated, the term “isotopic enrichment factor” refers to the ratio between the isotopic composition and the natural isotopic composition of a specified isotope.

With regard to the compounds provided herein, when a particular atom's position is designated as having deuterium or “D,” it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

As used herein, and unless otherwise specified to the contrary, the term “compound” is inclusive in that it encompasses a compound or a pharmaceutically acceptable salt, stereoisomer, isotopologue, and/or tautomer thereof. Thus, for instance, a compound includes a pharmaceutically acceptable salt of a tautomer of the compound. Similarly, a compound of includes a pharmaceutically acceptable salt of an isotopologue of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Compounds

As described in summary above, the present disclosure provides compounds as shown in Table 1, pharmaceutically acceptable salts, and/or tautomers and/or isotopologues thereof:

### Pharmaceutical Composition

The disclosure also provides a pharmaceutical composition comprising a therapeutically effective amount of one or more compounds as disclosed herein or a pharmaceutically acceptable salt, stereoisomer, isotopologue, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, stereoisomer, isotopologue, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, isotopologue, and/or tautomer thereof that is administered is governed by such considerations, and is the minimum amount necessary to repress T_{H}17 cell development, to exert an anti-inflammatory effect, to selectively agonize REV-ERBα over REV-ERBB, or any combination thereof. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula IA or Formula IB may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

The compounds of the present disclosure are surprisingly selective for agonizing REV-ERBα over REV-ERBβ by a factor of at least about 3, 4, 5, 6, 7, 8, 9, or 10, which is the ratio [EC₅₀ (REV-ERBα)]/[EC₅₀ (REV-ERBβ)]. Thus, in an embodiment, the present disclosure contemplates a method for selectively agonizing REV-ERBα over REV-ERBβ in a subject, comprising administering to the subject a compound described herein or pharmaceutically acceptable salt thereof.

Further, the selectivity of the compounds for REV-ERBα underscores their usefulness in repressing T_{H}17 cell development. The present disclosure provides, in an embodiment, a method for repressing T_{H}17 in tissue in vitro, or in a subject in vivo, comprising contacting the tissue or administering to subject a compound described herein or pharmaceutically acceptable salt thereof.

This mechanism of action is operative, for example, in the treatment of T_{H}17-mediated autoimmunity and inflammation disorders, among others. Thus, the present disclosure also provides in various embodiments a method for treating a subject suffering from hyperglycemia, dyslipidemia, atherosclerosis, an autoimmune or inflammatory disorder or disease, or a cancer. Examples of cancers include glioblastoma, colorectal cancer, and hepatocellular carcinoma. The method comprises administering to the subject a therapeutically effective amount of a compound described herein or pharmaceutically acceptable salt thereof.

### EXAMPLES

The following non-limiting examples are additional embodiments for illustrating the present disclosure.

Synthesis of Compounds. All compounds of the present disclosure are made by using or adapting synthetic procedures as described in detail below.

### Example 1: (S)—N-(1-(6-(2-cyanophenyl)-1-neopentyl-1H-pyrrolo[2,3-b]pyridin-3-yl)-2,2,2-trifluoroethyl)cyclopropanesulfonamide

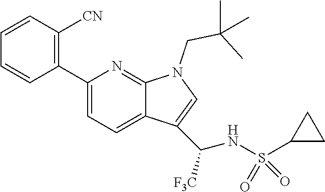 [SMILES: CC(C)(C)Cn1cc([C@H](NS(=O)(=O)C2CC2)C(F)(F)F)c2ccc(-c3ccccc3C#N)nc21]

### Step 1:6-bromo-1-(2,2-dimethylpropyl)pyrrolo[2,3-b]pyridine

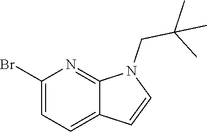

6-bromo-1H-pyrrolo[2,3-b]pyridine (1 g, 5.08 mmol) was added to a suspension of NaH (507.48 mg, 12.69 mmol, 60% purity) in anhydrous DMF (10 mL) in ice-water under nitrogen. The mixture was stirred for 1 h at room temperature, then cooling in ice-water for the addition of 1-bromo-2,2-dimethyl-propane (2.30 g, 15.23 mmol, 1.92 mL) and the resulting mixture was stirred at room temperature overnight under nitrogen. After quenching with water, the aqueous phase was extracted with ethyl acetate twice. The combined organic layer was wash with brine, dried over anhydrous sodium sulfate and filtered. The filtrate was concentrated to give the crude product, which was purified by silica gel column (EA/PE=0-3.3%) to get 6-bromo-1-(2,2-dimethylpropyl)pyrrolo[2,3-b]pyridine (939 mg, 3.51 mmol) as colorless oil. ¹HNMR (CDCl₃, 400 Hz) δ 8.45 (d, J=8.46 Hz, 1H), 7.91 (t, J=1.71 Hz, 1H), 6.74 (d, J=8.72 Hz, 1H), 6.10 (t, J=54.22 Hz, 1H), 4.09 (s, 2H), 1.01 (s, 9H).

### Step 2: (S,E)-2-methyl-N-(2,2,2-trifluoroethylidene) propane-2-sulfinamide

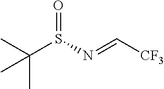

To a mixture of 2,2,2-trifluoroethane-1,1-diol (8 g, 51.71 mmol, 5.33 mL) and (S)-2-methylpropane-2-sulfinamide (8.15 g, 67.22 mmol) in anhydrous CH₂Cl₂ (120 mL) was added anhydrous MgSO₄ (11 g) and 4 A Molecular sieves (30 g). The mixture was heated at 40° C. oil bath overnight. The mixture was cooled to RT and filtered. The filtrate solution of (S,E)-2-methyl-N-(2,2,2-trifluoroethylidene) propane-2-sulfinamide in DCM was used with no further purification.

### Step 3: N—((S)-1-(6-bromo-1-neopentyl-1H-pyrrolo[2,3-b]pyridin-3-yl)-2,2,2-trifluoroethyl)-2-methylpropane-2-sulfinamide

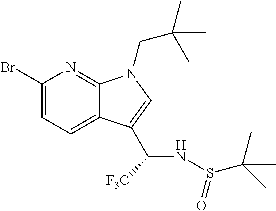 [SMILES: CC(C)(C)Cn1cc([C@H](NS(=O)C(C)(C)C)C(F)(F)F)c2ccc(Br)nc21]

To a solution of 6-bromo-1-neopentyl-1H-pyrrolo[2,3-b]pyridine (400 mg, 1.5 mmol) in DCM (3 mL) cooled to −10° C. was added BF₃·OEt₂ (0.5 mL) followed by addition of the imine solution (2.25 mmol, 1.5 equiv.). The solution was maintained at −10° C. for 1-2 h monitoring completion of the reaction as judged by TLC (consumption of indole starting material). The solution was diluted with DCM, followed by water. The aqueous layer was extracted with DCM twice. The combined organics were washed with brine, dried (MgSO₄) and concentrated in vacuo in a 25° C. water bath to obtain the crude which was purified by chromatography on silica gel (EtOAc/hex) to afford the title compound (97 mg). ESI-MS (m/z): 467.56 [M+1]⁺.

### Step 4:2-methyl-N—((S)-2,2,2-trifluoro-1-(1-neopentyl-6-(4,4,5,5-tetramethyl-1,3,2-dioxaborolan-2-yl)-1H-pyrrolo[2,3-b]pyridin-3-yl)ethyl)propane-2-sulfinamide

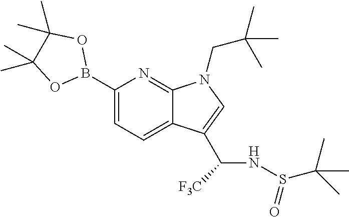 [SMILES: CC(C)(C)Cn1cc([C@H](NS(=O)C(C)(C)C)C(F)(F)F)c2ccc(B3OC(C)(C)C(C)(C)O3)nc21]

A solution of N—((S)-1-(6-bromo-1-neopentyl-1H-pyrrolo[2,3-b]pyridin-3-yl)-2,2,2-trifluoroethyl)-2-methylpropane-2-sulfinamide (97 mg, 0.21 mmol), 4,4,4′,4′,5,5,5′,5′-octamethyl-2,2′-bi(1,3,2-dioxaborolane) (97 mg, 0.37 mmol), KOAc (50 mg, 0.6 mmol) and Pd (dppf) 2Cl₂ (0.021 g, 0.025 mmol) in dioxane (3 mL) was heated in a 100° C. oil bath for 12 h. The solution was concentrated in vacuo. The crude product was purified by chromatography on silica gel (EtOAc/hex) to afford the title compound (35 mg). ESI-MS (m/z): 515.3 [M+1]⁺.

### Step 5: (S)—N—((S)-1-(6-(2-cyanophenyl)-1-neopentyl-1H-pyrrolo[2,3-b]pyridin-3-yl)-2,2,2-trifluoroethyl)-2-methylpropane-2-sulfinamide

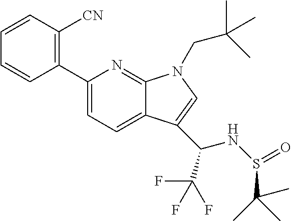 [SMILES: CC(C)(C)Cn1cc([C@H](N[S@@](=O)C(C)(C)C)C(F)(F)F)c2ccc(-c3ccccc3C#N)nc21]

A solution of(S)-2-methyl-N—((S)-2,2,2-trifluoro-1-(1-neopentyl-6-(4,4,5,5-tetramethyl-1,3,2-dioxaborolan-2-yl)-1H-pyrrolo[2,3-b]pyridin-3-yl)ethyl)propane-2-sulfinamide (1 eq), 2-bromobenzonitrile (1.2 eq), K₂CO₃ (3 eq), and Pd(PPh₃)₄ (0.1 eq) in dioxane/water (4/1) was degassed and heated in a 80° C. oil bath for 16 h. The solvent was removed in vacuo to obtain the crude which was purified by prep-HPLC to yield the title compound. ESI-MS (m/z): 491.2 [M+1]⁺.

### Step 6: (S)-2-(3-(1-amino-2,2,2-trifluoroethyl)-1-neopentyl-1H-pyrrolo[2,3-b]pyridin-6-yl)benzonitrile

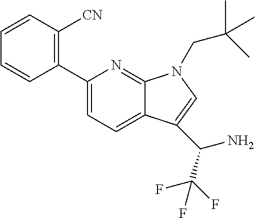 [SMILES: CC(C)(C)Cn1cc([C@H](N)C(F)(F)F)c2ccc(-c3ccccc3C#N)nc21]

To (S)—N—((S)-1-(6-(2-cyanophenyl)-1-neopentyl-1H-pyrrolo[2,3-b]pyridin-3-yl)-2,2,2-trifluoroethyl)-2-methylpropane-2-sulfinamide (1 eq) in MeOH (80 mL, 0.1M) at RT was slowly added con. HCl (3 eq). The solution was stirred for 1-2 h and the completion of reaction was monitored by reverse phase analytical HPLC. The solution was concentrated in vacuo in a 25° C. water bath to obtain the crude which was diluted with DCM, followed by addition of TEA (10 eq). The solution was stirred for 20 min and water was added. The layers were separated, and the aqueous layer was extracted with DCM (2×). The combined organics were washed with brine and concentrated in vacuo in a 25° C. water bath to obtain the crude which was purified by chromatography on silica gel (EtOAc/hex) to afford the title compound. ESI-MS (m/z): 370.2 [M-NH₂]⁺.

### Step 7: (S)—N-(1-(6-(2-cyanophenyl)-1-neopentyl-1H-pyrrolo[2,3-b]pyridin-3-yl)-2,2,2-trifluoroethyl)cyclopropanesulfonamide

To a solution of(S)-2-(3-(1-amino-2,2,2-trifluoroethyl)-1-neopentyl-1H-pyrrolo[2,3-b]pyridin-6-yl)benzonitrile (1.0 eq) in anhydrous pyridine (0.3 mL) at RT was added cyclopropanesulfonyl chloride (2.0 eq). The solution was stirred at RT overnight and the completion of reaction was monitored by reverse phase analytical HPLC. The solution was concentrated in vacuo in a 25° C. water bath to obtain the crude which was diluted with EtOAc, washed with 2N HCl, water, sat'd NaHCO₃, brine, dried (Na₂SO₄) and concentrated. The solvent was removed in vacuo in a 25° C. water bath to obtain the crude which was purified by chromatography on silica gel (EtOAc/hex) to afford the title compound. ESI-MS (m/z): 491.2 [M+1]⁺.

### Example 2: (S)—N-(1-(6-(2-cyano-5-fluorophenyl)-1-neopentyl-1H-indol-3-yl)-2,2,2-trifluoroethyl)cyclopropanesulfonamide

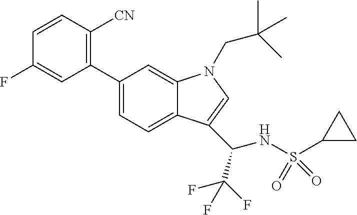 [SMILES: CC(C)(C)Cn1cc([C@H](NS(=O)(=O)C2CC2)C(F)(F)F)c2ccc(-c3cc(F)ccc3C#N)cc21]

### Step 1:6-bromo-1-neopentyl-1H-indole

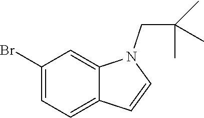

To a stirred solution of 6-bromoindole (5.8 g, 30 mmol) and 1-iodo-2,2-dimethylpropane (8.3 g 42 mmol) in DMF, was added K₂CO₃ (8.4 g, 60 mmol). The resulting reaction mixture was stirred at 80° C. for 12 h and cooled to room temperature. Water and ethyl acetate were added. The organic layer was separated. The aqueous layer was extracted with EA (2×100 mL). The combined organic layer was washed with brine (100 mL), dried over anhydrous Na₂SO₄ and evaporated to give crude product which was purified by chromatography on silica gel to give the title compound (6.8 g). ESI-MS (m/z): 265.68, 267.70 [M+1]⁺.

### Step 2: (S)—N—((S)-1-(6-bromo-1-neopentyl-1H-indol-3-yl)-2,2,2-trifluoroethyl)-2-methylpropane-2-sulfinamide

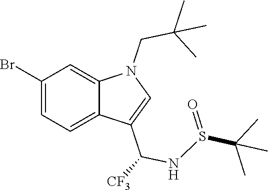

To a solution of 6-bromo-1-neopentyl-1H-indole (2.65 g, 10 mmol) in anhydrous CH₂Cl₂ cooled to −10° C., was added BF₃·OEt₂ (1.9 g, 14 mmol), followed by addition of a solution of (S,E)-2-methyl-N-(2,2,2-trifluoroethylidene) propane-2-sulfinamide (2.4 g, 12 mmol) in DCM. The reaction was stirred for 2 h and was then diluted with DCM and then water. The aqueous layer was extracted with DCM twice. The organic layers were combined and washed with brine and concentrated in vacuo to obtain the crude product, which was purified by chromatography on silica gel to give the title compound as yellow oil (3.1 g). ESI-MS (m/z): 466.62, 468.66 [M+1]⁺.

### Step 3: (S)-1-(6-bromo-1-neopentyl-1H-indol-3-yl)-2,2,2-trifluoroethan-1-amine

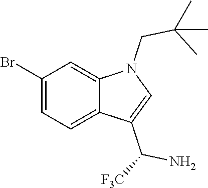 [SMILES: CC(C)(C)Cn1cc([C@H](N)C(F)(F)F)c2ccc(Br)cc21]

To a solution of(S)—N—((S)-1-(6-bromo-1-neopentyl-1H-indol-3-yl)-2,2,2-trifluoroethyl)-2-methylpropane-2-sulfinamide (2.33 g, 10 mmol) in MeOH was added 37% HCl (2.3 mL). The reaction was stirred at RT for 3 h. The mixture was concentrated, the residue was added sat. aqueous NaHCO₃ to control pH=8 and extracted with EtOAc (2×40 mL). The combined organic phases were washed with brine (30 mL), dried with anhydrous Na₂SO₄ and filtered. The filtrate was concentrated to give the crude product, which was purified silica gel chromatography to afford the title compound as a colorless solid (1.5 g).

### Step 4: (S)—N-(1-(6-bromo-1-neopentyl-1H-indol-3-yl)-2,2,2-trifluoroethyl)cyclopropanesulfonamide

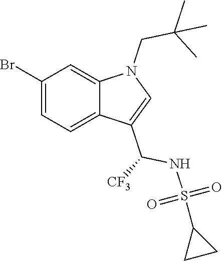 [SMILES: CC(C)(C)Cn1cc([C@H](NS(=O)(=O)C2CC2)C(F)(F)F)c2ccc(Br)cc21]

A mixture of ((S)-1-(6-bromo-1-neopentyl-1H-indol-3-yl)-2,2,2-trifluoroethan-1-amine (1.1 g, 3 mmol) and cyclopropanesulfonyl chloride (0.63 g, 4.5 mmol) in pyridine (6 mL) was stirred at RT overnight. The mixture was acidified with 2N HCl and adjusted to pH=8, then was extracted with EtOAc (3×20 mL). The combined organic phases were washed with saturated NH₄Cl (20 mL) and brine (30 mL), dried with Na₂SO₄, filtered and concentrated to afford the title compound N-[(1S)-1-[6-bromo-1-(2,2-dimethylpropyl)-5-fluoro-indol-3-yl]-2,2,2-trifluoro-ethyl]cyclopropanesulfonamide as a yellow solid (1.2 g). ESI-MS (m/z): 466.62, 468.66 [M+1]⁺.

### Step 5: (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide

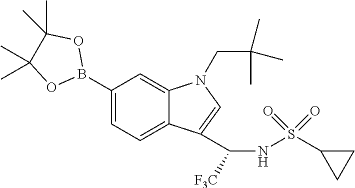 [SMILES: CC(C)(C)Cn1cc([C@H](NS(=O)(=O)C2CC2)C(F)(F)F)c2ccc(B3OC(C)(C)C(C)(C)O3)cc21]

To a solution of 4,4,4′,4′,5,5,5′,5′-octamethyl-2,2′-bi(1,3,2-dioxaborolane) (800 mg, 3.2 mmol), (S)—N-(1-(6-bromo-1-neopentyl-1H-indol-3-yl)-2,2,2-trifluoroethyl)cyclopropanesulfonamide (933 mg, 2.0 mmol) and KOAc (510 mg, 6 mmol) in toluene, was added Pd(dppf)Cl₂ (150 mg) under N₂ atmosphere. The reaction mixture was stirred at 110° C. for 2 h, at which time the solvent was removed. The residue was purified by chromatography on silica gel to give the desired product as a colorless solid (645 mg). ¹HNMR (CDCl₃, 400 MHz) δ 7.84 (s, 1H), 7.70 (d, J=8.0 Hz, 1H), 7.60 (d, J=8.0 Hz, 1H), 7.25 (s, 1H), 5.43-5.35 (q, J=8.0 Hz, 1H), 5.13 (d, J=8.0 Hz, 1H), 3.95 (s, 2H), 2.48-2.42 (m, 1H), 1.86-1.53 (m, 2H), 1.48 (s, 12H), 0.99 (s, 9H), 0.98-0.96 (m, 2H)

Step 6: A solution of(S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide (1 eq), 2-bromo-4-fluorobenzonitrile (1.2 eq), K₂CO₃ (3 eq), and Pd(PPh₃)₄ (0.1 eq) in dioxane/water (4/1) was degassed and heated in a 80° C. oil bath for 16 h. The solvent was removed in vacuo to obtain the crude which was purified by prep-HPLC to yield the title compound. ESI-MS (m/z): 508.2 [M+1]⁺.

### Example 3: (S)—N-(2,2,2-trifluoro-1-(6-(5-fluoro-2-(trifluoromethyl)phenyl)-1-neopentyl-1H-indol-3-yl)ethyl)cyclopropanesulfonamide

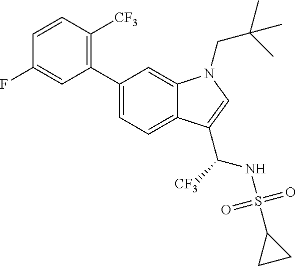 [SMILES: CC(C)(C)Cn1cc([C@H](NS(=O)(=O)C2CC2)C(F)(F)F)c2ccc(-c3cc(F)ccc3C(F)(F)F)cc21]

The title compound was prepared following the same general protocol as described for Step 6, Example 2, using (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide and 2-bromo-4-fluoro-1-(trifluoromethyl)benzene. ESI-MS (m/z): 550.58 [M+1]⁺.

### Example 4: (S)—N-(2,2-difluoro-1-(6-(4-fluoro-2-(trifluoromethyl)phenyl)-1-neopentyl-1H-pyrrolo[2,3-b]pyridin-3-yl)ethyl)cyclopropanesulfonamide

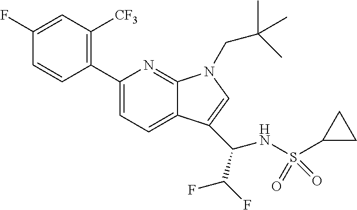 [SMILES: CC(C)(C)Cn1cc([C@H](NS(=O)(=O)C2CC2)C(F)F)c2ccc(-c3ccc(F)cc3C(F)(F)F)nc21]

### Step 1:1-(6-bromo-1-neopentyl-1H-pyrrolo[2,3-b]pyridin-3-yl)-2,2-difluoroethan-1-one

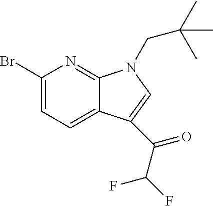 [SMILES: CC(C)(C)Cn1cc(C(=O)C(F)F)c2ccc(Br)nc21]

2,2-difluoroacetic anhydride (350 mg, 2 mmol) was added to a solution of 6-bromo-1-neopentyl-1H-pyrrolo[2,3-b]pyridine and 2,2-difluoroacetic anhydride (266 mg, 1 mmol) in DCM (1.5 mL). The reaction mixture was stirred at room temperature for 3 hours. Saturated NaHCO₃ aqueous solution was added and adjusted to pH=8. The organic layer was separated and the aqueous layer was extracted with DCM (2×5 mL). The combine organic layer was dried with Na₂SO₄, filtered and concentrated. The residue was purified by chromatography on silica gel to give the desired product as a colorless oil (240 mg). ¹HNMR (CDCl₃, 400 Hz) δ 8.45 (d, J=8.46 Hz, 1H), 7.91 (t, J=1.71 Hz, 1H), 6.74 (d, J=8.72 Hz, 1H), 6.10 (t, J=54.22 Hz, 1H), 4.09 (s, 2H), 1.01 (s, 9H).

### Step 2: (S)—N-(1-(6-bromo-1-neopentyl-1H-pyrrolo[2,3-b]pyridin-3-yl)-2,2-difluoroethylidene)-2-methylpropane-2-sulfinamide

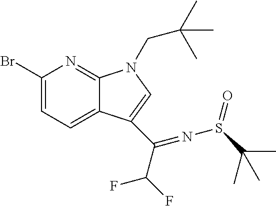 [SMILES: CC(C)(C)Cn1cc(C(=N[S@@](=O)C(C)(C)C)C(F)F)c2ccc(Br)nc21]

A solution of 1-(6-bromo-1-neopentyl-1H-pyrrolo[2,3-b]pyridin-3-yl)-2,2-difluoroethan-1-one (240 mg, 0.7 mmol) and (S)-2-methylpropane-2-sulfinamide (170 mg, 1.4 mmol) in Ti(OiPr)₄ (1.5 mL) was heated at 90° C. for 3-6 hours. The solution was cooled to RT and diluted with EtOAc (10 mL) and brine (3 mL). The mixture was filtered, the solid was washed with EtOAc (3×10 mL) and the filtrate was concentrated in vacuo to obtain the crude which was purified by chromatography on silica gel (EtOAc/hex) to afford the title compound (255 mg). ESI-MS (m/z): 448.51 [M+1]⁺.

### Step 3: (S)—N—((S)-1-(6-bromo-1-neopentyl-1H-pyrrolo[2,3-b]pyridin-3-yl)-2,2-difluoroethyl)-2-methylpropane-2-sulfinamide

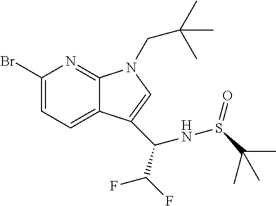 [SMILES: CC(C)(C)Cn1cc([C@H](N[S@@](=O)C(C)(C)C)C(F)F)c2ccc(Br)nc21]

NaBH₄ (42 mg, 1.1 mmol) was added in portions to a solution of(S)—N-(1-(6-bromo-1-neopentyl-1H-pyrrolo[2,3-b]pyridin-3-yl)-2,2-difluoroethylidene)-2-methylpropane-2-sulfinamide (255 mg, 0.57 mmol) in MeOH (3 mL) at 0° C. and the reaction mixture was stirred at room temperature for 1-2 hours. The solvent was removed under reduced pressure and the residue was purified by combi-flash on silica gel to obtain the title compound as an oil (120 mg). ESI-MS (m/z): 450.1 [M+1]⁺.

### Step 4: (S)—N—((S)-2,2-difluoro-1-(6-(4-fluoro-2-(trifluoromethyl)phenyl)-1-neopentyl-1H-pyrrolo[2,3-b]pyridin-3-yl)ethyl)-2-methylpropane-2-sulfinamide

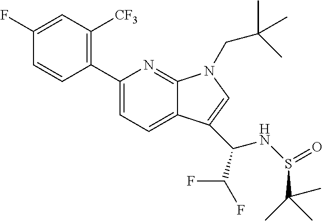 [SMILES: CC(C)(C)Cn1cc([C@H](N[S@@](=O)C(C)(C)C)C(F)F)c2ccc(-c3ccc(F)cc3C(F)(F)F)nc21]

(4-fluoro-2-(trifluoromethyl)phenyl)boronic acid (81 mg, 0.4 mmol), K₂CO₃ (52 mg, 0.4 mmol) and Pd catalyst (7 mg, 0.001 mmol) was added to a solution of(S)—N—((S)-1-(6-bromo-1-neopentyl-1H-pyrrolo[2,3-b]pyridin-3-yl)-2,2-difluoroethyl)-2-methylpropane-2-sulfinamide (90 mg, 0.2 mmol) in dioxane/water (1.0 mL, v/v=1:1) and the reaction mixture was degassed for three times. Followed by heating at 100° C. for 1-2 hours. The reaction mixture was extracted with EtOAc (2×5 mL). The combined organics were dried (Na₂SO₄) and filtered. The solvent was removed under reduced pressure and the residue was purified by combi-flash on silica gel to obtain the title compound as a solid (61 mg). ESI-MS (m/z): 534.2 [M+1]⁺.

### Step 5: (S)-2,2-difluoro-1-(6-(4-fluoro-2-(trifluoromethyl)phenyl)-1-neopentyl-1H-pyrrolo[2,3-b]pyridin-3-yl)ethan-1-amine

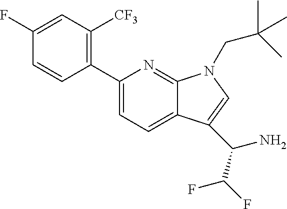 [SMILES: CC(C)(C)Cn1cc([C@H](N)C(F)F)c2ccc(-c3ccc(F)cc3C(F)(F)F)nc21]

37% HCl aqueous solution (60 μL) was added to a solution of(S)—N—((S)-2,2-difluoro-1-(6-(4-fluoro-2-(trifluoromethyl)phenyl)-1-neopentyl-1H-pyrrolo[2,3-b]pyridin-3-yl)ethyl)-2-methylpropane-2-sulfinamide (61 mg) in MeOH (1.0 mL) at 0° C. and the reaction mixture was stirred at room temperature for 3-4 hours. The solvent was removed under reduced pressure and the residue was dissolved in EtOAc (2 mL) and adjusted pH=8 with saturated NaHCO₃. The layers were separated, and the aqueous layer was extracted with EtOAc (2×3 mL). The combined organics were dried (Na₂SO₄) and filtered. The solvent was removed under reduced pressure and the residue was purified by combi-flash on silica gel to obtain the title compound as an oil (31 mg). ESI-MS (m/z): 413.2 [M-NH₂]⁺.

### Step 6: (S)—N-(2,2-difluoro-1-(6-(4-fluoro-2-(trifluoromethyl)phenyl)-1-neopentyl-1H-pyrrolo[2,3-b]pyridin-3-yl)ethyl)cyclopropanesulfonamide

Cyclopropanesulfonyl chloride (12 mg, 0.08 mmol) was added to a solution of(S)-2,2-difluoro-1-(6-(4-fluoro-2-(trifluoromethyl)phenyl)-1-neopentyl-1H-pyrrolo[2,3-b]pyridin-3-yl)ethan-1-amine (21 mg, 0.05 mmol) in pyridine (0.3 mL) at 0° C. and the reaction mixture was stirred at room temperature for 16-24 hours. The reaction mixture was diluted EtOAc (2 mL) and adjusted pH=8 with 4M HCl aqueous solution. The layers were separated, and the aqueous layer was extracted with EtOAc (5×2 mL). The combined organics were dried (Na₂SO₄) and filtered. The solvent was removed under reduced pressure and the residue was purified by combi-flash on silica gel to obtain the title compound (12 mg). ESI-MS (m/z): 534.2 [M+1]⁺.

### Example 5: (S)—N-(2,2,2-trifluoro-1-(5-fluoro-1-neopentyl-6-(2-(trifluoromethoxy)phenyl)-H-indol-3-yl)ethyl)cyclopropanesulfonamide

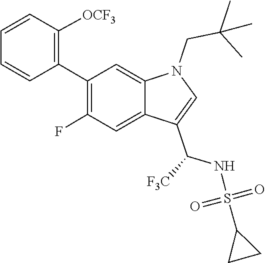 [SMILES: CC(C)(C)Cn1cc([C@H](NS(=O)(=O)C2CC2)C(F)(F)F)c2cc(F)c(-c3ccccc3OC(F)(F)F)cc21]

### Step 1: Synthesis of(S)-2,2,2-trifluoro-1-(5-fluoro-1-neopentyl-6-(2-(trifluoromethoxy)phenyl)-1H-indol-3-yl)ethanamine

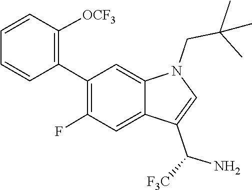 [SMILES: CC(C)(C)Cn1cc([C@H](N)C(F)(F)F)c2cc(F)c(-c3ccccc3OC(F)(F)F)cc21]

The title compound was synthesized in a similar fashion as(S)-2,2,2-trifluoro-1-(5-fluoro-1-neopentyl-6-(2-(trifluoromethyl)pyridin-3-yl)-1H-indol-3-yl)ethan-1-amine (Ex 8, Step 5) by simply replacing 3-bromo-2-(trifluoromethyl)pyridine with 2-(trifluoromethoxy)pyridine.

### Step 2: Synthesis of(S)—N-(2,2,2-trifluoro-1-(5-fluoro-1-neopentyl-6-(2-(trifluoromethoxy)phenyl)-1H-indol-3-yl)ethyl)cyclopropanesulfonamide

A mixture of(S)-2,2,2-trifluoro-1-(5-fluoro-1-neopentyl-6-(2-(trifluoromethoxy)phenyl)-1H-indol-3-yl)ethanamine (100 mg, 216.26 μmol) and cyclopropanesulfonyl chloride (91.21 mg, 648.78 μmol, 66.10 μL) in anhydrous pyridine (2 mL) was stirred at RT for 12 hours. To the mixture was then added CH₂Cl₂ and concentrated to get a black oil which was purified by Prep HPLC to give(S)—N-(2,2,2-trifluoro-1-(5-fluoro-1-neopentyl-6-(2-(trifluoromethoxy)phenyl)-1H-indol-3-yl)ethyl)cyclopropanesulfonamide (12.77 mg, 22.54 μmol, 10.42% yield) as a white solid. 1H NMR (400 MHz, CDCl₃) δ 7.49 (d, J=10.3 Hz, 1H), 7.46-7.41 (m, 2H), 7.40-7.35 (m, 2H), 7.29 (d, J=5.9 Hz, 1H), 7.25 (s, 1H), 5.41-5.33 (m, 1H), 4.84 (d, J=7.9 Hz, 1H), 3.89 (s, 2H), 2.49-2.41 (m, 1H), 1.22 (dd, J=8.7, 5.0 Hz, 1H), 1.14 (dd, J=10.0, 5.0 Hz, 1H), 0.99 (s, 9H), 0.97-0.91 (m, 2H). MS Found: 567.7 [M+1].

### Example 6: (S)—N-(1-(6-(2-cyano-6-methoxyphenyl)-1-neopentyl-1H-indol-3-yl)-2,2,2-trifluoroethyl)cyclobutanesulfonamide

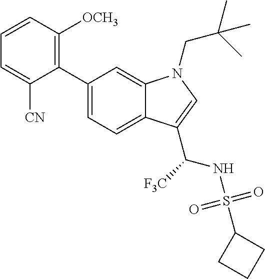 [SMILES: COc1cccc(C#N)c1-c1ccc2c([C@H](NS(=O)(=O)C3CCC3)C(F)(F)F)cn(CC(C)(C)C)c2c1]

### Step 1: Synthesis of(S)-2-(3-(1-amino-2,2,2-trifluoroethyl)-1-neopentyl-1H-indol-6-yl)-3-methoxybenzonitrile

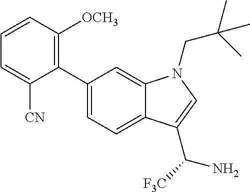 [SMILES: COc1cccc(C#N)c1-c1ccc2c([C@H](N)C(F)(F)F)cn(CC(C)(C)C)c2c1]

The title compound was synthesized in a similar fashion as(S)-2,2,2-trifluoro-1-(5-fluoro-1-neopentyl-6-(2-(trifluoromethyl)pyridin-3-yl)-1H-indol-3-yl)ethan-1-amine (Ex 8, Step 5) by simply replacing 3-bromo-2-(trifluoromethyl)pyridine with 2-bromo-3-methoxybenzonitrile.

### Step 2: Synthesis of(S)—N-(1-(6-(2-cyano-6-methoxyphenyl)-1-neopentyl-1H-indol-3-yl)-2,2,2-trifluoroethyl)cyclobutanesulfonamide

A mixture of(S)-2-(3-(1-amino-2,2,2-trifluoroethyl)-1-neopentyl-1H-indol-6-yl)-3-methoxybenzonitrile (165 mg, 397.16 μmol) and cyclobutanesulfonyl chloride (184.22 mg, 1.19 mmol) in anhydrous pyridine (3 mL) was stirred at 100° C. for 4 hours in the microwave. To the mixture was then added CH₂Cl₂ and concentrated to get black oil, which was purified by prep-HPLC to give(S)—N-(1-(6-(2-cyano-6-methoxyphenyl)-1-neopentyl-1H-indol-3-yl)-2,2,2-trifluoroethyl)cyclobutanesulfonamide (1.3 mg, 2.44 μmol, 6.13% yield) as a white solid. 1H NMR (400 MHz, CDCl3) δ 7.78 (d, J=8.3 Hz, 1H), 7.43 (s, 1H), 7.40 (d, J=7.9 Hz, 1H), 7.38-7.35 (m, 1H), 7.24-7.21 (m, 1H), 7.21-7.18 (m, 2H), 5.41-5.37 (m, 1H), 4.69 (d, J=8.1 Hz, 1H), 3.92 (s, 2H), 3.82 (d, J=8.3 Hz, 1H), 3.78 (s, 3H), 2.48 (d, J=11.7 Hz, 2H), 2.22 (d, J=27.4 Hz, 2H), 1.96 (dd, J=11.3, 6.0 Hz, 2H), 1.02 (s, 9H). MS found 550.8 [M+18].

### Example 7: (S)—N-(1-(6-(2-cyano-6-methoxyphenyl)-1-neopentyl-1H-indol-3-yl)-2,2,2-trifluoroethyl)cyclopropanesulfonamide

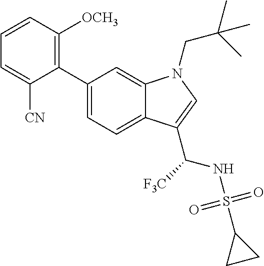 [SMILES: COc1cccc(C#N)c1-c1ccc2c([C@H](NS(=O)(=O)C3CC3)C(F)(F)F)cn(CC(C)(C)C)c2c1]

A mixture of(S)-2-(3-(1-amino-2,2,2-trifluoroethyl)-1-neopentyl-1H-indol-6-yl)-3-methoxybenzonitrile (100 mg, 240.70 μmol) and cyclopropanesulfonyl chloride (101.52 mg, 722.11 μmol, 73.57 μL) in anhydrous pyridine (2 mL) was stirred at RT for 12 hours. To the mixture was then added CH₂Cl₂ and then concentrated to give a black oil, which was purified by silica gel column (PE/EA=5:1) to give a brown oil. The crude was purified by Prep HPLC to get the title compound (45.3 mg, 87.19 μmol, 36.22% yield) as a white solid. 1H NMR (400 MHz, CDCl3) δ 7.80 (d, J=8.3 Hz, 1H), 7.43 (s, 1H), 7.40 (d, J=7.9 Hz, 1H), 7.36 (dd, J=7.7, 1.4 Hz, 1H), 7.24-7.17 (m, 3H), 5.49-5.39 (m, 1H), 4.87 (d, J=7.9 Hz, 1H), 3.92 (s, 2H), 3.78 (s, 3H), 2.47-2.37 (m, 1H), 1.17 (ddd, J=18.9, 4.8, 1.8 Hz, 2H), 1.00 (d, J=19.8 Hz, 9H), 0.94-0.87 (m, 2H). MS Found: 536.8 [M+18].

### Example 8: (S)—N-(2,2,2-trifluoro-1-(5-fluoro-1-neopentyl-6-(2-(trifluoromethyl)pyridin-3-yl)-1H-indol-3-yl)ethyl)cyclopropanesulfonamide

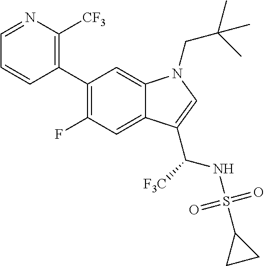 [SMILES: CC(C)(C)Cn1cc([C@H](NS(=O)(=O)C2CC2)C(F)(F)F)c2cc(F)c(-c3cccnc3C(F)(F)F)cc21]

### Step 1:6-bromo-5-fluoro-1-neopentyl-1H-indole

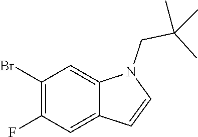 [SMILES: CC(C)(C)Cn1ccc2cc(F)c(Br)cc21]

6-Bromo-5-fluoro-1H-indole (10 g, 46.72 mmol) and potassium iodide (15.51 g, 93.44 mmol) in DMF (10 mL) were added to a suspension of NaH (7.47 g, 186.89 mmol, 60% purity) in anhydrous DMF (100 mL) in ice-water under nitrogen. The mixture was stirred for 1 h at room temperature then cooled with an ice batch, at which time, 1-bromo-2,2-dimethyl-propane (14.11 g, 93.44 mmol, 11.76 mL) was added and the resulting mixture was stirred at 40° C. overnight under nitrogen. After quenching with water the reaction mixture was partitioned between ethyl acetate (50 mL) and water (50 mL). The aqueous layer was extracted using ethyl acetate (2×50 mL), the combined organic layers were washed sequentially with sat. NH₄Cl and brine, then dried over anhydrous sodium sulfate. After the solvent was removed in vacuo, flash chromatography (silica gel, 100% Petroleum ether) yielded 6-bromo-1-(2,2-dimethylpropyl)-5-fluoro-indole (15.8 g, 55.60 mmol, 119.01% yield) as red brown oil.

### Step 2: (S)—N—((S)-1-(6-bromo-5-fluoro-1-neopentyl-1H-indol-3-yl)-2,2,2-trifluoroethyl)-2-methylpropane-2-sulfinamide

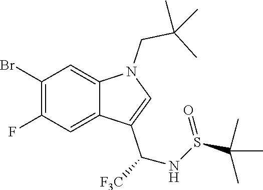 [SMILES: CC(C)(C)Cn1cc([C@H](N[S@@](=O)C(C)(C)C)C(F)(F)F)c2cc(F)c(Br)cc21]

The mixture of 6-bromo-1-(2,2-dimethylpropyl)-5-fluoro-indole (10.8 g, 38.01 mmol) in anhydrous CH₂Cl₂ (50 mL) was cooled to −10° C., at which time BF₃·OEt₂ (3.05 mL, 1.3 eq) was added slowly, followed by addition of a solution of (S,E)-2-methyl-N-(2,2,2-trifluoroethylidene) propane-2-sulfinamide in DCM. The mixture was then continued to stir for 2 h and then the mixture was diluted with DCM, followed by water. The aqueous layer was extracted with DCM twice. Organic layers were combined and washed with brine and concentrated in vacuo to obtain the crude product, which was purified by silica gel column (EA/PE=0-80%) to give(S)—N-[(1S)-1-[6-bromo-1-(2,2-dimethylpropyl)-5-fluoro-indol-3-yl]-2,2,2-trifluoro-ethyl]-2-methyl-propane-2-sulfinamide (12.47 g, 25.69 mmol) as yellow oil.

### Step 3: (S)-1-(6-bromo-5-fluoro-1-neopentyl-1H-indol-3-yl)-2,2,2-trifluoroethanamine

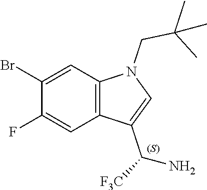

A mixture of(S)—N-[(1S)-1-[6-bromo-1-(2,2-dimethylpropyl)-5-fluoro-indol-3-yl]-2,2,2-trifluoro-ethyl]-2-methyl-propane-2-sulfinamide (11.97 g, 24.66 mmol) in 4M HCl (g)/MeOH (50 mL) was stirred at RT for 3 h. The mixture was concentrated, the residue was added sat. aqueous NaHCO₃ and EtOAc, stirred and separated, the aqueous was extracted with EtOAc×2. The combined organic phases were washed with brine, dried with anhydrous Na₂SO₄ and filtered. The filtrate was concentrated to give the crude product, which was purified by prep-HPLC to get (1S)-1-[6-bromo-1-(2,2-dimethylpropyl)-5-fluoro-indol-3-yl]-2,2,2-trifluoro-ethanamine (4.08 g, 10.70 mmol, 43.40% yield) as white solids. 1H NMR (400 MHz, d₆-DMSO) δ 7.96 (d, J=5.9 Hz, 1H), 7.66 (d, J=9.9 Hz, 1H), 7.54 (s, 1H), 4.85 (d, J=7.6 Hz, 1H), 4.00 (s, 2H), 3.7-3.8 (br s, 2H), 0.91 (s, 9H). LCMS: 365.8 m/z [M+H]⁺.

### Step 4: (1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine

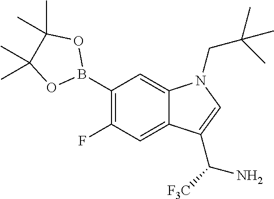 [SMILES: CC(C)(C)Cn1cc([C@H](N)C(F)(F)F)c2cc(F)c(B3OC(C)(C)C(C)(C)O3)cc21]

A mixture of (1S)-1-[6-bromo-1-(2,2-dimethylpropyl)-5-fluoro-indol-3-yl]-2,2,2-trifluoro-ethanamine (500 mg, 1.31 mmol), 4,4,5,5-tetramethyl-2-(4,4,5,5-tetramethyl-1,3,2-dioxaborolan-2-yl)-1,3,2-dioxaborolane (333.07 mg, 1.31 mmol), cyclopentyl(diphenyl)phosphanedichloropalladiumiron (287.92 mg, 393.49 μmol) and potassium acetate (386.18 mg, 3.93 mmol, 245.97 μL) in dioxane (10 mL) was stirred at 80° C. overnight under Nitrogen. The crude product was purified by silica gel column (EA/PE=0-25%) to get (1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (680 mg, 1.11 mmol, 84.74% yield) as yellow oil.

### Step 5: (S)-2,2,2-trifluoro-1-(5-fluoro-1-neopentyl-6-(2-(trifluoromethyl)pyridin-3-yl)-1H-indol-3-yl)ethan-1-amine

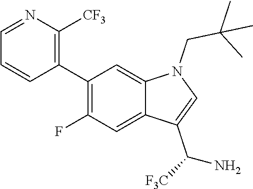 [SMILES: CC(C)(C)Cn1cc([C@H](N)C(F)(F)F)c2cc(F)c(-c3cccnc3C(F)(F)F)cc21]

A mixture of (1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (360 mg, 588.41 μmol) and 3-bromo-2-(trifluoromethyl)pyridine (172.87 mg, 764.94 μmol) in dioxane (10 mL) and H₂O (2 mL) was added Pd(dppf)Cl₂ (43.07 mg, 58.84 μmol) and K₂CO₃ (162.40 mg, 1.18 mmol). The mixture was purged by N₂ for 10 seconds, at which time the mixture was stirred at 85° C. for 5 hours. The product was purified by silica chromatograph (EA:PE, 0%-60%) to give (1S)-1-[1-(2,2-dimethylpropyl)-5-fluoro-6-[2-(trifluoromethyl)-3-pyridyl]indol-3-yl]-2,2,2-trifluoro-ethanamine (250 mg, 558.79 μmol, 94.97% yield) as a yellow gum was obtained.

### Step 6: (S)—N-(2,2,2-trifluoro-1-(5-fluoro-1-neopentyl-6-(2-(trifluoromethyl)pyridin-3-yl)-1H-indol-3-yl)ethyl)cyclopropanesulfonamide

The title compound was prepared following the same general protocol as described for Step 4, Example 2, using (S)-2,2,2-trifluoro-1-(5-fluoro-1-neopentyl-6-(2-(trifluoromethyl)pyridin-3-yl)-1H-indol-3-yl)ethan-1-amine and cyclopropanesulfonyl chloride. ¹H NMR (400 MHz, CDCl₃) δ 8.76 (dd, J=4.7, 1.2 Hz, 1H), 7.77 (d, J=7.7 Hz, 1H), 7.60-7.47 (m, 2H), 7.28 (s, 1H), 7.22 (d, J=5.9 Hz, 1H), 5.45-5.31 (m, 1H), 5.01 (s, 1H), 3.87 (s, 2H), 2.46 (d, J=4.2 Hz, 1H), 1.24 (dq, J=14.3, 4.8 Hz, 2H), 1.18-1.09 (m, 1H), 1.00-0.95 (m, 10H), 0.94-0.88 (m, 1H). MS Found: 552.1 [M+H]

### Example 9: (S)—N-(2,2,2-trifluoro-1-(5-fluoro-1-neopentyl-6-(3-(trifluoromethyl)pyridin-2-yl)-1H-indol-3-yl)ethyl)cyclopropanesulfonamide

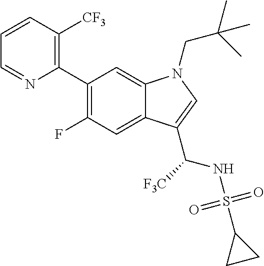 [SMILES: CC(C)(C)Cn1cc([C@H](NS(=O)(=O)C2CC2)C(F)(F)F)c2cc(F)c(-c3ncccc3C(F)(F)F)cc21]

### Step 1: (S)-2,2,2-trifluoro-1-(5-fluoro-1-neopentyl-6-(2-(trifluoromethyl)pyridine-3-yl)-1H-indol-3-yl)ethan-1-amine

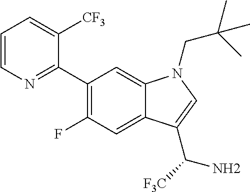 [SMILES: CC(C)(C)Cn1cc([C@H](N)C(F)(F)F)c2cc(F)c(-c3ncccc3C(F)(F)F)cc21]

A mixture of (1R)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (313.15 mg, 731.21 μmol) in dioxane (4 mL) and H₂O (1 mL) was added 2-bromo-3-(trifluoromethyl)pyridine (165.25 mg, 731.21 μmol). Then Pd(dppf)Cl₂ (53.52 mg, 73.12 μmol) and Na₂CO₃ (232.52 mg, 2.19 mmol) were added to the above mixture. The mixture was purged N₂ 2 times and stirred at 90° C. for 5 hours. The product was purified by flash (EA:PE, 0%-50%) to give (1R)-1-[1-(2,2-dimethylpropyl)-5-fluoro-6-[3-(trifluoromethyl)-2-pyridyl]indol-3-yl]-2,2,2-trifluoro-ethanamine (210 mg, 469.39 μmol, 64.19% yield) as a yellow oil was obtained.

### Step 2: (S)—N-(2,2,2-trifluoro-1-(5-fluoro-1-neopentyl-6-(3-(trifluoromethyl)pyridin-2-yl)-1H-indol-3-yl)ethyl)cyclopropanesulfonamide

The title compound was prepared following the same general protocol as described for Step 4, Example 2, using (S)-2,2,2-trifluoro-1-(5-fluoro-1-neopentyl-6-(2-(trifluoromethyl)pyridine-3-yl)-1H-indol-3-yl)ethan-1-amine and cyclopropanesulfonyl chloride. ¹H NMR (400 MHz, CDCl₃) δ 8.76 (dd, J=4.7, 1.2 Hz, 1H), 7.77 (d, J=7.7 Hz, 1H), 7.60-7.47 (m, 2H), 7.28 (s, 1H), 7.22 (d, J=5.9 Hz, 1H), 5.45-5.31 (m, 1H), 5.01 (s, 1H), 3.87 (s, 2H), 2.46 (d, J=4.2 Hz, 1H), 1.24 (dq, J=14.3, 4.8 Hz, 2H), 1.18-1.09 (m, 1H), 0.98 (d, J=7.3 Hz, 9H), 0.94-0.88 (m, 1H). MS Found: 552.1 [M+H]⁺

### Example 10: (S)—N-(1-(6-(3-cyanopyridin-2-yl)-5-fluoro-1-neopentyl-1H-indol-3-yl)-2,2,2-trifluoroethyl)cyclopropanesulfonamide

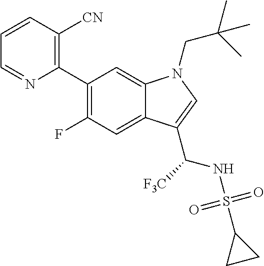 [SMILES: CC(C)(C)Cn1cc([C@H](NS(=O)(=O)C2CC2)C(F)(F)F)c2cc(F)c(-c3ncccc3C#N)cc21]

### Step 1: (S)-2-(3-(1-amino-2,2,2-trifluoroethyl)-5-fluoro-1-neopentyl-1H-pyrrolo[3,2-b]pyridin-6-yl)nicotinonitrile

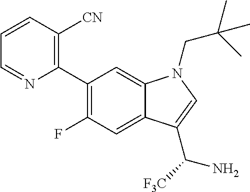 [SMILES: CC(C)(C)Cn1cc([C@H](N)C(F)(F)F)c2cc(F)c(-c3ncccc3C#N)cc21]

A mixture of (1R)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (313.15 mg, 731.21 μmol) in dioxane (4 mL) and H₂O (1 mL) was added 2-bromopyridine-3-carbonitrile (133.81 mg, 731.21 μmol). Then Pd(dppf)Cl₂ (53.52 mg, 73.12 μmol) and Na₂CO₃ (232.50 mg, 2.19 mmol) were added to the above mixture. The mixture was purged with N₂ 2 times and stirred at 90° C. for 5 hours. The product was purified by flash (EA:PE, 0%-50%) to give 2-[3-[(1R)-1-amino-2,2,2-trifluoro-ethyl]-1-(2,2-dimethylpropyl)-5-fluoro-indol-6-yl]pyridine-3-carbonitrile (180 mg, 445.10 μmol, 60.87% yield) as a yellow oi.

### Step 2: (S)—N-(1-(6-(3-cyanopyridin-2-yl)-5-fluoro-1-neopentyl-1H-indol-3-yl)-2,2,2-trifluoroethyl)cyclopropanesulfonamide

The title compound was prepared following the same general protocol as described for Step 4, Example 2, using (S)-2-(3-(1-amino-2,2,2-trifluoroethyl)-5-fluoro-1-neopentyl-1H-pyrrolo[3,2-b]pyridin-6-yl)nicotinonitrile and cyclopropanesulfonyl chloride. ¹H NMR (400 MHz, CDCl₃) δ 8.76 (dd, J=4.7, 1.2 Hz, 1H), 7.77 (d, J=7.7 Hz, 1H), 7.60-7.47 (m, 2H), 7.28 (s, 1H), 7.22 (d, J=5.9 Hz, 1H), 5.45-5.31 (m, 1H), 5.01 (s, 1H), 3.87 (s, 2H), 2.46 (d, J=4.2 Hz, 1H), 1.24 (dq, J=14.3, 4.8 Hz, 2H), 1.18-1.09 (m, 1H), 0.98 (d, J=7.3 Hz, 9H), 0.94-0.88 (m, 1H). MS Found: 509.2 [M+H]⁺.

### Example 11: (S)—N-(2,2-difluoro-1-(1-neopentyl-6-(2-(trifluoromethyl)phenyl)-1H-indol-3-yl)ethyl)-3,3-difluorocyclobutane-1-sulfonamide

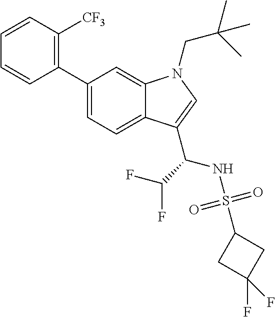 [SMILES: CC(C)(C)Cn1cc([C@H](NS(=O)(=O)C2CC(F)(F)C2)C(F)F)c2ccc(-c3ccccc3C(F)(F)F)cc21]

### Step 1:2,2-difluoro-1-(1-neopentyl-6-(2-(trifluoromethyl)phenyl)-1H-indol-3-yl)ethan-1-one

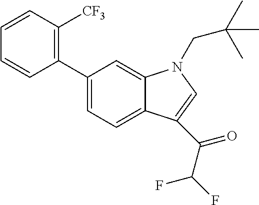 [SMILES: CC(C)(C)Cn1cc(C(=O)C(F)F)c2ccc(-c3ccccc3C(F)(F)F)cc21]

2,2-difluoroacetic anhydride (520 mg, 3 mmol) was added to a solution of 1-neopentyl-6-(2-(trifluoromethyl)phenyl)-1H-indole (660 mg, 2 mmol) in DMF (4 mL) at 0° C. and the reaction mixture was slowly warmed to room temperature. After stirring overnight, the reaction was diluted with ethyl acetate (10 mL) and saturated aqueous NaHCO₃ (10 mL). The layers were separated. The organic phase was dried (Na₂SO₄), filtered, and concentrated in vacuo. The crude was purified by chromatography on silica gel to afford the title compound (580 mg). ESI-MS (m/z): 410.1 [M+1]⁺.

### Step 2: (S)—N-(2,2-difluoro-1-(1-neopentyl-6-(2-(trifluoromethyl)phenyl)-1H-indol-3-yl)ethylidene)-2-methylpropane-2-sulfinamide

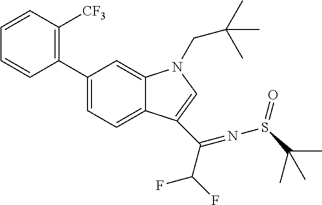 [SMILES: CC(C)(C)Cn1cc(C(=N[S@@](=O)C(C)(C)C)C(F)F)c2ccc(-c3ccccc3C(F)(F)F)cc21]

A solution of 2,2-difluoro-1-(1-neopentyl-6-(2-(trifluoromethyl)phenyl)-1H-indol-3-yl)ethan-1-one (580 mg, 1.4 mmol) and (S)-2-methylpropane-2-sulfinamide (340 mg, 2.8 mmol) in Ti(OiPr)₄ (1 mL) was heated at 2 h at 90° C. The solution was cooled to RT and diluted with EtOAc (10 mL) and brine (1 mL). The mixture was filtered and the filtrate was concentrated in vacuo to obtain the crude which was purified by chromatography on silica gel (EtOAc/hex) to afford the title compound (685 mg). ESI-MS (m/z): 513.2 [M+1]⁺.

### Step 3: (S)—N—((S)-2,2-difluoro-1-(1-neopentyl-6-(2-(trifluoromethyl)phenyl)-1H-indol-3-yl)ethyl)-2-methylpropane-2-sulfinamide

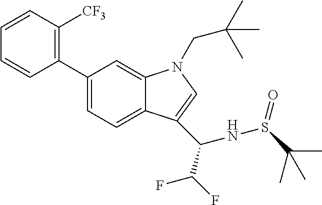 [SMILES: CC(C)(C)Cn1cc([C@H](N[S@@](=O)C(C)(C)C)C(F)F)c2ccc(-c3ccccc3C(F)(F)F)cc21]

NaBH₄ (100 mg, 2.66 mmol) was added to a solution of (S,E)-N-(2,2-difluoro-1-(1-neopentyl-6-(2-(trifluoromethyl)phenyl)-1H-indol-3-yl)ethylidene)-2-methylpropane-2-sulfinamide (685 mg, 1.33 mmol) in MeOH (4 mL) at 0° C. and the reaction mixture was stirred at room temperature for 1-2 hours. The solvent was removed under reduced pressure and the residue was dissolved in EtOAc (5 mL) and water (2 mL). The layers were separated and the aqueous layer was extracted with EtOAc (2×3 mL). The combined organics were dried (Na₂SO₄) and filtered. The solvent was removed under reduced pressure and the residue was purified by combi-flash on silica gel to obtain the title compound as an oil (350 mg). ESI-MS (m/z): 515.2 [M+1]⁺.

### Step 4: (S)-2,2-difluoro-1-(1-neopentyl-6-(2-(trifluoromethyl)phenyl)-1H-indol-3-yl)ethan-1-amine

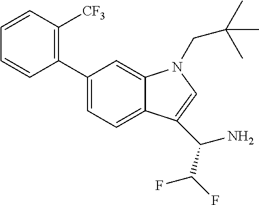 [SMILES: CC(C)(C)Cn1cc([C@H](N)C(F)F)c2ccc(-c3ccccc3C(F)(F)F)cc21]

The title compound was prepared following the same general protocol as described for Step 3, Example 2, using (S)—N—((S)-2,2-difluoro-1-(1-neopentyl-6-(2-(trifluoromethyl)-phenyl)-1H-indol-3-yl)ethyl)-2-methylpropane-2-sulfinamide. ESI-MS (m/z): 394.2 [M-NH₂]⁺.

### Step 5: (S)—N-(2,2-difluoro-1-(1-neopentyl-6-(2-(trifluoromethyl)phenyl)-1H-indol-3-yl)ethyl)cyclobutanesulfonamide

The title compound was prepared following the same general protocol as described for Step 4, Example 2, using (S)-2,2-difluoro-1-(1-neopentyl-6-(2-(trifluoromethyl)phenyl)-1H-indol-3-yl)ethan-1-amine and 3,3-difluorocyclobutane-1-sulfonyl chloride. ESI-MS (m/z): 564.41 [M+1]⁺.

### Example 12: (S)—N-(2,2-difluoro-1-(5-fluoro-1-neopentyl-6-(o-tolyl)-1H-indol-3yl)ethyl)cyclopropanesulfonamide

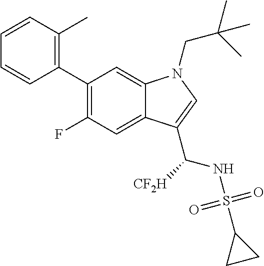 [SMILES: Cc1ccccc1-c1cc2c(cc1F)c([C@H](NS(=O)(=O)C1CC1)C(F)F)cn2CC(C)(C)C]

### Step 1: (S)—N—((S)-2,2-difluoro-1-(5-fluoro-1-neopentyl-6-(o-tolyl)-1H-indol-3-yl)ethyl)-2-methylpropane-2-sulfinamide

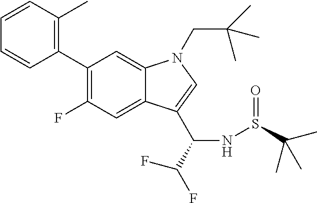 [SMILES: Cc1ccccc1-c1cc2c(cc1F)c([C@H](N[S@@](=O)C(C)(C)C)C(F)F)cn2CC(C)(C)C]

The title compound was prepared following the same general protocol as described for Step 6, Example 2, using (S)—N—((S)-1-(6-bromo-5-fluoro-1-neopentyl-1H-indol-3-yl)-2,2-difluoroethyl)-2-methylpropane-2-sulfinamide and o-tolylboronic acid. ESI-MS (m/z): ESI-MS (m/z): 478.6 [M+1]⁺.

### Step 2: (S)-2,2-difluoro-1-(5-fluoro-1-neopentyl-6-(o-tolyl)-1H-indol-3-yl)ethan-1-amine

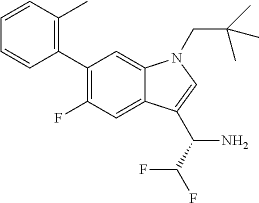 [SMILES: Cc1ccccc1-c1cc2c(cc1F)c([C@H](N)C(F)F)cn2CC(C)(C)C]

The title compound was prepared following the same general protocol as described for Step 3, Example 2, using (S)—N—((S)-2,2-difluoro-1-(5-fluoro-1-neopentyl-6-(o-tolyl)-1H-indol-3-yl)ethyl)-2-methylpropane-2-sulfinamide. ESI-MS (m/z): 358.4 [M-NH₂]⁺.

Step 3: The title compound was prepared following the same general protocol as described for Step 4, Example 2, using (S)-2,2-difluoro-1-(5-fluoro-1-neopentyl-6-(o-tolyl)-1H-indol-3-yl)ethan-1-amine and cyclopropanesulfonyl chloride. ESI-MS (m/z): 478.54 [M+1]⁺.

### Example 13: (S)—N-(2,2-difluoro-1-(1-neopentyl-6-(2-(trifluoromethyl)pyridin-3-yl)-1H-indol-3-yl)ethyl)cyclopropanesulfonamide

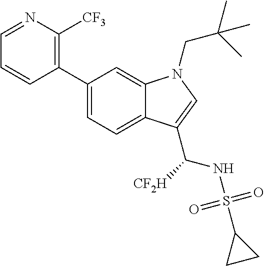 [SMILES: CC(C)(C)Cn1cc([C@H](NS(=O)(=O)C2CC2)C(F)F)c2ccc(-c3cccnc3C(F)(F)F)cc21]

### Step 1. 1-(6-bromo-1-neopentyl-1H-indol-3-yl)-2,2-difluoroethan-1-one

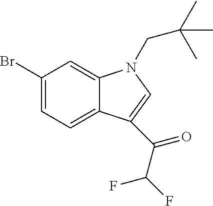 [SMILES: CC(C)(C)Cn1cc(C(=O)C(F)F)c2ccc(Br)cc21]

2,2-difluoroacetic anhydride (5.2 g, 30 mmol) was added to a solution of 6-bromo-1-neopentyl-1H-indole (5.3 g, 20 mmol) in DMF (30 mL) at 0° C. and the reaction mixture was slowly warmed to room temperature. After stirring overnight, the reaction was diluted with ethyl acetate (100 mL) and saturated aqueous NaHCO₃ (50 mL). The layers were separated. The organic phase was dried (Na₂SO₄), filtered, and concentrated in vacuo. The crude was purified by chromatography on silica gel to afford the title compound (5.9 g). ESI-MS (m/z): 343.4 [M+1]⁺.

### Step 2. (S)—N-(1-(6-bromo-1-neopentyl-1H-indol-3-yl)-2,2-difluoroethylidene)-2-methylpropane-2-sulfinamide

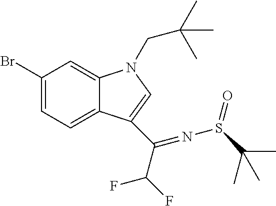 [SMILES: CC(C)(C)Cn1cc(C(=N[S@@](=O)C(C)(C)C)C(F)F)c2ccc(Br)cc21]

A solution of 1-(6-bromo-1-neopentyl-1H-indol-3-yl)-2,2-difluoroethan-1-one (5.9 g, 17.2 mmol) and (S)-2-methylpropane-2-sulfinamide (4.1 g, 34 mmol) in Ti(OiPr)₄ (20 mL) was heated at 2 h at 90° C. The solution was cooled to RT and diluted with EtOAc (100 mL) and brine (30 mL). The mixture was filtered, the solid was washed with EtOAc (100 mL×3) and the filtrate was concentrated in vacuo to obtain the crude which was purified by chromatography on silica gel (EtOAc/hex) to afford the title compound (6.3 g). ESI-MS (m/z): 446.4 [M+1]⁺.

### Step 3. (S)—N—((S)-1-(6-bromo-1-neopentyl-1H-indol-3-yl)-2,2-difluoroethyl)-2-methylpropane-2-sulfinamide

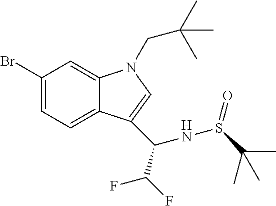 [SMILES: CC(C)(C)Cn1cc([C@H](N[S@@](=O)C(C)(C)C)C(F)F)c2ccc(Br)cc21]

NaBH₄ (1.1 g, 28 mmol) was added in portions to a solution of(S)—N-(1-(6-bromo-1-neopentyl-1H-indol-3-yl)-2,2-difluoroethylidene)-2-methylpropane-2-sulfinamide (6.3 g, 14 mmol) in MeOH (30 mL) at 0° C. and the reaction mixture was stirred at room temperature for 1-2 hours. The solvent was removed under reduced pressure and the residue was dissolved in EtOAc (50 mL) and water (20 mL). The layers were separated and the aqueous layer was extracted with EtOAc (3×20 mL). The combined organics were dried (Na₂SO₄) and filtered. The solvent was removed under reduced pressure and the residue was purified by combi-flash on silica gel to obtain the title compound as an oil (3.1 g). ESI-MS (m/z): 448.5 [M+1]⁺.

### Step 4. (S)-1-(6-bromo-1-neopentyl-1H-indol-3-yl)-2,2-difluoroethan-1-amine

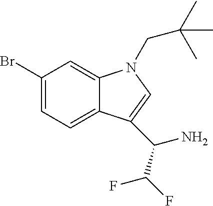 [SMILES: CC(C)(C)Cn1cc([C@H](N)C(F)F)c2ccc(Br)cc21]

37% HCl aqueous solution (3.0 mL) was added in portions to a solution of(S)—N—((S)-1-(6-bromo-1-neopentyl-1H-indol-3-yl)-2,2-difluoroethyl)-2-methylpropane-2-sulfinamide (3.1 g, 6.9 mmol) in MeOH (30 mL) at 0° C. and the reaction mixture was stirred at room temperature for 3-4 hours. The solvent was removed under reduced pressure and the residue was dissolved in EtOAc (50 mL) and adjusted pH=8 with saturated NaHCO₃. The layers were separated, and the aqueous layer was extracted with EtOAc (3×20 mL). The combined organics were dried (Na₂SO₄) and filtered. The solvent was removed under reduced pressure and the residue was purified by combi-flash on silica gel to obtain the title compound as an oil (2.1 g). ESI-MS (m/z): 328.2 [M-NH₂]⁺.

### Step 5. (S)—N-(1-(6-bromo-1-neopentyl-1H-indol-3-yl)-2,2-difluoroethyl)cyclopropanesulfonamide

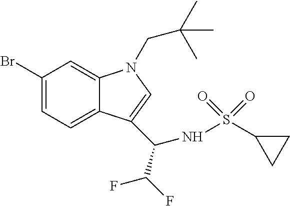 [SMILES: CC(C)(C)Cn1cc([C@H](NS(=O)(=O)C2CC2)C(F)F)c2ccc(Br)cc21]

Cyclopropanesulfonyl chloride (1.3 g, 9.1 mmol) was added in portions to a solution of(S)-1-(6-bromo-1-neopentyl-1H-indol-3-yl)-2,2-difluoroethan-1-amine (2.1 g, 6.1 mmol) in pyridine (12 mL) at 0° C. and the reaction mixture was stirred at room temperature for 16-24 hours. The reaction mixture was diluted EtOAc (50 mL) and adjusted pH=7 with 4M HCl aqueous solution. The layers were separated, and the aqueous layer was extracted with EtOAc (2×50 mL). The combined organics were dried (Na₂SO₄) and filtered. The solvent was removed under reduced pressure and the residue was purified by combi-flash on silica gel to obtain the title compound as a solid (2.7 g). ESI-MS (m/z): 448.56 [M+1]⁺.

### Step 6. (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide

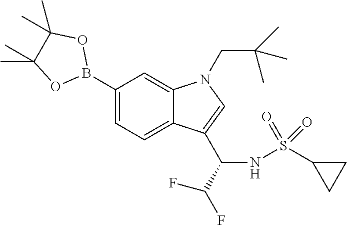 [SMILES: CC(C)(C)Cn1cc([C@H](NS(=O)(=O)C2CC2)C(F)F)c2ccc(B3OC(C)(C)C(C)(C)O3)cc21]

4,4,4′,4′,5,5,5′,5′-octamethyl-2,2′-bi(1,3,2-dioxaborolane) (510 mg, 2.0 mmol), KOAc (252 mg, 3.0 mmol) and Pd catalyst (70 mg, 0.1 mmol) was added to a solution of(S)—N-(2,2-difluoro-1-(1-neopentyl-6-(4,4,5,5-tetramethyl-1,3,2-dioxaborolan-2-yl)-1H-indol-3-yl)ethyl)cyclo-propanesulfonamide (448 mg, 1.0 mmol) in dioxane (5.0 mL) and the reaction mixture was degassed for three times. Followed by heating at 100° C. for 10-16 hours. The reaction mixture was diluted EtOAc (20 mL) and water (20 mL). The layers were separated, and the aqueous layer was extracted with EtOAc (2×15 mL). The combined organics were dried (Na₂SO₄) and filtered. The solvent was removed under reduced pressure and the residue was purified by combi-flash on silica gel to obtain the title compound as a solid (415 mg). ESI-MS (m/z): 496.6 [M+1]⁺.

Step 7: The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-2-(trifluoromethyl)pyridine. ESI-MS (m/z): 515.79 [M+1]⁺.

### Example 14: (S)—N-(2,2-difluoro-1-(1-neopentyl-6-(2-(trifluoromethyl)pyridin-3-yl)-1H-indol-3-yl)ethyl)cyclobutanesulfonamide

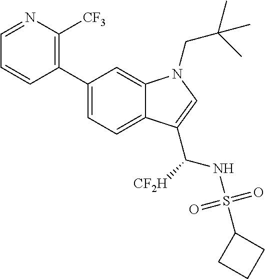 [SMILES: CC(C)(C)Cn1cc([C@H](NS(=O)(=O)C2CCC2)C(F)F)c2ccc(-c3cccnc3C(F)(F)F)cc21]

### Step 1: (S)—N—((S)-2,2-difluoro-1-(1-neopentyl-6-(2-(trifluoromethyl)pyridin-3-yl)-1H-indol-3-yl)ethyl)-2-methylpropane-2-sulfinamide

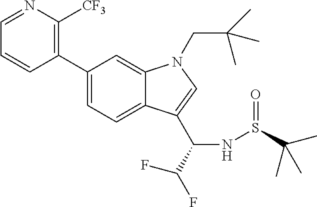 [SMILES: CC(C)(C)Cn1cc([C@H](N[S@@](=O)C(C)(C)C)C(F)F)c2ccc(-c3cccnc3C(F)(F)F)cc21]

The title compound was prepared following the same general protocol as described for Step 6, Example 2, using (S)—N—((S)-1-(6-bromo-1-neopentyl-1H-indol-3-yl)-2,2-difluoroethyl)-2-methylpropane-2-sulfinamide and (2-(trifluoromethyl)pyridin-3-yl)boronic acid. ESI-MS (m/z): 515.6 [M+1]⁺.

### Step 2: (S)-2,2-difluoro-1-(1-neopentyl-6-(2-(trifluoromethyl)pyridin-3-yl)-1H-indol-3-yl)ethan-1-amine

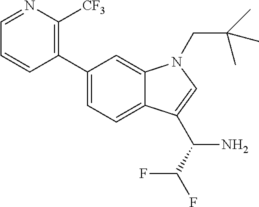 [SMILES: CC(C)(C)Cn1cc([C@H](N)C(F)F)c2ccc(-c3cccnc3C(F)(F)F)cc21]

The title compound was prepared following the same general protocol as described for Step 3, Example 2, using (S)—N—((S)-2,2-difluoro-1-(1-neopentyl-6-(2-(trifluoromethyl)pyridin-3-yl)-1H-indol-3-yl)ethyl)-2-methylpropane-2-sulfinamide. ESI-MS (m/z): 395.2 [M-NH₂]⁺.

Step 3: The title compound was prepared following the same general protocol as described for Step 4, Example 2, using (S)-2,2-difluoro-1-(5-fluoro-1-neopentyl-6-(2-(trifluoromethyl)pyridin-3-yl)-1H-indol-3-yl)ethan-1-amine and cyclobutanesulfonyl chloride. ESI-MS (m/z): 529.78 [M+1]⁺.

### Example 15: (S)—N-(2,2-difluoro-1-(1-neopentyl-6-(o-tolyl)-1H-indol-3-yl)ethyl)cyclopropanesulfonamide

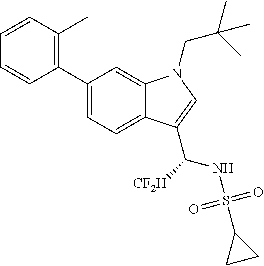 [SMILES: Cc1ccccc1-c1ccc2c([C@H](NS(=O)(=O)C3CC3)C(F)F)cn(CC(C)(C)C)c2c1]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 1-bromo-2-methylbenzene. ESI-MS (m/z): 460.53 [M+1]⁺.

### Example 16: (S)—N-(1-(6-(3,6-dihydro-2H-pyran-4-yl)-5-fluoro-1-neopentyl-1H-indol-3-yl)-2,2-difluoroethyl)cyclopropanesulfonamide

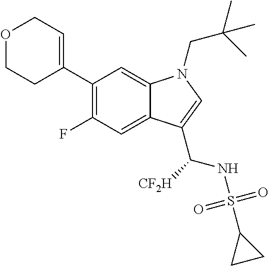 [SMILES: CC(C)(C)Cn1cc([C@H](NS(=O)(=O)C2CC2)C(F)F)c2cc(F)c(C3=CCOCC3)cc21]

The title compound was prepared following the same general protocol as described for Step 6, Example 2, using (S)—N-(1-(6-bromo-5-fluoro-1-neopentyl-1H-indol-3-yl)-2,2-difluoroethyl)cyclopropanesulfonamide and 2-(3,6-dihydro-2H-pyran-4-yl)-4,4,5,5-tetramethyl-1,3,2-dioxaborolane. ESI-MS (m/z): 470.50 [M+1]⁺.

### Example 17: (R or S)—N-(1-(5-fluoro-1-neopentyl-6-(3-(trifluoromethyl)pyridine-2-yl)-1H-indol-3-yl)ethyl)cyclopropane sulfonamide

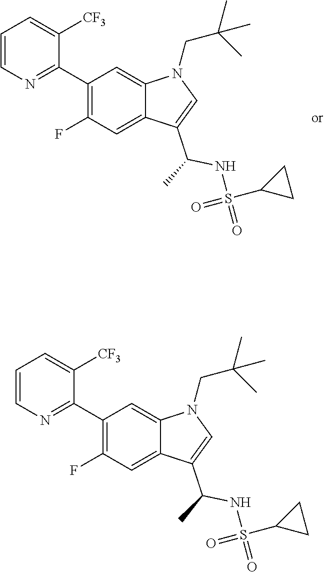 [SMILES: C[C@@H](NS(=O)(=O)C1CC1)c1cn(CC(C)(C)C)c2cc(-c3ncccc3C(F)(F)F)c(F)cc12 ; C[C@H](NS(=O)(=O)C1CC1)c1cn(CC(C)(C)C)c2cc(-c3ncccc3C(F)(F)F)c(F)cc12]

To A mixture of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (110 mg, 229.92 μmol) in dioxane (4 mL) and H₂O (1 mL) was added 2-bromo-3-(trifluoromethyl)pyridine (51.96 mg, 229.92 μmol). Then Pd(dppf)Cl₂ (168.30 mg, 229.92 μmol) and Na₂CO₃ (73.11 mg, 689.76 μmol) were added to the above mixture. The mixture was purged with N₂ 2 times and stirred at 90° C. for 5 hours. The product was purified by Prep-HPLC to give N-[1-[1-(2,2-dimethylpropyl)-5-fluoro-6-[3-(trifluoromethyl)-2-pyridyl]indol-3-yl]ethyl]cyclopropanesulfonamide (60 mg, 120.59 μmol, 52.45% yield) as a white solid and was purified again by chiral SFC separation (OD-20% MeOH) to give the title compound as a white solid (peak 1, 18 mg, 36.18 μmol, 30% yield). ¹H NMR (400 MHz, CDCl₃) δ 8.87 (d, J=4.0 Hz, 1H), 8.15-8.06 (m, 1H), 7.54-7.44 (m, 2H), 7.25 (s, 1H), 7.13 (s, 1H), 5.01-4.89 (m, 1H), 4.52 (d, J=6.5 Hz, 1H), 3.84 (s, 2H), 2.21 (dt, J=8.0, 3.1 Hz, 1H), 1.74 (d, J=6.8 Hz, 3H), 1.12-1.05 (m, 2H), 0.97 (s, 9H), 0.79 (dd, J=8.0, 4.7 Hz, 2H). MS Found: 498.2 [M+H]

### Example 18: (S or R)—N-(1-(5-fluoro-1-neopentyl-6-(3-(trifluoromethyl)pyridine-2-yl)-1H-indol-3-yl)ethyl)cyclopropane sulfonamide

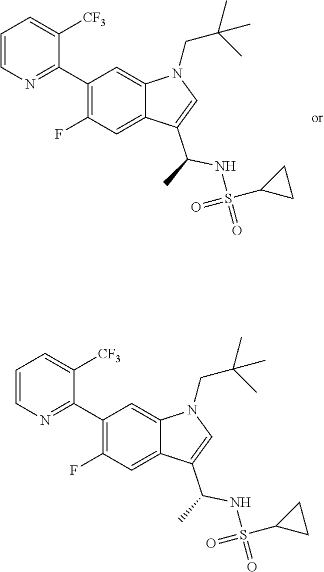 [SMILES: C[C@H](NS(=O)(=O)C1CC1)c1cn(CC(C)(C)C)c2cc(-c3ncccc3C(F)(F)F)c(F)cc12 ; C[C@@H](NS(=O)(=O)C1CC1)c1cn(CC(C)(C)C)c2cc(-c3ncccc3C(F)(F)F)c(F)cc12]

Example 18 was prepared as described in Example 17 to give the title compound as a white solid (peak 2, 18 mg, 36.18 μmol, 30% yield). ¹H NMR (400 MHz, CDCl₃) δ 8.86 (d, J=4.0 Hz, 1H), 8.10 (dd, J=8.0, 1.2 Hz, 1H), 7.54-7.44 (m, 2H), 7.26 (d, J=5.7 Hz, 1H), 7.13 (s, 1H), 4.95 (t, J=6.7 Hz, 1H), 4.65 (d, J=6.6 Hz, 1H), 3.84 (s, 2H), 2.20 (dq, J=8.1, 4.8 Hz, 1H), 1.74 (d, J=6.8 Hz, 3H), 1.12-1.03 (m, 2H), 0.97 (s, 9H), 0.78 (ddd, J=10.3, 6.4, 3.7 Hz, 2H). MS Found: 498.2 [M+H]⁺.

### Example 19: (R or S)—N-(1-(6-(3-cyanopyridin-2-yl)-5-fluoro-1-neopentyl-1H-indol-3-yl)ethyl)cyclopropanesulfonamide

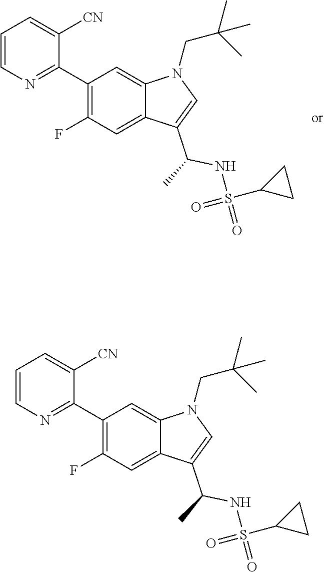 [SMILES: C[C@@H](NS(=O)(=O)C1CC1)c1cn(CC(C)(C)C)c2cc(-c3ncccc3C#N)c(F)cc12 ; C[C@H](NS(=O)(=O)C1CC1)c1cn(CC(C)(C)C)c2cc(-c3ncccc3C#N)c(F)cc12]

A mixture of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (110 mg, 229.92 μmol) in dioxane (4 mL) and H₂O (1 mL) was added 2-bromopyridine-3-carbonitrile (42.08 mg, 229.92 μmol). Then Pd(dppf)Cl₂ (16.83 mg, 22.99 μmol) and Na₂CO₃ (73.11 mg, 689.76 μmol) were added to the above mixture. The mixture was purged with N₂ for 2 times and stirred at 90° C. for 5 hours. The product was purified by Prep-HPLC to give N-[1-[6-(3-cyano-2-pyridyl)-1-(2,2-dimethylpropyl)-5-fluoro-indol-3-yl]ethyl]cyclopropanesulfonamide (40 mg, 88.00 μmol, 38.27% yield) as a white solid. The product was purified again by Chiral SFC (AD-20% MeOH) to give (R or S)—N-(1-(6-(3-cyanopyridin-2-yl)-5-fluoro-1-neopentyl-1H-indol-3-yl)ethyl)cyclopropanesulfonamide (Peak 1, 10 mg, 22.0 μmol, 25.0% yield) as a white solid. ¹H NMR (400 MHz, CDCl₃) δ 8.91 (dd, J=4.8, 1.5 Hz, 1H), 8.09 (dd, J=7.9, 1.5 Hz, 1H), 7.58 (d, J=10.9 Hz, 1H), 7.51 (d, J=5.8 Hz, 1H), 7.42 (dd, J=7.8, 4.9 Hz, 1H), 7.18 (s, 1H), 4.96 (d, J=6.7 Hz, 1H), 4.55 (s, 1H), 3.90 (s, 2H), 2.21 (dd, J=12.8, 4.8 Hz, 1H), 1.74 (d, J=6.8 Hz, 3H), 1.09 (dd, J=4.7, 2.0 Hz, 2H), 1.01 (s, 9H), 0.81 (t, J=8.3 Hz, 2H). MS Found: 455.1 [M+H]⁺.

### Example 20: (S or R)—N-(1-(6-(3-cyanopyridin-2-yl)-5-fluoro-1-neopentyl-1H-indol-3-yl)ethyl)cyclopropanesulfonamide

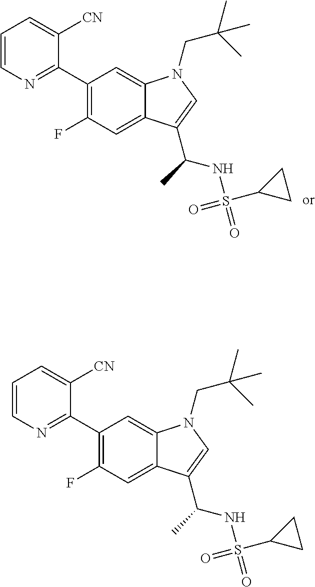 [SMILES: C[C@H](NS(=O)(=O)C1CC1)c1cn(CC(C)(C)C)c2cc(-c3ncccc3C#N)c(F)cc12 ; "or" ; C[C@@H](NS(=O)(=O)C1CC1)c1cn(CC(C)(C)C)c2cc(-c3ncccc3C#N)c(F)cc12]

Example 20 was prepared as described in example 19 to give the title compound as a white solid (Peak 2, 6.41 mg, 14.1 μmol, 16.0% yield. ¹H NMR (400 MHz, CDCl₃) δ 8.91 (d, J=3.5 Hz, 1H), 8.09 (d, J=7.5 Hz, 1H), 7.58 (d, J=10.8 Hz, 1H), 7.51 (d, J=5.7 Hz, 1H), 7.42 (dd, J=7.6, 5.0 Hz, 1H), 7.18 (s, 1H), 5.02-4.89 (m, 1H), 4.52 (d, J=6.0 Hz, 1H), 3.90 (s, 2H), 2.27-2.17 (m, 1H), 1.74 (d, J=6.7 Hz, 3H), 1.09 (d, J=2.1 Hz, 2H), 1.01 (s, 9H), 0.81 (t, J=8.0 Hz, 2H). MS Found: 455.2 [M+H]⁺.

### Example 21: (S)—N-(2,2-difluoro-1-(1-neopentyl-6-(2-(trifluoromethyl)phenyl)-1H-indol-3-yl)ethyl)-sulfamoyldimethylamine

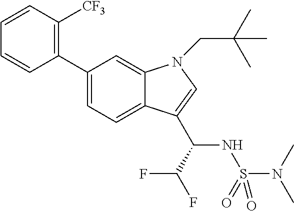 [SMILES: CN(C)S(=O)(=O)N[C@@H](c1cn(CC(C)(C)C)c2cc(-c3ccccc3C(F)(F)F)ccc12)C(F)F]

To the solution of(S)-2,2-difluoro-1-(1-neopentyl-6-(2-(trifluoromethyl)phenyl)-1H-indol-3-yl)ethan-1-amine (20 mmg, 0.05 mmol) in pyridine (0.3 mL) was added dimethylsulfamoyl chloride (15 mg, 0.1 mmol). The reaction was allowed to stir at rt overnight. The solution was diluted with DCM (2 mL), washed with HCl aqueous, water and brine. The organic layer was separated and concentrated in vacuo. The crude product was purified by chromatography on silica gel (EtOAc/hex) to afford the title compound. ESI-MS (m/z): 517.27 [M+1]⁺.

### Example 22: (S)—N-(2,2-difluoro-1-(1-neopentyl-6-(2-(trifluoromethyl)phenyl)-1H-indol-3-yl)ethyl)-3-fluorocyclobutane-1-sulfonamide

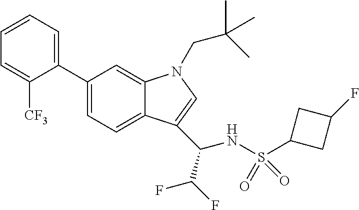 [SMILES: CC(C)(C)Cn1cc([C@H](NS(=O)(=O)C2CC(F)C2)C(F)F)c2ccc(-c3ccccc3C(F)(F)F)cc21]

The title compound was prepared following the same general protocol as described for Step 4, Example 2, using (S)-2,2-difluoro-1-(1-neopentyl-6-(2-(trifluoromethyl)phenyl)-1H-indol-3-yl)ethan-1-amine and 3-fluorocyclobutane-1-sulfonyl chloride. ESI-MS (m/z): 546.47 [M+1]⁺.

### Example 23: (R or S) N-(1-(1-neopentyl-6-(tetrahydro-2H-pyran-4-yl)-1H-indol-3-yl)ethyl)cyclopropanesulfonamide

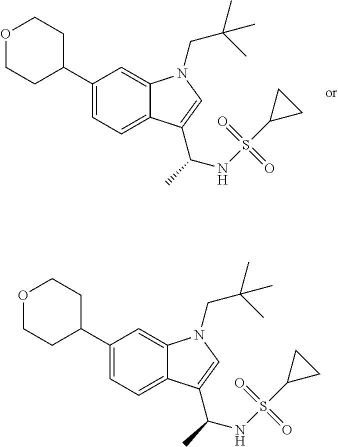 [SMILES: C[C@@H](NS(=O)(=O)C1CC1)c1cn(CC(C)(C)C)c2cc(C3CCOCC3)ccc12 ; C[C@H](NS(=O)(=O)C1CC1)c1cn(CC(C)(C)C)c2cc(C3CCOCC3)ccc12]

### Step 1: N-(1-(6-(3,6-dihydro-2H-pyran-4-yl)-1-neopentyl-1H-indol-3-yl)ethyl)cyclopropanesulfonamide

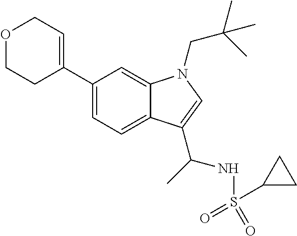 [SMILES: CC(NS(=O)(=O)C1CC1)c1cn(CC(C)(C)C)c2cc(C3=CCOCC3)ccc12]

To a mixture of N-[1-[6-bromo-1-(2,2-dimethylpropyl)indol-3-yl]ethyl]cyclopropanesulfonamide (72.0 mg, 174.18 μmol) in water (1 mL) and dioxane (5 mL) was added 2-(3,6-dihydro-2H-pyran-4-yl)-4,4,5,5-tetramethyl-1,3,2-dioxaborolane (36.59 mg, 174.18 μmol). Then Pd(dppf)Cl₂ (12.74 mg, 17.42 μmol) and sodium carbonate (55.38 mg, 522.53 μmol, 21.87 μL) were added to the above mixture. The mixture was purged by N₂ 2 times and stirred at 90° C. for 12 hours. The mixture was diluted with water and extracted with ethyl acetate. The organic layer was washed with brine, dried over sodium carbonate and concentrated under reduced pressure, which was purified by prep-HPLC to give N-(1-(6-(3,6-dihydro-2H-pyran-4-yl)-1-neopentyl-1H-indol-3-yl)ethyl)cyclopropanesulfonamide (58 mg, 139.23 μmol, 79.9% yield) as an off-white solid.

### Step 2: N-(1-(1-neopentyl-6-(tetrahydro-2H-pyran-4-yl)-1H-indol-3-yl)ethyl)cyclopropanesulfonamide

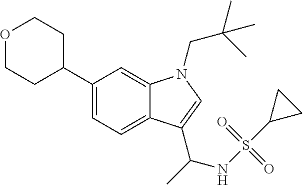 [SMILES: CC(NS(=O)(=O)C1CC1)c1cn(CC(C)(C)C)c2cc(C3CCOCC3)ccc12]

To a solution of N-[1-[6-(3,6-dihydro-2H-pyran-4-yl)-1-(2,2-dimethylpropyl)indol-3-yl]ethyl]cyclopropanesulfonamide (58 mg, 139.23 μmol) in EA (5 mL) was added Pd/C (10 mg, 13.92 μmol) under N₂. The suspension was degassed under vacuum and purged with H₂ several times. The mixture was stirred under H₂ balloon at RT for 2 hours. The suspension was filtered through a pad of silica gel and the filter cake was washed with EA (10 mL×3). The combined filtrates were evaporated to dryness to give the crude product. The crude product was purified by prep-HPLC to give N-[1-[1-(2,2-dimethylpropyl)-6-tetrahydropyran-4-yl-indol-3-yl]ethyl]cyclopropanesulfonamide (6.85 mg, 16.36 μmol, 11.75% yield) as a white solid. as a white solid. 1H NMR (400 MHz, DMSO) δ 7.58-7.35 (m, 3H), 7.26 (d, J=28.4 Hz, 1H), 4.77-4.65 (m, 1H), 3.96 (dd, J=15.8, 4.2 Hz, 4H), 3.48 (t, J=11.0 Hz, 2H), 3.10 (t, J=11.9 Hz, 1H), 2.31 (ddd, J=12.6, 7.8, 4.9 Hz, 1H), 1.81 (ddt, J=16.9, 11.4, 5.6 Hz, 2H), 1.67 (d, J=12.3 Hz, 2H), 1.57 (t, J=6.7 Hz, 3H), 0.92 (s, 9H), 0.89-0.68 (m, 4H).

### Step 3: N-(1-(1-neopentyl-6-(tetrahydro-2H-pyran-4-yl)-1H-indol-3-yl)ethyl)cyclopropanesulfonamide

N-(1-(1-neopentyl-6-(tetrahydro-2H-pyran-4-yl)-1H-indol-3-yl)ethyl)cyclopropanesulfonamide (40 mg, 95.56 μmol) was purified by SFC with an AD column using 20% MeOH to provide (R or S) N-[(1S)-1-[1-(2,2-dimethylpropyl)-6-tetrahydropyran-4-yl-indol-3-yl]ethyl]cyclopropanesulfonamide (Peak 1, 5.44 mg, 13.00 μmol, 27.20% yield) 1H NMR (400 MHz, CDCl3) δ 7.69 (d, J=8.2 Hz, 1H), 7.17 (s, 1H), 7.01 (dd, J=8.3, 1.3 Hz, 1H), 6.99 (s, 1H), 4.99-4.94 (m, 1H), 4.42 (d, J=6.7 Hz, 1H), 4.11 (dd, J=10.7, 3.8 Hz, 2H), 3.85 (s, 2H), 3.57 (td, J=11.6, 2.5 Hz, 2H), 2.90-2.83 (m, 1H), 2.28 (dd, J=8.8, 4.0 Hz, 1H), 1.89 (dd, J=12.3, 4.2 Hz, 2H), 1.82 (d, J=14.1 Hz, 2H), 1.73 (d, J=6.8 Hz, 3H), 1.17-1.10 (m, 2H), 1.00 (s, 9H), 0.85-0.80 (m, 2H). MS Found: 298.2 [M-cyclopropanesulfonamide].

### Example 24: (S or R) N-(1-(1-neopentyl-6-(tetrahydro-2H-pyran-4-yl)-1H-indol-3-yl)ethyl)cyclopropanesulfonamide

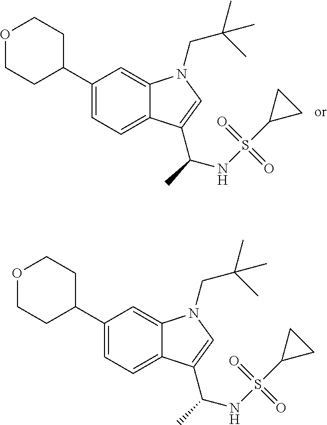 [SMILES: C[C@H](NS(=O)(=O)C1CC1)c1cn(CC(C)(C)C)c2cc(C3CCOCC3)ccc12 ; "or" ; C[C@@H](NS(=O)(=O)C1CC1)c1cn(CC(C)(C)C)c2cc(C3CCOCC3)ccc12]

N-(1-(1-neopentyl-6-(tetrahydro-2H-pyran-4-yl)-1H-indol-3-yl)ethyl)cyclopropanesulfonamide (40 mg, 95.56 μmol) was purified by SFC with an AD column using 20% MeOH to provide (S or R) (Peak 2, 5.55 mg, 13.26 μmol, 27.75% yield) as a white solid. 1H NMR (400 MHz, CDCl3) δ 7.68 (d, J=8.2 Hz, 1H), 7.17 (s, 1H), 7.01 (dd, J=8.2, 1.2 Hz, 1H), 6.99 (s, 1H), 5.00-4.94 (m, 1H), 4.42 (d, J=6.7 Hz, 1H), 4.11 (dd, J=10.8, 3.6 Hz, 2H), 3.85 (s, 2H), 3.57 (td, J=11.5, 2.5 Hz, 2H), 2.90-2.83 (m, 1H), 2.32-2.26 (m, 1H), 1.89 (dd, J=12.5, 4.2 Hz, 2H), 1.84 (s, 2H), 1.73 (d, J=6.8 Hz, 3H), 1.16-1.09 (m, 2H), 1.00 (s, 9H), 0.86-0.80 (m, 2H). MS Found: 298.2 [M-cyclopropanesulfonamide].

### Example 26: (R or S) N-(1-(6-(2-cyano-6-fluorophenyl)-1-neopentyl-1H-indol-3-yl)ethyl)cyclopropanesulfonamide

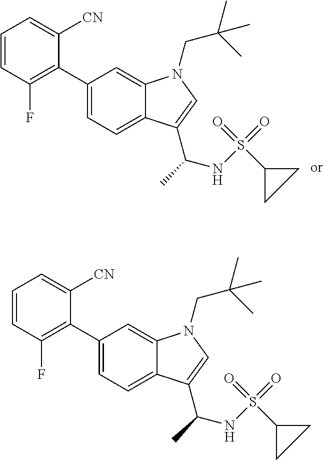 [SMILES: C[C@@H](NS(=O)(=O)C1CC1)c1cn(CC(C)(C)C)c2cc(-c3c(F)cccc3C#N)ccc12 ; "or" ; C[C@H](NS(=O)(=O)C1CC1)c1cn(CC(C)(C)C)c2cc(-c3c(F)cccc3C#N)ccc12]

### Step 1: synthesis of N-(1-(6-(2-cyano-6-fluorophenyl)-1-neopentyl-1H-indol-3-yl)ethyl)cyclopropanesulfonamide

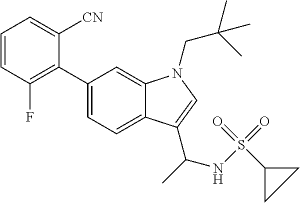 [SMILES: CC(NS(=O)(=O)C1CC1)c1cn(CC(C)(C)C)c2cc(-c3c(F)cccc3C#N)ccc12]

To a mixture of N-(1-(1-neopentyl-6-(4,4,5,5-tetramethyl-1,3,2-dioxaborolan-2-yl)-1H-indol-3-yl)ethyl)cyclopropanesulfonamide (400 mg, 868.74 μmol) in water (1 mL) and dioxane (5 mL) was added 2-bromo-3-fluorobenzonitrile (173.76 mg, 868.74 μmol) at which time Pd(dppf)Cl₂ (63.57 mg, 86.87 μmol) and sodium carbonate (276.23 mg, 2.61 mmol, 109.10 μL) were added to the above mixture. The mixture was purged with N₂ (2 times) and stirred at 90° C. for 12 hours. The mixture was diluted with water and extracted with ethyl acetate. The organic layer was washed with brine, dried over sodium carbonate and concentrated under reduced pressure. The crude was purified by prep-HPLC to give N-(1-(6-(2-cyano-6-fluorophenyl)-1-neopentyl-1H-indol-3-yl)ethyl)cyclopropanesulfonamide (200 mg, 440.95 μmol, 50.76% yield) as a white solid.

### Step 2: (R or S) N-(1-(6-(2-cyano-6-fluorophenyl)-1-neopentyl-1H-indol-3-yl)ethyl)cyclopropanesulfonamide

N-(1-(6-(2-cyano-6-fluorophenyl)-1-neopentyl-1H-indol-3-yl)ethyl)cyclopropanesulfonamide (120 mg, 264.57 μmol) was purified by SFC (column AD_20% MeOH) to give (R or S) N-(1-(6-(2-cyano-6-fluorophenyl)-1-neopentyl-1H-indol-3-yl)ethyl)cyclopropanesulfonamide (10.4 mg, 22.93 μmol, 17.3% yield) as a white solid. ₁H NMR (400 MHz, CDCl3) δ 7.87 (d, J=8.2 Hz, 1H), 7.61-7.57 (m, 1H), 7.45 (s, 1H), 7.44-7.37 (m, 2H), 7.22 (d, J=8.2 Hz, 1H), 7.13 (s, 1H), 5.05-4.98 (m, 1H), 4.50 (d, J=6.6 Hz, 1H), 3.90 (s, 2H), 2.28-2.21 (m, 1H), 1.77 (d, J=6.8 Hz, 3H), 1.14-1.06 (m, 2H), 1.02 (s, 9H), 0.81 (d, J=8.2 Hz, 2H). MS Found: 471.2 [M+18]⁺.

### Example 27: (S or R) N-[(1R)-1-[6-(2-cyano-6-fluoro-phenyl)-1-(2,2-dimethylpropyl)indol-3-yl]ethyl]cyclo

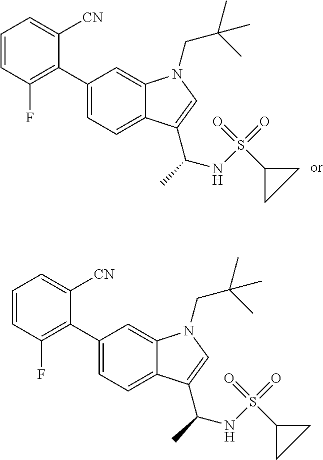 [SMILES: C[C@@H](NS(=O)(=O)C1CC1)c1cn(CC(C)(C)C)c2cc(-c3c(F)cccc3C#N)ccc12 ; "or" ; C[C@H](NS(=O)(=O)C1CC1)c1cn(CC(C)(C)C)c2cc(-c3c(F)cccc3C#N)ccc12]

N-[(1R)-1-[6-(2-cyano-6-fluoro-phenyl)-1-(2,2-dimethylpropyl)indol-3-yl]ethyl]cyclopropanesulfonamide (120 mg, 264.57 μmol) was purified by SFC (column AD_20% MeOH) to give (S or R) N-[(1R)-1-[6-(2-cyano-6-fluoro-phenyl)-1-(2,2-dimethylpropyl)indol-3-yl]ethyl]cyclo (Peak 2, 11.5 mg, 25.35 μmol, 19.2% yield) as a white solid. ¹H NMR (400 MHz, CDCl3) δ 7.87 (d, J=8.3 Hz, 1H), 7.61-7.57 (m, 1H), 7.45 (s, 1H), 7.44-7.37 (m, 2H), 7.24-7.20 (m, 1H), 7.13 (s, 1H), 5.02 (t, J=6.7 Hz, 1H), 4.49 (d, J=6. Hz, 1H), 3.90 (s, 2H), 2.24 (ddd, J=8.0, 4.8, 3.2 Hz, 1H), 1.77 (d, J=6.8 Hz, 3H), 1.10 (qd, J=5.3, 3.1 Hz, 2H), 1.02 (s, 9H), 0.82 (t, J=6.7 Hz, 2H). 417.2 [M+18]⁺.

### Example 28: (R or S) N-[1-[1-(2,2-dimethylpropyl)-5-fluoro-6-[2-(trifluoromethyl)-3-pyridyl]indol-3-yl]ethyl]cyclopropanesulfonamide

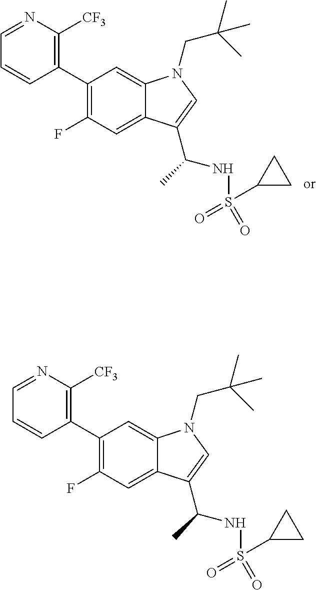 [SMILES: C[C@@H](NS(=O)(=O)C1CC1)c1cn(CC(C)(C)C)c2cc(-c3cccnc3C(F)(F)F)c(F)cc12 ; "or" ; C[C@H](NS(=O)(=O)C1CC1)c1cn(CC(C)(C)C)c2cc(-c3cccnc3C(F)(F)F)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (110 mg, 229.92 μmol) and 3-bromo-2-(trifluoromethyl)pyridine (51.96 mg, 229.92 μmol) in water (496.15 μL) and dioxane (1.98 mL) was added sodium carbonate (48.74 mg, 459.84 μmol, 19.25 μL) and Pd(dppf)Cl₂ (25.24 mg, 34.49 μmol). The solution was then stirred at 90° C. for 5 hr. at which time the reaction was diluted with water (30 ml) and extracted with EA (3×50 mL). The combined organic layers were washed with brine (50 ml), dried over Na₂SO₄, filtered and concentrated. The residue obtained was purified by silica gel chromatography to give N-[1-[1-(2,2-dimethylpropyl)-5-fluoro-6-[2-(trifluoromethyl)-3-pyridyl]indol-3-yl]ethyl]cyclopropanesulfonamide 40 mg as a white solid which was purified again by chiral SFC purification (column AS with 15% MeOH) to give the title compound (Peak 1, 3.25 mg, 6.53 μmol, 2.84% yield) as a white solid. ¹H NMR (400 MHz, CDCl3) δ 8.69 (s, 1H), 7.71 (d, J=7.5 Hz, 1H), 7.46 (d, J=9.5 Hz, 2H), 7.09 (d, J=18.7 Hz, 2H), 4.89 (s, 1H), 4.39 (s, 1H), 3.77 (s, 2H), 1.94 (s, 1H), 1.69 (d, J=6.0 Hz, 3H), 1.06 (s, 2H), 0.91 (s, 9H), 0.79 (s, 2H). MS Found: 497.55 [M+H]⁺.

### Example 29: (S or R) N-[1-[1-(2,2-dimethylpropyl)-5-fluoro-6-[2-(trifluoromethyl)-3-pyridyl]indol-3-yl]ethyl]cyclopropanesulfonamide

Example 29 was prepared as described in Example 28 to give the title compound ((Peak 2, 3.3 mg, 6.63 μmol, 2.88% yield) as white solid. ¹H NMR (400 MHz, CDCl3) δ 8.70 (s, 1H), 7.71 (d, J=8.0 Hz, 1H), 7.52-7.48 (m, 1H), 7.46 (d, J=10.4 Hz, 1H), 7.10 (d, J=5.9 Hz, 1H), 7.06 (s, 1H), 4.92-4.87 (m, 1H), 4.38 (d, J=6.5 Hz, 1H), 3.77 (s, 2H), 1.94 (d, J=5.1 Hz, 1H), 1.69 (d, J=6.8 Hz, 3H), 1.07 (s, 2H), 0.91 (s, 9H), 0.80 (d, J=7.1 Hz, 2H) Found MS+1:497.55

### Example 30: (S)—N-(2,2-difluoro-1-(1-neopentyl-6-(o-tolyl)-1H-indol-3-yl)ethyl)-3,3-difluorocyclobutane-1-sulfonamide

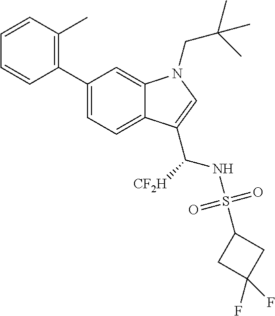 [SMILES: Cc1ccccc1-c1ccc2c([C@H](NS(=O)(=O)C3CC(F)(F)C3)C(F)F)cn(CC(C)(C)C)c2c1]

The title compound was prepared following the same general protocol as described for Step 4, Example 2, using (S)-2,2-difluoro-1-(1-neopentyl-6-(o-tolyl)-1H-indol-3-yl)ethan-1-amine and 3,3-difluorocyclobutane-1-sulfonyl chloride. ESI-MS (m/z): 510.63 [M+1]⁺.

### Example 31: (S)—N-(2,2-difluoro-1-(5-fluoro-1-neopentyl-6-(2-(trifluoromethyl)phenyl)-1H-indol-3-yl)ethyl)azetidine-1-sulfonamide

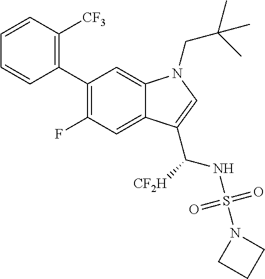 [SMILES: CC(C)(C)Cn1cc([C@H](NS(=O)(=O)N2CCC2)C(F)F)c2cc(F)c(-c3ccccc3C(F)(F)F)cc21]

### Step 1:1-(6-bromo-5-fluoro-1-neopentyl-1H-indol-3-yl)-2,2-difluoroethan-1-one

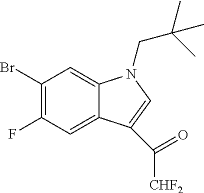 [SMILES: CC(C)(C)Cn1cc(C(=O)C(F)F)c2cc(F)c(Br)cc21]

The title compound was prepared following the same general protocol as described for Step 1, Example 11, using 6-bromo-5-fluoro-1-neopentyl-1H-indole and 2,2-difluoroacetic anhydride. ESI-MS (m/z): 361.5, 363.5 [M+1]⁺.

### Step 2: (S,E)-N-(1-(6-bromo-5-fluoro-1-neopentyl-1H-indol-3-yl)-2,2-difluoroethylidene)-2-methylpropane-2-sulfinamide

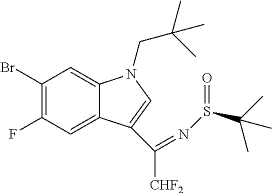 [SMILES: CC(C)(C)Cn1cc(/C(=N\[S@@](=O)C(C)(C)C)C(F)F)c2cc(F)c(Br)cc21]

The title compound was prepared following the same general protocol as described for Step 2, Example 11, using 1-(6-bromo-5-fluoro-1-neopentyl-1H-indol-3-yl)-2,2-difluoroethan-1-one and (S)-2-methylpropane-2-sulfinamide, and the reaction was stirred for 2 h at 90° C. ESI-MS (m/z): 464.7, 466.5 [M+1]⁺.

### Step 3: (S)—N—((S)-1-(6-bromo-5-fluoro-1-neopentyl-1H-indol-3-yl)-2,2-difluoroethyl)-2-methylpropane-2-sulfinamide

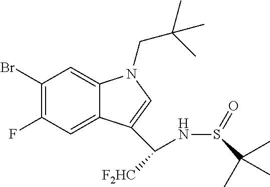 [SMILES: CC(C)(C)Cn1cc([C@H](N[S@@](=O)C(C)(C)C)C(F)F)c2cc(F)c(Br)cc21]

(S,E)-N-(1-(6-bromo-5-fluoro-1-neopentyl-1H-indol-3-yl)-2,2-difluoroethylidene)-2-methylpropane-2-sulfinamide (6.8 g, 14.7 mmol) in THF (40 mL) was cooled to at −10° C. and L-selectride (1M in THF, 29 mL, 29.0 mmol) was added slowly. The reaction was allowed to stir for 1 h. The completion was monitored by anal. HPLC. The reaction was quenched with cold water and extracted with EtOAc twice. The organic layer was washed with brine and dried over Na₂SO₄. The solvent was removed and the crude was purified by silica gel to obtain the title compound. ¹H NMR (CDCl₃, 400 MHz) δ 7.51 (d, J=5.6 Hz, 1H), 7.34 (d, J=9.28 Hz, 1H), 7.22 (s, 1H), 7.48 (t, J=8.0 Hz, 1H), 7.37 (d, J=8.0 Hz, 1H), 7.32 (s, 1H), 7.17 (s, 1H), 7.11 (d, J=8.0 Hz, 1H), 6.06 (td, J=55.8 Hz, 2.2 Hz, 1H), 4.88-4.80 (m, 1H), 3.89-3.80 (m, 3H), 1.21 (s, 9H), 0.99 (s, 9H)

### Step 4: (S)—N—((S)-2,2-difluoro-1-(5-fluoro-1-neopentyl-6-(2-(trifluoromethyl)phenyl)-1H-indol-3-yl)ethyl)-2-methylpropane-2-sulfinamide

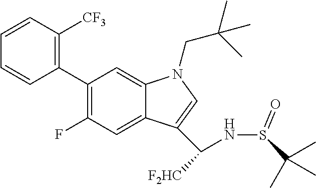 [SMILES: CC(C)(C)Cn1cc([C@H](N[S@@](=O)C(C)(C)C)C(F)F)c2cc(F)c(-c3ccccc3C(F)(F)F)cc21]

The title compound was prepared following the same general protocol as described for Step 6, Example 2, using (S)—N—((S)-1-(6-bromo-5-fluoro-1-neopentyl-1H-indol-3-yl)-2,2-difluoroethyl)-2-methylpropane-2-sulfinamide and (2-(trifluoromethyl)phenyl)boronic acid. ESI-MS (m/z): 532.6 [M+1]⁺.

### Step 5: (S)-2,2-difluoro-1-(5-fluoro-1-neopentyl-6-(2-(trifluoromethyl)phenyl)-1H-indol-3-yl)ethan-1-amine

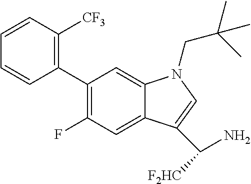 [SMILES: CC(C)(C)Cn1cc([C@H](N)C(F)F)c2cc(F)c(-c3ccccc3C(F)(F)F)cc21]

The title compound was prepared following the same general protocol as described for Step 3, Example 2, using (S)—N—((S)-2,2-difluoro-1-(5-fluoro-1-neopentyl-6-(2-(trifluoromethyl)phenyl)-1H-indol-3-yl)ethyl)-2-methylpropane-2-sulfinamide.

Step 6: The title compound was prepared following the same general protocol as described for Step 4, Example 2, using (S)-2,2-difluoro-1-(5-fluoro-1-neopentyl-6-(2-(trifluoromethyl)phenyl)-1H-indol-3-yl)ethan-1-amine and azetidine-1-sulfonyl chloride. ESI-MS (m/z): 547.30 [M+1]⁺.

### Example 32: (R or S) N-(1-(6-(4-cyanopyridin-3-yl)-5-fluoro-1-neopentyl-1H-indol-3-yl)ethyl)cyclopropanesulfonamide

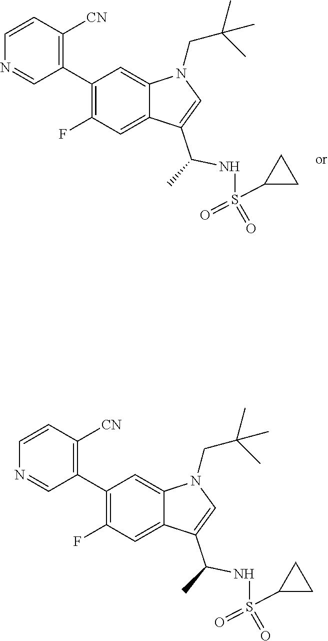 [SMILES: C[C@@H](NS(=O)(=O)C1CC1)c1cn(CC(C)(C)C)c2cc(-c3cnccc3C#N)c(F)cc12 ; C[C@H](NS(=O)(=O)C1CC1)c1cn(CC(C)(C)C)c2cc(-c3cnccc3C#N)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (150 mg, 313.53 μmol) and 3-bromopyridine-4-carbonitrile (57.38 mg, 313.53 μmol) in water (494.75 μL) and dioxane (1.98 mL) was added Pd(dppf)Cl₂ (34.41 mg, 47.03 μmol) and sodium carbonate (66.46 mg, 627.06 μmol, 26.25 μL). The solution was stirred at 90° C. for 5 hr. at which time the reaction was diluted with water (30 ml) and extracted with EA (50 ml×3). The combined organic layers were washed with brine (50 ml), dried over Na₂SO₄, filtered and concentrated. The residue obtained was purified by Prep-HPLC to give N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide 40 mg as a white solid. The product was purified again by chiral SFC (AS_15% MeOH) to give the title compound (Peak 1, 4.17 mg, 9.17 μmol, 2.93% yield) as a white solid. ¹H NMR (400 MHz, CDCl₃) δ 8.80 (s, 1H), 8.70 (d, J=5.0 Hz, 1H), 7.58 (d, J=5.1 Hz, 1H), 7.55 (d, J=10.7 Hz, 1H), 7.27 (d, J=5.9 Hz, 1H), 7.11 (s, 1H), 4.89 (dd, J=13.5, 6.7 Hz, 1H), 4.45 (d, J=6.7 Hz, 1H), 3.82 (s, 2H), 2.22 (ddd, J=8.0, 4.8, 3.2 Hz, 1H), 1.68 (d, J=6.8 Hz, 3H), 1.06 (d, J=4.8 Hz, 2H), 0.95 (s, 9H), 0.82-0.79 (m, 2H). MS Found: 455.2 [M+H]⁺.

### Example 33: (S or R) N-(1-(6-(4-cyanopyridin-3-yl)-5-fluoro-1-neopentyl-1H-indol-3-yl)ethyl)cyclopropanesulfonamide

Example 33 was prepared as described in Example 32 to give the title compound (Peak 2, 3.11 mg, 6.84 μmol, 2.18% yield) as a white solid. ¹H NMR (400 MHz, CDCl₃) δ 8.80 (s, 1H), 8.70 (d, J=5.0 Hz, 1H), 7.58 (d, J=5.0 Hz, 1H), 7.55 (d, J=10.7 Hz, 1H), 7.27 (d, J=5.9 Hz, 1H), 7.11 (s, 1H), 4.95-4.86 (m, 1H), 4.42 (d, J=6.4 Hz, 1H), 3.82 (s, 2H), 2.23 (ddd, J=12.7, 7.9, 4.7 Hz, 1H), 1.68 (d, J=6.8 Hz, 3H), 1.07 (d, J=4.7 Hz, 2H), 0.95 (s, 9H), 0.83-0.78 (m, 2H). MS Found: 455.2 [M+H]⁺.

### Example 34: N-(1-(5-fluoro-1-neopentyl-6-(tetrahydrofuran-3-yl)-1H-indol-3-yl)ethyl)cyclopropanesulfonamide

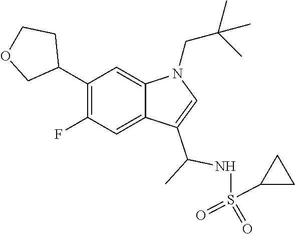 [SMILES: CC(NS(=O)(=O)C1CC1)c1cn(CC(C)(C)C)c2cc(C3CCOC3)c(F)cc12]

### Step 1: N-(1-(6-(2,5-dihydrofuran-3-yl)-5-fluoro-1-neopentyl-1H-indol-3-yl)ethyl)cyclopropanesulfonamide

To a solution of N-[1-[6-bromo-1-(2,2-dimethylpropyl)-5-fluoro-indol-3-yl]ethyl]cyclopropanesulfonamide (100 mg, 231.82 μmol) in a dioxane water mixture (4:1) (10 mL) was added 2-(2,5-dihydrofuran-3-yl)-4,4,5,5-tetramethyl-1,3,2-dioxaborolane (45.45 mg, 231.82 μmol). Then sodium carbonate (49.14 mg, 463.65 μmol, 19.41 μL) and Pd(dppf)Cl₂ were added to the above mixture. The mixture was then purged with N₂ for 2 times and stirred at 90° C. for 12 hours. The mixture was diluted with water and extracted with ethyl acetate. The organic layer was washed with brine, dried over sodium carbonate and concentrated under reduced pressure. The crude was then purified by normal phase silica gel chromatography to give N-[1-[6-(2,5-dihydrofuran-3-yl)-1-(2,2-dimethylpropyl)-5-fluoro-indol-3-yl]ethyl]cyclopropanesulfonamide (89 mg, 211.63 μmol, 91.29% yield) as a yellow solid.

### Step 2: N-(1-(5-fluoro-1-neopentyl-6-(tetrahydrofuran-3-yl)-1H-indol-3-yl)ethyl)cyclopropanesulfonamide

To a solution of N-[1-[6-(2,5-dihydrofuran-3-yl)-1-(2,2-dimethylpropyl)-5-fluoro-indol-3-yl]ethyl]cyclopropanesulfonamide (89 mg, 211.63 μmol) EA (5 mL) was added Pd (22.52 mg, 211.63 μmol) under an atmosphere of Nitrogen. The suspension was degassed under vacuum and purged with H₂ several times. The mixture was stirred under an H₂ balloon at RT for 2 hours, at which time the suspension was filtered through a pad of silica gel and the filter cake was washed with EA (10 mL×3). The combined filtrates were evaporated to dryness to give the crude product which was then by prep-HPLC to give N-[1-[1-(2,2-dimethylpropyl)-5-fluoro-6-tetrahydrofuran-3-yl-indol-3-yl]ethyl]cyclopropanesulfonamide (6.76 mg, 15.68 μmol, 7.41% yield, 98% purity) as a white solid. 1H NMR (400 MHz, DMSO) δ 7.56-7.37 (m, 3H), 7.23 (s, 1H), 4.72 (dd, J=14.7, 7.4 Hz, 1H), 4.05 (s, 1H), 3.94 (s, 2H), 3.84 (dd, J=15.4, 7.5 Hz, 1H), 3.64 (dd, J=9.9, 6.5 Hz, 2H), 2.29 (dd, J=15.1, 10.1 Hz, 2H), 2.11-1.93 (m, 1H), 1.55 (d, J=6.8 Hz, 2H), 1.23 (s, 2H), 0.92 (s, 7H), 0.89-0.67 (m, 4H). MS Found: 302.2 [M+H]⁺.

### Example 36: N-(1-(5-fluoro-6-(2-methylpyridin-3-yl)-1-neopentyl-1H-indol-3-yl)ethyl)cyclopropanesulfonamide

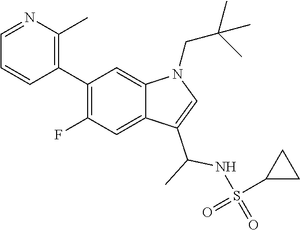 [SMILES: Cc1ncccc1-c1cc2c(cc1F)c(C(C)NS(=O)(=O)C1CC1)cn2CC(C)(C)C]

To a mixture of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (100 mg, 209.02 μmol) in dioxane (1.98 mL) and water (495 μL) was added 3-bromo-2-methyl-pyridine (35.96 mg, 209.02 μmol, 24.05 μL) and sodium carbonate (44.31 mg, 418.04 μmol, 17.50 μL). Cyclopentyl(diphenyl)phosphane;dichloropalladium;iron (22.94 mg, 31.35 μmol) was then added to the mixture and purged with N₂ for 3 min. at which time the reaction was stirred at 90 C for 5 h. The reaction was diluted with water (30 mL) and extracted with EA (20 mL×2). The organic layer was combined, washed with brine, dried over Na₂SO₄ and concentrated to give crude product. The crude product was purified with silica gel column and concentrated to give N-[1-[1-(2,2-dimethylpropyl)-5-fluoro-6-(2-methyl-3-pyridyl)indol-3-yl]ethyl]cyclopropanesulfonamide (18 mg, 40.58 μmol, 19.41% yield). ¹H NMR (400 MHz, CDCl₃) δ 8.55 (dd, J=4.9, 1.7 Hz, 1H), 7.57 (d, J=7.6 Hz, 1H), 7.52 (d, J=10.3 Hz, 1H), 7.22 (dd, J=7.6, 4.9 Hz, 1H), 7.15-7.09 (m, 2H), 4.96 (dd, J=13.5, 6.8 Hz, 1H), 4.46 (d, J=6.5 Hz, 1H), 3.86 (s, 2H), 2.47 (s, 3H), 2.34 (tt, J=8.0, 4.9 Hz, 1H), 1.76 (d, J=6.8 Hz, 3H), 1.17 (dt, J=6.5, 4.8 Hz, 2H), 1.00 (s, 9H), 0.89 (qd, J=5.6, 1.3 Hz, 2H). MS Found: 444.3 [M+H]⁺.

### Example 37: N-[1-[6-(5-cyano-3-pyridyl)-1-(2,2-dimethylpropyl)-5-fluoro-indol-3-yl]ethyl]cyclopropanesulfonamide

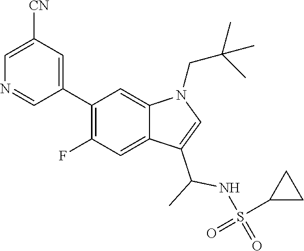 [SMILES: CC(NS(=O)(=O)C1CC1)c1cn(CC(C)(C)C)c2cc(-c3cncc(C#N)c3)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (20 mg, 41.80 μmol) and 3-bromo-5-cyano-pyridine (7.65 mg, 41.80 μmol) in dioxane (2.01 mL) and water (503 μL) was added sodium carbonate (8.86 mg, 83.61 μmol, 3.50 μL) and cyclopentyl(diphenyl)phosphanedichloropalladium;iron (4.59 mg, 6.27 μmol). The reaction mixture was stirred at 90° C. for 5 hr at which time the reaction was diluted with water (30 ml) and extracted with EA (50 ml×3). The combined organic layers were washed with brine (50 ml), dried over Na₂SO₄, filtered and concentrated. The crude residue was purified by Prep-HPLC to give the title compound (4.55 mg, 10.01 μmol, 23.94% yield) as a white solid. 1H NMR (400 MHz, CDCl3) δ 8.99 (s, 1H), 8.85 (d, J=1.6 Hz, 1H), 8.16 (d, J=1.7 Hz, 1H), 7.60 (d, J=11.3 Hz, 1H), 7.30 (d, J=6.2 Hz, 1H), 7.16 (s, 1H), 5.00-4.93 (m, 1H), 4.45 (d, J=6.8 Hz, 1H), 3.91 (s, 2H), 2.31 (td, J=8.0, 4.0 Hz, 1H), 1.75 (d, J=6.8 Hz, 3H), 1.20-1.12 (m, 2H), 1.02 (s, 9H), 0.89 (td, J=8.2, 2.0 Hz, 2H). MS Found: 454.56 [M+H]⁺.

### Example 38: (S)—N-(2,2-difluoro-1-(5-fluoro-1-neopentyl-6-(2-(trifluoromethyl)pyridin-3-yl)-1H-indol-3-yl)ethyl)cyclopropanesulfonamide

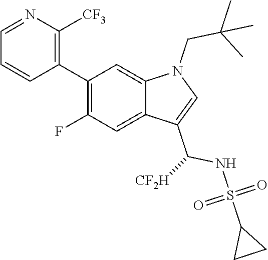 [SMILES: CC(C)(C)Cn1cc([C@H](NS(=O)(=O)C2CC2)C(F)F)c2cc(F)c(-c3cccnc3C(F)(F)F)cc21]

### Step 1: (S)—N-(1-(6-bromo-5-fluoro-1-neopentyl-1H-indol-3-yl)-2,2-difluoroethyl)cyclopropanesulfonamide

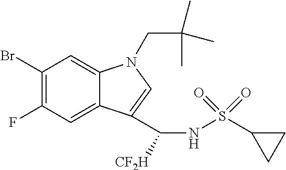 [SMILES: CC(C)(C)Cn1cc([C@H](NS(=O)(=O)C2CC2)C(F)F)c2cc(F)c(Br)cc21]

The title compound was prepared following the same general protocols as described for Steps 3 and 4, Example 2, using (S)—N—((S)-1-(6-bromo-5-fluoro-1-neopentyl-1H-indol-3-yl)-2,2-difluoroethyl)-2-methylpropane-2-sulfinamide and cyclopropanesulfonyl chloride ESI-MS (m/z): 466.5, 468.6 [M+1]⁺.

Step 2: The title compound was prepared following the same general protocol as described for Step 6, Example 2, using (S)—N-(1-(6-bromo-5-fluoro-1-neopentyl-1H-indol-3-yl)-2,2-difluoroethyl)cyclopropanesulfonamide and (2-(trifluoromethyl)pyridin-3-yl)boronic acid with anhydrous dioxane as solvent, and the reaction temperature 140° C. ESI-MS (m/z): 533.5 [M+1]⁺.

### Example 39: (S)—N-(2,2-difluoro-1-(1-neopentyl-6-(3-(trifluoromethyl)pyridin-2-yl)-1H-indol-3-yl)ethyl)cyclopropanesulfonamide

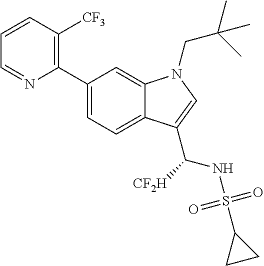 [SMILES: CC(C)(C)Cn1cc([C@H](NS(=O)(=O)C2CC2)C(F)F)c2ccc(-c3ncccc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-(trifluoromethyl)pyridine. ESI-MS (m/z): 515.98 [M+1]⁺.

### Example 40: (S)—N-(1-(6-(3-cyanopyridin-2-yl)-1-neopentyl-1H-indol-3-yl)-2,2-difluoroethyl)cyclopropanesulfonamide

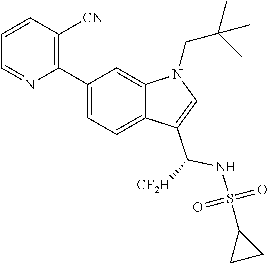 [SMILES: CC(C)(C)Cn1cc([C@H](NS(=O)(=O)C2CC2)C(F)F)c2ccc(-c3ncccc3C#N)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromonicotinonitrile. ESI-MS (m/z): 473.03 [M+1]⁺.

### Example 41: N-(2,2-difluoro-1-(5-fluoro-1-neopentyl-6-(2-(trifluoromethyl)phenyl)-1H-indol-3-yl)ethyl)bicyclo[1.1.1]pentane-1-sulfonamide

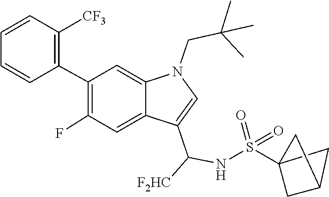 [SMILES: CC(C)(C)Cn1cc(C(NS(=O)(=O)C23CC(C2)C3)C(F)F)c2cc(F)c(-c3ccccc3C(F)(F)F)cc21]

### Step 1:2,2-difluoro-1-(5-fluoro-1-neopentyl-6-(2-(trifluoromethyl)phenyl)-1H-indol-3-yl)ethan-1-one

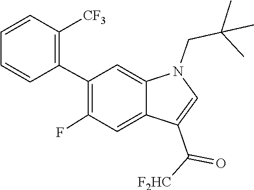 [SMILES: CC(C)(C)Cn1cc(C(=O)C(F)F)c2cc(F)c(-c3ccccc3C(F)(F)F)cc21]

The title compound was prepared following the same general protocol as described for Step 1, Example 11, using 5-fluoro-1-neopentyl-6-(2-(trifluoromethyl)phenyl)-1H-indole and 2,2-difluoroacetic anhydride. ESI-MS (m/z): 427.8 [M+1]⁺.

### Step 2: (E)-N-(2,2-difluoro-1-(5-fluoro-1-neopentyl-6-(2-(trifluoromethyl)phenyl)-1H-indol-3-yl)ethylidene) bicyclo[1.1.1]pentane-1-sulfonamide

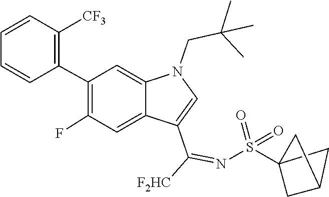 [SMILES: CC(C)(C)Cn1cc(/C(=N\S(=O)(=O)C23CC(C2)C3)C(F)F)c2cc(F)c(-c3ccccc3C(F)(F)F)cc21]

The title compound was prepared following the same general protocol as described for Step 2, Example 11, using 2,2-difluoro-1-(5-fluoro-1-neopentyl-6-(2-(trifluoromethyl)phenyl)-1H-indol-3-yl)ethan-1-one and bicyclo[1.1.1]pentane-1-sulfonamide, and the reaction was stirred for 1 h at 120° C. ESI-MS (m/z): 556.8 [M+1]⁺.

### Step 3: N-(2,2-difluoro-1-(5-fluoro-1-neopentyl-6-(2-(trifluoromethyl)phenyl)-1H-indol-3-yl)ethyl)bicyclo[1.1.1]pentane-1-sulfonamide

(E)-N-(2,2-difluoro-1-(5-fluoro-1-neopentyl-6-(2-(trifluoromethyl)phenyl)-1H-indol-3-yl)ethylidene) bicyclo[1.1.1]pentane-1-sulfonamide (0.031 g, 0.0557 mmol) in THF/water (10/1, 1 mL) was added NaBH₄ (0.004 g, 0.111 mmol). The completion was monitored by anal. HPLC. The reaction was quenched with MeOH. The solvent was removed and the crude was purified by silica gel to obtain the title compound. ESI-MS (m/z): 558.6 [M+1]⁺.

### Example 42 (S)—N-(2,2-difluoro-1-(5-fluoro-1-neopentyl-6-(2-(trifluoromethyl)pyridin-3-yl)-1H-indol-3-yl)ethyl)propane-2-sulfonamide

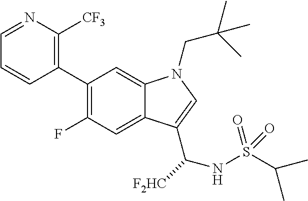 [SMILES: CC(C)S(=O)(=O)N[C@@H](c1cn(CC(C)(C)C)c2cc(-c3cccnc3C(F)(F)F)c(F)cc12)C(F)F]

### Step 1: (S)—N—((S)-2,2-difluoro-1-(5-fluoro-1-neopentyl-6-(2-(trifluoromethyl)pyridin-3-yl)-1H-indol-3-yl)ethyl)-2-methylpropane-2-sulfinamide

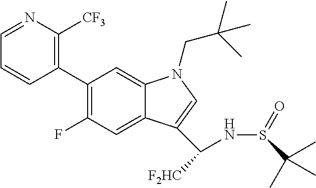 [SMILES: CC(C)(C)Cn1cc([C@H](N[S@@](=O)C(C)(C)C)C(F)F)c2cc(F)c(-c3cccnc3C(F)(F)F)cc21]

The title compound was prepared following the same general protocol as described for Step 6, Example 2, using (S)—N—((S)-1-(6-bromo-5-fluoro-1-neopentyl-1H-indol-3-yl)-2,2-difluoroethyl)-2-methylpropane-2-sulfinamide and (2-(trifluoromethyl)pyridin-3-yl)boronic acid. ESI-MS (m/z): 533.6 [M+1]⁺.

### Step 2: (S)-2,2-difluoro-1-(5-fluoro-1-neopentyl-6-(2-(trifluoromethyl)pyridin-3-yl)-1H-indol-3-yl)ethan-1-amine

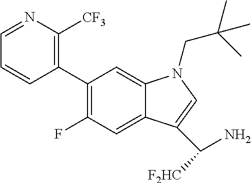 [SMILES: CC(C)(C)Cn1cc([C@H](N)C(F)F)c2cc(F)c(-c3cccnc3C(F)(F)F)cc21]

The title compound was prepared following the same general protocol as described for Step 3, Example 2, using (S)—N—((S)-2,2-difluoro-1-(5-fluoro-1-neopentyl-6-(2-(trifluoromethyl)pyridin-3-yl)-1H-indol-3-yl)ethyl)-2-methylpropane-2-sulfinamide.

Step 3: The title compound was prepared following the same general protocol as described for Step 4, Example 2, using (S)-2,2-difluoro-1-(5-fluoro-1-neopentyl-6-(2-(trifluoromethyl)pyridin-3-yl)-1H-indol-3-yl)ethan-1-amine and propane-2-sulfonyl chloride. ESI-MS (m/z): 535.6 [M+1]⁺.

### Example 43: (S)—N-(2,2-difluoro-1-(1-neopentyl-6-(2-(trifluoromethyl)pyridin-3-yl)-1H-indol-3-yl)ethyl)-sulfamoyldimethylamine

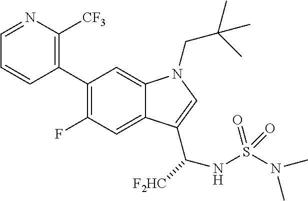 [SMILES: CN(C)S(=O)(=O)N[C@@H](c1cn(CC(C)(C)C)c2cc(-c3cccnc3C(F)(F)F)c(F)cc12)C(F)F]

The title compound was prepared following the same general protocol as described for Example 21 using (S)-2,2-difluoro-1-(5-fluoro-1-neopentyl-6-(2-(trifluoromethyl)pyridin-3-yl)-1H-indol-3-yl)ethan-1-amine and dimethylsulfamoyl chloride. ESI-MS (m/z): 536.5 [M+1]⁺.

### Example 44: (S)—N-(2,2-difluoro-1-(1-neopentyl-6-(2-(trifluoromethyl)pyridin-3-yl)-1H-indol-3-yl)ethyl)cyclobutane-1-sulfonamide-3,3-d2

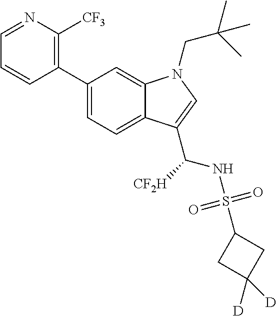 [SMILES: [2H]C1([2H])CC(S(=O)(=O)N[C@@H](c2cn(CC(C)(C)C)c3cc(-c4cccnc4C(F)(F)F)ccc23)C(F)F)C1]

The title compound was prepared following the same general protocol as described for Step 4, Example 2, using (S)-2,2-difluoro-1-(1-neopentyl-6-(2-(trifluoromethyl)pyridin-3-yl)-1H-indol-3-yl)ethan-1-amine and cyclobutane-1-sulfonyl chloride-3,3-d2. ESI-MS (m/z): 532.04 [M+1]⁺.

### Example 45: (S)—N-(2,2-difluoro-1-(5-fluoro-1-neopentyl-6-(2-(trifluoromethyl)pyridin-3-yl)-1H-indol-3-yl)ethyl)cyclobutane-1-sulfonamide-3,3-d2

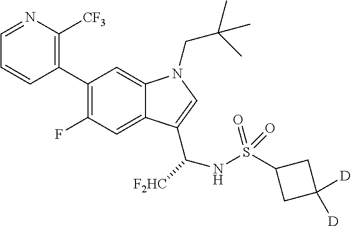 [SMILES: [2H]C1([2H])CC(S(=O)(=O)N[C@@H](c2cn(CC(C)(C)C)c3cc(-c4cccnc4C(F)(F)F)c(F)cc23)C(F)F)C1]

The title compound was prepared following the same general protocol as described for Step 4, Example 2, using (S)-2,2-difluoro-1-(5-fluoro-1-neopentyl-6-(2-(trifluoromethyl)pyridin-3-yl)-1H-indol-3-yl)ethan-1-amine and cyclobutane-1-sulfonyl chloride-3,3-d2. ESI-MS (m/z): 549.6 [M+1]⁺.

### Example 46: (S)—N-(2,2-difluoro-1-(1-neopentyl-6-(2-(trifluoromethyl)pyridin-3-yl)-1H-indol-3-yl)ethyl)-3-fluorocyclobutane-1-sulfonamide

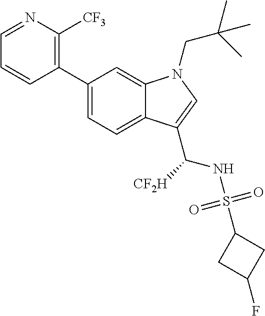 [SMILES: CC(C)(C)Cn1cc([C@H](NS(=O)(=O)C2CC(F)C2)C(F)F)c2ccc(-c3cccnc3C(F)(F)F)cc21]

The title compound was prepared following the same general protocol as described for Step 4, Example 2, using (S)-2,2-difluoro-1-(1-neopentyl-6-(2-(trifluoromethyl)pyridin-3-yl)-1H-indol-3-yl)ethan-1-amine and 3-fluorocyclobutane-1-sulfonyl chloride. ESI-MS (m/z): 548.13 [M+1]⁺.

### Example 47: (S)—N-(2,2-difluoro-1-(1-neopentyl-6-(3-(trifluoromethyl)pyrazin-2-yl)-1H-indol-3-yl)ethyl)cyclopropanesulfonamide

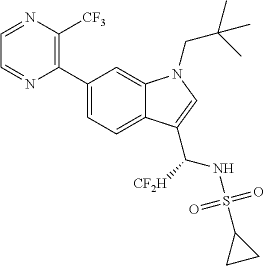 [SMILES: CC(C)(C)Cn1cc([C@H](NS(=O)(=O)C2CC2)C(F)F)c2ccc(-c3nccnc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-(trifluoromethyl)pyrazine. ESI-MS (m/z): 516.78 [M+1]⁺.

### Example 48: (S)—N-(1-(6-(3-cyanopyrazin-2-yl)-1-neopentyl-1H-indol-3-yl)-2,2-difluoroethyl)cyclopropanesulfonamide

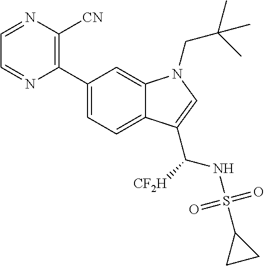 [SMILES: CC(C)(C)Cn1cc([C@H](NS(=O)(=O)C2CC2)C(F)F)c2ccc(-c3nccnc3C#N)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromopyrazine-2-carbonitrile. ¹H-NMR (CDCl₃, 400 MHz) δ 8.31 (d, J=2.5 Hz, 1H), 8.18 (d, J=2.5 Hz, 1H), 7.70 (d, J=8.9 Hz, 1H), 7.14 (s, 1H), 7.13 (s, 1H), 6.91 (dd, J1=2.2 Hz, J2=8.7 Hz, 1H), 6.15 (dd, J1=2.2 Hz, J2=55.96 Hz, 1H), 5.10 (dt, J1=2.2 Hz, J2=9.9 Hz, 1H), 4.76 (d, J=7.9 Hz, 1H), 3.79 (s, 2H), 2.24-2.32 (m, 1H), 1.09-1.06 (m, 2H), 0.92 (s, 9H), 0.84-0.80 (m, 2H).

### Example 49: (S)—N-(1-(6-(2-cyanopyridin-3-yl)-1-neopentyl-1H-indol-3-yl)-2,2-difluoroethyl)cyclopropanesulfonamide

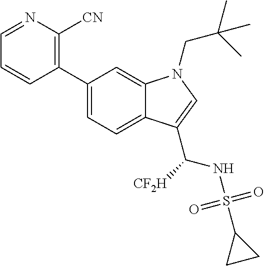 [SMILES: CC(C)(C)Cn1cc([C@H](NS(=O)(=O)C2CC2)C(F)F)c2ccc(-c3cccnc3C#N)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromopicolinonitrile. ESI-MS (m/z): 472.73 [M+1]⁺.

### Example 51: (S)—N-(2,2-difluoro-1-(1-neopentyl-6-(3-(trifluoromethyl)pyrazin-2-yl)-1H-indol-3-yl)ethyl)-sulfamoyldimethylamine

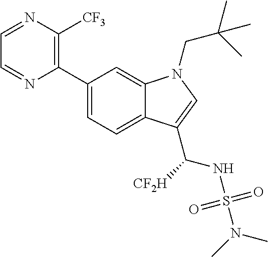 [SMILES: CN(C)S(=O)(=O)N[C@@H](c1cn(CC(C)(C)C)c2cc(-c3nccnc3C(F)(F)F)ccc12)C(F)F]

### Step 1: (S)—N—((S)-2,2-difluoro-1-(1-neopentyl-6-(4,4,5,5-tetramethyl-1,3,2-dioxaborolan-2-yl)-1H-indol-3-yl)ethyl)-2-methylpropane-2-sulfinamide

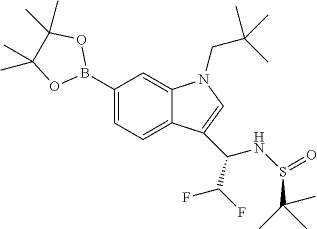 [SMILES: CC(C)(C)Cn1cc([C@H](N[S@@](=O)C(C)(C)C)C(F)F)c2ccc(B3OC(C)(C)C(C)(C)O3)cc21]

4,4,4′,4′,5,5,5′,5′-octamethyl-2,2′-bi(1,3,2-dioxaborolane) (128 mg, 0.5 mmol), KOAc (63 mg, 0.7 mmol) and Pd(dppf)Cl₂·DCM (18 mg, 0.03 mmol) was added to a solution of(S)—N—((S)-1-(6-bromo-1-neopentyl-1H-indol-3-yl)-2,2-difluoroethyl)-2-methylpropane-2-sulfinamide (112 mg, 0.25 mmol) in dioxane (2.5 mL) and the reaction mixture was degassed three times. The reaction was warmed to 100° C. for 10-16 hours. The reaction mixture was cooled and diluted with EtOAc (10 mL) and water (3 mL). The layers were separated, and the aqueous layer was extracted with EtOAc (2×5 mL). The combined organics were dried (Na₂SO₄) and filtered. The solvent was removed under reduced pressure and the residue was purified by combi-flash on silica gel to obtain the title compound as a solid (98 mg). ESI-MS (m/z): 497.3 [M+1]⁺.

### Step 2: (S)—N—((S)-2,2-difluoro-1-(1-neopentyl-6-(3-(trifluoromethyl)pyrazin-2-yl)-1H-indol-3-yl)ethyl)-2-methylpropane-2-sulfinamide

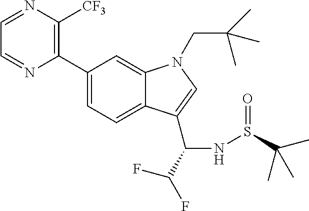 [SMILES: CC(C)(C)Cn1cc([C@H](N[S@@](=O)C(C)(C)C)C(F)F)c2ccc(-c3nccnc3C(F)(F)F)cc21]

Step 2: The title compound was prepared following the same general protocol as described for Step 6, Example 2, using (S)—N—((S)-2,2-difluoro-1-(1-neopentyl-6-(4,4,5,5-tetramethyl-1,3,2-dioxaborolan-2-yl)-1H-indol-3-yl)ethyl)-2-methylpropane-2-sulfinamide and 2-chloro-3-(trifluoromethyl)pyrazine. ESI-MS (m/z): 516.7 [M+1]⁺.

### Step 3: (S)-2,2-difluoro-1-(1-neopentyl-6-(3-(trifluoromethyl)pyrazin-2-yl)-1H-indol-3-yl)ethan-1-amine

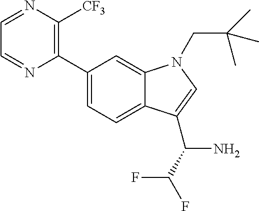 [SMILES: CC(C)(C)Cn1cc([C@H](N)C(F)F)c2ccc(-c3nccnc3C(F)(F)F)cc21]

The title compound was prepared following the same general protocol as described for Step 3, Example 2, using (S)—N—((S)-2,2-difluoro-1-(1-neopentyl-6-(3-(trifluoromethyl) pyrazin-2-yl)-1H-indol-3-yl)ethyl)-2-methylpropane-2-sulfinamide. ESI-MS (m/z): 395.89 [M-NH₂]⁺.

Step 4: The title compound was prepared following the same general protocol as described for Example 21, using (S)-2,2-difluoro-1-(1-neopentyl-6-(3-(trifluoromethyl)pyrazin-2-yl)-1H-indol-3-yl)ethan-1-amine and dimethylsulfamoyl chloride. ESI-MS (m/z): 519.67 [M+1]⁺.

### Example 52: (S)—N-(1-(6-(4-cyano-2-(trifluoromethyl)pyridin-3-yl)-1-neopentyl-1H-indol-3-yl)-2,2-difluoroethyl)cyclopropanesulfonamide

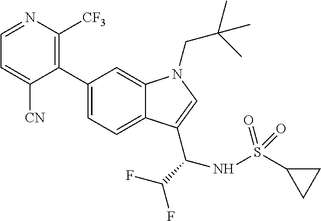 [SMILES: CC(C)(C)Cn1cc([C@H](NS(=O)(=O)C2CC2)C(F)F)c2ccc(-c3c(C#N)ccnc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-2-(trifluoromethyl)isonicotinonitrile. ESI-MS (m/z): 540.49 [M+1]⁺.

### Example 53: (S)—N-(2,2-difluoro-1-(6-(5-fluoro-3-(trifluoromethyl)pyridin-2-yl)-1-neopentyl-1H-indol-3-yl)ethyl)cyclopropanesulfonamide

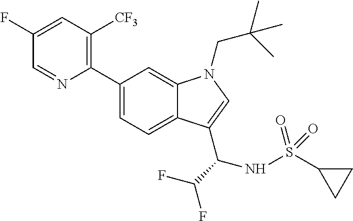 [SMILES: CC(C)(C)Cn1cc([C@H](NS(=O)(=O)C2CC2)C(F)F)c2ccc(-c3ncc(F)cc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-5-fluoro-3-(trifluoromethyl)pyridine. ESI-MS (m/z): 533.79 [M+1]⁺.

### Example 54: (S)—N-(2,2-difluoro-1-(5-fluoro-1-neopentyl-6-(2-(trifluoromethyl)phenyl)-1H-indol-3-yl)ethyl)-sulfamoyldimethylamine

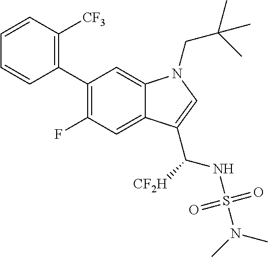 [SMILES: CN(C)S(=O)(=O)N[C@@H](c1cn(CC(C)(C)C)c2cc(-c3ccccc3C(F)(F)F)c(F)cc12)C(F)F]

The title compound was prepared following the same general protocol as described for Example 21 using (S)-2,2-difluoro-1-(5-fluoro-1-neopentyl-6-(2-(trifluoromethyl)phenyl)-1H-indol-3-yl)ethan-1-amine and dimethylsulfamoyl chloride. ESI-MS (m/z): 535.6 [M+1]⁺.

### Example 55: N-((1S)-1-(6-(4-cyano-2-(trifluoromethyl)pyridin-3-yl)-5-fluoro-1-neopentyl-1H-indol-3-yl)-2,2-difluoroethyl)cyclopropanesulfonamide

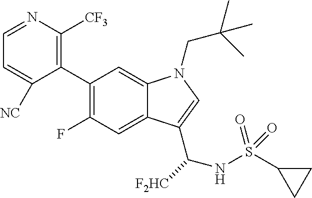 [SMILES: CC(C)(C)Cn1cc([C@H](NS(=O)(=O)C2CC2)C(F)F)c2cc(F)c(-c3c(C#N)ccnc3C(F)(F)F)cc21]

### Step 1:3-bromo-2-(trifluoromethyl)isonicotinic acid

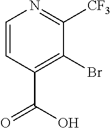 [SMILES: O=C(O)c1ccnc(C(F)(F)F)c1Br]

3-bromo-2-(trifluoromethyl)pyridine (0.85 g, 3.77 mmol) in THF (15 mL) was cooled to −78° C. under argon, LDA (1M in THF, 4.2 mL, 4.2 mmol) was added dropwise. The mixture was continued to stir at −78° C. for 30 min, and then dry ice was added. The reaction was warm to rt and the adjust to pH 5-6. The solvent was removed and the crude was purified by silica gel to obtain the title compound. ESI-MS (m/z): 269.4, 271.3 [M+1]⁺.

### Step 2:3-bromo-2-(trifluoromethyl)isonicotinamide

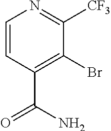 [SMILES: NC(=O)c1ccnc(C(F)(F)F)c1Br]

3-bromo-2-(trifluoromethyl)isonicotinic acid (0.741 g, 2.7 mmol) in DCM (10 mL) was added DMF (0.04 mL, 0.549 mmol), followed by slow addition of oxalyl chloride (0.72 mL, 8.23 mmol). The mixture was continued to stir for 2 h at rt. Then solvent was removed and the crude was redissolved with fresh DCM and added slowly to cold ammonia hydroxide (24%, 10 mL). The mixture was stirred at rt for 1 h, and DCM was added to dilute the reaction. The organic layer was separated and the aqueous layer was extracted with DCM twice. The organic layers were combined and dried over Na₂SO₄. The solvent was removed and the crude was purified by silica gel to obtain the title compound. ¹H NMR (CDCl₃, 400 MHz) δ 8.51 (d, J=5.2 Hz, 1H), 7.76 (d, J=4.7 Hz, 1H), 6.59 (s, 1H), 6.09 (s, 1H)

### Step 3:3-bromo-2-(trifluoromethyl)isonicotinonitrile

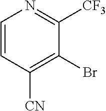 [SMILES: N#Cc1ccnc(C(F)(F)F)c1Br]

To a solution of 3-bromo-2-(trifluoromethyl)isonicotinamide (0.203 g, 0.755 mmol) in DCM (3 mL) was added Burgess reagent (0.27 g, 1.13 mmol), and then the mixture was stirred at 35° C. oil bath for 3 h. The completion was monitored by anal. HPLC. The mixture was diluted with DCM and washed with sat'd NaHCO₃, and brine and dried over Na₂SO₄. The solvent was removed and the crude was purified by silica gel to obtain the title compound. 1H NMR (CDCl₃, 400 MHz) δ 8.67 (d, J=5.2 Hz, 1H), 7.91 (d, J=5.2 Hz, 1H)

### Step 4: (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide

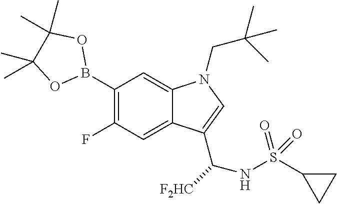 [SMILES: CC(C)(C)Cn1cc([C@H](NS(=O)(=O)C2CC2)C(F)F)c2cc(F)c(B3OC(C)(C)C(C)(C)O3)cc21]

To a mixture of 4,4,4′,4′,5,5,5′,5′-octamethyl-2,2′-bi(1,3,2-dioxaborolane) (1.3 eq), (S)—N-(1-(6-bromo-5-fluoro-1-neopentyl-1H-indol-3-yl)-2,2-difluoroethyl)cyclopropanesulfonamide (1 eq) and KOAc (1.5 eq) in toluene, was added Pd(dppf)Cl₂ (0.1 eq) under N₂ atmosphere. The reaction mixture was stirred at 110° C. for 2 h, at which time the solvent was removed. The residue was purified by chromatography on silica gel to give the desired product. ESI-MS (m/z): 514.5 [M+1]⁺.

### Step 5: N-((1S)-1-(6-(4-cyano-2-(trifluoromethyl)pyridin-3-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-2-(trifluoromethyl)isonicotinonitrile with anhydrous dioxane as solvent, and the reaction temperature 140° C. 1H NMR (CDCl₃, 400 MHz) δ 8.89 (d, J=5.2 Hz, 1H), 7.79 (d, J=5.2 Hz, 1H), 7.62 (d, J=10.7 Hz, 1H), 7.44 (d, J=5.8 Hz, 1H), 7.34 (s, 1H), 6.16 (td, J=55.8 Hz, 2.2 Hz, 1H), 5.29-5.09 (m, 1H), 4.82 (d, J=8.0 Hz, 1H), 3.92 (s, 2H), 2.44-2.41 (m, 1H), 2.21-2.28 (m, 2H), 0.93 (s, 9H), 0.92-0.83 (m, 2H)

### Example 56: (S)—N-(2,2-difluoro-1-(5-fluoro-6-(5-fluoro-3-(trifluoromethyl)pyridin-2-yl)-1-neopentyl-1H-indol-3-yl)ethyl)cyclopropanesulfonamide

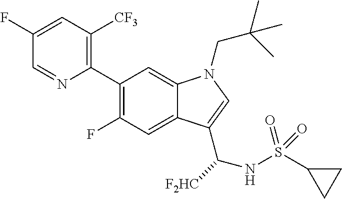 [SMILES: CC(C)(C)Cn1cc([C@H](NS(=O)(=O)C2CC2)C(F)F)c2cc(F)c(-c3ncc(F)cc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-chloro-5-fluoro-3-(trifluoromethyl)pyridine with anhydrous dioxane as solvent, and the reaction temperature 140° C. ESI-MS (m/z): 551.5 [M+1]⁺.

### Example 57: (S)—N-(2,2-difluoro-1-(5-fluoro-1-neopentyl-6-(3-(trifluoromethyl)pyrazin-2-yl)-1H-indol-3-yl)ethyl)cyclopropanesulfonamide

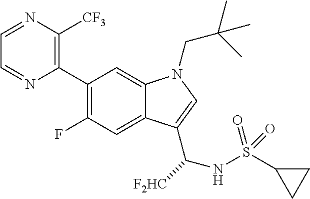 [SMILES: CC(C)(C)Cn1cc([C@H](NS(=O)(=O)C2CC2)C(F)F)c2cc(F)c(-c3nccnc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-chloro-3-(trifluoromethyl)pyrazine with anhydrous dioxane as solvent, and the reaction temperature 140° C. ESI-MS (m/z): 534.6 [M+1]⁺.

### Example 58: N-[1-[6-(3-chloro-2-pyridyl)-1-(2,2-dimethylpropyl)-5-fluoro-indol-3-yl]ethyl]cyclopropanesulfonamide

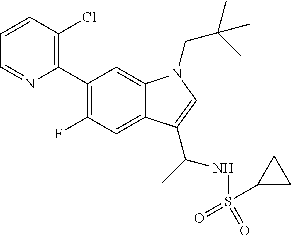 [SMILES: CC(NS(=O)(=O)C1CC1)c1cn(CC(C)(C)C)c2cc(-c3ncccc3Cl)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (120 mg, 250.82 μmol) and 2,3-dichloropyridine (37.12 mg, 250.82 μmol) in dioxane (2 mL) and water (499.30 μL) was added sodium carbonate (53.17 mg, 501.64 μmol, 21.00 μL) and cyclopentyl(diphenyl)phosphane;dichloropalladiumiron (27.53 mg, 37.62 μmol). It was stirred at 90° C. for 5 hr., at which time the reaction was diluted with water (30 mL) and extracted with EA (3×50 mL). The combined organic layers were washed with brine (50 mL), dried over Na₂SO₄, filtered and concentrated. The residue obtained was purified by Prep-HPLC to give the title compound (15 mg, 32.33 μmol, 12.89% yield) as a white solid. ¹H NMR (400 MHz, CDCl₃) δ 8.62 (dd, J=4.7, 1.5 Hz, 1H), 7.82 (dd, J=8.1, 1.5 Hz, 1H), 7.51 (d, J=10.5 Hz, 1H), 7.38 (d, J=5.7 Hz, 1H), 7.29 (dd, J=8.1, 4.7 Hz, 1H), 7.13 (s, 1H), 4.96 (t, J=6.7 Hz, 1H), 4.46 (d, J=6.6 Hz, 1H), 3.87 (s, 2H), 2.23 (ddd, J=8.0, 4.8, 3.2 Hz, 1H), 1.75 (d, J=6.8 Hz, 3H), 1.12-1.07 (m, 2H), 1.00 (s, 9H), 0.84-0.79 (m, 2H). MS Found: 462.2 [M+H]⁺.

### Example 59: N-[1-[1-(2,2-dimethylpropyl)-5-fluoro-6-(3-fluoro-2-pyridyl)indol-3-yl]ethyl]cyclopropanesulfonamide

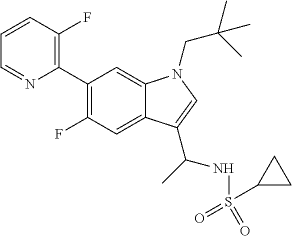 [SMILES: CC(NS(=O)(=O)C1CC1)c1cn(CC(C)(C)C)c2cc(-c3ncccc3F)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (120 mg, 250.82 μmol) and 2-bromo-3-fluoro-pyridine (32.99 mg, 250.82 μmol) in dioxane (1.99 mL) and water (497.53 μL) was added sodium carbonate (53.17 mg, 501.64 μmol, 21.00 μL) and cyclopentyl(diphenyl)phosphane;dichloropalladium;iron (27.53 mg, 37.62 μmol). The solution was stirred at 90° C. for 5 hr., at which time the reaction was diluted with water (30 mL) and extracted with EA (3×50 mL). The combined organic layers were washed with brine (50 mL), dried over Na₂SO₄, filtered and concentrated. The residue obtained was purified by Prep-HPLC to give the title compound (29 mg, 64.80 μmol, 25.83% yield) as a white solid. ¹H NMR (400 MHz, CDCl3) δ 8.49 (dt, J=4.6, 1.4 Hz, 1H), 7.46 (s, 1H), 7.45 (d, J=1.4 Hz, 1H), 7.44-7.42 (m, 1H), 7.30-7.25 (m, 1H), 7.07 (s, 1H), 4.91-4.86 (m, 1H), 4.38 (d, J=6.5 Hz, 1H), 3.83 (d, J=1.7 Hz, 2H), 2.16 (ddd, J=8.0, 4.8, 3.2 Hz, 1H), 1.68 (d, J=6.8 Hz, 3H), 1.03 (dt, J=7.8, 2.5 Hz, 2H), 0.93 (s, 9H), 0.75 (ddd, J=8.0, 3.0, 1.0 Hz, 2H). MS Found: 448.2 [M+H]⁺.

### Example 60: N-(1-(6-(3-cyclopropylpyridin-2-yl)-5-fluoro-1-neopentyl-1H-indol-3-yl)ethyl)cyclopropanesulfonamide

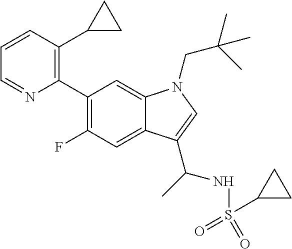 [SMILES: CC(NS(=O)(=O)C1CC1)c1cn(CC(C)(C)C)c2cc(-c3ncccc3C3CC3)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (100 mg, 209.02 μmol) and 2-chloro-3-cyclopropanepyridine (34.69 mg, 209.02 μmol) in water (0.5 mL) and dioxane (2 mL) was added sodium carbonate (22.15 mg, 209.02 μmol, 8.75 μL) and Pd(dppf)Cl₂ (23 mg, 31.43 μmol) and stirred at 90° C. for 5 hr. The mixture was then poured into water and extracted with ethyl acetate. The organic layer dried over Na₂SO₄ and concentrated under press to give crude product. The crude product was purified by Prep HPLC to give the title compound (13 mg, 27.68 μmol, 13.24% yield). ¹H NMR (400 MHz, CDCl₃) δ 8.50 (dd, J=4.6, 1.7 Hz, 1H), 7.49 (d, J=10.4 Hz, 1H), 7.37 (d, J=5.8 Hz, 1H), 7.31-7.27 (m, 1H), 7.24 (d, J=4.6 Hz, 1H), 7.11 (s, 1H), 4.96 (p, J=6.6 Hz, 1H), 4.46 (d, J=6.4 Hz, 1H), 3.87 (d, J=1.6 Hz, 2H), 2.24 (td, J=7.9, 3.9 Hz, 1H), 1.89-1.80 (m, 1H), 1.75 (d, J=6.8 Hz, 3H), 1.15-1.09 (m, 2H), 0.99 (s, 9H), 0.86-0.79 (m, 4H), 0.63 (d, J=5.1 Hz, 2H). MS Found: 470.2 [M+H]⁺.

### Example 61: N-(1-(6-(3-(difluoromethoxy)pyridin-2-yl)-5-fluoro-1-neopentyl-1H-indol-3-yl)ethyl)cyclopropanesulfonamide

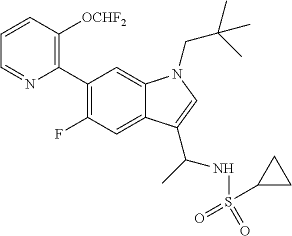 [SMILES: CC(NS(=O)(=O)C1CC1)c1cn(CC(C)(C)C)c2cc(-c3ncccc3OC(F)F)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (100 mg, 209.02 μmol) and 2-chloro-3-(difluoromethoxy)pyridine (37.53 mg, 209.02 μmol) in water (0.5 mL) and dioxane (2 mL) was added sodium carbonate (22.15 mg, 209.02 μmol, 8.75 μL), Pd(dppf)Cl₂ (23 mg, 31.43 μmol) and stirred at 90° C. for 5 hr. The reaction mixture was poured into water and extracted with ethyl acetate. The organic layer dried over Na₂SO₄ and concentrated under press to give crude product. The crude product was purified by Prep HPLC to give the title compound (5 mg, 10.09 μmol, 4.83% yield). ¹H NMR (400 MHz, CDCl₃) δ 8.50 (dd, J=4.6, 1.7 Hz, 1H), 7.49 (d, J=10.4 Hz, 1H), 7.37 (d, J=5.8 Hz, 1H), 7.31-7.27 (m, 1H), 7.24 (d, J=4.6 Hz, 1H), 7.11 (s, 1H), 4.96 (p, J=6.6 Hz, 1H), 4.46 (d, J=6.4 Hz, 1H), 3.87 (d, J=1.6 Hz, 2H), 2.24 (td, J=7.9, 3.9 Hz, 1H), 1.89-1.80 (m, 1H), 1.75 (d, J=6.8 Hz, 3H), 1.15-1.09 (m, 2H), 0.99 (s, 9H), 0.86-0.79 (m, 4H), 0.63 (d, J=5.1 Hz, 2H). MS Found: 496.2 [M+H]⁺.

### Example 62: N-(1-(6-(3-chloro-5-fluoropyridin-2-yl)-5-fluoro-1-neopentyl-1H-indol-3-yl)ethyl)cyclopropanesulfonamide

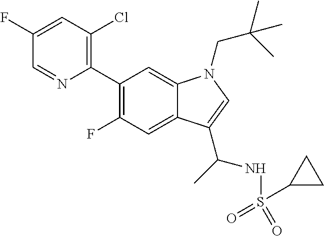 [SMILES: CC(NS(=O)(=O)C1CC1)c1cn(CC(C)(C)C)c2cc(-c3ncc(F)cc3Cl)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (100 mg, 209.02 μmol) and 2,3-dichloro-5-fluoropyridine (34.69 mg, 209.02 μmol) in water (0.5 mL) and dioxane (2 mL) was added sodium carbonate (34.69 mg, 209.02 μmol, 8.75 μL), Pd(dppf)Cl₂ (23 mg, 31.43 μmol) and stirred at 90° C. for 5 hr. The mixture was then poured into water and extracted with ethyl acetate. The organic layer dried over Na₂SO₄ and concentrated under press to give crude product. The crude product was purified by Prep HPLC to give the title compound (13 mg, 26.97 μmol, 12.90% yield). ¹H NMR (400 MHz, CDCl₃) δ 8.51 (d, J=2.6 Hz, 1H), 7.62 (d, J=7.8 Hz, 1H), 7.51 (d, J=10.5 Hz, 1H), 7.34 (d, J=5.7 Hz, 1H), 7.13 (s, 1H), 4.98-4.93 (m, 1H), 4.45 (d, J=6.6 Hz, 1H), 3.87 (s, 2H), 2.25 (ddd, J=8.0, 4.8, 3.2 Hz, 1H), 1.74 (d, J=6.8 Hz, 3H), 1.13-1.09 (m, 2H), 1.00 (s, 9H), 0.85-0.80 (m, 2H). MS Found: 482.1 [M+H]⁺.

### Example 63: N-(1-(6-(3-cyano-5-fluoropyridin-2-yl)-5-fluoro-1-neopentyl-1H-indol-3-yl)ethyl)cyclopropanesulfonamide

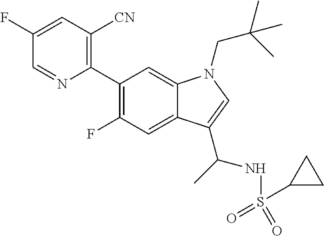 [SMILES: CC(NS(=O)(=O)C1CC1)c1cn(CC(C)(C)C)c2cc(-c3ncc(F)cc3C#N)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (100 mg, 209.02 μmol) and 2-chloro-5-fluoronicotinonitrile (32.71 mg, 209.02 μmol) in water (0.5 mL) and dioxane (2 mL) was added sodium carbonate (22.15 mg, 209.02 μmol, 8.75 μL), Pd(dppf)Cl₂ (23 mg, 31.43 μmol) and stirred at 90° C. for 5 hr. The mixture was poured into water and extracted with ethyl acetate. The organic layer dried over Na₂SO₄ and concentrated under press to give crude product. The crude product was purified by Prep HPLC to give the title compound (5 mg, 10.58 μmol, 5.06% yield). ¹H NMR (400 MHz, CDCl₃) δ 8.78 (d, J=2.8 Hz, 1H), 7.81 (dd, J=7.5, 2.8 Hz, 1H), 7.58 (d, J=10.9 Hz, 1H), 7.47 (d, J=5.8 Hz, 1H), 7.18 (s, 1H), 5.00-4.90 (m, 1H), 4.49 (d, J=6.3 Hz, 1H), 3.90 (s, 2H), 2.26-2.21 (m, 1H), 1.74 (d, J=6.8 Hz, 3H), 1.13-1.07 (m, 2H), 1.01 (s, 9H), 0.82 (t, J=8.3 Hz, 2H). MS Found: 473.2 [M+H]⁺.

### Example 64: N-(1-(6-(2-cyano-6-fluorophenyl)-1-neopentyl-1H-indol-3-yl)ethyl)cyclopropanesulfonamide

### Step 1:6-bromo-1-(2,2-dimethylpropyl)-5-fluoro-indole

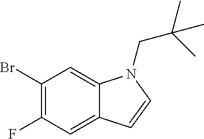 [SMILES: CC(C)(C)Cn1ccc2cc(F)c(Br)cc21]

To a stirred solution of 6-bromo-5-fluoro-1H-indole (5.5 g, 25.70 mmol) and Potassium Iodide (4.27 g, 25.70 mmol, 1.37 mL) in DMF (50 mL) was added sodium hydride (4.11 g, 102.79 mmol, 60% purity) in an ice-bath under an N₂ Atmosphere. The mixture was stirred at 50° C. for 15 min., at which time 1-bromo-2,2-dimethyl-propane (7.76 g, 51.39 mmol) was added to the mixture. The mixture was stirred at 80° C. for 16 h. TLC, at which time the reaction was poured into ice water (100 mL) and extracted with EA (100 mL×2). The combined organic layers were dried over anhydrous Na₂SO₄, filtered and concentrated. The residue obtained was purified by silica gel column chromatography (PE) to give 6-bromo-1-(2,2-dimethylpropyl)-5-fluoro-indole (6 g, 21.11 mmol, 82.17% yield) as a light-yellow oil.

### Step 2:1-[6-bromo-1-(2,2-dimethylpropyl)-5-fluoro-indol-3-yl]ethanone

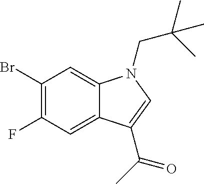 [SMILES: CC(=O)c1cn(CC(C)(C)C)c2cc(Br)c(F)cc12]

A mixture of 6-bromo-1-(2,2-dimethylpropyl)-5-fluoro-indole (6 g, 21.11 mmol) in DCM (60 mL) was added Diethylaluminium chloride (2 M, 15.84 mL) at 0° C. slowly under an atmosphere of N₂. After addition, the mixture was stirred at 0° C. for 1 hours, at which time Acetyl chloride (2.49 g, 31.67 mmol, 1.92 mL) in DCM (10 mL) was added to the above mixture slowly and stirred at 0° C. for 2 hours. The mixture was then quenched with sat. Na₂HCO₃ (20 mL). The mixture was then with DCM (30 mL×3). The organic layers were concentrated in vacuo and the product was purified by silica gel chromatograph (EA:PE, 0% to 10%) to give 1-[6-bromo-1-(2,2-dimethylpropyl)-5-fluoro-indol-3-yl]ethanone (5 g, 15.33 mmol, 72.59% yield) as a white solid.

### Step 3: N-(1-(6-bromo-5-fluoro-1-neopentyl-1H-indol-3-yl)ethylidene)cyclopropanesulfonamide

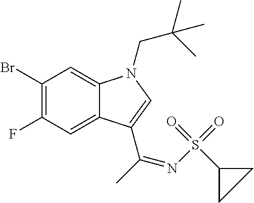 [SMILES: C/C(=N/S(=O)(=O)C1CC1)c1cn(CC(C)(C)C)c2cc(Br)c(F)cc12]

To a mixture of 1-(6-bromo-5-fluoro-1-neopentyl-1H-indol-3-yl)ethanone (5 g, 15.33 mmol) in Ti(OiPr)₄ (20 mL) was added cyclopropanesulfonamide (3.71 g, 30.66 mmol). The mixture was then stirred at 130° C. for 12 hours, at which time the mixture was poured into the H₂O (100 mL). Then sat·NaHCO₃ (20 mL) was added and extracted with EtOAc (50 mL×3), the organic layers were washed by brine and concentrated in vacuo to give a crude product. The crude product was purified by silica gel chromatography eluting with PE:EA=10:1 to afford (Z)—N-(1-(6-bromo-5-fluoro-1-neopentyl-1H-indol-3-yl)ethylidene)cyclopropanesulfonamide (5.1 g, 11.88 mmol, 77.50% yield) as a brown solid.

### Step 4: N-(1-(6-bromo-5-fluoro-1-neopentyl-1H-indol-3-yl)ethyl)cyclopropanesulfonamide

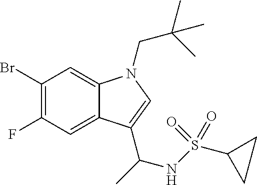 [SMILES: CC(NS(=O)(=O)C1CC1)c1cn(CC(C)(C)C)c2cc(Br)c(F)cc12]

To A mixture of (Z)—N-(1-(6-bromo-5-fluoro-1-neopentyl-1H-indol-3-yl)ethylidene)cyclopropanesulfonamide (9.5 g, 22.13 mmol) in methanol (100 mL) was added Sodium borohydride (1.67 g, 44.25 mmol, 1.56 mL) slowly at 0° C. under an atmosphere of N₂. After addition, the mixture was stirred at 0° C. for 2 hours and stirred at RT for 12 hours. The mixture was then diluted with water and extracted with ethyl acetate. The organic layer was washed with brine, dried over sodium carbonate and concentrated under reduced pressure. The crude product was purified by silica gel chromatography eluting with PE:EA=10:1 to afford N-(1-(6-bromo-5-fluoro-1-neopentyl-1H-indol-3-yl)ethyl)cyclopropanesulfonamide (7.6 g, 17.62 mmol, 79.63% yield) as a brown solid.

### Step 5: N-(1-(5-fluoro-1-neopentyl-6-(4,4,5,5-tetramethyl-1,3,2-dioxaborolan-2-yl)-1H-indol-3-yl)ethyl)cyclopropanesulfonamid

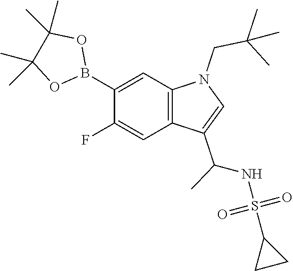 [SMILES: CC(NS(=O)(=O)C1CC1)c1cn(CC(C)(C)C)c2cc(B3OC(C)(C)C(C)(C)O3)c(F)cc12]

To a mixture of N-(1-(6-bromo-5-fluoro-1-neopentyl-1H-indol-3-yl)ethyl)cyclopropanesulfonamide (7.5 g, 17.39 mmol) and 4,4,5,5-tetramethyl-2-(4,4,5,5-tetramethyl-1,3,2-dioxaborolan-2-yl)-1,3,2-dioxaborolane (6.62 g, 26.08 mmol), Pd(dppf)Cl₂ (1.27 g, 1.74 mmol) and potassium acetate (5.12 g, 52.16 mmol, 3.26 mL) in anhydrous dioxane (100 mL) was stirred at 80° C. for 3 h under Nitrogen. The mixture was diluted with water and extracted with ethyl acetate. The organic layer was washed with brine, dried over sodium carbonate and concentrated under reduced pressure. The crude product was purified by silica chromatography eluting with PE:EA=5:1 to afford N-(1-(5-fluoro-1-neopentyl-6-(4,4,5,5-tetramethyl-1,3,2-dioxaborolan-2-yl)-1H-indol-3-yl)ethyl)cyclopropanesulfonamide (5.5 g, 8.05 mmol, 46.28% yield, 70% purity) as a light yellow oil.

### Step 6: N-(1-(6-(2-cyano-6-fluorophenyl)-1-neopentyl-1H-indol-3-yl)ethyl)cyclopropanesulfonamide

To a mixture of N-(1-(5-fluoro-1-neopentyl-6-(4,4,5,5-tetramethyl-1,3,2-dioxaborolan-2-yl)-1H-indol-3-yl)ethyl)cyclopropanesulfonamide (100 mg, 209.02 μmol) in water (1 mL) and dioxane (5 mL) was added 2-chloro-5-fluoropyridine (27.49 mg, 209.02 μmol, 21.20 μL). Then Pd(dppf)Cl₂ (15.29 mg, 20.90 μmol) and Sodium Carbonate (66.46 mg, 627.06 μmol, 26.25 μL) were added to the above mixture. The mixture was purged by N₂ for 2 times and stirred at 90° C. for 5 hours. The mixture was diluted with water and extracted with ethyl acetate. The organic layer was washed with brine, dried over sodium carbonate and concentrated under reduced pressure, which was purified by Prep HPLC to get N-(1-(5-fluoro-6-(5-fluoropyridin-2-yl)-1-neopentyl-1H-indol-3-yl)ethyl)cyclopropanesulfonamide (30 mg, 67.03 μmol, 32.1% yield) as a white solid. ¹H NMR (400 MHz, CDCl₃) δ 8.51 (d, J=2.9 Hz, 1H), 7.85 (d, J=6.4 Hz, 1H), 7.80 (ddd, J=8.8, 4.4, 2.0 Hz, 1H), 7.46-7.38 (m, 2H), 7.06 (s, 1H), 4.88 (t, J=6.8 Hz, 1H), 4.37 (d, J=6.7 Hz, 1H), 3.87 (d, J=1.7 Hz, 2H), 2.17 (tt, J=8.0, 4.9 Hz, 1H), 1.67 (d, J=6.8 Hz, 3H), 1.08-1.02 (m, 2H), 0.94 (s, 9H), 0.78-0.72 (m, 2H). MS Found: 448.2 [M+1]⁺.

### Example 65: N-(1-(6-(2-(difluoromethyl)pyridin-3-yl)-5-fluoro-1-neopentyl-1H-indol-3-yl)ethyl)cyclopropanesulfonamide

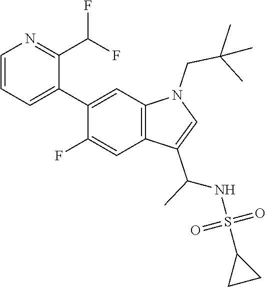 [SMILES: CC(NS(=O)(=O)C1CC1)c1cn(CC(C)(C)C)c2cc(-c3cccnc3C(F)F)c(F)cc12]

To a mixture of N-(1-(5-fluoro-1-neopentyl-6-(4,4,5,5-tetramethyl-1,3,2-dioxaborolan-2-yl)-1H-indol-3-yl)ethyl)cyclopropanesulfonamide (100 mg, 209.02 μmol) in water (1 mL) and dioxane (5 mL) was added 3-bromo-2-(difluoromethyl)pyridine (43.48 mg, 209.02 μmol), at which time Pd(dppf)Cl₂ (15.29 mg, 20.90 μmol) and Sodium Carbonate (66.46 mg, 627.06 μmol, 26.25 μL) were added to the above mixture. The mixture was purged with N₂ 2 times and stirred at 90° C. for 5 hours. The mixture was then diluted with water and extracted with ethyl acetate. The organic layer was washed with brine, dried over sodium carbonate, concentrated under reduced pressure and purified by Prep HPLC to give N-(1-(6-(2-(difluoromethyl)pyridin-3-yl)-5-fluoro-1-neopentyl-1H-indol-3-yl)ethyl)cyclopropanesulfonamide (32 mg, 66.73 μmol, 31.92% yield) as a white solid. ¹H NMR (400 MHz, CDCl₃) δ 8.77 (dd, J=4.7, 1.5 Hz, 1H), 7.77 (d, J=7.8 Hz, 1H), 7.57 (d, J=10.3 Hz, 1H), 7.49 (dd, J=7.8, 4.7 Hz, 1H), 7.22 (d, J=6.0 Hz, 1H), 7.14 (s, 1H), 6.60 (t, J=54.2 Hz, 1H), 4.97 (t, J=6.8 Hz, 1H), 4.44 (d, J=6.8 Hz, 1H), 3.86 (s, 2H), 2.33 (tt, J=8.0, 4.8 Hz, 1H), 1.76 (d, J=6.8 Hz, 3H), 1.16 (d, J=4.8 Hz, 2H), 0.99 (s, 9H), 0.92-0.86 (m, 2H). MS Found: 480.2 [M+H]⁺.

### Example 66: N-(1-(5-fluoro-6-(5-fluoro-2-methylpyridin-3-yl)-1-neopentyl-1H-indol-3-yl)ethyl)cyclopropanesulfonamide

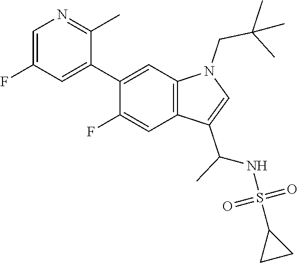 [SMILES: Cc1ncc(F)cc1-c1cc2c(cc1F)c(C(C)NS(=O)(=O)C1CC1)cn2CC(C)(C)C]

To a mixture of N-(1-(5-fluoro-1-neopentyl-6-(4,4,5,5-tetramethyl-1,3,2-dioxaborolan-2-yl)-1H-indol-3-yl)ethyl)cyclopropanesulfonamide (100 mg, 209.02 μmol) in dioxane (5 mL) and water (1 mL) was added 3-bromo-5-fluoro-2-methylpyridine (39.72 mg, 209.02 μmol), at which time Pd(dppf)Cl₂ (15.29 mg, 20.90 μmol) and Sodium Carbonate (66.46 mg, 627.06 μmol, 26.25 μL) were added to the above mixture. The mixture was then purged with N₂ 2 times and stirred at 90° C. for 5 hours. The mixture was diluted with water, extracted with ethyl acetate, the organic layer was washed with brine, dried over sodium carbonate and concentrated under reduced pressure. The crude product was purified by Prep HPLC to give N-(1-(5-fluoro-6-(5-fluoro-2-methylpyridin-3-yl)-1-neopentyl-1H-indol-3-yl)ethyl)cyclopropanesulfonamide (30 mg, 65.00 μmol, 31.1% yield) as a white solid. ¹H NMR (400 MHz, CDCl₃) δ 8.41 (d, J=2.8 Hz, 1H), 7.54 (d, J=10.3 Hz, 1H), 7.30 (dd, J=8.7, 2.8 Hz, 1H), 7.13 (t, J=2.9 Hz, 2H), 4.96 (dd, J=13.5, 6.8 Hz, 1H), 4.44 (d, J=6.9 Hz, 1H), 3.86 (s, 2H), 2.42 (s, 3H), 2.34 (ddd, J=8.0, 4.8, 3.1 Hz, 1H), 1.76 (d, J=6.8 Hz, 3H), 1.17 (dd, J=4.9, 2.3 Hz, 2H), 1.00 (s, 9H), 0.92-0.87 (m, 2H). MS Found: 446.2 [M+H]⁺.

### Example 67: N-(1-(6-(2-cyano-6-fluorophenyl)-1-neopentyl-1H-indol-3-yl)ethyl)cyclopropanesulfonamide

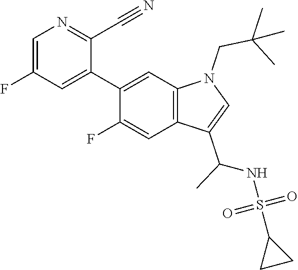 [SMILES: CC(NS(=O)(=O)C1CC1)c1cn(CC(C)(C)C)c2cc(-c3cc(F)cnc3C#N)c(F)cc12]

To a mixture of N-(1-(5-fluoro-1-neopentyl-6-(4,4,5,5-tetramethyl-1,3,2-dioxaborolan-2-yl)-1H-indol-3-yl)ethyl)cyclopropanesulfonamide (100 mg, 209.02 μmol) in water (1 mL) and dioxane (5 mL) was added 3-bromo-5-fluoropicolinonitrile (42.01 mg, 209.02 μmol), at which time Pd(dppf)Cl₂ (15.29 mg, 20.90 μmol) and Sodium Carbonate (66.46 mg, 627.06 μmol, 26.25 μL) were added to the above mixture. The mixture was purged with N₂ 2 times and stirred at 90° C. for 5 hours. The mixture was diluted with water, extracted with ethyl acetate, the organic layer was washed with brine, dried over sodium carbonate and concentrated under reduced pressure. The crude was purified by Prep HPLC to give N-(1-(6-(2-cyano-5-fluoropyridin-3-yl)-5-fluoro-1-neopentyl-1H-indol-3-yl)ethyl)cyclopropanesulfonamide (30 mg, 63.49 μmol, 30.37% yield) as a white solid. ¹H NMR (400 MHz, CDCl3) δ 8.58 (d, J=2.7 Hz, 1H), 7.67-7.61 (m, 2H), 7.41 (d, J=6.0 Hz, 1H), 7.19 (s, 1H), 5.01-4.94 (m, 1H), 4.44 (d, J=6.7 Hz, 1H), 3.90 (s, 2H), 2.34-2.26 (m, 1H), 1.75 (d, J=6.8 Hz, 3H), 1.14 (dd, J=5.2, 3.0 Hz, 2H), 1.02 (s, 9H), 0.92-0.84 (m, 2H). MS Found: 490.2 [M+18]⁺.

### Example 68: N-(1-(5-fluoro-6-(5-fluoropyridin-3-yl)-1-neopentyl-1H-indol-3-yl)ethyl)cyclopropanesulfonamide

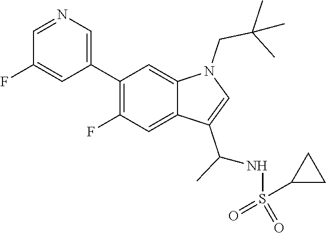 [SMILES: CC(NS(=O)(=O)C1CC1)c1cn(CC(C)(C)C)c2cc(-c3cncc(F)c3)c(F)cc12]

To a mixture of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (200 mg, 418 μmol) in dioxane:H₂O=4:1 (10 mL) was added 3-bromo-5-fluoropyridine (73.60 mg, 418 μmol), at which time sodium carbonate (88.6 mg, 831.6 μmol, 38.82 μL) and Pd(dppf)Cl₂ were added to the above mixture. The mixture was then purged with N₂ 2 times and stirred at 90° C. for 12 hours. The mixture was then diluted with water, extracted with ethyl acetate, the organic layer was washed with brine, dried over sodium carbonate and concentrated under reduced pressure. The crude product was purified by Prep HPLC to give N-(1-(5-fluoro-6-(5-fluoropyridin-3-yl)-1-neopentyl-1H-indol-3-yl)ethyl)cyclopropanesulfonamide (9.25 mg, 2.067 μmol) as a yellow solid. ¹H NMR (400 MHz, CDCl3) d 8.60 (s, 1H), 8.45 (d, J=2.6 Hz, 1H), 7.64 (dd, J=9.6, 2.4 Hz, 1H), 7.57 (d, J=11.3 Hz, 1H), 7.32 (d, J=6.2 Hz, 1H), 7.14 (s, 1H), 4.95 (p, J=6.8 Hz, 1H), 4.62 (d, J=6.8 Hz, 1H), 3.89 (s, 2H), 2.35-2.26 (m, 1H), 1.74 (d, J=6.8 Hz, 3H), 1.14 (d, J=4.5 Hz, 2H), 1.01 (s, 9H), 0.89-0.83 (m, 2H). MS Found: 448.2 [M+1]⁺.

### Example 69: N-(1-(6-(6-cyanopyridin-3-yl)-5-fluoro-1-neopentyl-1H-indol-3-yl)ethyl)cyclopropanesulfonamide

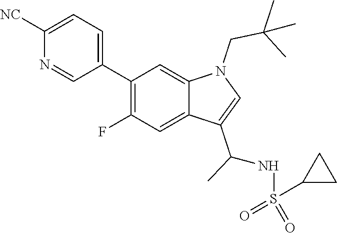 [SMILES: CC(NS(=O)(=O)C1CC1)c1cn(CC(C)(C)C)c2cc(-c3ccc(C#N)nc3)c(F)cc12]

To a solution of N-(1-(5-fluoro-1-neopentyl-6-(4,4,5,5-tetramethyl-1,3,2-dioxaborolan-2-yl)-1H-indol-3-yl)ethyl)cyclopropanesulfonamide (100 mg, 209.02 μmol) and 5-bromopicolinonitrile (38.25 mg, 209.02 μmol) in water (0.5 mL) and dioxane (2 mL) was added sodium carbonate (22.15 mg, 209.02 μmol, 8.75 μL), Pd(dppf)Cl₂ (23 mg, 31.43 μmol) and stirred at 90° C. for 5 hr. The mixture was then poured into water and extracted with ethyl acetate. The organic layer dried over Na₂SO₄ and concentrated under reduced press to give crude product. The crude product was purified by Prep HPLC to give the title compound (8 mg, 17.60 μmol, 8.42% yield). ¹H NMR (400 MHz, CDCl₃) δ 8.67 (d, J=9.9 Hz, 1H), 8.60 (dd, J=7.6, 5.6 Hz, 1H), 7.56 (d, J=10.5 Hz, 1H), 7.29 (d, J=5.8 Hz, 1H), 7.18-7.14 (m, 1H), 7.14 (s, 1H), 4.97 (dd, J=13.3, 6.5 Hz, 1H), 4.45 (s, 1H), 3.88 (s, 2H), 2.31 (ddd, J=8.0, 4.8, 3.1 Hz, 1H), 1.75 (d, J=6.8 Hz, 3H), 1.15 (dd, J=4.9, 2.1 Hz, 2H), 1.01 (s, 9H), 0.89-0.86 (m, 2H). MS Found: 455.2 [M+1]⁺.

### Example 70: N-(1-(5-fluoro-1-neopentyl-6-(6-(trifluoromethyl)pyridin-3-yl)-1H-indol-3-yl)ethyl)cyclopropanesulfonamide

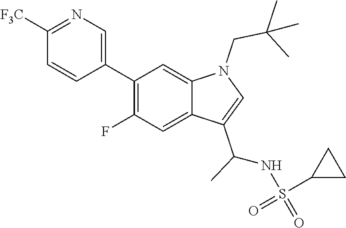 [SMILES: CC(NS(=O)(=O)C1CC1)c1cn(CC(C)(C)C)c2cc(-c3ccc(C(F)(F)F)nc3)c(F)cc12]

To a mixture of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (110 mg, 229.92 μmol) and 5-bromo-2-(trifluoromethyl)pyridine (51.96 mg, 229.92 μmol) in dioxane (2.00 mL) was added water (499.65 μL) at which time sodium carbonate (48.74 mg, 459.84 μmol, 19.25 μL) was added to the mixture. Cyclopentyl(diphenyl)phosphane;dichloropalladium;iron (25.24 mg, 34.49 μmol) was then added and bubbled with N₂ for 3 min. at which time the reaction was stirred at 90° C. for 5 h. The reaction was then diluted with water (20 mL) and extracted with EA (20 mL×3), the organic layer was combined, washed with brine, dried over Na₂SO₄ and concentrated to give crude product. The crude product was purified with a silica gel column (EA:PE=0%-70%) and concentrated to give N-[1-[1-(2,2-dimethylpropyl)-5-fluoro-6-[6-(trifluoromethyl)-3-pyridyl]indol-3-yl]ethyl]cyclopropanesulfonamide (24 mg, 48.24 μmol, 20.98% yield) ¹H NMR (400 MHz, CDCl₃) δ 8.91 (s, 1H), 8.09 (d, J=8.1 Hz, 1H), 7.77 (d, J=8.1 Hz, 1H), 7.59 (d, J=11.2 Hz, 1H), 7.33 (d, J=6.2 Hz, 1H), 7.15 (s, 1H), 5.00-4.94 (m, 1H), 4.40 (d, J=6.7 Hz, 1H), 3.90 (s, 2H), 2.32 (ddd, J=9.7, 6.4, 4.0 Hz, 1H), 2.02 (dd, J=9.6, 6.2 Hz, 1H), 1.75 (d, J=6.8 Hz, 3H), 1.16 (dd, J=4.3, 2.9 Hz, 2H), 1.02 (d, J=4.3 Hz, 9H), 0.91-0.85 (m, 2H). MS Found: 377.2 [M+1]⁺.

### Example 71: N-(1-(5-fluoro-6-(4-methylpyridin-3-yl)-1-neopentyl-1H-indol-3-yl)ethyl)cyclopropanesulfonamide

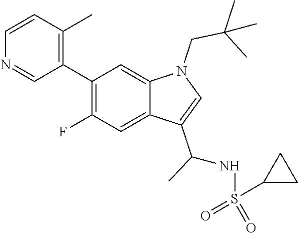 [SMILES: Cc1ccncc1-c1cc2c(cc1F)c(C(C)NS(=O)(=O)C1CC1)cn2CC(C)(C)C]

To a mixture of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (200 mg, 418 μmol) in dioxane:H₂O=4:1 (10 mL) was added 3-bromo-4-methyl-pyridine (73.60 mg, 418 μmol). Then sodium carbonate (88.6 mg, 831.6 μmol, 38.82 μL) and Pd(dppf)Cl₂ were added to the above mixture. The mixture was purged by N₂ 2 times and stirred at 90° C. for 12 hours. The mixture was diluted with water and extracted with ethyl acetate. The organic layer was washed with brine, dried over sodium carbonate and concentrated under reduced pressure. The crude was purified by Prep HPLC to give N-(1-(5-fluoro-6-(4-methylpyridin-3-yl)-1-neopentyl-1H-indol-3-yl)ethyl)cyclopropanesulfonamide (3.84 mg, 8.66 μmol) as a yellow solid. ¹H NMR (400 MHz, CDCl₃) d 8.49 (d, J=5.1 Hz, 1H), 8.44 (s, 1H), 7.52 (d, J=10.3 Hz, 1H), 7.23 (d, J=5.1 Hz, 1H), 7.15 (d, J=5.9 Hz, 1H), 7.12 (s, 1H), 4.97 (p, J=6.9 Hz, 1H), 4.48 (d, J=6.8 Hz, 1H), 3.86 (s, 2H), 2.38-2.30 (m, 1H), 2.25 (s, 3H), 1.76 (d, J=6.8 Hz, 3H), 1.16 (dd, J=4.8, 1.8 Hz, 2H), 0.99 (s, 9H), 0.93-0.86 (m, 2H). MS Found: 444.2 [M+1]⁺.

### Example 72: N-(1-(5-fluoro-6-(4-fluoropyridin-3-yl)-1-neopentyl-1H-indol-3-yl)ethyl)cyclopropanesulfonamide

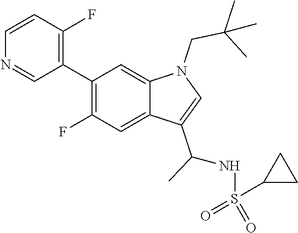 [SMILES: CC(NS(=O)(=O)C1CC1)c1cn(CC(C)(C)C)c2cc(-c3cnccc3F)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (100 mg, 209.02 μmol) and 3-bromo-4-fluoropyridine (36.79 mg, 209.02 μmol) in water (0.5 mL) and dioxane (2 mL) was added sodium carbonate (22.15 mg, 209.02 μmol, 8.75 μL) and Pd(dppf)Cl₂ (23 mg, 31.43 μmol) and stirred at 90° C. for 5 hr. The mixture was poured into water and extracted with ethyl acetate. The organic layer dried over Na₂SO₄ and concentrated under reduced pressure to give the crude product. The crude product was purified by Prep HPLC to give the title compound (23 mg, 51.39 μmol, 24.6% yield). ¹H NMR (400 MHz, CDCl₃) δ 8.90 (s, 1H), 8.05 (d, J=8.1 Hz, 1H), 7.77 (d, J=8.1 Hz, 1H), 7.60 (d, J=11.4 Hz, 1H), 7.32 (d, J=6.2 Hz, 1H), 7.16 (s, 1H), 4.99-4.94 (m, 1H), 4.43 (d, J=6.9 Hz, 1H), 3.90 (s, 2H), 2.32 (ddd, J=8.0, 4.8, 3.1 Hz, 1H), 1.75 (d, J=6.8 Hz, 3H), 1.16 (dd, J=7.4, 3.7 Hz, 2H), 1.02 (s, 9H), 0.89 (td, J=8.2, 2.1 Hz, 2H). MS Found: 448.2 [M+1]⁺.

### Example 73: N-[1-[6-(4-chloro-3-pyridyl)-1-(2,2-dimethylpropyl)-5-fluoro-indol-3-yl]ethyl]cyclopropanesulfonamide

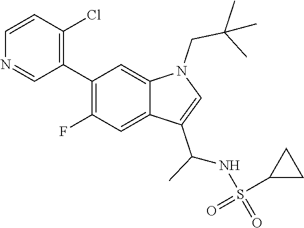 [SMILES: CC(NS(=O)(=O)C1CC1)c1cn(CC(C)(C)C)c2cc(-c3cnccc3Cl)c(F)cc12]

To a solution of N-[1-[1-(2,2-dimethylpropyl)-5-fluoro-6-(4,4,5,5-tetramethyl-1,3,2-dioxaborolan-2-yl)indol-3-yl]ethyl]cyclopropanesulfonamide (80 mg, 167.21 μmol) and 3,4-dichloropyridine (32.18 mg, 167.21 μmol) in water 500.7 μL) and dioxane (2.00 mL) was added sodium carbonate (35.45 mg, 334.43 μmol, 14.00 μL) and cyclopentyl(diphenyl)phosphanedichloropalladiumiron (18.35 mg, 25.08 μmol). Then the solution was stirred at 90° C. for 5 hr at which time the reaction was diluted with water (30 ml) and extracted with EA (50 ml×3). The combined organic layers were washed with brine (50 ml), dried over Na₂SO₄, filtered and concentrated. The residue obtained was purified by Prep HPLC to give the title compounds (4.2 mg, 10.99 μmol, 6.57% yield) as a white solid. 1H NMR (400 MHz, CDCl3) δ 8.57 (s, 1H), 8.51 (d, J=5.3 Hz, 1H), 7.55 (d, J=10.3 Hz, 1H), 7.46 (d, J=5.3 Hz, 1H), 7.23 (d, J=5.8 Hz, 1H), 7.14 (s, 1H), 5.01-4.93 (m, 1H), 4.50 (d, J=6.7 Hz, 1H), 3.87 (s, 2H), 2.32 (ddd, J=12.9, 8.1, 4.9 Hz, 1H), 1.75 (d, J=6.8 Hz, 3H), 1.15 (dd, J=4.9, 1.5 Hz, 2H), 1.00 (s, 9H), 0.87 (dd, J=7.8, 2.1 Hz, 2H). MS Found: 464.1 [M+1]⁺.

### Example 74: (S)—N-(2,2-difluoro-1-(6-(5-fluoro-3-(trifluoromethyl)pyridin-2-yl)-1-neopentyl-1H-indol-3-yl)ethyl)-sulfamoyldimethylamine

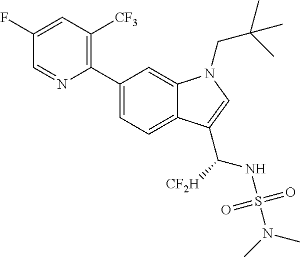 [SMILES: CN(C)S(=O)(=O)N[C@@H](c1cn(CC(C)(C)C)c2cc(-c3ncc(F)cc3C(F)(F)F)ccc12)C(F)F]

### Step 1: (S)—N—((S)-2,2-difluoro-1-(6-(5-fluoro-3-(trifluoromethyl)pyridin-2-yl)-1-neopentyl-1H-indol-3-yl)ethyl)-2-methylpropane-2-sulfinamide

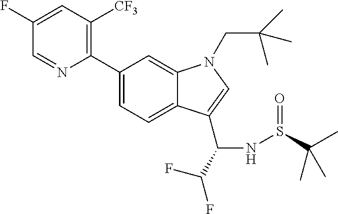 [SMILES: CC(C)(C)Cn1cc([C@H](N[S@@](=O)C(C)(C)C)C(F)F)c2ccc(-c3ncc(F)cc3C(F)(F)F)cc21]

The title compound was prepared following the same general protocol as described for Step 6 Example 2, using (S)—N—((S)-2,2-difluoro-1-(1-neopentyl-6-(4,4,5,5-tetramethyl-1,3,2-dioxaborolan-2-yl)-1H-indol-3-yl)ethyl)-2-methylpropane-2-sulfinamide and 2-chloro-5-fluoro-3-(trifluoromethyl)pyridine. ESI-MS (m/z): 533.75 [M+1]⁺.

### Step 2: (S)-2,2-difluoro-1-(6-(5-fluoro-3-(trifluoromethyl)pyridin-2-yl)-1-neopentyl-1H-indol-3-yl)ethan-1-amine

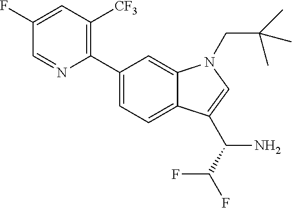 [SMILES: CC(C)(C)Cn1cc([C@H](N)C(F)F)c2ccc(-c3ncc(F)cc3C(F)(F)F)cc21]

The title compound was prepared following the same general protocol as described for Step 3, Example 2, using (S)—N—((S)-2,2-difluoro-1-(6-(5-fluoro-3-(trifluoromethyl)pyridin-2-yl)-1-neopentyl-1H-indol-3-yl)ethyl)-2-methylpropane-2-sulfinamide. ESI-MS (m/z): 413.2 [M-NH₂]⁺.

Step 3: The title compound was prepared following the same general protocol as described for Example 21 using (S)-2,2-difluoro-1-(6-(5-fluoro-3-(trifluoromethyl)pyridin-2-yl)-1-neopentyl-1H-indol-3-yl)ethan-1-amine and dimethylsulfamoyl chloride. ESI-MS (m/z): 536.68 [M+1]⁺.

### Example 75: (S)—N-(1-(6-(2-cyano-6-(trifluoromethyl)phenyl)-1-neopentyl-1H-indol-3-yl)-2,2-difluoroethyl)-sulfamoyldimethylamine

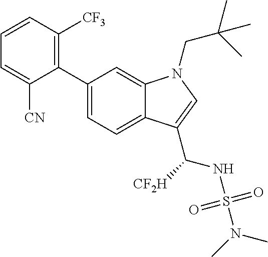 [SMILES: CN(C)S(=O)(=O)N[C@@H](c1cn(CC(C)(C)C)c2cc(-c3c(C#N)cccc3C(F)(F)F)ccc12)C(F)F]

### Step 1: N—((S)-1-(6-(2-cyano-6-(trifluoromethyl)phenyl)-1-neopentyl-1H-indol-3-yl)-2,2-difluoroethyl)-2-methylpropane-2-sulfinamide

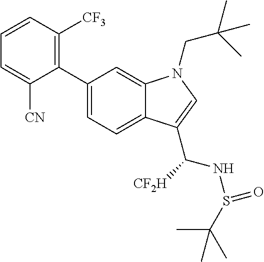 [SMILES: CC(C)(C)Cn1cc([C@H](NS(=O)C(C)(C)C)C(F)F)c2ccc(-c3c(C#N)cccc3C(F)(F)F)cc21]

2-bromo-3-(trifluoromethyl)benzonitrile (100 mg, 0.4 mmol), K₂CO₃ (52 mg, 0.4 mmol) and Pd(dppf)Cl₂ (7 mg, 0.01 mmol) were added to a solution of(S)—N—((S)-2,2-difluoro-1-(1-neopentyl-6-(4,4,5,5-tetramethyl-1,3,2-dioxaborolan-2-yl)-1H-indol-3-yl)ethyl)-2-methylpropane-2-sulfinamide (100 mg, 0.2 mmol) in dioxane/water (1.0 mL, v/v=1:1) and the reaction mixture was degassed (three times). The reaction mixture was warmed to 100° C. for 1-2 hours, and then cooled to rt. The reaction mixture was diluted with water (10 mL) and extracted with EtOAc (2×5 mL). The combined organics were dried (Na₂SO₄) and filtered. The solvent was removed under reduced pressure and the residue was purified by combi-flash on silica gel to obtain the title compound as a solid (81 mg). ESI-MS (m/z): 539.71 [M+1]⁺.

### Step 2: (S)-2-(3-(1-amino-2,2-difluoroethyl)-1-neopentyl-1H-indol-6-yl)-3-(trifluoromethyl)benzonitrile

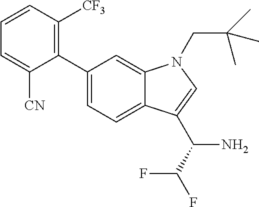 [SMILES: CC(C)(C)Cn1cc([C@H](N)C(F)F)c2ccc(-c3c(C#N)cccc3C(F)(F)F)cc21]

37% HCl aqueous solution (80 μL) was added to a solution of(S)—N—((S)-1-(6-(2-cyano-6-(trifluoromethyl)phenyl)-1-neopentyl-1H-indol-3-yl)-2,2-difluoroethyl)-2-methylpropane-2-sulfinamide (81 mg) in MeOH (1.0 mL) at 0° C. and the reaction mixture was stirred at room temperature for 3-4 hours. The solvent was removed under reduced pressure and the residue was dissolved in EtOAc (2 mL) and adjusted pH=8 with saturated NaHCO₃. The layers were separated, and the aqueous layer was extracted with EtOAc (3×2 mL). The combined organics were dried (Na₂SO₄) and filtered. The solvent was removed under reduced pressure and the residue was purified by combi-flash on silica gel to obtain the title compound as an oil (35 mg). ESI-MS (m/z): 418.96 [M-NH₂]⁺.

Step 3: The title compound was prepared following the same general protocol as described for Example 21 using (S)-2-(3-(1-amino-2,2-difluoroethyl)-1-neopentyl-1H-indol-6-yl)-3-(trifluoromethyl)benzonitrile and dimethylsulfamoyl chloride. ESI-MS (m/z): 542.51 [M+1]⁺.

### Example 76: (S)—N-(2,2-difluoro-1-(5-fluoro-1-neopentyl-6-(2-(trifluoromethyl)phenyl)-1H-indol-3-yl)ethyl)-3-fluoroazetidine-1-sulfonamide

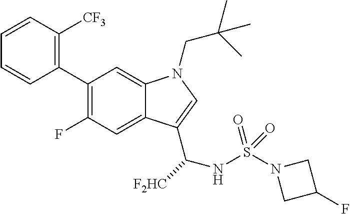 [SMILES: CC(C)(C)Cn1cc([C@H](NS(=O)(=O)N2CC(F)C2)C(F)F)c2cc(F)c(-c3ccccc3C(F)(F)F)cc21]

The title compound was prepared following the same general protocol as described for Step 4, Example 2, using (S)-2,2-difluoro-1-(5-fluoro-1-neopentyl-6-(2-(trifluoromethyl)phenyl)-1H-indol-3-yl)ethan-1-amine and 3-fluoroazetidine-1-sulfonyl chloride. ESI-MS (m/z): 565.6 [M+1]⁺.

### Example 77: (S)—N-(1-(6-(5-chloro-3-fluoropyridin-2-yl)-1-neopentyl-1H-indol-3-yl)-2,2-difluoroethyl)cyclopropanesulfonamide

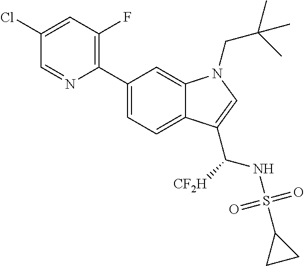 [SMILES: CC(C)(C)Cn1cc([C@H](NS(=O)(=O)C2CC2)C(F)F)c2ccc(-c3ncc(Cl)cc3F)cc21]

The title compound was prepared following the same general protocol as described for Step 6, Example 2, using (S)—N-(2,2-difluoro-1-(1-neopetyl-6-(4,4,5,5-tetramethyl-1,3,2-dioxaborolan-2-yl)-1H-indol-3-yl)ethyl)cyclopropanesulfonamide and 2-bromo-5-chloro-3-fluoropyridine. ESI-MS (m/z): 499.79 [M+1]⁺.

### Example 78: (S)—N-(1-(6-(3-chloro-5-fluoropyridin-2-yl)-1-neopentyl-1H-indol-3-yl)-2,2-difluoroethyl)cyclopropanesulfonamide

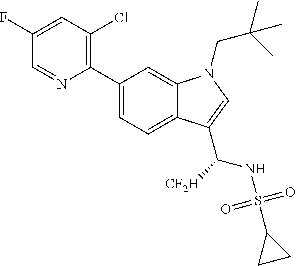 [SMILES: CC(C)(C)Cn1cc([C@H](NS(=O)(=O)C2CC2)C(F)F)c2ccc(-c3ncc(F)cc3Cl)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-chloro-5-fluoropyridine. ESI-MS (m/z): 499.81 [M+1]⁺.

### Example 79: (S)—N-(2,2-difluoro-1-(1-neopentyl-6-(4-(trifluoromethyl)pyridin-3-yl)-1H-indol-3-yl)ethyl)cyclopropanesulfonamide

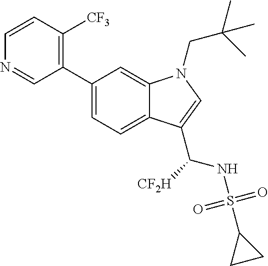 [SMILES: CC(C)(C)Cn1cc([C@H](NS(=O)(=O)C2CC2)C(F)F)c2ccc(-c3cnccc3C(F)(F)F)cc21]

The title compound was prepared following the same general protocol as described for Step 6, Example 2, using (S)—N-(2,2-difluoro-1-(1-neopetyl-6-(4,4,5,5-tetramethyl-1,3,2-dioxaborolan-2-yl)-1H-indol-3-yl)ethyl)cyclopropanesulfonamide and 3-bromo-4-(trifluoromethyl)pyridine. ESI-MS (m/z): 515.99 [M+1]⁺.

### Example 80: (S)—N-(2,2-difluoro-1-(1-neopentyl-6-(4-(trifluoromethyl)pyrimidin-5-yl)-1H-indol-3-yl)ethyl)cyclopropanesulfonamide

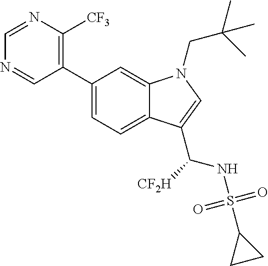 [SMILES: CC(C)(C)Cn1cc([C@H](NS(=O)(=O)C2CC2)C(F)F)c2ccc(-c3cncnc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 5-bromo-4-(trifluoromethyl)pyrimidine. ESI-MS (m/z): 516.99 [M+1]⁺.

### Example 81: (S)—N-(1-(6-(2-(difluoromethyl)phenyl)-1-neopentyl-1H-indol-3-yl)-2,2-difluoroethyl)cyclopropanesulfonamide

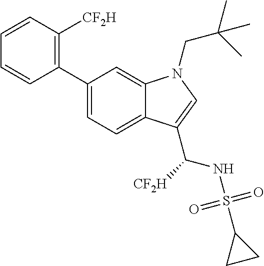 [SMILES: CC(C)(C)Cn1cc([C@H](NS(=O)(=O)C2CC2)C(F)F)c2ccc(-c3ccccc3C(F)F)cc21]

The title compound was prepared following the same general protocol as described for Step 6, Example 2, using (S)—N-(2,2-difluoro-1-(1-neopetyl-6-(4,4,5,5-tetramethyl-1,3,2-dioxaborolan-2-yl)-1H-indol-3-yl)ethyl)cyclopropanesulfonamide and 1-bromo-2-(difluoromethyl)benzene. ESI-MS (m/z): 496.65 [M+1]⁺.

### Example 82: (S)—N-(1-(6-(2-cyano-4-(trifluoromethyl)pyridin-3-yl)-1-neopentyl-1H-indol-3-yl)-2,2-difluoroethyl)cyclopropanesulfonamide

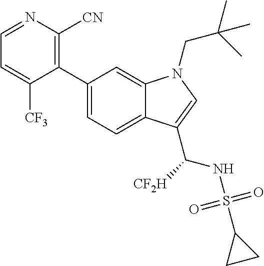 [SMILES: CC(C)(C)Cn1cc([C@H](NS(=O)(=O)C2CC2)C(F)F)c2ccc(-c3c(C(F)(F)F)ccnc3C#N)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-4-(trifluoromethyl)picolinonitrile. ESI-MS (m/z): 540.55 [M+1]⁺.

### Example 83: (S)—N-(2,2-difluoro-1-(6-(3-fluoropyridin-2-yl)-1-neopentyl-1H-indol-3-yl)ethyl)cyclopropanesulfonamide

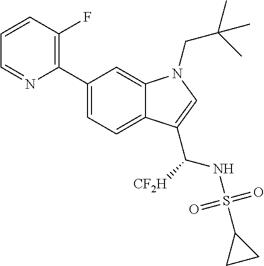 [SMILES: CC(C)(C)Cn1cc([C@H](NS(=O)(=O)C2CC2)C(F)F)c2ccc(-c3ncccc3F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-fluoropyridine. ESI-MS (m/z): 465.89 [M+1]⁺.

### Example 84: (S)—N-(1-(6-(3,5-difluoropyridin-2-yl)-1-neopentyl-1H-indol-3-yl)-2,2-difluoroethyl)cyclopropanesulfonamide

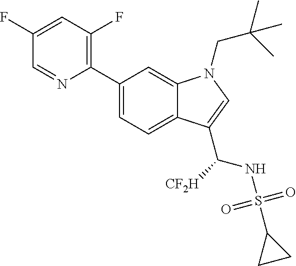 [SMILES: CC(C)(C)Cn1cc([C@H](NS(=O)(=O)C2CC2)C(F)F)c2ccc(-c3ncc(F)cc3F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3,5-difluoropyridine. ESI-MS (m/z): 483.83 [M+1]⁺.

### Example 85: (S)—N-(1-(6-(3-chloropyridin-2-yl)-1-neopentyl-1H-indol-3-yl)-2,2-difluoroethyl)cyclopropanesulfonamide

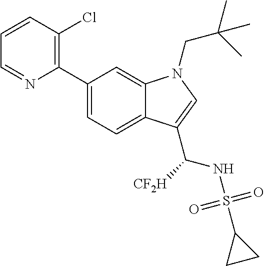 [SMILES: CC(C)(C)Cn1cc([C@H](NS(=O)(=O)C2CC2)C(F)F)c2ccc(-c3ncccc3Cl)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-chloropyridine. ESI-MS (m/z): 481.94 [M+1]⁺.

### Example 86: (S)—N-(1-(6-(2-cyano-5-fluoropyridin-3-yl)-1-neopentyl-1H-indol-3-yl)-2,2-difluoroethyl)cyclopropanesulfonamide

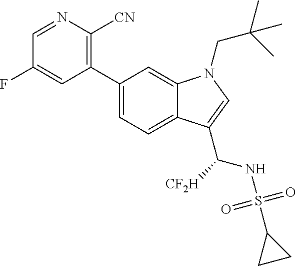 [SMILES: CC(C)(C)Cn1cc([C@H](NS(=O)(=O)C2CC2)C(F)F)c2ccc(-c3cc(F)cnc3C#N)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-5-fluoropicolinonitrile. ESI-MS (m/z): 490.61 [M+1]⁺.

### Example 87: (S)—N-(1-(6-(2-(difluoromethyl)pyridin-3-yl)-1-neopentyl-1H-indol-3-yl)-2,2-difluoroethyl)cyclopropanesulfonamide

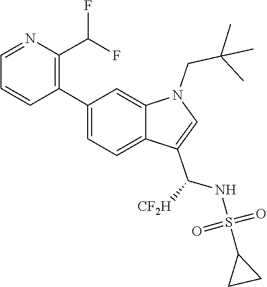 [SMILES: CC(C)(C)Cn1cc([C@H](NS(=O)(=O)C2CC2)C(F)F)c2ccc(-c3cccnc3C(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-2-(difluoromethyl)pyridine. ESI-MS (m/z): 497.97 [M+1]⁺.

### Example 88: (S)—N-(1-(6-(3-cyano-5-fluoropyridin-2-yl)-1-neopentyl-1H-indol-3-yl)-2,2-difluoroethyl)cyclopropanesulfonamide

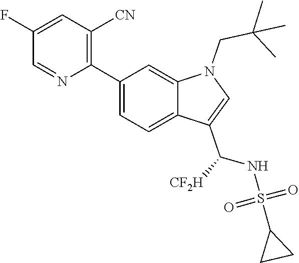 [SMILES: CC(C)(C)Cn1cc([C@H](NS(=O)(=O)C2CC2)C(F)F)c2ccc(-c3ncc(F)cc3C#N)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-5-fluoronicotinonitrile. ESI-MS (m/z): 490.72 [M+1]⁺.

### Example 89: (S)—N-(2,2-difluoro-1-(5-fluoro-6-(3-fluoropyridin-2-yl)-1-neopentyl-1H-indol-3-yl)ethyl)cyclopropanesulfonamide

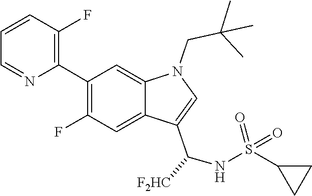 [SMILES: CC(C)(C)Cn1cc([C@H](NS(=O)(=O)C2CC2)C(F)F)c2cc(F)c(-c3ncccc3F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-3-fluoropyridine with anhydrous dioxane as solvent, and the reaction temperature 140° C. ESI-MS (m/z): 483.9 [M+1]⁺.

### Example 90: (S)—N-(1-(6-(3-chloropyridin-2-yl)-5-fluoro-1-neopentyl-1H-indol-3-yl)-2,2-difluoroethyl)cyclopropanesulfonamide

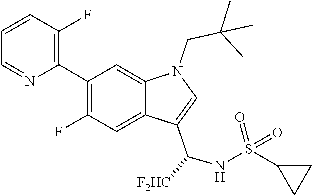 [SMILES: CC(C)(C)Cn1cc([C@H](NS(=O)(=O)C2CC2)C(F)F)c2cc(F)c(-c3ncccc3F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-3-chloropyridine with anhydrous dioxane as solvent, and the reaction temperature 140° C. ESI-MS (m/z): 501.4 [M+1]⁺.

### Example 91: (S)—N-(1-(6-(3,5-difluoropyridin-2-yl)-5-fluoro-1-neopentyl-1H-indol-3-yl)-2,2-difluoroethyl)cyclopropanesulfonamide

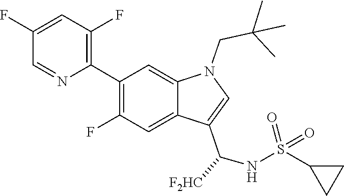 [SMILES: CC(C)(C)Cn1cc([C@H](NS(=O)(=O)C2CC2)C(F)F)c2cc(F)c(-c3ncc(F)cc3F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-3,5-difluoropyridine with anhydrous dioxane as solvent, and the reaction temperature 140° C. ESI-MS (m/z): 502.1 [M+1]⁺.

### Example 92: (S)—N-(1-(6-(5-chloro-3-fluoropyridin-2-yl)-5-fluoro-1-neopentyl-1H-indol-3-yl)-2,2-difluoroethyl)cyclopropanesulfonamide

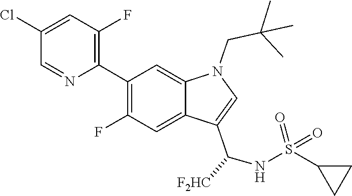 [SMILES: CC(C)(C)Cn1cc([C@H](NS(=O)(=O)C2CC2)C(F)F)c2cc(F)c(-c3ncc(Cl)cc3F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-5-chloro-3-fluoropyridine with anhydrous dioxane as solvent, and the reaction temperature 140° C. ESI-MS (m/z): 518.5 [M+1]⁺.

### Example 93: (S)—N-(1-(6-(3-chloro-5-fluoropyridin-2-yl)-5-fluoro-1-neopentyl-1H-indol-3-yl)-2,2-difluoroethyl)cyclopropanesulfonamide

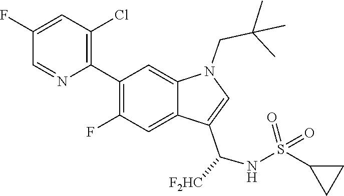 [SMILES: CC(C)(C)Cn1cc([C@H](NS(=O)(=O)C2CC2)C(F)F)c2cc(F)c(-c3ncc(F)cc3Cl)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-3-chloro-5-fluoropyridine with anhydrous dioxane as solvent, and the reaction temperature 140° C. ESI-MS (m/z): 518.5 [M+1]⁺.

### Example 94: (S)—N-(1-(6-(3-cyano-5-fluoropyridin-2-yl)-5-fluoro-1-neopentyl-1H-indol-3-yl)-2,2-difluoroethyl)cyclopropanesulfonamide

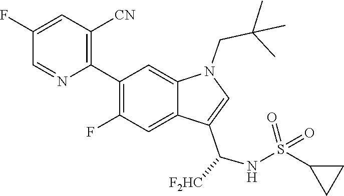 [SMILES: CC(C)(C)Cn1cc([C@H](NS(=O)(=O)C2CC2)C(F)F)c2cc(F)c(-c3ncc(F)cc3C#N)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-5-fluoronicotinonitrile with anhydrous dioxane as solvent, and the reaction temperature 140° C. ESI-MS (m/z): 508.5 [M+1]⁺.

### Example 95: (S)—N-(2,2-difluoro-1-(5-fluoro-6-(5-fluoro-2-methylpyridin-3-yl)-1-neopentyl-1H-indol-3-yl)ethyl)cyclopropanesulfonamide

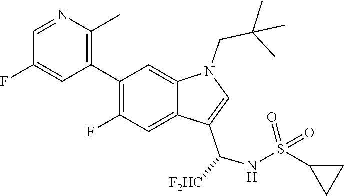 [SMILES: Cc1ncc(F)cc1-c1cc2c(cc1F)c([C@H](NS(=O)(=O)C1CC1)C(F)F)cn2CC(C)(C)C]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-5-fluoro-2-methylpyridine with anhydrous dioxane as solvent, and the reaction temperature 140° C. ESI-MS (m/z): 498.0 [M+1]⁺.

### Example 96: (S)—N-(1-(6-(2-cyano-5-fluoropyridin-3-yl)-5-fluoro-1-neopentyl-1H-indol-3-yl)-2,2-difluoroethyl)cyclopropanesulfonamide

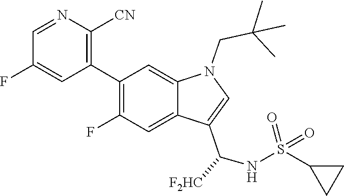 [SMILES: CC(C)(C)Cn1cc([C@H](NS(=O)(=O)C2CC2)C(F)F)c2cc(F)c(-c3cc(F)cnc3C#N)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-5-fluoropicolinonitrile with anhydrous dioxane as solvent, and the reaction temperature 140° C. ¹H NMR (CDCl₃, 400 MHz) δ 8.57 (d, J=2.7 Hz, 1H), 7.65 (dt, J=8.4 Hz, 1.9 Hz, 1H), 7.57 (d, J=10.7 Hz, 1H), 7.44 (d, J=5.9 Hz, 1H), 7.33 (s, 1H), 6.15 (td, J=55.8 Hz, 2.2 Hz, 1H), 5.20-5.05 (m, 2H), 3.90 (s, 2H), 2.42-2.35 (m, 1H), 1.15-1.11 (m, 2H), 0.98 (s, 9H), 0.90-0.88 (m, 2H)

### Example 97: (S)—N-(1-(6-(2-(difluoromethyl)pyridin-3-yl)-5-fluoro-1-neopentyl-1H-indol-3-yl)-2,2-difluoroethyl)cyclopropanesulfonamide

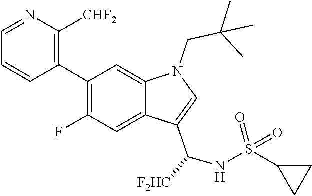 [SMILES: CC(C)(C)Cn1cc([C@H](NS(=O)(=O)C2CC2)C(F)F)c2cc(F)c(-c3cccnc3C(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-2-(difluoromethyl)pyridine with anhydrous dioxane as solvent, and the reaction temperature 140° C. ESI-MS (m/z): 515.7 [M+1]⁺.

### Example 98: (S)—N-(1-(6-(2-(difluoromethyl)phenyl)-5-fluoro-1-neopentyl-1H-indol-3-yl)-2,2-difluoroethyl)cyclopropanesulfonamide

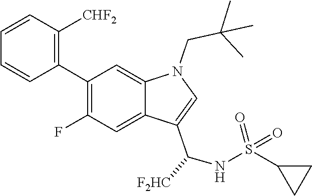 [SMILES: CC(C)(C)Cn1cc([C@H](NS(=O)(=O)C2CC2)C(F)F)c2cc(F)c(-c3ccccc3C(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 1-bromo-2-(difluoromethyl)benzene with anhydrous dioxane as solvent, and the reaction temperature 140° C. ESI-MS (m/z): 514.6 [M+1]⁺.

### Example 99: N-((1S)-1-(6-(2-cyano-4-(trifluoromethyl)pyridin-3-yl)-5-fluoro-1-neopentyl-1H-indol-3-yl)-2,2-difluoroethyl)cyclopropanesulfonamide

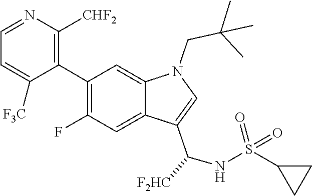 [SMILES: CC(C)(C)Cn1cc([C@H](NS(=O)(=O)C2CC2)C(F)F)c2cc(F)c(-c3c(C(F)(F)F)ccnc3C(F)F)cc21]

### Step 1:3-bromo-4-(trifluoromethyl)pyridine 1-oxide

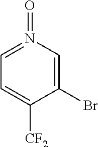

To a solution of 3-bromo-4-(trifluoromethyl)pyridine (1.12 g, 4.96 mmol) in DCM (20 mL) was added mCPBA (075%, 2.28 g, 9.92 mmol), and then the mixture was stirred overnight. The completion was monitored by anal. HPLC. The mixture was diluted with DCM and washed with sat'd NaHCO₃, and brine and dried over Na₂SO₄. The solvent was removed and the crude was purified by silica gel to obtain the title compound. ESI-MS (m/z): 241.4, 243.4 [M+1]⁺.

### Step 2:3-bromo-4-(trifluoromethyl)picolinonitrile and 5-bromo-4-(trifluoromethyl)picolinonitrile

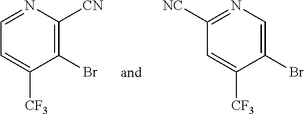 [SMILES: N#Cc1nccc(C(F)(F)F)c1Br ; "and" ; N#Cc1cc(C(F)(F)F)c(Br)cn1]

3-bromo-4-(trifluoromethyl)pyridine 1-oxide (0.25 g, 1.03 mmol) in ACN (5 mL) was added TEA (0.58 mL, 4.12 mmol), followed by addition of TMSCN (0.39 mL, 3.09 mmol). The mixture was stirred at 80° C. overnight. The reaction was cooled to rt and diluted with EtOAc, washed with sat'd NaHCO₃, and brine and dried over Na₂SO₄. The solvent was removed and the crude was purified by silica gel to obtain the title compounds. 3-bromo-4-(trifluoromethyl)picolinonitrile: 1H NMR (CDCl₃, 400 MHz) δ 8.82 (dd, J=4.88 Hz, 0.56 Hz, 1H), 7.79 (dd, J=5.92 Hz, 0.4 Hz, 1H); 5-bromo-4-(trifluoromethyl)picolinonitrile: 1H NMR (CDCl₃, 400 MHz) δ 9.02 (s, 1H), 7.93 (s, 1H)

### Step 3: N-((1S)-1-(6-(2-cyano-4-(trifluoromethyl)pyridin-3-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-4-(trifluoromethyl)picolinonitrile with anhydrous dioxane as solvent, and the reaction temperature of 140° C. 1H NMR (CDCl₃, 400 MHz) δ 8.93 (d, J=4.8 Hz, 1H), 7.89 (dd, J=4.8 Hz, 1.8 Hz, 1H), 7.58 (d, J=10.0 Hz, 1H), 7.60-7.54 (m, 1H), 7.32 (d, J=6.4 Hz, 1H), 7.25-7.23 (m, 1H), 6.18 (td, J=55.8 Hz, 2.2 Hz, 1H), 5.17-4.99 (m, 2H), 3.94-3.78 (m, 2H), 2.35-2.27 (m, 1H), 1.15-1.11 (m, 2H), 0.98 (s, 9H), 0.90-0.88 (m, 2H).

### Example 100: (S)—N-(1-(6-(3-(difluoromethyl)pyridin-2-yl)-1-neopentyl-1H-indol-3-yl)-2,2-difluoroethyl)cyclopropanesulfonamide

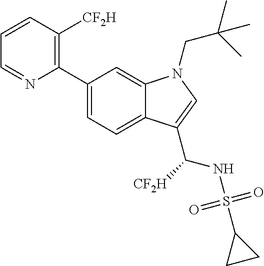 [SMILES: CC(C)(C)Cn1cc([C@H](NS(=O)(=O)C2CC2)C(F)F)c2ccc(-c3ncccc3C(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-(difluoromethyl)pyridine. ESI-MS (m/z): 497.90 [M+1]⁺.

### Example 101: (S)—N-(1-(6-(3-cyclopropylpyridin-2-yl)-1-neopentyl-1H-indol-3-yl)-2,2-difluoroethyl)cyclopropanesulfonamide

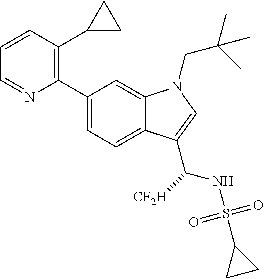 [SMILES: CC(C)(C)Cn1cc([C@H](NS(=O)(=O)C2CC2)C(F)F)c2ccc(-c3ncccc3C3CC3)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-cyclopropylpyridine. ESI-MS (m/z): 487.97 [M+1]⁺.

### Example 102: (S)—N-(2,2-difluoro-1-(1-neopentyl-6-(3-(trifluoromethyl)pyridin-4-yl)-1H-indol-3-yl)ethyl)cyclopropanesulfonamide

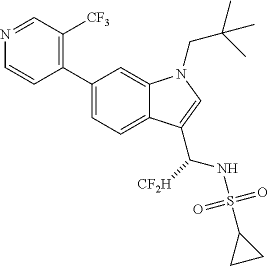 [SMILES: CC(C)(C)Cn1cc([C@H](NS(=O)(=O)C2CC2)C(F)F)c2ccc(-c3ccncc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 4-bromo-3-(trifluoromethyl)pyridine. ESI-MS (m/z): 515.95 [M+1]⁺.

### Example 103: (S)—N-(1-(6-(2-cyano-6-(difluoromethyl)phenyl)-1-neopentyl-1H-indol-3-yl)-2,2-difluoroethyl)cyclopropanesulfonamide

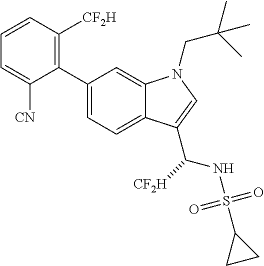

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-(difluoromethyl)benzonitrile. ESI-MS (m/z): 521.69 [M+1]⁺.

### Example 104: (S)—N-(1-(6-(2-(difluoromethyl)-6-fluorophenyl)-1-neopentyl-1H-indol-3-yl)-2,2-difluoroethyl)cyclopropanesulfonamide

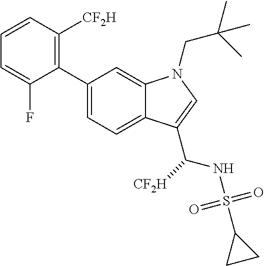 [SMILES: CC(C)(C)Cn1cc([C@H](NS(=O)(=O)C2CC2)C(F)F)c2ccc(-c3c(F)cccc3C(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-1-(difluoromethyl)-3-fluorobenzene. ESI-MS (m/z): 514.57 [M+1]⁺.

### Example 105: (S)—N-(1-(6-(2-(difluoromethyl)-4-fluorophenyl)-1-neopentyl-1H-indol-3-yl)-2,2-difluoroethyl)cyclopropanesulfonamide

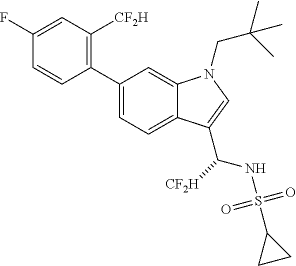 [SMILES: CC(C)(C)Cn1cc([C@H](NS(=O)(=O)C2CC2)C(F)F)c2ccc(-c3ccc(F)cc3C(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 1-bromo-2-(difluoromethyl)-4-fluorobenzene. ESI-MS (m/z): 514.77 [M+1]⁺.

### Example 106: (S)-2-(3-(1-(cyclopropanesulfonamido)-2,2-difluoroethyl)-1-neopentyl-1H-indol-6-yl)-3-(trifluoromethyl)pyridine 1-oxide

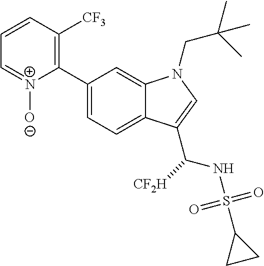 [SMILES: CC(C)(C)Cn1cc([C@H](NS(=O)(=O)C2CC2)C(F)F)c2ccc(-c3c(C(F)(F)F)ccc[n+]3[O-])cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 2-bromo-3-(trifluoromethyl)pyridine 1-oxide. ESI-MS (m/z): 531.73 [M+1]⁺.

### Example 107: (S)-3-(3-(1-(cyclopropanesulfonamido)-2,2-difluoroethyl)-1-neopentyl-1H-indol-6-yl)-4-(trifluoromethyl)pyridine 1-oxide

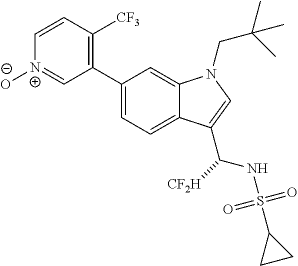 [SMILES: CC(C)(C)Cn1cc([C@H](NS(=O)(=O)C2CC2)C(F)F)c2ccc(-c3c[n+]([O-])ccc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-4-(trifluoromethyl)pyridine 1-oxide. ESI-MS (m/z): 531.72 [M+1]⁺.

### Example 108: N-((1S)-1-(6-(2-cyano-6-(trifluoromethyl)phenyl)-5-fluoro-1-neopentyl-1H-indol-3-yl)-2,2-difluoroethyl)cyclopropanesulfonamide

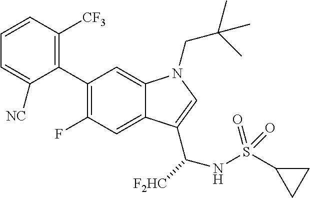 [SMILES: CC(C)(C)Cn1cc([C@H](NS(=O)(=O)C2CC2)C(F)F)c2cc(F)c(-c3c(C#N)cccc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-3-(trifluoromethyl)benzonitrile with anhydrous dioxane as solvent, and the reaction temperature 140° C. ESI-MS (m/z): 557.8 [M+1]⁺.

### Example 109: N-((1S)-1-(6-(2-cyano-6-(difluoromethyl)phenyl)-5-fluoro-1-neopentyl-1H-indol-3-yl)-2,2-difluoroethyl)cyclopropanesulfonamide

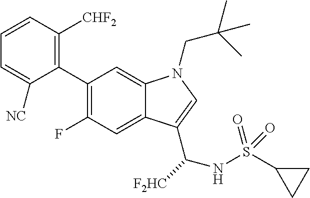 [SMILES: CC(C)(C)Cn1cc([C@H](NS(=O)(=O)C2CC2)C(F)F)c2cc(F)c(-c3c(C#N)cccc3C(F)F)cc21]

### Step 1:2-bromo-3-(difluoromethyl)benzonitrile

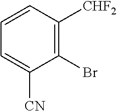 [SMILES: N#Cc1cccc(C(F)F)c1Br]

To a solution of 2-bromo-3-formylbenzonitrile (0.066 g, 0.314 mmol) in DCM (3 mL) at 0° C. was added slowly DAST (0.12 mL, 0.94 mmol). The completion was monitored by anal. HPLC. The mixture was diluted with DCM and washed with sat'd NaHCO₃, and brine and dried over Na₂SO₄. The solvent was removed and the crude was purified by silica gel to obtain the title compound. 1H NMR (CDCl₃, 400 MHz) δ 7.88 (m, 1H), 7.79 (m, 1H), 6.92 (t, J=54.4 Hz, 1H)

### Step 2: N-((1S)-1-(6-(2-cyano-6-(difluoromethyl)phenyl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-3-(difluoromethyl)benzonitrile with anhydrous dioxane as solvent, and the reaction temperature of 140° C. ¹H NMR (CDCl₃, 400 MHz) δ 8.01 (m, 1H), 7.89 (d, J=7.8 Hz, 1H), 7.66 (t, J=7.8 Hz, 1H), 7.58 (m, 1H), 7.31 (d, J=7.8 Hz, 1H), 7.26-7.23 (m, 1H), 6.57-6.03 (m, 2H), 5.16-5.07 (m, 2H), 3.93-3.84 (m, 2H), 2.42-2.30 (m, 1H), 1.17-1.05 (m, 2H), 0.99 (s, 9H), 0.96-0.83 (m, 2H)

### Example 110: (S)—N-(1-(6-(6-cyano-2-(trifluoromethyl)pyridin-3-yl)-5-fluoro-1-neopentyl-1H-indol-3-yl)-2,2-difluoroethyl)cyclopropanesulfonamide

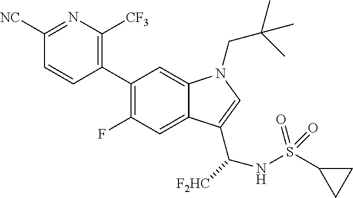 [SMILES: CC(C)(C)Cn1cc([C@H](NS(=O)(=O)C2CC2)C(F)F)c2cc(F)c(-c3ccc(C#N)nc3C(F)(F)F)cc21]

### Step 1:3-bromo-2-(trifluoromethyl)pyridine 1-oxide

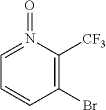 [SMILES: [O-][n+]1cccc(Br)c1C(F)(F)F]

To a mixture of 3-bromo-2-(trifluoromethyl)pyridine (1.1 g, 4.87 mmol) and H₂O₂-urea (1.05 g, 11.2 mmol) in DCM (50 mL) at 0° C. was added slowly 2,2,2-trifluoroacetic anhydride (1.4 mL, 9.74 mmol) in DCM (20 mL), and then the mixture was stirred overnight. The completion was monitored by anal. HPLC. The mixture was diluted with DCM and washed with sat'd NaHCO₃, and brine and dried over Na₂SO₄. The solvent was removed and the crude was purified by silica gel to obtain the title compound. ESI-MS (m/z): 241.4, 243.3 [M+1]⁺.

### Step 2:5-bromo-6-(trifluoromethyl)picolinonitrile

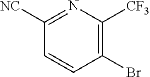 [SMILES: N#Cc1ccc(Br)c(C(F)(F)F)n1]

The title compound was obtained by applying the procedure of synthesis of 3-bromo-4-(trifluoromethyl)picolinonitrile. ¹H NMR (CDCl₃, 400 MHz) δ 8.26 (d, J=8.2 Hz, 1H), 7.73 (d, J=8.2 Hz, 1H)

### Step 3: (S)—N-(1-(6-(6-cyano-2-(trifluoromethyl)pyridin-3-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-6-(trifluoromethyl)picolinonitrile with anhydrous dioxane as solvent, and the reaction temperature 140° C. ESI-MS (m/z): 558.5 [M+1]⁺.

### Example 111: (S)—N-(1-(6-(3-(difluoromethyl)pyridin-2-yl)-5-fluoro-1-neopentyl-1H-indol-3-yl)-2,2-difluoroethyl)cyclopropanesulfonamide

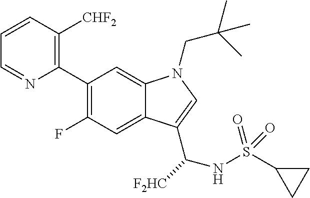 [SMILES: CC(C)(C)Cn1cc([C@H](NS(=O)(=O)C2CC2)C(F)F)c2cc(F)c(-c3ncccc3C(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-3-(difluoromethyl)pyridine with anhydrous dioxane as solvent, and the reaction temperature 140° C. ESI-MS (m/z): 516.0 [M+1]⁺.

### Example 112: (S)—N-(2,2-difluoro-1-(5-fluoro-1-neopentyl-6-(4-(trifluoromethyl)pyrimidin-5-yl)-1H-indol-3-yl)ethyl)cyclopropanesulfonamide

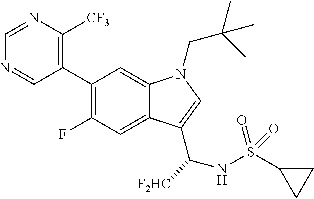 [SMILES: CC(C)(C)Cn1cc([C@H](NS(=O)(=O)C2CC2)C(F)F)c2cc(F)c(-c3cncnc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-4-(trifluoromethyl)pyrimidine with anhydrous dioxane as solvent, and the reaction temperature 140° C. ESI-MS (m/z): 534.9 [M+1]⁺.

### Example 113: (S)—N-(2,2-difluoro-1-(5-fluoro-1-neopentyl-6-(4-(trifluoromethyl)pyridin-3-yl)-1H-indol-3-yl)ethyl)cyclopropanesulfonamide

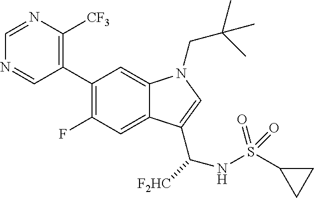 [SMILES: CC(C)(C)Cn1cc([C@H](NS(=O)(=O)C2CC2)C(F)F)c2cc(F)c(-c3cncnc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-4-(trifluoromethyl)pyridine with anhydrous dioxane as solvent, and the reaction temperature 140° C. ESI-MS (m/z): 534.0 [M+1]⁺.

### Example 114: (S)—N-(2,2-difluoro-1-(5-fluoro-1-neopentyl-6-(3-(trifluoromethyl)pyridin-4-yl)-1H-indol-3-yl)ethyl)cyclopropanesulfonamide

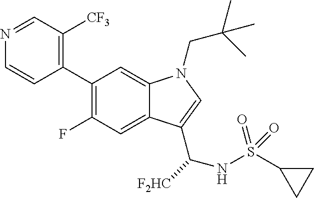 [SMILES: CC(C)(C)Cn1cc([C@H](NS(=O)(=O)C2CC2)C(F)F)c2cc(F)c(-c3ccncc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 4-bromo-3-(trifluoromethyl)pyridine HBr salt with anhydrous dioxane as solvent, and the reaction temperature of 140° C. ESI-MS (m/z): 534.0 [M+1]⁺.

### Example 115: (S)—N-(1-(6-(3-cyclopropylpyridin-2-yl)-5-fluoro-1-neopentyl-1H-indol-3-yl)-2,2-difluoroethyl)cyclopropanesulfonamide

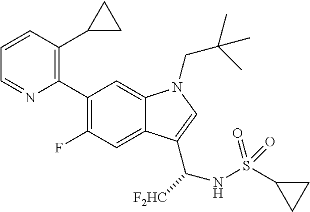 [SMILES: CC(C)(C)Cn1cc([C@H](NS(=O)(=O)C2CC2)C(F)F)c2cc(F)c(-c3ncccc3C3CC3)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-chloro-3-cyclopropylpyridine with anhydrous dioxane as solvent, and the reaction temperature of 140° C. ESI-MS (m/z): 506.0 [M+1]⁺.

### Example 116: N-((1S)-1-(6-(2-(difluoromethyl)-6-fluorophenyl)-5-fluoro-1-neopentyl-1H-indol-3-yl)-2,2-difluoroethyl)cyclopropanesulfonamide

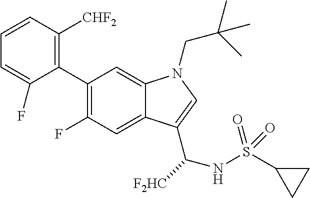 [SMILES: CC(C)(C)Cn1cc([C@H](NS(=O)(=O)C2CC2)C(F)F)c2cc(F)c(-c3c(F)cccc3C(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-bromo-1-(difluoromethyl)-3-fluorobenzene with anhydrous dioxane as solvent, and the reaction temperature of 140° C. ESI-MS (m/z): 533.1 [M+1]⁺.

### Example 117: (S)—N-(1-(6-(2-(difluoromethyl)-4-fluorophenyl)-5-fluoro-1-neopentyl-1H-indol-3-yl)-2,2-difluoroethyl)cyclopropanesulfonamide

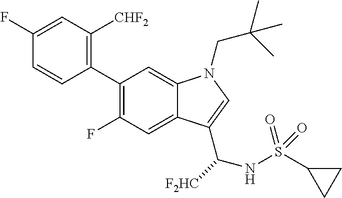 [SMILES: CC(C)(C)Cn1cc([C@H](NS(=O)(=O)C2CC2)C(F)F)c2cc(F)c(-c3ccc(F)cc3C(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 1-bromo-2-(difluoromethyl)-4-fluorobenzene with anhydrous dioxane as solvent, and the reaction temperature of 140° C. ESI-MS (m/z): 532.6 [M+1]⁺.

### Example 118: (S)—N-(1-(6-(2-cyano-4-(difluoromethyl)pyridin-3-yl)-1-neopentyl-1H-indol-3-yl)-2,2-difluoroethyl)cyclopropanesulfonamide

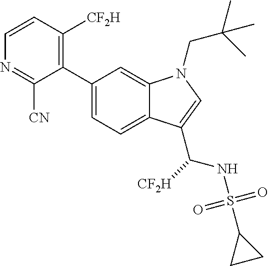 [SMILES: CC(C)(C)Cn1cc([C@H](NS(=O)(=O)C2CC2)C(F)F)c2ccc(-c3c(C(F)F)ccnc3C#N)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-4-(difluoromethyl)picolinonitrile. ESI-MS (m/z): 522.59 [M+1]⁺.

### Example 119: (S)—N-(1-(6-(2-(difluoromethyl)-5-fluoropyridin-3-yl)-1-neopentyl-1H-indol-3-yl)-2,2-difluoroethyl)cyclopropanesulfonamide

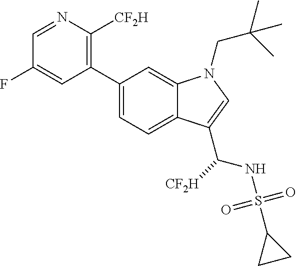 [SMILES: CC(C)(C)Cn1cc([C@H](NS(=O)(=O)C2CC2)C(F)F)c2ccc(-c3cc(F)cnc3C(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-2-(difluoromethyl)-5-fluoropyridine. ESI-MS (m/z): 515.83 [M+1]⁺.

### Example 120: (S)—N-(1-(6-(4-(difluoromethyl)-6-methylpyrimidin-5-yl)-1-neopentyl-1H-indol-3-yl)-2,2-difluoroethyl)cyclopropanesulfonamide

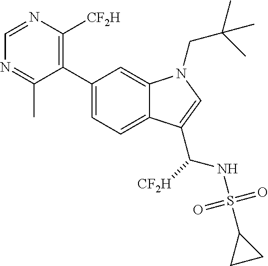 [SMILES: Cc1ncnc(C(F)F)c1-c1ccc2c([C@H](NS(=O)(=O)C3CC3)C(F)F)cn(CC(C)(C)C)c2c1]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 5-bromo-4-(difluoromethyl)-6-methylpyrimidine. ESI-MS (m/z): 512.96 [M+1]⁺.

### Example 121: (S)—N-(1-(6-(4-(difluoromethyl)-1-methyl-1H-pyrazol-5-yl)-1-neopentyl-1H-indol-3-yl)-2,2-difluoroethyl)cyclopropanesulfonamide

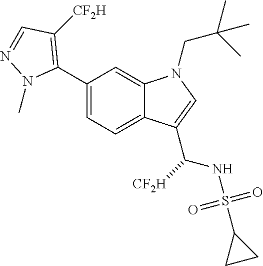 [SMILES: Cn1ncc(C(F)F)c1-c1ccc2c([C@H](NS(=O)(=O)C3CC3)C(F)F)cn(CC(C)(C)C)c2c1]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 5-bromo-4-(difluoromethyl)-1-methyl-1H-pyrazole. ESI-MS (m/z): 500.97 [M+1]⁺.

### Example 122: N-((1S)-1-(6-(2-cyano-4-(difluoromethyl)pyridin-3-yl)-5-fluoro-1-neopentyl-1H-indol-3-yl)-2,2-difluoroethyl)cyclopropanesulfonamide

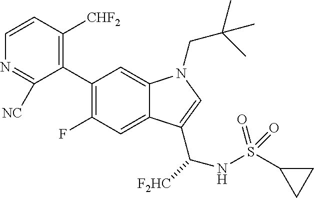 [SMILES: CC(C)(C)Cn1cc([C@H](NS(=O)(=O)C2CC2)C(F)F)c2cc(F)c(-c3c(C(F)F)ccnc3C#N)cc21]

### Step 1:3-bromo-4-(difluoromethyl)pyridine

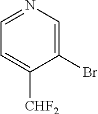

The title compound was obtained by applying the procedure of synthesis of 2-bromo-3-(difluoromethyl)benzonitrile. ESI-MS (m/z): 207.3, 209.4 [M+1]⁺.

### Step 2:3-bromo-4-(difluoromethyl)pyridine 1-oxide

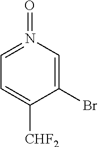 [SMILES: [O-][n+]1ccc(C(F)F)c(Br)c1]

The title compound was obtained by applying the procedure of synthesis of 3-bromo-4-(trifluoromethyl)pyridine 1-oxide. ESI-MS (m/z): 223.3, 225.3 [M+1]⁺.

### Step 3:3-bromo-4-(difluoromethyl)picolinonitrile

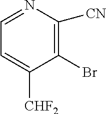 [SMILES: N#Cc1nccc(C(F)F)c1Br]

The title compound was obtained by applying the procedure of synthesis of 3-bromo-4-(trifluoromethyl)picolinonitrile. 1H NMR (CDCl₃, 400 MHz) δ 8.79 (d, J=4.8 Hz, 1H), 7.76 (d, J=4.8 Hz, 1H), 6.86 (t, J=54.0 Hz, 1H)

### Step 4: N-((1S)-1-(6-(2-cyano-4-(difluoromethyl)pyridin-3-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-4-(difluoromethyl)picolinonitrile with anhydrous dioxane as solvent, and the reaction temperature 140° C. ESI-MS (m/z): 540.6 [M+1]⁺.

### Example 123: (S)—N-(1-(6-(2-(difluoromethyl)-5-fluoropyridin-3-yl)-5-fluoro-1-neopentyl-1H-indol-3-yl)-2,2-difluoroethyl)cyclopropanesulfonamide

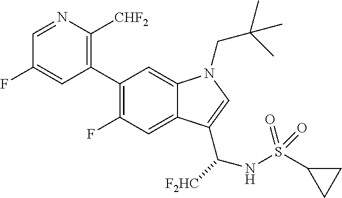 [SMILES: CC(C)(C)Cn1cc([C@H](NS(=O)(=O)C2CC2)C(F)F)c2cc(F)c(-c3cc(F)cnc3C(F)F)cc21]

### Step 1: methyl 3-bromo-5-fluoropicolinate

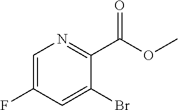

To a solution of 3-bromo-5-fluoropicolinic acid (2.09 g) in MeOH was added a few drops of con. H₂SO₄ and refluxed overnight. The completion was monitored by anal. HPLC. The mixture was concentrated and diluted with EtOAc, washed with sat'd NaHCO₃, brine and dried over Na₂SO₄. The solvent was removed and the crude was used for the next step with no purification. ESI-MS (m/z): 233.4, 235.4 [M+1]⁺.

### Step 2: (3-bromo-5-fluoropyridin-2-yl)methanol

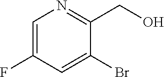

The title compound was obtained by applying the procedure of synthesis of (5-bromo-1-methyl-1H-pyrazol-4-yl)methanol. ESI-MS (m/z): 205.4, 207.4 [M+1]⁺.

### Step 3:3-bromo-5-fluoropicolinaldehyde

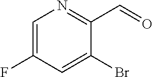

The title compound was obtained by applying the procedure of synthesis of 5-bromo-1-methyl-1H-pyrazole-4-carbaldehyde. ¹H NMR (CDCl₃, 400 MHz) δ 10.18 (s, 1H), 8.63 (d, J=2.2 Hz, 1H), 7.81 (m, 1H)

### Step 4:3-bromo-2-(difluoromethyl)-5-fluoropyridine

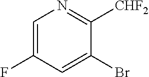 [SMILES: Fc1cnc(C(F)F)c(Br)c1]

The title compound was obtained by applying the procedure of synthesis of 2-bromo-3-(difluoromethyl)benzonitrile. ¹H NMR (CDCl₃, 400 MHz) δ 8.51 (d, J=2.4 Hz, 1H), 7.77-7.75 (m, 1H), 6.88 (t, J=55.2 Hz, 1H)

### Step 5: (S)—N-(1-(6-(2-(difluoromethyl)-5-fluoropyridin-3-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-2-(difluoromethyl)-5-fluoropyridine with anhydrous dioxane as solvent, and the reaction temperature 140° C. ESI-MS (m/z): 533.5 [M+1]⁺.

### Example 124: N-((1S)-1-(6-(4-(difluoromethyl)-6-methylpyrimidin-5-yl)-5-fluoro-1-neopentyl-1H-indol-3-yl)-2,2-difluoroethyl)cyclopropanesulfonamide

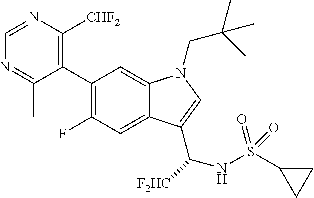 [SMILES: Cc1ncnc(C(F)F)c1-c1cc2c(cc1F)c([C@H](NS(=O)(=O)C1CC1)C(F)F)cn2CC(C)(C)C]

### Step 1: ethyl 6-methylpyrimidine-4-carboxylate

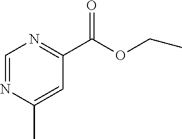

The mixture of ethyl (E)-4-methoxy-2-oxopent-3-enoate (12.0 g, 69.5 mmol) and potassium carbonate (17.3 g, 125.1 mmol) in acetonitrile (150 mL) was heat to 70° C., then formimidamide HCl salt (13 g, 125.1 mmol) was added portionwise. The mixture was then refluxed for 2 h. The completion was monitored by anal. HPLC. The mixture was cooled to rt and concentrated, the crude was dissolved with DCM and water. The aqueous layer was exacted with DCM twice. The organic layers were combined and dried over Na₂SO₄. The solvent was removed and the crude was purified by silica gel to obtain the title compound. ESI-MS (m/z): 166.7 [M+1]⁺.

### Step 2: ethyl 5-bromo-6-methylpyrimidine-4-carboxylate

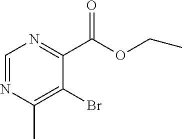

To a solution of ethyl 6-methylpyrimidine-4-carboxylate (2.0 g, 12.04 mmol) in EtOH (12 mL) at 0° C. was slowly added Br₂ (2.4 g, 15.65 mmol) in EtOH (4 mL). The reaction was continued to stir at 0° C. for 1 h. The solvent was removed and the crude was purified by silica gel to obtain the title compound. ¹H NMR (CDCl₃, 400 MHz) δ 9.0 (s, 1H), 4.48 (q, J=8.0 Hz, 2H), 2.72 (s, 3H), 1.44 (t, J=8.0 Hz, 3H)

### Step 3: (5-bromo-6-methylpyrimidin-4-yl)methanol

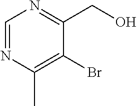

To a solution of ethyl 5-bromo-6-methylpyrimidine-4-carboxylate (0.71 g, 2.89 mmol) in EtOH (15 mL) at 0° C. was added NaBH₄ (0.219 g, 5.78 mmol) portion wise. The reaction was continued to stir at 0° C. for 1 h. The solvent was removed and the crude was purified by silica gel to obtain the title compound. ESI-MS (m/z): 202.3, 204.1 [M+1]⁺.

### Step 4:5-bromo-6-methylpyrimidine-4-carbaldehyde

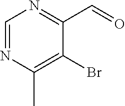

To a solution of (5-bromo-6-methylpyrimidin-4-yl)methanol (0.25 g, 1.25 mmol) in DCM (5 mL) at 0° C. was added Dess-martin reagent (0.8 g, 1.88 mmol). The reaction was stirred at rt for 1 h. The completion was monitored by anal. HPLC. The reaction was diluted with DCM and washed with sat'd NaHCO₃ and brine and dried over Na₂SO₄. The solvent was removed and the crude was purified by silica gel to obtain the title compound. 1H NMR (CDCl₃, 400 MHz) δ 10.21 (s, 1H), 9.16 (s, 1H), 2.78 (s, 3H)

### Step 5:5-bromo-4-(difluoromethyl)-6-methylpyrimidine

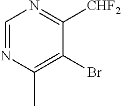

The title compound was obtained by applying the procedure of synthesis of 2-bromo-3-(difluoromethyl)benzonitrile. ¹H NMR (CDCl₃, 400 MHz) δ 9.06 (s, 1H), 6.85 (t, J=55.2 Hz, 1H), 2.74 (s, 3H)

### Step 6: N-((1S)-1-(6-(4-(difluoromethyl)-6-methylpyrimidin-5-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-4-(difluoromethyl)-6-methylpyrimidine with anhydrous dioxane as solvent, and the reaction temperature 140° C. ESI-MS (m/z): 530.6 [M+1]⁺.

### Example 125: (S)—N-(1-(6-(4-(difluoromethyl)-1-methyl-1H-pyrazol-5-yl)-5-fluoro-1-neopentyl-1H-indol-3-yl)-2,2-difluoroethyl)cyclopropanesulfonamide

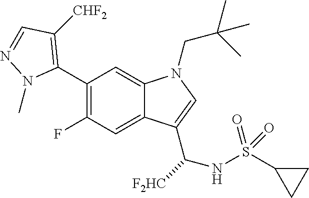 [SMILES: Cn1ncc(C(F)F)c1-c1cc2c(cc1F)c([C@H](NS(=O)(=O)C1CC1)C(F)F)cn2CC(C)(C)C]

### Step 1: ethyl 5-bromo-1-methyl-1H-pyrazole-4-carboxylate

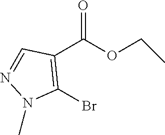

To a mixture of ethyl 5-amino-1-methyl-1H-pyrazole-4-carboxylate (3.35 g, 19.80 mmol) and CuBr₂ (6.63 g, 29.70 mmol) in acetonitrile (60 mL) at 50° C. was added slowly t-BuONO (3.54 mL, 29.7 mmol). The mixture was continued to stir for 2 h at the same temperature. The completion was monitored by anal. HPLC. The mixture was diluted with EtOAc and washed with sat'd NaHCO₃, and brine and dried over Na₂SO₄. The solvent was removed and the crude was purified by silica gel to obtain the title compound. ESI-MS (m/z): 232.4, 234.3 [M+1]⁺.

### Step 2: (5-bromo-1-methyl-1H-pyrazol-4-yl)methanol

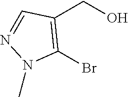

To a solution of ethyl 5-bromo-1-methyl-1H-pyrazole-4-carboxylate (2.59 g, 11.12 mmol) in THF (25 mL) at 0° C. was added slowly DIBAL (1M in DCM, 24 mL, 24.0 mmol). The solution was continued to stir overnight at room temperature. The completion was monitored by anal. HPLC. The reaction was quenched with water and diluted with EtOAc and washed with sat'd NaHCO₃, and brine and dried over Na₂SO₄. The solvent was removed and the crude was used for the next step with no further purification. ESI-MS (m/z): 190.4, 192.3 [M+1]⁺.

### Step 3:5-bromo-1-methyl-1H-pyrazole-4-carbaldehyde

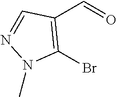

To the crude from Step 2 in DCM (20 mL) was added MnO₂ (9.67 g). The mixture was continued to stir overnight at room temperature. The completion was monitored by anal. HPLC. The mixture was diluted with DCM and filtered through celite pad. The solvent was removed and the crude was purified by silica gel to obtain the title compound. ESI-MS (m/z): 188.3, 190.4 [M+1]⁺.

### Step 4:5-bromo-4-(difluoromethyl)-1-methyl-1H-pyrazole

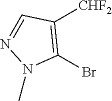

The title compound was obtained by applying the procedure of synthesis of 2-bromo-3-(difluoromethyl)benzonitrile. ¹H NMR (CDCl₃, 400 MHz) δ 7.69 (s, 1H), 6.58 (t, J=55.2 Hz, 1H), 3.90 (s, 3H)

### Step 5: (S)—N-(1-(6-(4-(difluoromethyl)-1-methyl-1H-pyrazol-5-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-4-(difluoromethyl)-1-methyl-1H-pyrazole with anhydrous dioxane as solvent, and the reaction temperature 140° C. ESI-MS (m/z): 518.6 [M+1]⁺.

### Example 126: N-((1S)-1-(6-(4-(difluoromethyl)-6-methylpyrimidin-5-yl)-5-fluoro-1-neopentyl-1H-indol-3-yl)-2,2-difluoroethyl)cyclopropanesulfonamide

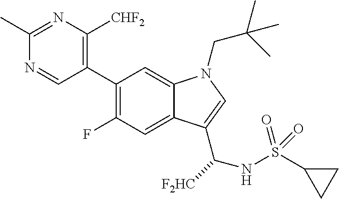 [SMILES: Cc1ncc(-c2cc3c(cc2F)c([C@H](NS(=O)(=O)C2CC2)C(F)F)cn3CC(C)(C)C)c(C(F)F)n1]

### Step 1: methyl 5-bromo-2-methylpyrimidine-4-carboxylate

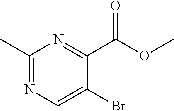

A mixture of acetimidamide (4.02 g, 42.52 mmol) and NaOEt in EtOH (21% by wt, 16 mL, 42.52 mmol) was heated to 50° C., then (Z)-2,3-dibromo-4-oxobut-2-enoic acid (5.4 g, 21.26 mmol) in EtOH (10 mL) was added slowly. The mixture was continued to stir for 1 h at the same temperature. NaOEt in EtOH (21% by wt, 8 mL, 21.26 mmol) was added. The mixture was slowly cooled to rt overnight. The completion was monitored by anal. HPLC. The mixture was filtered through celite pad and adjusted to pH4 by addition of 2N HCl. The solvent was removed and the crude was redissolved with MeOH, and a few drops of conc. H₂SO₄ was added. The mixture was refluxed overnight. The completion was monitored by anal. HPLC. The mixture was cooled to rt and the solvent was removed. The crude was diluted with EtOAc, washed with sat'd NaHCO₃ and brine, dried over Na₂SO₄. The solvent was removed and the crude was purified by silica gel to obtain the title compound. ESI-MS (m/z): 230.3, 231.2 [M+1]⁺.

### Step 2: (5-bromo-2-methylpyrimidin-4-yl)methanol

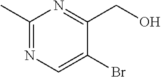

The title compound was obtained by applying the procedure of synthesis of (5-bromo-6-methylpyrimidin-4-yl)methanol. ESI-MS (m/z): 202.5, 204.1 [M+1]⁺.

### Step 3:5-bromo-2-methylpyrimidine-4-carbaldehyde

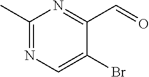

The title compound was obtained by applying the procedure of 5-bromo-6-methylpyrimidine-4-carbaldehyde. ESI-MS (m/z): 200.5, 203.4 [M+1]⁺.

### Step 4:5-bromo-4-(difluoromethyl)-2-methylpyrimidine

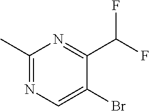

The title compound was obtained by applying the procedure of synthesis of 2-bromo-3-(difluoromethyl)benzonitrile. ESI-MS (m/z): 222.3, 224.3 [M+1]⁺.

### Step 5: N-((1S)-1-(6-(4-(difluoromethyl)-6-methylpyrimidin-5-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-4-(difluoromethyl)-2-methylpyrimidine with anhydrous dioxane as solvent, and the reaction temperature 140° C. ESI-MS (m/z): 530.6 [M+1]⁺.

### Example 127: N-((1S)-1-(6-(2-(difluoromethyl)-4-methylpyridin-3-yl)-5-fluoro-1-neopentyl-1H-indol-3-yl)-2,2-difluoroethyl)cyclopropanesulfonamide

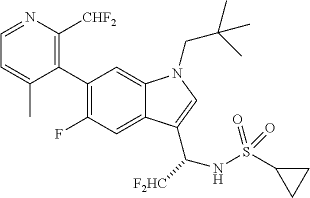 [SMILES: Cc1ccnc(C(F)F)c1-c1cc2c(cc1F)c([C@H](NS(=O)(=O)C1CC1)C(F)F)cn2CC(C)(C)C]

### Step 1:3-bromo-4-methylpyridine 1-oxide

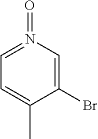

The title compound was obtained by applying the procedure of synthesis of 3-bromo-4-(trifluoromethyl)pyridine 1-oxide. ESI-MS (m/z): 187.3, 189.4 [M+1]⁺.

### Step 2:3-bromo-4-methylpicolinonitrile

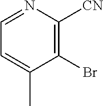

The title compound was obtained by applying the procedure of synthesis of 3-bromo-4-(trifluoromethyl)picolinonitrile. ¹H NMR (CDCl₃, 400 MHz) δ 8.48 (d, J=4.8 Hz, 1H), 7.37 (d, J=4.8 Hz, 1H), 2.51 (s, 3H)

### Step 3: methyl 3-bromo-4-methylpicolinate

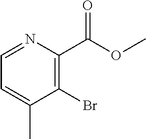

To a solution of 3-bromo-4-methylpicolinonitrile (0.559 g, 2.84 mmol) in MeOH (10 mL) was added NaOH (4N, 4 mL, 14.2 mmol) and heated to 110° C. for 2 h. The completion was monitored by anal. HPLC. The reaction was cooled to rt and acidified to pH 4 by addition of HCl (6N). The solvent was removed and the crude was redissolved with MeOH, and a few drops of con. H₂SO₄ was added. The mixture was refluxed overnight. The completion was monitored by anal. HPLC. The mixture was cooled to rt and the solvent was removed. The crude was diluted with EtOAc, washed with sat'd NaHCO₃ and brine, dried over Na₂SO₄. The solvent was removed and the crude was purified by silica gel to obtain the title compound. ESI-MS (m/z): 229.4, 231.2 [M+1]⁺.

### Step 4: (3-bromo-4-methylpyridin-2-yl)methanol

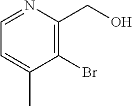

The title compound was obtained by applying the procedure of synthesis of (5-bromo-6-methylpyrimidin-4-yl)methanol. ESI-MS (m/z): 201.5, 203.2 [M+1]⁺.

### Step 5:3-bromo-4-methylpicolinaldehyde

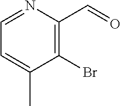

The title compound was obtained by applying the procedure of 5-bromo-6-methylpyrimidine-4-carbaldehyde. ESI-MS (m/z): 199.4, 201.4 [M+1]⁺.

### Step 6:3-bromo-2-(difluoromethyl)-4-methylpyridine

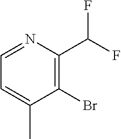

The title compound was obtained by applying the procedure of synthesis of 2-bromo-3-(difluoromethyl)benzonitrile. ¹H NMR (CDCl₃, 400 MHz) δ 8.48 (d, J=4.8 Hz, 1H), 7.30 (d, J=4.8 Hz, 1H), 6.98 (t, J=55.2 Hz, 1H), 2.48 (s, 3H)

### Step 7: N-((1S)-1-(6-(2-(difluoromethyl)-4-methylpyridin-3-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-2-(difluoromethyl)-4-methylpyridine with anhydrous dioxane as solvent, and the reaction temperature of 140° C. ESI-MS (m/z): 529.6 [M+1]⁺.

### Example 128: (S)—N-(1-(6-(2-(difluoromethyl)-4-methylpyridin-3-yl)-1-neopentyl-1H-indol-3-yl)-2,2-difluoroethyl)cyclopropanesulfonamide

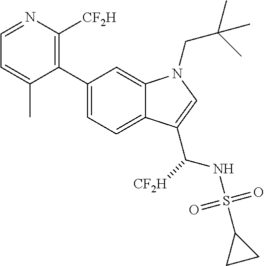 [SMILES: Cc1ccnc(C(F)F)c1-c1ccc2c([C@H](NS(=O)(=O)C3CC3)C(F)F)cn(CC(C)(C)C)c2c1]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 3-bromo-2-(difluoromethyl)-4-methylpyridine with anhydrous dioxane as solvent, and the reaction temperature of 140° C. ESI-MS (m/z): 511.6 [M+1]⁺.

### Example 129: N-((1S)-2,2-difluoro-1-(5-fluoro-6-(4-methyl-2-(trifluoromethyl)pyridin-3-yl)-1-neopentyl-1H-indol-3-yl)ethyl)cyclopropanesulfonamide

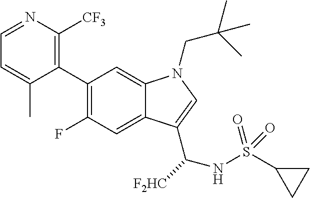 [SMILES: Cc1ccnc(C(F)(F)F)c1-c1cc2c(cc1F)c([C@H](NS(=O)(=O)C1CC1)C(F)F)cn2CC(C)(C)C]

### Step 1:3-bromo-2-iodo-4-methylpyridine

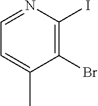

To a solution of 3-bromo-2-chloro-4-methylpyridine (0.73 g, 3.536 mmol) in acetonitrile (10 mL) was added NaI (1.59 g, 10.608 mmol) and TMSCl (0.45 mL, 3.536 mmol), and then heated to 90° C. overnight. The completion was monitored by anal. HPLC. The mixture was cooled to rt and diluted with EtOAc, washed with sat'd NaHCO₃, sat'd NaS₂O₃ and brine and dried over Na₂SO₄. The solvent was removed and the crude was purified by silica gel to obtain the title compound. ESI-MS (m/z): 297.9, 299.5 [M+1]⁺.

### Step 2:3-bromo-4-methyl-2-(trifluoromethyl)pyridine

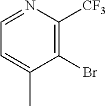

To a solution of 3-bromo-2-iodo-4-methylpyridine (0.2278 g, 0.764 mmol) in DMF (2 mL) was added CuI (0.218 g, 1.146 mmol) and methyl 2,2-difluoro-2-(fluorosulfonyl)acetate (0.15 mL, 1.146 mmol), and then heated to 80° C. overnight. The completion was monitored by anal. HPLC. The mixture was cooled to rt and diluted with EtOAc, washed with sat'd NaHCO₃ and brine and dried over Na₂SO₄. The solvent was removed and the crude was purified by silica gel to obtain the title compound. ESI-MS (m/z): 239.4, 240.9 [M+1]⁺.

### Step 3: N-((1S)-2,2-difluoro-1-(5-fluoro-6-(4-methyl-2-(trifluoromethyl)pyridin-3-yl)-1-neopentyl-1H-indol-3-yl)ethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-4-methyl-2-(trifluoromethyl)pyridine with anhydrous dioxane as solvent, and the reaction temperature of 140° C. ESI-MS (m/z): 547.6 [M+1]⁺.

### Example 130: N-((1S)-2,2-difluoro-1-(5-fluoro-6-(2-methyl-4-(trifluoromethyl)pyridin-3-yl)-1-neopentyl-1H-indol-3-yl)ethyl)cyclopropanesulfonamide

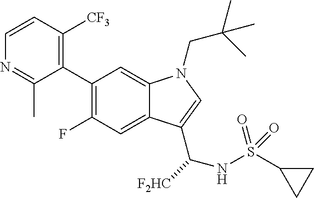 [SMILES: Cc1nccc(C(F)(F)F)c1-c1cc2c(cc1F)c([C@H](NS(=O)(=O)C1CC1)C(F)F)cn2CC(C)(C)C]

### Step 1:3-bromo-4-iodo-2-methylpyridine

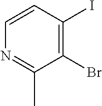

To a solution of 3-bromo-2-methylpyridin-4-amine (4.18 g, 22.348 mmol) and diiodomethane (12 g, 44.696 mmol) in acetonitrile (40 mL) at 0° C. was added slowly isopentyl nitrite (6.6 mL (49.17 mmol) in acetonitrile (10 mL). the reaction was continued to stir for 1 h, then to 60° C. overnight. The completion was monitored by anal. HPLC. The mixture was cooled to rt and diluted with EtOAc, washed with sat'd NaHCO₃ and brine and dried over Na₂SO₄. The solvent was removed and the crude was purified by silica gel to obtain the title compound. ESI-MS (m/z): 297.6, 299.5 [M+1]⁺.

### Step 2:3-bromo-2-methyl-4-(trifluoromethyl)pyridine

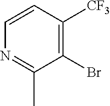

The title compound was obtained by applying the procedure of synthesis of 3-bromo-4-methyl-2-(trifluoromethyl)pyridine. ESI-MS (m/z): 239.4, 240.9 [M+1]⁺.

### Step 3: N-((1S)-2,2-difluoro-1-(5-fluoro-6-(2-methyl-4-(trifluoromethyl)pyridin-3-yl)-1-neopentyl-1H-indol-3-yl)ethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-2-methyl-4-(trifluoromethyl)pyridine with anhydrous dioxane as solvent, and the reaction temperature 140° C. ESI-MS (m/z): 547.6 [M+1]⁺.

### Example 131: N-((1S)-2,2-difluoro-1-(5-fluoro-6-(4-fluoro-2-(trifluoromethyl)pyridin-3-yl)-1-neopentyl-1H-indol-3-yl)ethyl)cyclopropanesulfonamide

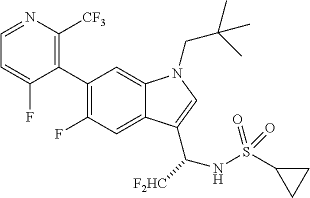 [SMILES: CC(C)(C)Cn1cc([C@H](NS(=O)(=O)C2CC2)C(F)F)c2cc(F)c(-c3c(F)ccnc3C(F)(F)F)cc21]

### Step 1: 3-bromo-2-chloro-4-fluoropyridine

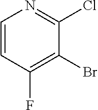

To a solution of 2-chloro-4-fluoropyridine (2.11 g, 16.0 mmol) in THF (40 mL) cooled to −78° C. under argon was added LDA (1M in THE, 16.5 mL, 16.5 mmol) dropwise. The mixture was continued to stir at −78° C. for 30 min. 1,2-dibromo-1,1,2,2-tetrachloroethane (5.68 g, 17.44 mmol) in THF (23 mL) was added dropwise. The mixture was continued to stir at −78° C. for 30 min and then warm to rt. The mixture was diluted with ether, washed with water and brine and dried over Na₂SO₄. The solvent was removed and the crude was purified by silica gel to obtain the title compound. 1H NMR (CDCl₃, 400 MHz) δ 8.30 (m, 1H), 7.05 (m, 1H)

### Step 2: 3-bromo-4-fluoro-2-iodopyridine

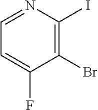

The title compound was obtained by applying the procedure of synthesis of 3-bromo-2-iodo-4-methylpyridine, using 3-bromo-2-chloro-4-fluoropyridine. ESI-MS (m/z): 301.8, 303.2 [M+1]⁺.

### Step 3: 3-bromo-4-fluoro-2-(trifluoromethyl)pyridine

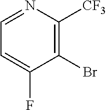 [SMILES: Fc1ccnc(C(F)(F)F)c1Br]

The title compound was obtained by applying the procedure of synthesis of 3-bromo-4-methyl-2-(trifluoromethyl)pyridine. ¹H NMR (CDCl₃, 400 MHz) δ 8.58 (m, 1H), 7.29 (m, 1H)

### Step 4: N-((1S)-2,2-difluoro-1-(5-fluoro-6-(4-fluoro-2-(trifluoromethyl)pyridin-3-yl)-1-neopentyl-1H-indol-3-yl)ethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-4-fluoro-2-(trifluoromethyl)pyridine with anhydrous dioxane as solvent, and the reaction temperature of 140° C. ESI-MS (m/z): 551.5 [M+1]⁺.

### Example 132: (S)—N-(2,2-difluoro-1-(5-fluoro-6-(5-fluoro-2-(trifluoromethyl)pyridin-3-yl)-1-neopentyl-1H-indol-3-yl)ethyl)cyclopropanesulfonamide

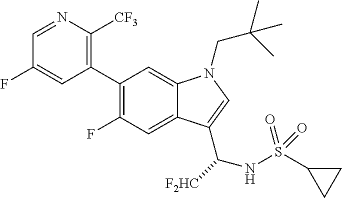 [SMILES: CC(C)(C)Cn1cc([C@H](NS(=O)(=O)C2CC2)C(F)F)c2cc(F)c(-c3cc(F)cnc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-5-fluoro-2-(trifluoromethyl)pyridine with anhydrous dioxane as solvent, and the reaction temperature of 140° C. ESI-MS (m/z): 551.5 [M+1]⁺.

### Example 133: (S)—N-(1-(6-(6-cyano-4-(trifluoromethyl)pyridin-3-yl)-5-fluoro-1-neopentyl-1H-indol-3-yl)-2,2-difluoroethyl)cyclopropanesulfonamide

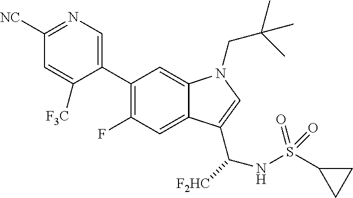 [SMILES: CC(C)(C)Cn1cc([C@H](NS(=O)(=O)C2CC2)C(F)F)c2cc(F)c(-c3cnc(C#N)cc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-4-(trifluoromethyl)picolinonitrile with anhydrous dioxane as solvent, and the reaction temperature of 140° C. ESI-MS (m/z): 559.4 [M+1]⁺.

### Example 134: (S)—N-(1-(6-(3-(difluoromethyl)-5-fluoropyridin-2-yl)-5-fluoro-1-neopentyl-1H-indol-3-yl)-2,2-difluoroethyl)cyclopropanesulfonamide

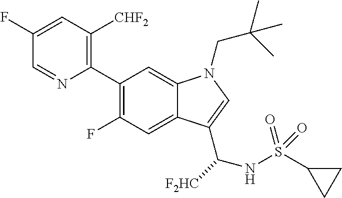 [SMILES: CC(C)(C)Cn1cc([C@H](NS(=O)(=O)C2CC2)C(F)F)c2cc(F)c(-c3ncc(F)cc3C(F)F)cc21]

### Step 1:2-chloro-3-(difluoromethyl)-5-fluoropyridine

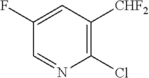 [SMILES: Fc1cnc(Cl)c(C(F)F)c1]

The title compound was obtained by applying the procedure of synthesis of 2-bromo-3-(difluoromethyl)benzonitrile. ESI-MS (m/z): 181.5 [M+1]⁺.

### Step 2: (S)—N-(1-(6-(3-(difluoromethyl)-5-fluoropyridin-2-yl)-5-fluoro-1-neopentyl-1H-indol-3-yl)-2,2-difluoroethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-chloro-3-(difluoromethyl)-5-fluoropyridine with anhydrous dioxane as solvent, and the reaction temperature of 140° C. ESI-MS (m/z): 533.5 [M+1]⁺.

### Example 135: N-((1S)-1-(6-(2-cyano-4-(trifluoromethyl)pyridin-3-yl)-5-fluoro-1-neopentyl-1H-indol-3-yl)-2,2-difluoroethyl)cyclobutanesulfonamide

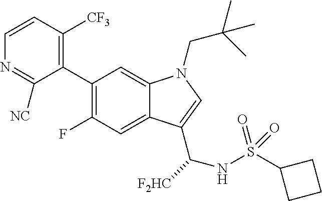 [SMILES: CC(C)(C)Cn1cc([C@H](NS(=O)(=O)C2CCC2)C(F)F)c2cc(F)c(-c3c(C(F)(F)F)ccnc3C#N)cc21]

### Step 1: (S)—N-(1-(6-bromo-5-fluoro-1-neopentyl-1H-indol-3-yl)-2,2-difluoroethyl)cyclobutanesulfonamide

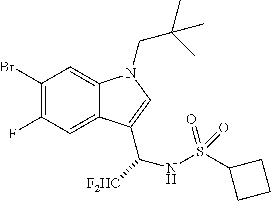 [SMILES: CC(C)(C)Cn1cc([C@H](NS(=O)(=O)C2CCC2)C(F)F)c2cc(F)c(Br)cc21]

The title compound was prepared following the same general protocol as described for Step 4, Example 2, using (S)-1-(6-bromo-5-fluoro-1-neopentyl-1H-indol-3-yl)-2,2-difluoroethan-1-amine and cyclobutanesulfonyl chloride. ESI-MS (m/z): 481.3, 482.7 [M+1]⁺.

### Step 2: (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclobutanesulfonamide

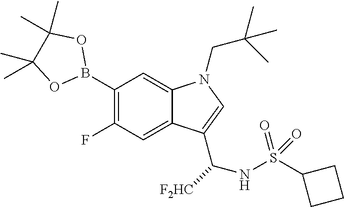 [SMILES: CC(C)(C)Cn1cc([C@H](NS(=O)(=O)C2CCC2)C(F)F)c2cc(F)c(B3OC(C)(C)C(C)(C)O3)cc21]

The title compound was prepared following the same general protocol as described for Step 5, Example 2, using (S)—N-(1-(6-bromo-5-fluoro-1-neopentyl-1H-indol-3-yl)-2,2-difluoroethyl)cyclobutanesulfonamide. ESI-MS (m/z): 528.7 [M+1]⁺.

### Step 3: N-((1S)-1-(6-(2-cyano-4-(trifluoromethyl)pyridin-3-yl)-5-fluoro-1-neopentyl-1H-indol-3-yl)-2,2-difluoroethyl)cyclobutanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclobutanesulfonamide and 3-bromo-4-(trifluoromethyl)picolinonitrile with anhydrous dioxane as solvent, and the reaction temperature of 140° C. ¹H NMR (CDCl₃, 400 MHz) δ 8.93 (d, J=4.8 Hz, 1H), 7.88 (d, J=4.8 Hz, 1H), 7.55 (m, 1H), 7.29-7.24 (m, 2H), 6.18 (td, J=55.8 Hz, 2.2 Hz, 1H), 5.13-5.03 (m, 1H), 4.85 (dd, J=26.6 Hz, 8.0 Hz, 1H), 3.94-3.78 (m, 2H), 3.71-3.63 (m, 1H), 2.49-2.40 (m, 2H), 2.14-2.05 (m, 2H), 1.91-1.86 (m, 2H), 0.97 (s, 9H).

### Example 136: N-((1S)-1-(6-(2-cyano-6-(trifluoromethyl)phenyl)-5-fluoro-1-neopentyl-1H-indol-3-yl)-2,2-difluoroethyl)cyclobutanesulfonamide

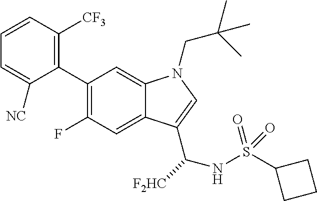 [SMILES: CC(C)(C)Cn1cc([C@H](NS(=O)(=O)C2CCC2)C(F)F)c2cc(F)c(-c3c(C#N)cccc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclobutanesulfonamide and 2-bromo-3-(trifluoromethyl)benzonitrile with anhydrous dioxane as solvent, and the reaction temperature 140° C. 1H NMR (CDCl₃, 400 MHz) δ 8.02 (d, J=8.0 Hz, 1H), 7.94-7.93 (m, 1H), 7.67 (t, J=8.0 Hz, 1H), 7.51-7.46 (m, 1H), 7.26-7.21 (m, 2H), 6.14 (tt, J=55.8 Hz, 2.2 Hz, 1H), 5.11-5.02 (m, 1H), 4.78 (dd, J=26.6 Hz, 8.0 Hz, 1H), 3.93-3.82 (m, 2H), 3.69-3.61 (m, 1H), 2.48-2.41 (m, 2H), 2.14-2.05 (m, 2H), 1.91-1.82 (m, 2H), 0.97 (s, 9H).

### Example 137: (S)—N-(2,2-difluoro-1-(5-fluoro-6-(5-fluoro-2-(trifluoromethyl)pyridin-3-yl)-1-neopentyl-1H-indol-3-yl)ethyl)cyclobutanesulfonamide

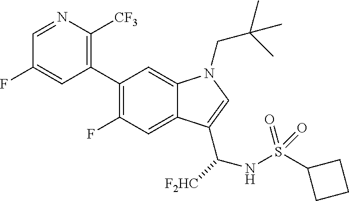 [SMILES: CC(C)(C)Cn1cc([C@H](NS(=O)(=O)C2CCC2)C(F)F)c2cc(F)c(-c3cc(F)cnc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclobutanesulfonamide and 3-bromo-5-fluoro-2-(trifluoromethyl)pyridine with anhydrous dioxane as solvent, and the reaction temperature 140° C. ESI-MS (m/z): 564.4 [M+1]⁺.

### Example 138: (S)—N-(1-(6-(5-cyano-3-(trifluoromethyl)pyridin-2-yl)-5-fluoro-1-neopentyl-1H-indol-3-yl)-2,2-difluoroethyl)cyclopropanesulfonamide

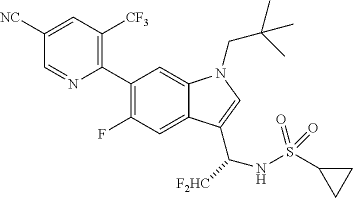 [SMILES: CC(C)(C)Cn1cc([C@H](NS(=O)(=O)C2CC2)C(F)F)c2cc(F)c(-c3ncc(C#N)cc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 6-chloro-5-(trifluoromethyl)nicotinonitrile with anhydrous dioxane as solvent, and the reaction temperature 140° C. ESI-MS (m/z): 558.7 [M+1]⁺.

### Example 139: (S)—N-(2,2-difluoro-1-(5-fluoro-6-(6-methyl-2-(trifluoromethyl)pyridin-3-yl)-1-neopentyl-1H-indol-3-yl)ethyl)cyclopropanesulfonamide

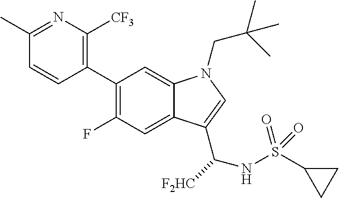 [SMILES: Cc1ccc(-c2cc3c(cc2F)c([C@H](NS(=O)(=O)C2CC2)C(F)F)cn3CC(C)(C)C)c(C(F)(F)F)n1]

### Step 1:3-bromo-5-fluoro-2-(trifluoromethyl)pyridine and 5-bromo-2-methyl-4-(trifluoromethyl)pyridine

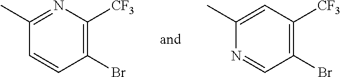

To a mixture of 5-bromo-2-methylpyridine (0.25 g, 1.457 mmol) and zinc sulfinate salt (0.966 g, 2.91 mmol) in CHCl₃ (8 mL) and water (2.5 mL) at 0° C. was added TBHP (~80% solution in water, 0.55 mL, 4.37 mmol) dropwise. The reaction was then warm to room temperature and heated to 50° C. overnight. The reaction was cooled to rt, neutralized with sat'd NaHCO₃. The aqueous layer was extracted with DCM and the combined organic layers were washed with brine and dried over Na₂SO₄, and concentrated to get the crude, which was purified by silica gel to obtain the title compounds: 3-bromo-6-methyl-2-(trifluoromethyl)pyridine: ¹H NMR (CDCl₃, 400 MHz) δ 7.90 (d, J=8.2 Hz, 1H), 7.20 (d, J=8.2 Hz, 1H), 2.58 (s, 3H); 5-bromo-2-methyl-4-(trifluoromethyl)pyridine: ¹H NMR (CDCl₃, 400 MHz) δ 8.76 (s, 1H), 7.28 (s, 1H), 2.60 (s, 3H)

### Step 2: (S)—N-(2,2-difluoro-1-(5-fluoro-6-(6-methyl-2-(trifluoromethyl)pyridin-3-yl)-1-neopentyl-1H-indol-3-yl)ethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-5-fluoro-2-(trifluoromethyl)pyridine with anhydrous dioxane as solvent, and the reaction temperature of 140° C. ESI-MS (m/z): 547.9 [M+1]⁺.

### Example 140: (S)—N-(2,2-difluoro-1-(5-fluoro-6-(6-methyl-4-(trifluoromethyl)pyridin-3-yl)-1-neopentyl-1H-indol-3-yl)ethyl)cyclopropanesulfonamide

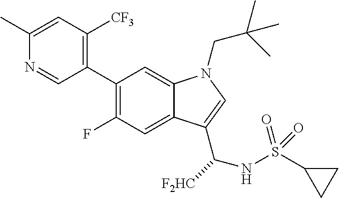 [SMILES: Cc1cc(C(F)(F)F)c(-c2cc3c(cc2F)c([C@H](NS(=O)(=O)C2CC2)C(F)F)cn3CC(C)(C)C)cn1]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-2-methyl-4-(trifluoromethyl)pyridine with anhydrous dioxane as solvent, and the reaction temperature of 140° C. ESI-MS (m/z): 547.9 [M+1]⁺.

### Example 141: (S)—N-(2,2-difluoro-1-(5-fluoro-6-(5-methyl-2-(trifluoromethyl)pyridin-3-yl)-1-neopentyl-1H-indol-3-yl)ethyl)cyclopropanesulfonamide

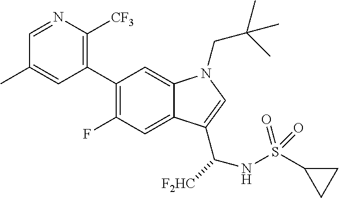 [SMILES: Cc1cnc(C(F)(F)F)c(-c2cc3c(cc2F)c([C@H](NS(=O)(=O)C2CC2)C(F)F)cn3CC(C)(C)C)c1]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-5-methyl-2-(trifluoromethyl)pyridine with anhydrous dioxane as solvent, and the reaction temperature of 140° C. ESI-MS (m/z): 547.9 [M+1]⁺.

### Example 142: (S)—N-(2,2-difluoro-1-(5-fluoro-6-(5-methyl-2-(trifluoromethyl)pyridin-3-yl)-1-neopentyl-1H-pyrrolo[2,3-b]pyridin-3-yl)ethyl)cyclopropanesulfonamide

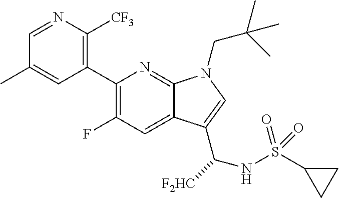 [SMILES: Cc1cnc(C(F)(F)F)c(-c2nc3c(cc2F)c([C@H](NS(=O)(=O)C2CC2)C(F)F)cn3CC(C)(C)C)c1]

The title compound was prepared following the same general protocol as described for Step 6, Example 2, using (S)—N-(1-(6-chloro-5-fluoro-1-neopentyl-1H-pyrrolo[2,3-b]pyridin-3-yl)-2,2-difluoroethyl)cyclopropanesulfonamide and 5-methyl-3-(4,4,5,5-tetramethyl-1,3,2-dioxaborolan-2-yl)-2-(trifluoromethyl)pyridine with anhydrous dioxane as solvent, and the reaction temperature of 140° C. ESI-MS (m/z): 548.8 [M+1]⁺.

### Example 143: (S)—N-(1-(6-(5-chloro-2-(trifluoromethyl)pyridin-3-yl)-5-fluoro-1-neopentyl-1H-indol-3-yl)-2,2-difluoroethyl)cyclopropanesulfonamide

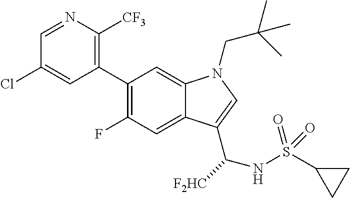 [SMILES: CC(C)(C)Cn1cc([C@H](NS(=O)(=O)C2CC2)C(F)F)c2cc(F)c(-c3cc(Cl)cnc3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 3-bromo-5-chloro-2-(trifluoromethyl)pyridine with anhydrous dioxane as solvent, and the reaction temperature of 140° C. ESI-MS (m/z): 567.8 [M+1]⁺.

### Example 144: (S)—N-(2,2-difluoro-1-(5-fluoro-6-(5-methyl-3-(trifluoromethyl)pyrazin-2-yl)-1-neopentyl-1H-indol-3-yl)ethyl)cyclopropanesulfonamide

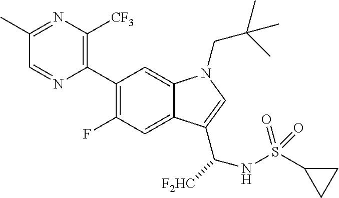 [SMILES: Cc1cnc(-c2cc3c(cc2F)c([C@H](NS(=O)(=O)C2CC2)C(F)F)cn3CC(C)(C)C)c(C(F)(F)F)n1]

### Step 1:5-bromo-3-(trifluoromethyl)pyrazin-2-amine

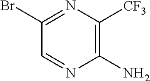 [SMILES: Nc1ncc(Br)nc1C(F)(F)F]

To the mixture of 3-(trifluoromethyl)pyrazin-2-amine (0.179 g, 1.1 mmol) in ACN (5 mL) was added NBS (0.294 g, 1.65 mmol), then the reaction was continued overnight at rt. The completion of reaction was monitored by anal·HPLC. The solvent was removed and the crude was purified by silica gel to obtain the title compound. ESI-MS (m/z): 241.3, 243.3 [M+1]⁺.

### Step 2:5-methyl-3-(trifluoromethyl)pyrazin-2-amine

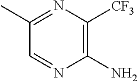

A solution of 5-bromo-3-(trifluoromethyl)pyrazin-2-amine (1 eq), 2,4,6-trimethyl-1,3,5,2,4,6-trioxatriborinane (2 eq), K₂CO₃ (3 eq), and Pd(PPh₃)₄ (0.1 eq) in dioxane/water (4/1) was degassed and heated in a 80° C. oil bath for 16 h. The solvent was removed in vacuo to obtain the crude which was purified by prep-HPLC to yield the title compound. ESI-MS (m/z): 177.4 [M+1]⁺.

### Step 3:2-iodo-5-methyl-3-(trifluoromethyl)pyrazine

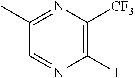

To a mixture of 5-methyl-3-(trifluoromethyl)pyrazin-2-amine (0.045 g, 0.254 mmol), CuI (0.015 g, 0.076 mmol), I₂ (0.032 g, 0.127 mmol) and KI (0.042 g, 0.254 mmol) in THF (2.5 mL) was added slowly isopentyl nitrite (0.11 mL, 0.762 mmol). The reaction was heated to 70° C. for 4 h. The completion was monitored by anal. HPLC. The mixture was cooled to rt and diluted with EtOAc, washed with sat'd NaHCO₃ and brine and dried over Na₂SO₄. The solvent was removed and the crude was purified by silica gel to obtain the title compound. ESI-MS (m/z): 288.6 [M+1]⁺.

### Step 4: (S)—N-(2,2-difluoro-1-(5-fluoro-6-(5-methyl-3-(trifluoromethyl)pyrazin-2-yl)-1-neopentyl-1H-indol-3-yl)ethyl)cyclopropanesulfonamide

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 2-iodo-5-methyl-3-(trifluoromethyl)pyrazine with anhydrous dioxane as solvent, and the reaction temperature 120° C. ESI-MS (m/z): 548.7 [M+1]⁺.

### Example 145: (S)—N-(2,2-difluoro-1-(5-fluoro-1-neopentyl-6-(2-(trifluoromethyl)pyridin-3-yl)-1H-pyrrolo[2,3-b]pyridin-3-yl)ethyl)cyclopropanesulfonamide

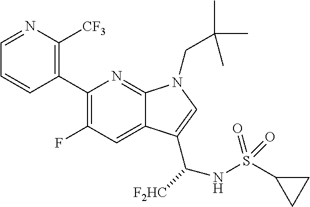 [SMILES: CC(C)(C)Cn1cc([C@H](NS(=O)(=O)C2CC2)C(F)F)c2cc(F)c(-c3cccnc3C(F)(F)F)nc21]

### Step 1:1-(6-chloro-5-fluoro-1-neopentyl-1H-pyrrolo[2,3-b]pyridin-3-yl)-2,2-difluoroethan-1-one

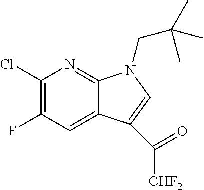 [SMILES: CC(C)(C)Cn1cc(C(=O)C(F)F)c2cc(F)c(Cl)nc21]

The title compound was prepared following the same general protocol as described for Step 1, Example 11, using 6-chloro-5-fluoro-1-neopentyl-1H-pyrrolo[2,3-b]pyridine and 2,2-difluoroacetic anhydride. ESI-MS (m/z): 240.7 [M+1]⁺.

### Step 2: (S,E)-N-(1-(6-chloro-5-fluoro-1-neopentyl-1H-pyrrolo[2,3-b]pyridin-3-yl)-2,2-difluoroethylidene)-2-methylpropane-2-sulfinamide

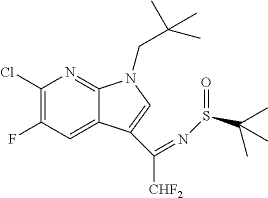 [SMILES: CC(C)(C)Cn1cc(/C(=N\[S@@](=O)C(C)(C)C)C(F)F)c2cc(F)c(Cl)nc21]

The title compound was prepared following the same general protocol as described for Step 2, Example 11, using 1-(6-chloro-5-fluoro-1-neopentyl-1H-pyrrolo[2,3-b]pyridin-3-yl)-2,2-difluoroethan-1-one and (S)-2-methylpropane-2-sulfinamide, and the reaction was stirred for 2 h at 90° C. ESI-MS (m/z): 421.8 [M+1]⁺.

### Step 3: (S)—N—((S)-1-(6-chloro-5-fluoro-1-neopentyl-1H-pyrrolo[2,3-b]pyridin-3-yl)-2,2-difluoroethyl)-2-methylpropane-2-sulfinamide

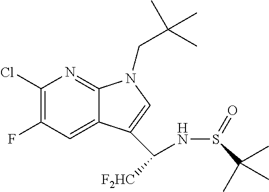 [SMILES: CC(C)(C)Cn1cc([C@H](N[S@@](=O)C(C)(C)C)C(F)F)c2cc(F)c(Cl)nc21]

The title compound was prepared following the same general protocol as described for Step 3, Example 31, using (S,E)-N-(1-(6-chloro-5-fluoro-1-neopentyl-1H-pyrrolo[2,3-b]pyridin-3-yl)-2,2-difluoroethylidene)-2-methylpropane-2-sulfinamide. 1H NMR (CDCl3, 400 MHz) δ 7.68 (d, J=8.7 Hz, 1H), 7.37 (s, 1H), 6.03 (td, J=55.8 Hz, 2.2 Hz, 1H), 4.88-4.81 (m, 1H), 4.15-4.09 (m, 3H), 1.21 (s, 9H), 0.98 (S, 9H)

### Step 4: (S)-1-(6-chloro-5-fluoro-1-neopentyl-1H-pyrrolo[2,3-b]pyridin-3-yl)-2,2-difluoroethan-1-amine

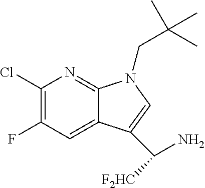 [SMILES: CC(C)(C)Cn1cc([C@H](N)C(F)F)c2cc(F)c(Cl)nc21]

The title compound was prepared following the same general protocol as described for Step 3, Example 2, using (S)—N—((S)-1-(6-chloro-5-fluoro-1-neopentyl-1H-pyrrolo[2,3-b]pyridin-3-yl)-2,2-difluoroethyl)-2-methylpropane-2-sulfinamide

### Step 5: (S)—N-(1-(6-chloro-5-fluoro-1-neopentyl-1H-pyrrolo[2,3-b]pyridin-3-yl)-2,2-difluoroethyl)cyclopropanesulfonamide

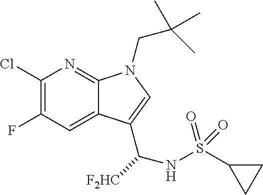 [SMILES: CC(C)(C)Cn1cc([C@H](NS(=O)(=O)C2CC2)C(F)F)c2cc(F)c(Cl)nc21]

The title compound was prepared following the same general protocol as described for Step 4, Example 2, using (S)-1-(6-chloro-5-fluoro-1-neopentyl-1H-pyrrolo[2,3-b]pyridin-3-yl)-2,2-difluoroethan-1-amine and cyclopropanesulfonyl chloride. ESI-MS (m/z): 423.9 [M+1]⁺.

Step 6: The title compound was prepared following the same general protocol as described for Step 6, Example 2, using (S)—N-(1-(6-chloro-5-fluoro-1-neopentyl-1H-pyrrolo[2,3-b]pyridin-3-yl)-2,2-difluoroethyl)cyclopropanesulfonamide and (2-(trifluoromethyl)pyridin-3-yl)boronic acid with anhydrous dioxane as solvent, and the reaction temperature 140° C. ESI-MS (m/z): 534.8 [M+1]⁺.

### Example 146: (S)—N-(1-(1-cyclobutyl-5-fluoro-6-(2-(trifluoromethyl)pyridin-3-yl)-1H-indol-3-yl)-2,2-difluoroethyl)cyclopropanesulfonamide

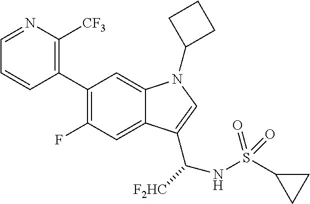 [SMILES: O=S(=O)(N[C@@H](c1cn(C2CCC2)c2cc(-c3cccnc3C(F)(F)F)c(F)cc12)C(F)F)C1CC1]

### Step 1:6-bromo-1-cyclobutyl-5-fluoro-1H-indole

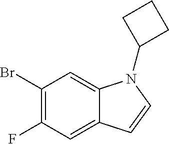 [SMILES: Fc1cc2ccn(C3CCC3)c2cc1Br]

The title compound was prepared following the same general protocol as described for Step 1, Example 2, using 6-bromo-5-fluoro-1H-indole and bromocyclobutane. ESI-MS (m/z): 267.5, 269.3 [M+1]⁺.

### Step 2:1-(6-bromo-1-cyclobutyl-5-fluoro-1H-indol-3-yl)-2,2-difluoroethan-1-one

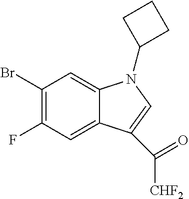 [SMILES: O=C(c1cn(C2CCC2)c2cc(Br)c(F)cc12)C(F)F]

The title compound was prepared following the same general protocol as described for Step 1, Example 11, using 6-bromo-1-cyclobutyl-5-fluoro-1H-indole and 2,2-difluoroacetic anhydride. ESI-MS (m/z): 345.5, 347.6 [M+1]⁺.

### Step 3: (S,E)-N-(1-(6-bromo-1-cyclobutyl-5-fluoro-1H-indol-3-yl)-2,2-difluoroethylidene)-2-methylpropane-2-sulfinamide

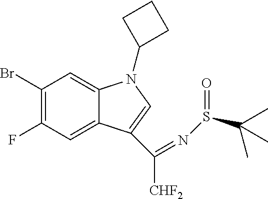 [SMILES: CC(C)(C)[S@](=O)/N=C(\c1cn(C2CCC2)c2cc(Br)c(F)cc12)C(F)F]

The title compound was prepared following the same general protocol as described for Step 2, Example 11, using 1-(6-bromo-1-cyclobutyl-5-fluoro-1H-indol-3-yl)-2,2-difluoroethan-1-one and (S)-2-methylpropane-2-sulfinamide, and the reaction was stirred for 2 h at 90° C. ESI-MS (m/z): 448.5, 450.6 [M+1]⁺.

### Step 4: (S)—N—((S)-1-(6-bromo-1-cyclobutyl-5-fluoro-1H-indol-3-yl)-2,2-difluoroethyl)-2-methylpropane-2-sulfinamide

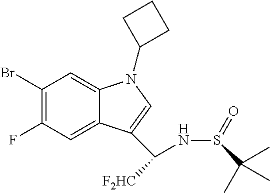 [SMILES: CC(C)(C)[S@](=O)N[C@@H](c1cn(C2CCC2)c2cc(Br)c(F)cc12)C(F)F]

The title compound was prepared following the same general protocol as described for Step 3, Example 31, using (S,E)-N-(1-(6-bromo-1-cyclobutyl-5-fluoro-1H-indol-3-yl)-2,2-difluoroethylidene)-2-methylpropane-2-sulfinamide. ¹H NMR (CDCl₃, 400 MHz) δ 7.51 (d, J=5.6 Hz, 1H), 7.40 (s, 1H), 7.35 (d, J=9.32 Hz, 1H), 6.06 (td, J=55.8 Hz, 2.2 Hz, 1H), 4.83-4.72 (m, 2H), 3.85-3.83 (m, 1H), 2.63-2.59 (m, 2H), 2.47-2.39 (m, 2H), 2.08-1.91 (m, 2H), 1.21 (s, 9H)

### Step 5: (S)-1-(6-bromo-1-cyclobutyl-5-fluoro-1H-indol-3-yl)-2,2-difluoroethan-1-amine

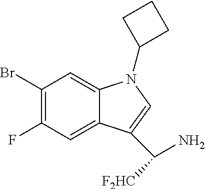 [SMILES: N[C@@H](c1cn(C2CCC2)c2cc(Br)c(F)cc12)C(F)F]

The title compound was prepared following the same general protocol as described for Step 3, Example 2, using (S)—N—((S)-1-(6-bromo-1-cyclobutyl-5-fluoro-1H-indol-3-yl)-2,2-difluoroethyl)-2-methylpropane-2-sulfinamide

### Step 6: (S)—N-(1-(6-bromo-1-cyclobutyl-5-fluoro-1H-indol-3-yl)-2,2-difluoroethyl)cyclopropanesulfonamide

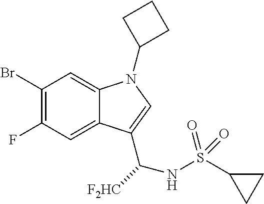 [SMILES: O=S(=O)(N[C@@H](c1cn(C2CCC2)c2cc(Br)c(F)cc12)C(F)F)C1CC1]

The title compound was prepared following the same general protocol as described for Step 4, Example 2, using (S)—N-(1-(6-bromo-1-cyclobutyl-5-fluoro-1H-indol-3-yl)-2,2-difluoroethyl)cyclopropanesulfonamide and cyclopropanesulfonyl chloride. ESI-MS (m/z): 450.5, 452.6 [M+1]⁺.

### Step 7: (S)—N-(1-(1-cyclobutyl-5-fluoro-6-(2-(trifluoromethyl)pyridin-3-yl)-1H-indol-3-yl)-2,2-difluoroethyl)cyclopropanesulfonamide

The title compound was prepared following the same general protocol as described for Step 6, Example 2, using (S)—N-(1-(6-bromo-1-cyclobutyl-5-fluoro-1H-indol-3-yl)-2,2-difluoroethyl)cyclopropanesulfonamide and (2-(trifluoromethyl)pyridin-3-yl)boronic acid with anhydrous dioxane as solvent, and the reaction temperature 140° C. ESI-MS (m/z): 518.5 [M+1]⁺.

### Example 147: ((S)—N-(1-(1-cyclobutyl-5-fluoro-6-(2-(trifluoromethyl)pyridin-3-yl)-1H-pyrrolo[2,3-b]pyridin-3-yl)-2,2-difluoroethyl)cyclopropanesulfonamide

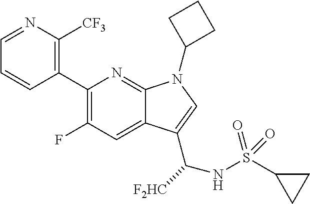 [SMILES: O=S(=O)(N[C@@H](c1cn(C2CCC2)c2nc(-c3cccnc3C(F)(F)F)c(F)cc12)C(F)F)C1CC1]

### Step 1:6-chloro-1-cyclobutyl-5-fluoro-1H-pyrrolo[2,3-b]pyridine

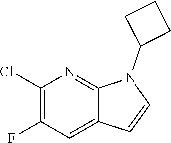 [SMILES: Fc1cc2ccn(C3CCC3)c2nc1Cl]

The title compound was prepared following the same general protocol as described for Step 1, Example 2, using 6-chloro-5-fluoro-1H-pyrrolo[2,3-b]pyridine and bromocyclobutane. ESI-MS (m/z): 224.8 [M+1]⁺.

### Step 2:1-(6-chloro-1-cyclobutyl-5-fluoro-1H-pyrrolo[2,3-b]pyridin-3-yl)-2,2-difluoroethan-1-one

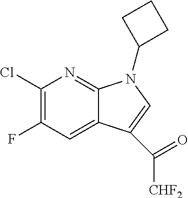 [SMILES: O=C(c1cn(C2CCC2)c2nc(Cl)c(F)cc12)C(F)F]

The title compound was prepared following the same general protocol as described for Step 1, Example 11, using 6-chloro-1-cyclobutyl-5-fluoro-1H-pyrrolo[2,3-b]pyridine and 2,2-difluoroacetic anhydride. ESI-MS (m/z): 302.7 [M+1]⁺.

### Step 3: (S,E)-N-(1-(6-chloro-1-cyclobutyl-5-fluoro-1H-pyrrolo[2,3-b]pyridin-3-yl)-2,2-difluoroethylidene)-2-methylpropane-2-sulfinamide

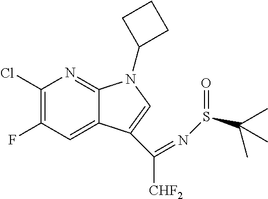 [SMILES: CC(C)(C)[S@](=O)/N=C(\c1cn(C2CCC2)c2nc(Cl)c(F)cc12)C(F)F]

The title compound was prepared following the same general protocol as described for Step 2, Example 11, using 1-(6-chloro-1-cyclobutyl-5-fluoro-1H-pyrrolo[2,3-b]pyridin-3-yl)-2,2-difluoroethan-1-one and (S)-2-methylpropane-2-sulfinamide, and the reaction was stirred for 2 h at 90° C. ESI-MS (m/z): 405.9 [M+1]⁺.

### Step 4: (S)—N—((S)-1-(6-chloro-1-cyclobutyl-5-fluoro-1H-pyrrolo[2,3-b]pyridin-3-yl)-2,2-difluoroethyl)-2-methylpropane-2-sulfinamide

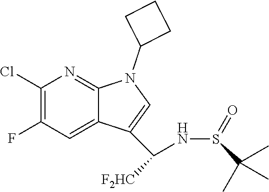 [SMILES: CC(C)(C)[S@](=O)N[C@@H](c1cn(C2CCC2)c2nc(Cl)c(F)cc12)C(F)F]

The title compound was prepared following the same general protocol as described for Step 3, Example 31, using (S,E)-N-(1-(6-chloro-1-cyclobutyl-5-fluoro-1H-pyrrolo[2,3-b]pyridin-3-yl)-2,2-difluoroethylidene)-2-methylpropane-2-sulfinamide. ¹H NMR (CDCl3, 400 MHz) δ 7.70 (d, J=8.7 Hz, 1H), 7.60 (s, 1H), 6.05 (td, J=55.8 Hz, 2.2 Hz, 1H), 5.29 (q, J=8.0 Hz, 1H), 4.90-4.83 (m, 1H), 3.91-3.88 (m, 1H), 2.62-2.43 (m, 4H), 1.97-1.90 (m, 2H), 1.21 (s, 9H)

### Step 5: (S)-1-(6-chloro-1-cyclobutyl-5-fluoro-1H-pyrrolo[2,3-b]pyridin-3-yl)-2,2-difluoroethan-1-amine

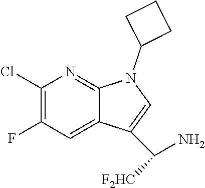 [SMILES: N[C@@H](c1cn(C2CCC2)c2nc(Cl)c(F)cc12)C(F)F]

The title compound was prepared following the same general protocol as described for Step 3, Example 2, using (S)—N—((S)-1-(6-chloro-1-cyclobutyl-5-fluoro-1H-pyrrolo[2,3-b]pyridin-3-yl)-2,2-difluoroethyl)-2-methylpropane-2-sulfinamide

### Step 6: (S)—N-(1-(6-chloro-1-cyclobutyl-5-fluoro-1H-pyrrolo[2,3-b]pyridin-3-yl)-2,2-difluoroethyl)cyclopropanesulfonamide

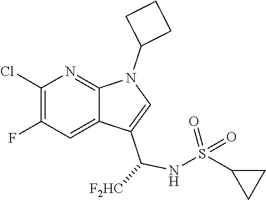 [SMILES: O=S(=O)(N[C@@H](c1cn(C2CCC2)c2nc(Cl)c(F)cc12)C(F)F)C1CC1]

The title compound was prepared following the same general protocol as described for Step 4, Example 2, using (S)-1-(6-chloro-1-cyclobutyl-5-fluoro-1H-pyrrolo[2,3-b]pyridin-3-yl)-2,2-difluoroethan-1-amine and cyclopropanesulfonyl chloride. ESI-MS (m/z): 407.8 [M+1]⁺.

### Step 7: ((S)—N-(1-(1-cyclobutyl-5-fluoro-6-(2-(trifluoromethyl)pyridin-3-yl)-1H-pyrrolo[2,3-b]pyridin-3-yl)-2,2-difluoroethyl)cyclopropanesulfonamide

The title compound was prepared following the same general protocol as described for Step 6, Example 2, using (S)—N-(1-(6-chloro-1-cyclobutyl-5-fluoro-1H-pyrrolo[2,3-b]pyridin-3-yl)-2,2-difluoroethyl)cyclopropanesulfonamide and (2-(trifluoromethyl)pyridin-3-yl)boronic acid with anhydrous dioxane as solvent, and the reaction temperature 140° C. ESI-MS (m/z): 536.5 [M+1]⁺.

### Example 148: (S)—N-(2,2-difluoro-1-(5-fluoro-6-(5-fluoro-2-(trifluoromethyl)pyridin-3-yl)-1-neopentyl-1H-pyrrolo[2,3-b]pyridin-3-yl)ethyl)cyclopropanesulfonamide

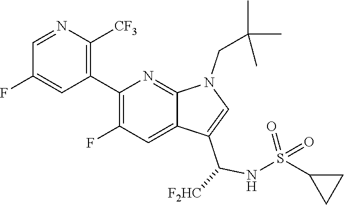 [SMILES: CC(C)(C)Cn1cc([C@H](NS(=O)(=O)C2CC2)C(F)F)c2cc(F)c(-c3cc(F)cnc3C(F)(F)F)nc21]

The title compound was prepared following the same general protocol as described for Step 6, Example 2, using (S)—N-(1-(6-chloro-5-fluoro-1-neopentyl-1H-pyrrolo[2,3-b]pyridin-3-yl)-2,2-difluoroethyl)cyclopropanesulfonamide and 5-fluoro-3-(4,4,5,5-tetramethyl-1,3,2-dioxaborolan-2-yl)-2-(trifluoromethyl)pyridine with anhydrous dioxane as solvent, and the reaction temperature of 140° C. ESI-MS (m/z): 552.8 [M+1]⁺.

### Example 149: (S)—N-(1-(1-cyclobutyl-5-fluoro-6-(5-fluoro-2-(trifluoromethyl)pyridin-3-yl)-1H-indol-3-yl)-2,2-difluoroethyl)cyclopropanesulfonamide

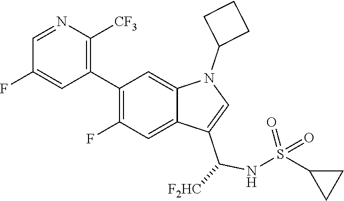 [SMILES: O=S(=O)(N[C@@H](c1cn(C2CCC2)c2cc(-c3cc(F)cnc3C(F)(F)F)c(F)cc12)C(F)F)C1CC1]

### Step 1:5-fluoro-3-(4,4,5,5-tetramethyl-1,3,2-dioxaborolan-2-yl)-2-(trifluoromethyl)pyridine

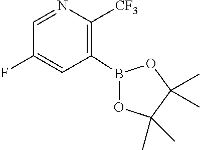 [SMILES: CC1(C)OB(c2cc(F)cnc2C(F)(F)F)OC1(C)C]

The title compound was prepared following the same general protocol as described for Step 5, Example 2, using 3-bromo-5-fluoro-2-(trifluoromethyl)pyridine. ESI-MS (m/z): 291.5 [M+1]⁺.

### Step 2: (S)—N-(1-(1-cyclobutyl-5-fluoro-6-(5-fluoro-2-(trifluoromethyl)pyridin-3-yl)-1H-indol-3-yl)-2,2-difluoroethyl)cyclopropanesulfonamide

The title compound was prepared following the same general protocol as described for Step 6, Example 2, using (S)—N-(1-(6-bromo-1-cyclobutyl-5-fluoro-1H-indol-3-yl)-2,2-difluoroethyl)cyclopropanesulfonamide and 5-fluoro-3-(4,4,5,5-tetramethyl-1,3,2-dioxaborolan-2-yl)-2-(trifluoromethyl)pyridine with anhydrous dioxane as solvent, and the reaction temperature 140° C. ESI-MS (m/z): 535.7 [M+1]⁺.

### Example 150: (S)—N-(1-(1-cyclobutyl-5-fluoro-6-(5-fluoro-2-(trifluoromethyl)pyridin-3-yl)-1H-pyrrolo[2,3-b]pyridin-3-yl)-2,2-difluoroethyl)cyclopropanesulfonamide

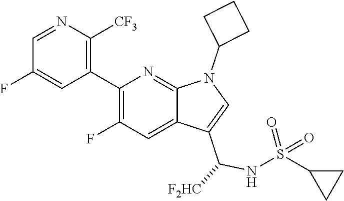 [SMILES: O=S(=O)(N[C@@H](c1cn(C2CCC2)c2nc(-c3cc(F)cnc3C(F)(F)F)c(F)cc12)C(F)F)C1CC1]

The title compound was prepared following the same general protocol as described for Step 6, Example 2, using (S)—N-(1-(6-chloro-1-cyclobutyl-5-fluoro-1H-pyrrolo[2,3-b]pyridin-3-yl)-2,2-difluoroethyl)cyclopropanesulfonamide and 5-fluoro-3-(4,4,5,5-tetramethyl-1,3,2-dioxaborolan-2-yl)-2-(trifluoromethyl)pyridine with anhydrous dioxane as solvent, and the reaction temperature of 140° C. ESI-MS (m/z): 536.5 [M+1]⁺.

### Example 151: (S)—N-(1-(1-(3-cyanocyclobutyl)-5-fluoro-6-(5-fluoro-2-(trifluoromethyl)pyridin-3-yl)-1H-indol-3-yl)-2,2-difluoroethyl)cyclopropanesulfonamide

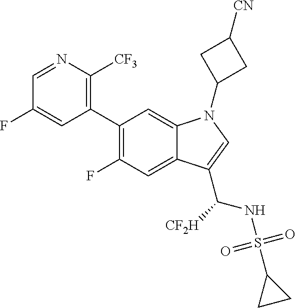 [SMILES: N#CC1CC(n2cc([C@H](NS(=O)(=O)C3CC3)C(F)F)c3cc(F)c(-c4cc(F)cnc4C(F)(F)F)cc32)C1]

### Step 1:3-cyanocyclobutyl 4-methylbenzenesulfonate

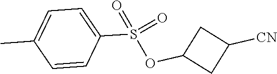 [SMILES: Cc1ccc(S(=O)(=O)OC2CC(C#N)C2)cc1]

To the mixture of 3-hydroxycyclobutane-1-carbonitrile (1.0 g, 10.29 mmol) and TEA (5.3 mL, 30.87 mmol) in DCM (20 mL) was added 4-methylbenzenesulfonyl chloride (2.35 g, 12.34 mml). The reaction was stirred at rt overnight. The completion was monitored by anal. HPLC. The mixture was diluted with DCM, washed with sat'd NaHCO₃, water and brine and dried over Na₂SO₄. The mixture was concentrated and the crude was purified by silica gel to obtain the title compound. ¹H NMR (CDCl₃, 400 MHz) δ 7.77 (d, J=8.4 Hz, 2H), 7.36 (d, J=8.0 Hz, 2H), 4.76 (q, J=8.0 Hz, 1H), 2.73-2.63 (m, 3H), 2.59-2.52 (m, 2H), 2.49 (s, 3H)

### Step 2:1-(6-bromo-5-fluoro-1H-indol-3-yl)-2,2-difluoroethan-1-one

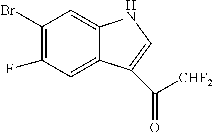 [SMILES: O=C(c1c[nH]c2cc(Br)c(F)cc12)C(F)F]

The title compound was prepared following the same general protocol as described for Step 1, Example 11, using 6-bromo-5-fluoro-1H-indole and 2,2-difluoroacetic anhydride. ESI-MS (m/z): 291.5, 293.5 [M+1]⁺.

### Step 3:3-(6-bromo-3-(2,2-difluoroacetyl)-5-fluoro-1H-indol-1-yl)cyclobutane-1-carbonitrile

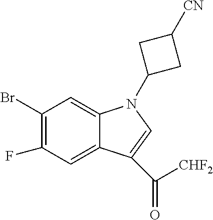 [SMILES: N#CC1CC(n2cc(C(=O)C(F)F)c3cc(F)c(Br)cc32)C1]

The title compound was prepared following the same general protocol as described for Step 1, Example 2, using 1-(6-bromo-5-fluoro-1H-indol-3-yl)-2,2-difluoroethan-1-one and 3-cyanocyclobutyl 4-methylbenzenesulfonate. ¹H NMR (CDCl₃, 400 MHz) δ 8.16 (d, J=8.9 Hz, 1H), 8.11 (s, 1H), 7.52 (d, J=4.0 Hz, 1H), 6.11 (t, J=54.0 Hz, 1H), 4.81-4.69 (m, 1H), 3.15-3.12 (m, 3H), 2.95-2.88 (m, 2H)

### Step 4: (S,E)-N-(1-(6-bromo-1-(3-cyanocyclobutyl)-5-fluoro-1H-indol-3-yl)-2,2-difluoroethylidene)-2-methylpropane-2-sulfinamide

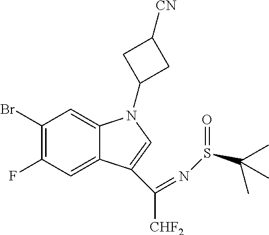 [SMILES: CC(C)(C)[S@](=O)/N=C(\c1cn(C2CC(C#N)C2)c2cc(Br)c(F)cc12)C(F)F]

The title compound was prepared following the same general protocol as described for Step 2, Example 11, using 3-(6-bromo-3-(2,2-difluoroacetyl)-5-fluoro-1H-indol-1-yl)cyclobutane-1-carbonitrile and (S)-2-methylpropane-2-sulfinamide, and the reaction was stirred for 2 h at 90° C. ¹H NMR (CDCl₃, 400 MHz) δ 7.99 (d, J=9.6 Hz, 1H), 7.95 (s, 1H), 7.49 (d, J=5.5 Hz, 1H), 4.85-4.79 (m, 1H), 3.17-3.08 (m, 3H), 2.94-2.87 (m, 2H), 1.36 (S, 9h)

### Step 5: (S)—N—((S)-1-(6-bromo-1-(3-cyanocyclobutyl)-5-fluoro-1H-indol-3-yl)-2,2-difluoroethyl)-2-methylpropane-2-sulfinamide

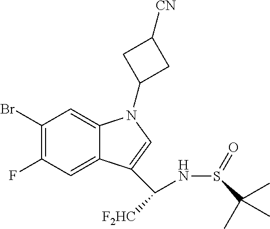 [SMILES: CC(C)(C)[S@](=O)N[C@@H](c1cn(C2CC(C#N)C2)c2cc(Br)c(F)cc12)C(F)F]

The title compound was prepared following the same general protocol as described for Step 3, Example 31, using (S,E)-N-(1-(6-bromo-1-(3-cyanocyclobutyl)-5-fluoro-1H-indol-3-yl)-2,2-difluoroethylidene)-2-methylpropane-2-sulfinamide. ¹H NMR (CDCl₃, 400 MHz) δ 7.47 (d, J=5.6 Hz, 1H), 7.40 (s, 1H), 7.37 (d, J=9.2 Hz, 1H), 6.07 (td, J=55.8 Hz, 2.2 Hz, 1H), 4.89-4.74 (m, 2H), 3.88 (d, J=3.5 Hz, 1H), 3.11-3.07 (m, 3H), 2.89-2.78 (m, 2H), 1.22 (s, 9H)

### Step 6: (S)—N-(1-(6-bromo-1-(3-cyanocyclobutyl)-5-fluoro-1H-indol-3-yl)-2,2-difluoroethyl)formamide

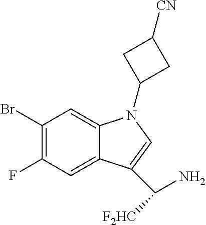 [SMILES: N#CC1CC(n2cc([C@H](N)C(F)F)c3cc(F)c(Br)cc32)C1]

The title compound was prepared following the same general protocol as described for Step 3, Example 2, using (S)—N—((S)-1-(6-bromo-1-(3-cyanocyclobutyl)-5-fluoro-1H-indol-3-yl)-2,2-difluoroethyl)-2-methylpropane-2-sulfinamide

### Step 7: (S)—N-(1-(6-bromo-1-(3-cyanocyclobutyl)-5-fluoro-1H-indol-3-yl)-2,2-difluoroethyl)cyclopropanesulfonamide

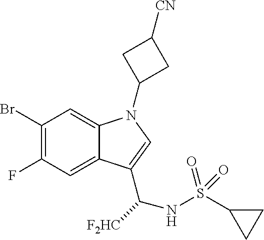 [SMILES: N#CC1CC(n2cc([C@H](NS(=O)(=O)C3CC3)C(F)F)c3cc(F)c(Br)cc32)C1]

The title compound was prepared following the same general protocol as described for Step 4, Example 2, using (S)—N-(1-(6-bromo-1-(3-cyanocyclobutyl)-5-fluoro-1H-indol-3-yl)-2,2-difluoroethyl)formamide and cyclopropanesulfonyl chloride. ¹H NMR (CDCl₃, 400 MHz) δ 7.47-7.43 (m, 3H), 6.09 (td, J=55.8 Hz, 2.2 Hz, 1H), 5.45 (d, J=8.7 Hz, 1H), 5.11-5.01 (m, 1H), 4.78-4.70 (m, 1H), 3.04-2.98 (m, 3H), 2.89-2.71 (m, 2H), 2.41-2.34 (m, 1H), 1.19-1.09 (m, 2H), 0.93-0.83 (m, 2H)

### Step 8: (S)—N-(1-(1-(3-cyanocyclobutyl)-5-fluoro-6-(5-fluoro-2-(trifluoromethyl)pyridin-3-yl)-1H-indol-3-yl)-2,2-difluoroethyl)cyclopropanesulfonamide

The title compound was prepared following the same general protocol as described for Step 6, Example 2, using (S)—N-(1-(6-bromo-1-(3-cyanocyclobutyl)-5-fluoro-1H-indol-3-yl)-2,2-difluoroethyl)cyclopropanesulfonamide and 5-fluoro-3-(4,4,5,5-tetramethyl-1,3,2-dioxaborolan-2-yl)-2-(trifluoromethyl)pyridine with anhydrous dioxane as solvent, and the reaction temperature 140. ¹H NMR (CDCl₃, 400 MHz) δ 8.60 (d, J=2.6 Hz, 1H), 7.55-7.48 (m, 3H), 7.15 (d, 5.7 Hz, 1H), 6.17 (t, J=56.0 Hz, 1H), 5.20-5.11 (m, 2H), 4.83-4.75 (m, 1H), 3.08-3.01 (m, 3H), 2.89-2.79 (m, 2H), 2.47-2.44 (m, 1H), 1.19-1.09 (m, 2H), 0.93-0.83 (m, 2H)

### Example 152: (S)—N-(1-(1-(azetidin-3-yl)-5-fluoro-6-(4-fluoro-2-(trifluoromethyl)phenyl)-1H-indol-3-yl)-2,2-difluoroethyl)cyclopropanesulfonamide

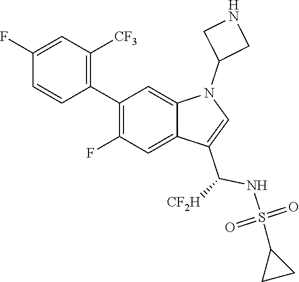 [SMILES: O=S(=O)(N[C@@H](c1cn(C2CNC2)c2cc(-c3ccc(F)cc3C(F)(F)F)c(F)cc12)C(F)F)C1CC1]

### Step 1: tert-butyl 3-(6-bromo-3-(2,2-difluoroacetyl)-5-fluoro-1H-indol-1-yl)azetidine-1-carboxylate

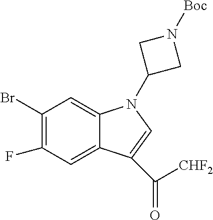 [SMILES: CC(C)(C)OC(=O)N1CC(n2cc(C(=O)C(F)F)c3cc(F)c(Br)cc32)C1]

The title compound was prepared following the same general protocol as described for Step 1, Example 2, using 1-(6-bromo-5-fluoro-1H-indol-3-yl)-2,2-difluoroethan-1-one and tert-butyl 3-bromoazetidine-1-carboxylate. ¹H NMR (CDCl₃, 400 MHz) δ 8.22 (t, J=1.6 Hz, 1H), 8.19 (d, J=8.9 Hz, 1H), 7.66 (d, J=5.4 Hz, 1H), 6.12 (t, J=54.0 Hz, 1H), 5.32-5.18 (m, 1H), 4.60 (t, J=7.2 Hz, 2H), 4.33 (dd, J=6.0 Hz, 6.0 Hz, 2H), 1.51 (s, 9H)

### Step 2: tert-butyl (S,E)-3-(6-bromo-3-(1-((tert-butylsulfinyl)imino)-2,2-difluoroethyl)-5-fluoro-1H-indol-1-yl)azetidine-1-carboxylate

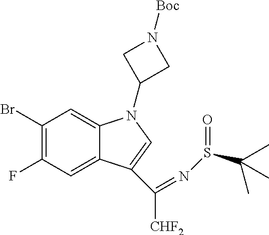 [SMILES: CC(C)(C)OC(=O)N1CC(n2cc(/C(=N\[S@@](=O)C(C)(C)C)C(F)F)c3cc(F)c(Br)cc32)C1]

The title compound was prepared following the same general protocol as described for Step 2, Example 11, using tert-butyl 3-(6-bromo-3-(2,2-difluoroacetyl)-5-fluoro-1H-indol-1-yl)azetidine-1-carboxylate and (S)-2-methylpropane-2-sulfinamide, and the reaction was stirred for 2 h at 90° C. ¹H NMR (CDCl₃, 400 MHz) δ 8.03-8.00 (m, 3H), 7.61 (d, J=5.4 Hz, 1H), 5.15-5.11 (m, 1H), 4.54 (t, J=7.2 Hz, 2H), 4.35-4.30 (m, 2H), 1.50 (s, 9H), 1.36 (s, 9H)

### Step 3: tert-butyl 3-(6-bromo-3-((S)-1-(((S)-tert-butylsulfinyl)amino)-2,2-difluoroethyl)-5-fluoro-1H-indol-1-yl)azetidine-1-carboxylate

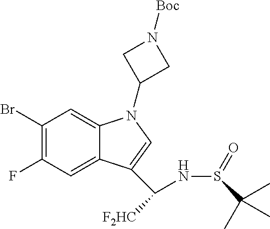 [SMILES: CC(C)(C)OC(=O)N1CC(n2cc([C@H](N[S@@](=O)C(C)(C)C)C(F)F)c3cc(F)c(Br)cc32)C1]

The title compound was prepared following the same general protocol as described for Step 3, Example 31, using tert-butyl (S,E)-3-(6-bromo-3-(1-((tert-butylsulfinyl)imino)-2,2-difluoroethyl)-5-fluoro-1H-indol-1-yl)azetidine-1-carboxylate. ¹H NMR (CDCl₃, 400 MHz) δ 7.59 (d, J=5.5 Hz, 1H), 7.49 (s, 1H), 7.40 (d, J=9.1 Hz, 1H), 6.09 (td, J=55.8 Hz, 2.2 Hz, 1H), 5.11-5.07 (m, 1H), 4.89-4.83 (m, 1H), 4.52 (t, J=6.1 Hz, 2H), 4.33-4.27 (m, 2H), 3.89 (d, J=3.5 Hz, 1H), 1.51 (s, 9H), 1.23 (s, 9H)

### Step 4: tert-butyl(S)-3-(3-(1-amino-2,2-difluoroethyl)-6-bromo-5-fluoro-1H-indol-1-yl)azetidine-1-carboxylate

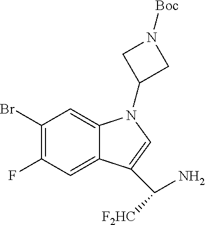 [SMILES: CC(C)(C)OC(=O)N1CC(n2cc([C@H](N)C(F)F)c3cc(F)c(Br)cc32)C1]

To the mixture of tert-butyl 3-(6-bromo-3-((S)-1-(((S)-tert-butylsulfinyl)amino)-2,2-difluoroethyl)-5-fluoro-1H-indol-1-yl)azetidine-1-carboxylate (0.7156 g, 1.295 mmol) in MeOH (1.3 mL) was added dropwise HCl (4N in dioxane, 0.5 mL, 2.0 mmol). The reaction was continued to stir at rt for 30 min. The completion of reaction was monitored by anal HPLC. The mixture was diluted with EtOAc, washed with sat'd NaHCO₃, and brine, and dried over Na₂SO₄. The solvent was removed to obtain the title compound with no further purification. ¹H NMR (CDCl₃, 400 MHz) δ 7.59 (d, J=5.5 Hz, 1H), 7.96 (s, 1H), 7.50-7.48 (m, 2H), 6.20 (t, J=55.8 Hz, 1H), 5.22-5.21 (m, 1H), 5.07-5.03 (m, 1H), 4.45 (t, J=8.8 Hz, 2H), 4.29-4.27 (m, 2H), 1.42 (s, 9H)

### Step 5: tert-butyl(S)-3-(6-bromo-3-(1-(cyclopropanesulfonamido)-2,2-difluoroethyl)-5-fluoro-1H-indol-1-yl)azetidine-1-carboxylate

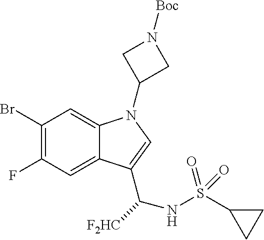 [SMILES: CC(C)(C)OC(=O)N1CC(n2cc([C@H](NS(=O)(=O)C3CC3)C(F)F)c3cc(F)c(Br)cc32)C1]

The title compound was prepared following the same general protocol as described for Step 4, Example 2, using tert-butyl(S)-3-(3-(1-amino-2,2-difluoroethyl)-6-bromo-5-fluoro-1H-indol-1-yl)azetidine-1-carboxylate and cyclopropanesulfonyl chloride. ¹H NMR (CDCl₃, 400 MHz) δ 7.54-7.48 (m, 3H), 6.09 (td, J=55.8 Hz, 2.2 Hz, 1H), 5.52 (d, J=8.4 Hz, 1H), 5.10-5.00 (m, 2H), 4.48-4.45 (m, 2H), 4.23-4.20 (m, 2H), 2.37-2.30 (m, 1H), 1.55 (S, 9h), 1.16-1.09 (m, 2H), 0.93-0.79 (m, 2H).

### Step 6: tert-butyl(S)-3-(3-(1-(cyclopropanesulfonamido)-2,2-difluoroethyl)-5-fluoro-6-(4-fluoro-2-(trifluoromethyl)phenyl)-1H-indol-1-yl)azetidine-1-carboxylate

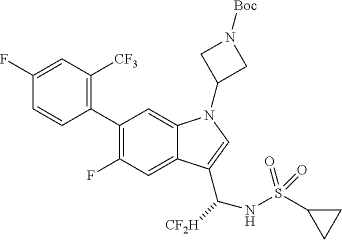 [SMILES: CC(C)(C)OC(=O)N1CC(n2cc([C@H](NS(=O)(=O)C3CC3)C(F)F)c3cc(F)c(-c4ccc(F)cc4C(F)(F)F)cc32)C1]

The title compound was prepared following the same general protocol as described for Step 6, Example 2, using tert-butyl(S)-3-(6-bromo-3-(1-(cyclopropanesulfonamido)-2,2-difluoroethyl)-5-fluoro-1H-indol-1-yl)azetidine-1-carboxylate and (4-fluoro-2-(trifluoromethyl)phenyl)boronic acid with anhydrous dioxane as solvent, and the reaction temperature of 140° C. ESI-MS (m/z): 635.6 [M+1]⁺.

### Step 7: (S)—N-(1-(1-(azetidin-3-yl)-5-fluoro-6-(4-fluoro-2-(trifluoromethyl)phenyl)-1H-indol-3-yl)-2,2-difluoroethyl)cyclopropanesulfonamide

tert-butyl(S)-3-(3-(1-(cyclopropanesulfonamido)-2,2-difluoroethyl)-5-fluoro-6-(4-fluoro-2-(trifluoromethyl)phenyl)-1H-indol-1-yl)azetidine-1-carboxylate obtained from the above step was dissolved with TFA/DCM (30%, 5 mL) and stirred at rt for 1 h. The completion of the reaction was monitored by anal HPLC. The mixture was concentrated and the crude was purified by preparative HPLC to obtain the title compound. ¹H NMR (methanol-d₄, 400 MHz) δ 7.87 (s, 1H), 7.58-7.48 (m, 2H), 7.39-7.35 (m, 2H), 7.32 (d, J=5.7 Hz, 1H), 6.16 (td, J=55.8 Hz, 2.2 Hz, 1H), 5.64 (q, J=8.4 Hz, 1H), 5.11 (t,d, J=13.9 Hz, 2.6 Hz, 1H), 4.56-4.50 (m, 4H), 2.40-2.36 (m, 1H), 1.09-1.03 (m, 2H), 0.88-0.78 (m, 2H)

### Example 153: (S)—N-(2,2-difluoro-1-(5-fluoro-6-(4-fluoro-2-(trifluoromethyl)phenyl)-1-neopentyl-1H-pyrrolo[2,3-b]pyridin-3-yl)ethyl)cyclopropanesulfonamide

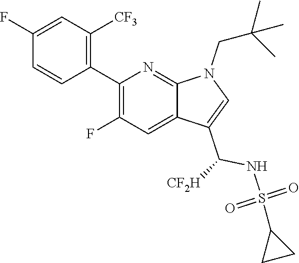 [SMILES: CC(C)(C)Cn1cc([C@H](NS(=O)(=O)C2CC2)C(F)F)c2cc(F)c(-c3ccc(F)cc3C(F)(F)F)nc21]

The title compound was prepared following the same general protocol as described for Step 6, Example 2, using (S)—N-(1-(6-chloro-1-cyclobutyl-5-fluoro-1H-pyrrolo[2,3-b]pyridin-3-yl)-2,2-difluoroethyl)cyclopropanesulfonamide and (4-fluoro-2-(trifluoromethyl)phenyl)boronic acid with anhydrous dioxane as solvent, and the reaction temperature of 140° C. ESI-MS (m/z): 535.5 [M+1]⁺.

### Example 154: (S)—N-(1-(1-cyclobutyl-5-fluoro-6-(4-fluoro-2-(trifluoromethyl)phenyl)-1H-indol-3-yl)-2,2-difluoroethyl)cyclopropanesulfonamide

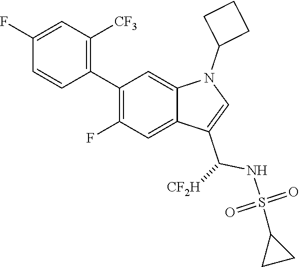 [SMILES: O=S(=O)(N[C@@H](c1cn(C2CCC2)c2cc(-c3ccc(F)cc3C(F)(F)F)c(F)cc12)C(F)F)C1CC1]

The title compound was prepared following the same general protocol as described for Step 6, Example 2, using (S)—N-(1-(6-bromo-1-cyclobutyl-5-fluoro-1H-indol-3-yl)-2,2-difluoroethyl)cyclopropanesulfonamide and (4-fluoro-2-(trifluoromethyl)phenyl)boronic acid with anhydrous dioxane as solvent, and the reaction temperature 140° C. ESI-MS (m/z): 534.5 [M+1]⁺.

### Example 155: (S)—N-(1-(1-cyclobutyl-5-fluoro-6-(4-fluoro-2-(trifluoromethyl)phenyl)-1H-pyrrolo[2,3-b]pyridin-3-yl)-2,2-difluoroethyl)cyclopropanesulfonamide

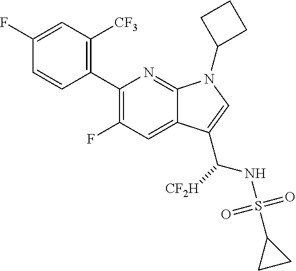 [SMILES: O=S(=O)(N[C@@H](c1cn(C2CCC2)c2nc(-c3ccc(F)cc3C(F)(F)F)c(F)cc12)C(F)F)C1CC1]

The title compound was prepared following the same general protocol as described for Step 6, Example 2, using (S)—N-(1-(6-chloro-5-fluoro-1-neopentyl-1H-pyrrolo[2,3-b]pyridin-3-yl)-2,2-difluoroethyl)cyclopropanesulfonamide and (4-fluoro-2-(trifluoromethyl)phenyl)boronic acid with anhydrous dioxane as solvent, and the reaction temperature of 140° C. ESI-MS (m/z): 551.5 [M+1]⁺.

### Example 156: (S)—N-(1-(1-cyclobutyl-5-fluoro-6-(5-fluoro-3-(trifluoromethyl)pyridin-2-yl)-1H-indol-3-yl)-2,2-difluoroethyl)cyclopropanesulfonamide

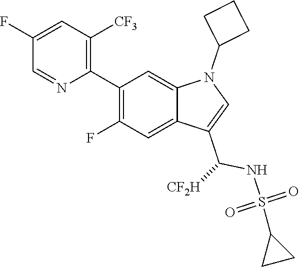 [SMILES: O=S(=O)(N[C@@H](c1cn(C2CCC2)c2cc(-c3ncc(F)cc3C(F)(F)F)c(F)cc12)C(F)F)C1CC1]

### Step 1: (S)—N-(1-(1-cyclobutyl-5-fluoro-6-(4,4,5,5-tetramethyl-1,3,2-dioxaborolan-2-yl)-1H-indol-3-yl)-2,2-difluoroethyl)cyclopropanesulfonamide

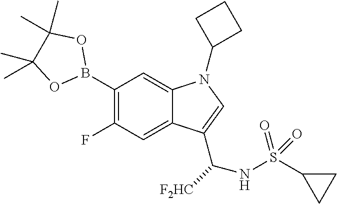 [SMILES: CC1(C)OB(c2cc3c(cc2F)c([C@H](NS(=O)(=O)C2CC2)C(F)F)cn3C2CCC2)OC1(C)C]

The title compound was prepared following the same general protocol as described for Step 5, Example 2, using (S)—N-(1-(6-bromo-1-cyclobutyl-5-fluoro-1H-indol-3-yl)-2,2-difluoroethyl)cyclopropanesulfonamide. ESI-MS (m/z): 498.5 [M+1]⁺.

### Step 2: (S)—N-(1-(1-cyclobutyl-5-fluoro-6-(5-fluoro-3-(trifluoromethyl)pyridin-2-yl)-1H-indol-3-yl)-2,2-difluoroethyl)cyclopropanesulfonamide

The title compound was prepared following the same general protocol as described for Step 6, Example 2, using (S)—N-(1-(1-cyclobutyl-5-fluoro-6-(4,4,5,5-tetramethyl-1,3,2-dioxaborolan-2-yl)-1H-indol-3-yl)-2,2-difluoroethyl)cyclopropanesulfonamide and 2-chloro-5-fluoro-3-(trifluoromethyl)pyridine with anhydrous dioxane as solvent, and the reaction temperature 140° C. ESI-MS (m/z): 535.5 [M+1]⁺.

### Example 157: (S)—N-(1-(1-cyclobutyl-5-fluoro-6-(5-methyl-2-(trifluoromethyl)pyridin-3-yl)-1H-pyrrolo[2,3-b]pyridin-3-yl)-2,2-difluoroethyl)cyclopropanesulfonamide

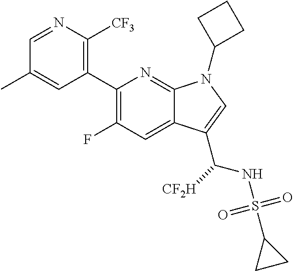 [SMILES: Cc1cnc(C(F)(F)F)c(-c2nc3c(cc2F)c([C@H](NS(=O)(=O)C2CC2)C(F)F)cn3C2CCC2)c1]

The title compound was prepared following the same general protocol as described for Step 6, Example 2, using (S)—N-(1-(6-chloro-1-cyclobutyl-5-fluoro-1H-pyrrolo[2,3-b]pyridin-3-yl)-2,2-difluoroethyl)cyclopropanesulfonamide and 5-methyl-3-(4,4,5,5-tetramethyl-1,3,2-dioxaborolan-2-yl)-2-(trifluoromethyl)pyridine with anhydrous dioxane as solvent, and the reaction temperature of 140° C. ESI-MS (m/z): 532.5 [M+1]⁺.

### Example 158: N-((1S)-1-(6-(2-cyano-6-(trifluoromethyl)phenyl)-1-cyclobutyl-5-fluoro-1H-pyrrolo[2,3-b]pyridin-3-yl)-2,2-difluoroethyl)cyclopropanesulfonamide

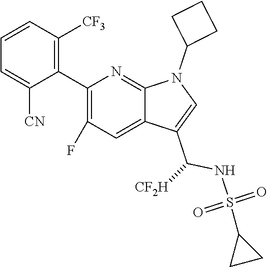 [SMILES: N#Cc1cccc(C(F)(F)F)c1-c1nc2c(cc1F)c([C@H](NS(=O)(=O)C1CC1)C(F)F)cn2C1CCC1]

### Step 1: (S)—N-(1-(1-cyclobutyl-5-fluoro-6-(tributylstannyl)-1H-pyrrolo[2,3-b]pyridin-3-yl)-2,2-difluoroethyl)cyclopropanesulfonamide

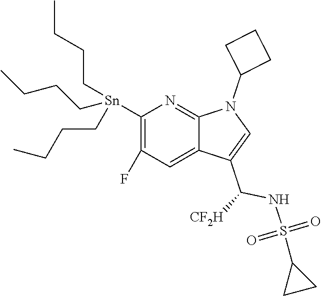 [SMILES: CCC[CH2][Sn]([CH2]CCC)([CH2]CCC)[c]1nc2c(cc1F)c([C@H](NS(=O)(=O)C1CC1)C(F)F)cn2C1CCC1]

The mixture of(S)—N-(1-(6-chloro-1-cyclobutyl-5-fluoro-1H-pyrrolo[2,3-b]pyridin-3-yl)-2,2-difluoroethyl)cyclopropanesulfonamide (0.6 g, 1.47 mmol) and 1,1,1,2,2,2-hexabutyldistannane (1.02 g, 1.76 mmol) and Pd(PPh₃)₄ (0.085 g, 0.074 mmol) in dioxane (15 mL) was degassed and heated at 130° C. overnight. The completion was monitored by anal. HPLC. The mixture was concentrated and the crude was purified by silica gel to obtain the title compound. ¹H NMR (CDCl₃, 400 MHz) δ 7.52 (d, J=7.0 Hz, 1H), 7.42 (s, 1H), 7.35 (d, J=9.32 Hz, 1H), 6.11 (td, J=55.8 Hz, 2.2 Hz, 1H), 5.25 (q, J=8.0 Hz, 1H), 5.08-4.98 (m, 1H), 4.87 (d, J=8.1 Hz, 1H), 2.56-2.52 (m, 4H), 2.35-2.33 (m, 1H), 1.95-1.89 (m, 2H), 1.63-1.58 (m, 9H), 1.41-1.11 (m, 13H), 0.93-0.81 (m, 11H)

### Step 2: N-((1S)-1-(6-(2-cyano-6-(trifluoromethyl)phenyl)-1-cyclobutyl-5-fluoro-1H-pyrrolo[2,3-b]pyridin-3-yl)-2,2-difluoroethyl)cyclopropanesulfonamide

The mixture of(S)—N-(1-(1-cyclobutyl-5-fluoro-6-(tributylstannyl)-1H-pyrrolo[2,3-b]pyridin-3-yl)-2,2-difluoroethyl)cyclopropanesulfonamide (0.092 g, 0.139 mmol), 2-bromo-3-(trifluoromethyl)benzonitrile (0.052 g, 0.21 mmol), CsF (0.042 g, 0.278 mmol), CuI (0.003 g, 0.014 mmol) and Pd(PPh₃)₄ (0.016 g, 0.014 mmol) in DMF (1.5 mL) was degassed and heated at 140° C. overnight. The completion was monitored by anal. HPLC. The mixture was concentrated and the crude was purified by silica gel to obtain the title compound. ESI-MS (m/z): 542.5 [M+1]⁺.

### Example 159: N-((1S)-1-(6-(2-cyano-4-(trifluoromethyl)pyridin-3-yl)-1-cyclobutyl-5-fluoro-1H-indol-3-yl)-2,2-difluoroethyl)cyclopropanesulfonamide

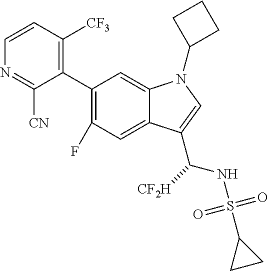 [SMILES: N#Cc1nccc(C(F)(F)F)c1-c1cc2c(cc1F)c([C@H](NS(=O)(=O)C1CC1)C(F)F)cn2C1CCC1]

The title compound was prepared following the same general protocol as described for Step 6, Example 2, using (S)—N-(1-(1-cyclobutyl-5-fluoro-6-(4,4,5,5-tetramethyl-1,3,2-dioxaborolan-2-yl)-1H-indol-3-yl)-2,2-difluoroethyl)cyclopropanesulfonamide and 3-bromo-4-(trifluoromethyl)picolinonitrile with anhydrous dioxane as solvent, and the reaction temperature of 140° C. ¹H NMR (CDCl₃, 400 MHz) δ 8.73 (d, J=5.0 Hz, 1H), 7.77 (d, J=4.8 Hz, 1H), 7.41-7.37 (m, 2H), 7.05 (d, J=5.8 Hz, 1H), 5.92 (td, J=55.8 Hz, 2.2 Hz, 1H), 4.85 (t, J=14.4 Hz, 1H), 4.61 (q, J=8.1 Hz, 1H), 2.63-2.59 (m, 2H), 2.47-2.39 (m, 2H), 2.08-1.91 (m, 1H), 1.79-1.76 (m, 2H), 1.15-1.11 (m, 2H), 0.90-0.88 (m, 2H).

### Example 160: N-((1S)-1-(6-(2-cyano-4-(trifluoromethyl)pyridin-3-yl)-1-cyclobutyl-5-fluoro-1H-pyrrolo[2,3-b]pyridin-3-yl)-2,2-difluoroethyl)cyclopropanesulfonamide

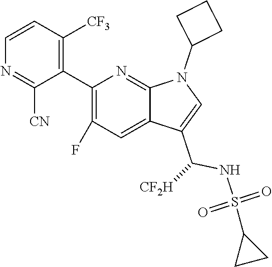 [SMILES: N#Cc1nccc(C(F)(F)F)c1-c1nc2c(cc1F)c([C@H](NS(=O)(=O)C1CC1)C(F)F)cn2C1CCC1]

The title compound was prepared following the same general protocol as described for Step 2, Example 158, using (S)—N-(1-(1-cyclobutyl-5-fluoro-6-(tributylstannyl)-1H-pyrrolo[2,3-b]pyridin-3-yl)-2,2-difluoroethyl)cyclopropanesulfonamide and 3-bromo-4-(trifluoromethyl)picolinonitrile. ESI-MS (m/z): 543.6 [M+1]⁺.

### Example 161: N-((1S)-1-(6-(2-cyano-4-(difluoromethyl)pyridin-3-yl)-1-cyclobutyl-5-fluoro-1H-indol-3-yl)-2,2-difluoroethyl)cyclopropanesulfonamide

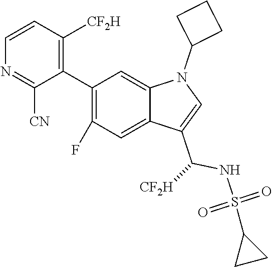 [SMILES: N#Cc1nccc(C(F)F)c1-c1cc2c(cc1F)c([C@H](NS(=O)(=O)C1CC1)C(F)F)cn2C1CCC1]

The title compound was prepared following the same general protocol as described for Step 6, Example 2, using (S)—N-(1-(1-cyclobutyl-5-fluoro-6-(4,4,5,5-tetramethyl-1,3,2-dioxaborolan-2-yl)-1H-indol-3-yl)-2,2-difluoroethyl)cyclopropanesulfonamide and 3-bromo-4-(difluoromethyl)picolinonitrile with anhydrous dioxane as solvent, and the reaction temperature of 140° C. ESI-MS (m/z): 524.5 [M+1]⁺.

### Example 162: N-((1S)-1-(6-(2-cyano-4-(difluoromethyl)pyridin-3-yl)-1-cyclobutyl-5-fluoro-1H-pyrrolo[2,3-b]pyridin-3-yl)-2,2-difluoroethyl)cyclopropanesulfonamide

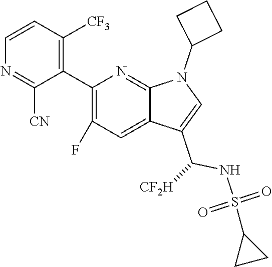 [SMILES: N#Cc1nccc(C(F)(F)F)c1-c1nc2c(cc1F)c([C@H](NS(=O)(=O)C1CC1)C(F)F)cn2C1CCC1]

The title compound was prepared following the same general protocol as described for Step 2, Example 158, using (S)—N-(1-(1-cyclobutyl-5-fluoro-6-(tributylstannyl)-1H-pyrrolo[2,3-b]pyridin-3-yl)-2,2-difluoroethyl)cyclopropanesulfonamide and 3-bromo-4-(difluoromethyl)picolinonitrile. ESI-MS (m/z): 525.5 [M+1]⁺.

### Example 163: (S)—N-(2,2-difluoro-1-(1-neopentyl-6-(2-(trifluoromethyl)phenyl)-1H-indol-3-yl)ethyl)cyclobutane-1-sulfonamide-3,3-d₂

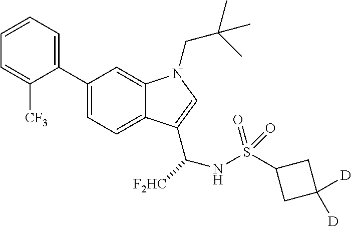 [SMILES: [2H]C1([2H])CC(S(=O)(=O)N[C@@H](c2cn(CC(C)(C)C)c3cc(-c4ccccc4C(F)(F)F)ccc23)C(F)F)C1]

### Step 1: diethyl malonate-d₂

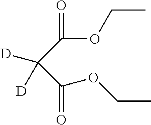

The mixture of diethyl malonate (24 g, 149.85 mmol) and potassium carbonate (4.14 g, 29.95 mmol) in deuterated water (100 mL) was stirred overnight at room temperature. The reaction was extracted with diethyl ether (2×). The ether layer was dried with anhydrous Na₂SO₄ and solvent was removed to obtain the title compound with no further purification. ¹H NMR (CDCl₃, 400 MHz) δ 4.23 (q, J=8.0 Hz, 4H), 1.28 (t, J=8.0 Hz, 6H)

### Step 2: propane-2,2-d2-1,3-diol

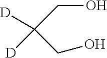

LAH (9.47 g, 249.48 mmol) was added portion wise to anhydrous THF (200 mL) at ice cold bath. Then diethyl malonate-d₂ (33.71 g, 207.90 mmol) in THF (70 mL) was added dropwise. The reaction was stirred overnight at RT. The mixture was cooled to 0° C., water (9.47 mL) was added dropwise, followed by NaOH (15%, 9.47 mL) and water (28.4 mL). The above mixture was stirred 30 min at rt, and then filtered to obtain the filtrate. The filter cake was stirred at THE/MeOH (3:1, 500 mL) for 30 min, and filtered to obtain the second batch of filtrate, this step was repeated twice. The filtrates were combined and the solvent was removed to obtain the crude, which was purified by silica gel to obtain the title compound. ¹H NMR (methanol-d4, 400 MHz) δ 4.38-4.28 (m, 2H), 3.46 (s, 2H), 3.44 (s, 2H)

### Step 3:1,3-dibromopropane-2,2-d2

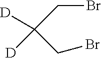

The mixture of propane-2,2-d2-1,3-diol (11.679 g, 149.52 mmol) and PPh₃ (117.65 g, 448.56 mmol) in DCM/acetonitrile (310 mL, 1 v/1 v) was cooled to 0° C., NBS (79.83 g, 448.56 mmol) was added portionwise. The reaction was stirred overnight at rt. Pentane was applied to extract the reaction several times, and then washed with 2N NaOH, water and brine and dried over anhydrous Na₂SO₄. The solvent was then removed, and the title compound was obtained by distillation under vacuum pressure. 1H NMR (CDCl₃, 400 MHz) δ 3.58 (s, 4H)

### Step 4: diethyl cyclobutane-1,1-dicarboxylate-3,3-d2

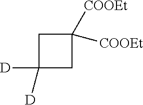 [SMILES: [2H]C1([2H])CC(C(=O)OCC)(C(=O)OCC)C1]

The mixture of 1,3-dibromopropane-2,2-d2 (18.53 g, 90.86 mmol) and diethyl malonate (13.86 g, 86.536 mmol) in EtOH (87 mL) was heated at 65° C., and then NaOEt (21% by wt in EtOH, 65 mL, 173.07 mmol) was added. The reaction was continued to stir for 2 h at same temperature. The mixture was concentrated and redissolved with EtOAc and washed with sat'd NaHCO₃, and brine. The mixture was concentrated and the crude was purified by silica gel to obtain the title compound. ¹H NMR (CDCl₃, 400 MHz) δ 4.22 (q, J=8.0 Hz, 4H), 2.54 (s, 4H), 1.27 (t, J=8.0 Hz, 6H)

### Step 5: cyclobutane-1,1-dicarboxylic-3,3-d2 acid

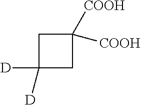 [SMILES: [2H]C1([2H])CC(C(=O)O)(C(=O)O)C1]

To a solution of diethyl cyclobutane-1,1-dicarboxylate-3,3-d2 (7.82 g, 38.68 mmol) in ethanol (40 mL) was added aqueous sodium hydroxide (4N, 22.2 mL, 88.96 mmol). The reaction was then heated to reflux and stirred for 2 h. Upon cooling to room temperature, the reaction was concentrated in vacuo and diluted with 6 N HCl (100 mL). The resulting aqueous solution was extracted with EtOAc (3×200 mL), and the combined organic layers were dried with Na₂SO₄, filtered, and concentrated. The resulting residue was then recrystallized from EtOAc/heptane to obtain the title compound as white crystals. This material was carried forward into the next step without further purification. 1H NMR (methanol-d4, 400 MHz) δ 2.51 (s, 4H)

### Step 6: N-methoxy-N-methylcyclobutane-1-carboxamide-3,3-d2

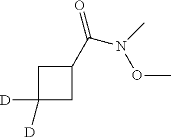

Cyclobutane-1,1-dicarboxylic-3,3-d2 acid (3.16 g, 21.67 mmol) was heated to 160° C. for 2 h. After cooling to room temperature, the reaction was dissolved with DCM and then cooled to 0° C., DMF (0.34 mL, 4.33 mmol) was added, followed by slow addition of oxalyl chloride (2.46 mL, 28.17 mmol). The mixture was warmed to rt and continued to stir for 2 h and then slowly added to the mixture of N,O-dimethylhydroxylamine HCl salt (6.34 g, 65.01 mmol) and NaHCO₃ (18.2 g, 210.67 mmol) in DCM (100 mL) at 0° C. The mixture was stirred overnight at rt. Water was added and organic layer was separated. The aqueous layer was extracted with DCM. The organic layers were combined and dried over Na₂SO₄. The solvent was removed and the crude was purified by silica gel to obtain the title compound. ¹H NMR (CDCl₃, 400 MHz) δ 3.64 (s, 3H), 3.48-3.42 (m, 1H), 3.16 (s, 3H), 2.31 (t, J=8.0 Hz, 2H), 2.15 (t, J=8.0 Hz, 2H)

### Step 7: (cyclobutyl-3,3-d2)(phenyl)methanone

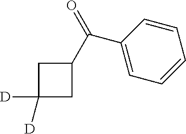 [SMILES: [2H]C1([2H])CC(C(=O)c2ccccc2)C1]

To the solution of N-methoxy-N-methylcyclobutane-1-carboxamide-3,3-d2 (1.274 g, 8.776 mmol) in THF (40 mL) at −78° C. was added PhLi (1.9 M in dibutyl ether, 7.0 mL, 13.3 mmol) dropwise. Then the mixture was gradually warmed to rt and stirred overnight. Sat′d NH₄Cl was added and the mixture was extracted with EtOAc, washed with brine and dried over Na₂SO₄. The solvent was removed and the crude was purified by silica gel to obtain the title compound. ¹H NMR (CDCl₃, 400 MHz) δ 7.91-7.88 (m, 2H), 7.56-7.54 (m, 1H), 7.52-7.42 (m, 2H), 4.00 (q, J=8.0 hZ, 1H), 2.40 (t, J=8.0 Hz, 2H), 2.28 (t, J=8.0 Hz, 2H)

### Step 8: cyclobutyl-3,3-d2 benzoate

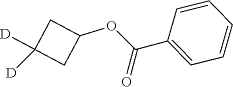 [SMILES: [2H]C1([2H])CC(OC(=O)c2ccccc2)C1]

To the solution of (cyclobutyl-3,3-d2)(phenyl)methanone (2.53 g, 15.61 mmol) in DCM (80 mL) was added mCPBA (75%, 7.2 g, 31.22 mmol). The mixture was stirred at 50° C. overnight. The reaction was monitored by anal. HPLC. Additional mCPBA (75%, 5.4 g, 23.41 mmol) was added and stirred another day at 50° C. The mixture was diluted with DCM and washed with sat'd NaHCO₃, water, brine and dried over Na₂SO₄. The solvent was removed and the crude was purified by silica gel to obtain the title compound. ¹H NMR (CDCl₃, 400 MHz) δ 8.06-8.03 (m, 2H), 7.57-7.53 (m, 1H), 7.45-7.41 (m, 2H), 5.21 (q, J=8.0 Hz, 1H), 2.47-2.42 (m, 2H), 2.22-2.17 (m, 2H)

### Step 9: cyclobutan-3,3-d2-1-ol

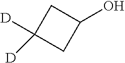

To the solution of cyclobutyl-3,3-d2 benzoate (0.713 g, 4.0 mmol) in THF (10 mL) at 0° C. was added MeMgBr (3.0 M in ether, 4.0 mL, 12.0 mmol) dropwise. The mixture was warmed to rt and stirred overnight. The reaction was monitored by anal. HPLC. The mixture was diluted with ether and quenched with sat'd NH₄Cl. The aqueous layer was extracted with ether 3 times. The ether layers were combined and washed with brine and dried over Na₂SO₄. The solvent was removed and the crude was purified by silica gel to obtain the title compound. ¹H NMR (CDCl₃, 400 MHz) δ 8.06-8.03 (m, 2H), 7.57-7.53 (m, 1H), 7.45-7.41 (m, 2H), 5.21 (q, J=8.0 Hz, 1H), 2.47-2.42 (m, 2H), 2.22-2.17 (m, 2H)

### Step 10: S-(cyclobutyl-3,3-d2) ethanethioate

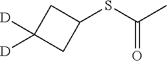

PPh₃ (0.433 g, 1.65 mmol) in THF (4 mL) at 0° C. was added DIAD (0.32 mL, 1.65 mmol) dropwise. The mixture was continued to stir at 0° C. for 30 min, then cyclobutan-3,3-d2-1-ol (0.0817 g, 1.1 mmol) and ethanethioic S-acid (0.126 g, 1.65 mmol) in THF (1 mL) was added dropwise. The mixture was stirred overnight. The mixture was diluted with ether and quenched with NaHCO₃. The aqueous layer was extracted with ether 3 times. The ether layers were combined and washed with brine and dried over Na₂SO₄. The solvent was removed and the crude was purified by silica gel to obtain the title compound. ¹H NMR (acetonitrile-d, 400 MHz) δ 4.06 (q, J=8.0 Hz, 1H), 2.41-2.37 (m, 2H), 2.25 (s, 3H), 2.07-2.01 (m, 2H)

### Step 11: cyclobutane-1-sulfonyl chloride-3,3-d2

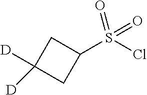

Con HCl (2 mL) was added NCS (1.143 g, 8.56 mmol) in ACN (12 mL) at room temperature, followed by addition of S-(cyclobutyl-3,3-d2) ethanethioate (0.282 g, 2.14 mmol) in ACN (2 mL). The mixture was continued to stir for 1 h. The reaction was diluted with ether and water, the aqueous layer was extracted with ether twice. The ether layers were combined and washed with brine and dried over Na₂SO₄. The solvent was removed and the crude was purified by silica gel to obtain the title compound. ¹H NMR (DCM-d2, 400 MHz) δ 4.43 (q, J=8.0 Hz, 1H), 2.73-2.64 (m, 2H), 2.55-2.47 (m, 2H).

### Step 12: (S)—N-(2,2-difluoro-1-(1-neopentyl-6-(2-(trifluoromethyl)phenyl)-1H-indol-3-yl)ethyl)cyclobutane-1-sulfonamide-3,3-d2

The title compound was prepared following the same general protocol as described for Step 4, Example 2, using (S)-2,2-difluoro-1-(1-neopentyl-6-(2-(trifluoromethyl)phenyl)-1H-indol-3-yl)ethan-1-amine and cyclobutane-1-sulfonyl chloride-3,3-d2. ¹H NMR (CDCl₃, 400 MHz) δ 7.76 (d, J=8.0 Hz, 1H), 7.69 (d, J=8.0 Hz, 1H), 7.57 (t, J=8.0 Hz, 1H), 7.48 (t, J=8.0 Hz, 1H), 7.37 (d, J=8.0 Hz, 1H), 7.32 (s, 1H), 7.17 (s, 1H), 7.11 (d, J=8.0 Hz, 1H), 6.15 (td, J=55.8 Hz, 2.2 Hz, 1H), 5.18-5.08 (m, 1H), 4.69 (d, J=8.0 Hz, 1H), 3.88 (s, 2H), 3.70 (q, J=8.0 Hz, 1H), 2.47-2.39 (m, 2H), 2.20-2.14 (m, 1H), 2.07-2.02 (m, 1H), 0.99 (s, 9H).

### Example 164: (S)—N-(2,2-difluoro-1-(5-fluoro-6-(4-fluoro-2-(trifluoromethyl)phenyl)-1-neopentyl-1H-indol-3-yl)ethyl)cyclopropanesulfonamide

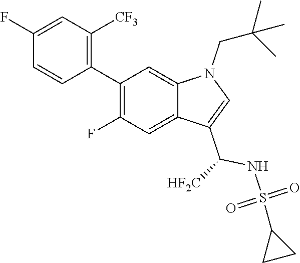

The title compound was prepared following the same general protocol as described for Step 6, Example 2, using (S)—N-(1-(6-bromo-5-fluoro-1-neopentyl-1H-indol-3-yl)-2,2-difluoroethyl)cyclopropanesulfonamide and (4-fluoro-2-(trifluoromethyl)phenyl)boronic acid with anhydrous dioxane as solvent, and the reaction temperature of 140° C. ESI-MS (m/z): 550.5 [M+1]⁺.

### Example 165: (S)—N-(1-(6-(5-chloro-2-(trifluoromethyl)pyridin-3-yl)-1-cyclobutyl-5-fluoro-1H-pyrrolo[2,3-b]pyridin-3-yl)-2,2-difluoroethyl)cyclopropanesulfonamide

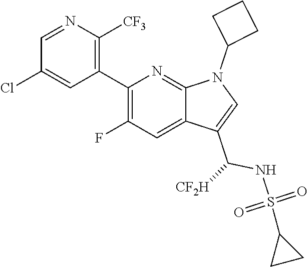 [SMILES: O=S(=O)(N[C@@H](c1cn(C2CCC2)c2nc(-c3cc(Cl)cnc3C(F)(F)F)c(F)cc12)C(F)F)C1CC1]

The title compound was prepared following the same general protocol as described for Step 2, Example 158, using (S)—N-(1-(1-cyclobutyl-5-fluoro-6-(tributylstannyl)-1H-pyrrolo[2,3-b]pyridin-3-yl)-2,2-difluoroethyl)cyclopropanesulfonamide and 3-bromo-5-chloro-2-(trifluoromethyl)pyridine. ESI-MS (m/z): 552.9 [M+1]⁺.

### Example 166: (S)—N-(2,2-difluoro-1-(1-neopentyl-6-(6-oxo-2-(trifluoromethyl)-1,6-dihydropyridin-3-yl)-1H-indol-3-yl)ethyl)cyclopropanesulfonamide

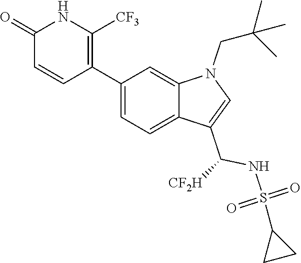 [SMILES: CC(C)(C)Cn1cc([C@H](NS(=O)(=O)C2CC2)C(F)F)c2ccc(-c3ccc(=O)[nH]c3C(F)(F)F)cc21]

The title compound was prepared following the same general protocol as described for Step 6, Example 2, using (S)—N-(2,2-difluoro-1-(1-neopetyl-6-(4,4,5,5-tetramethyl-1,3,2-dioxaborolan-2-yl)-1H-indol-3-yl)ethyl)cyclopropanesulfonamide and 5-bromo-6-(trifluoromethyl)pyridin-2 (1H)-one. ESI-MS (m/z): 531.75 [M+1]⁺.

### Example 167: (S)—N-(1-(6-(6-amino-2-(trifluoromethyl)pyridin-3-yl)-1-neopentyl-1H-indol-3-yl)-2,2-difluoroethyl)cyclopropanesulfonamide

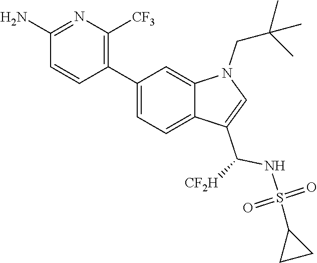 [SMILES: CC(C)(C)Cn1cc([C@H](NS(=O)(=O)C2CC2)C(F)F)c2ccc(-c3ccc(N)nc3C(F)(F)F)cc21]

The title compound was prepared following the same general protocol as described for Step 6, Example 2, using (S)—N-(2,2-difluoro-1-(1-neopentyl-6-(4,4,5,5-tetramethyl-1,3,2-dioxaborolan-2-yl)-1H-indol-3-yl)ethyl)cyclopropanesulfonamide and 5-bromo-6-(trifluoromethyl)pyridin-2-amine. ESI-MS (m/z): 530.90 [M+1]⁺.

### Example 168: (S)—N-(2,2-difluoro-1-(5-fluoro-6-(4-fluoro-2-(trifluoromethyl)phenyl)-1-(1-methylazetidin-3-yl)-1H-indol-3-yl)ethyl)cyclopropanesulfonamide

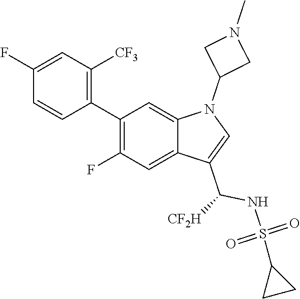 [SMILES: CN1CC(n2cc([C@H](NS(=O)(=O)C3CC3)C(F)F)c3cc(F)c(-c4ccc(F)cc4C(F)(F)F)cc32)C1]

(S)—N-(1-(1-(azetidin-3-yl)-5-fluoro-6-(4-fluoro-2-(trifluoromethyl)phenyl)-1H-indol-3-yl)-2,2-difluoroethyl)cyclopropanesulfonamide (0.0296 g, 0.055 mmol) in MeOH (0.4 mL) and dichloroethane (0.2 mL) was added HCHO (37% in water, 0.09 g, 0.11 mmol) and AcOH (0.001 g, 0.016 mmol), and the mixture was stirred at rt for 30 min, NaBH(OAc)₃ (0.023 g, 0.11 mmol) was added. The reaction was continued to stir at rt overnight. The completion of reaction was monitored by anal. HPLC. The mixture was concentrated and the crude was purified by preparative HPLC to obtain the title compound. ¹H NMR (methanol-d₄, 400 MHz) δ 7.89 (s, 1H), 7.66-7.60 (m, 2H), 7.47 (d, J=6.8 Hz, 2H), 7.38 (d, J=5.8 Hz, 1H), 6.27 (td, J=55.8 Hz, 2.2 Hz, 1H), 5.64 (q, J=8.4 Hz, 1H), 5.11 (td, J=13.9 Hz, 2.6 Hz, 1H), 5.18-5.10 (m, 2H), 4.56-4.50 (m, 1H), 3.99-3.95 (m, 2H), 3.61-3.57 (m, 2H), 2.52 (s, 3H), 2.39-2.33 (m, 1H), 1.03-0.91 (m, 2H), 0.79-0.74 (m, 2H)

### Example 169: (S)—N-(2,2-difluoro-1-(5-fluoro-1-neopentyl-6-(6-oxo-2-(trifluoromethyl)-1,6-dihydropyridin-3-yl)-1H-indol-3-yl)ethyl)cyclopropanesulfonamide

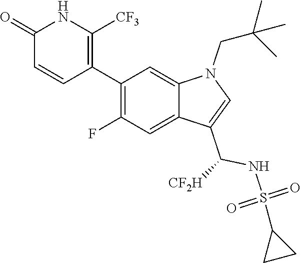 [SMILES: CC(C)(C)Cn1cc([C@H](NS(=O)(=O)C2CC2)C(F)F)c2cc(F)c(-c3ccc(=O)[nH]c3C(F)(F)F)cc21]

The title compound was prepared following the same general protocol as described for Step 6, Example 2, using (S)—N-(2,2-difluoro-1-(5-fluoro-1-neopentyl-6-(4,4,5,5-tetramethyl-1,3,2-dioxaborolan-2-yl)-1H-indol-3-yl)ethyl)cyclopropanesulfonamide and 5-bromo-6-(trifluoromethyl)pyridin-2 (1H)-one with anhydrous dioxane as solvent, and the reaction temperature of 140° C. ¹H NMR (CDCl₃, 400 MHz) δ 7.56-7.48 (m, 3H), 7.20 (d, J=5.9 Hz, 1H), 6.93 (d, J=9.0 Hz, 1H)), 6.15 (td, J=55.8 Hz, 2.2 Hz, 1H), 3.88 (s, 2H), 2.41-2.30 (m, 1H), 1.17-1.05 (m, 2H), 0.98 (s, 9H), 0.96-0.83 (m, 2H)

### Example 170: (S)—N-(2,2-difluoro-1-(5-fluoro-6-(4-fluoro-2-(trifluoromethyl)phenyl)-1-neopentyl-1H-indol-3-yl)ethyl)cyclobutanesulfonamide

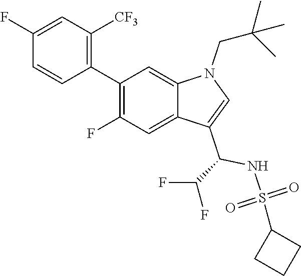 [SMILES: CC(C)(C)Cn1cc([C@H](NS(=O)(=O)C2CCC2)C(F)F)c2cc(F)c(-c3ccc(F)cc3C(F)(F)F)cc21]

### Step 1: (S)—N—((S)-2,2-difluoro-1-(5-fluoro-6-(4-fluoro-2-(trifluoromethyl)phenyl)-1-neopentyl-1H-indol-3-yl)ethyl)-2-methylpropane-2-sulfinamide

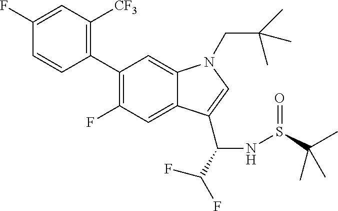 [SMILES: CC(C)(C)Cn1cc([C@H](N[S@@](=O)C(C)(C)C)C(F)F)c2cc(F)c(-c3ccc(F)cc3C(F)(F)F)cc21]

The title compound was prepared following the same general protocol as described for Step 6, Example 2, using (S)—N—((S)-1-(6-bromo-5-fluoro-1-neopentyl-1H-indol-3-yl)-2,2-difluoroethyl)-2-methylpropane-2-sulfinamide and (4-fluoro-2-(trifluoromethyl)phenyl)boronic acid. ESI-MS (m/z): 551.2 [M+1]⁺.

### Step 2: (S)-2,2-difluoro-1-(5-fluoro-6-(4-fluoro-2-(trifluoromethyl)phenyl)-1-neopentyl-1H-indol-3-yl)ethan-1-amine

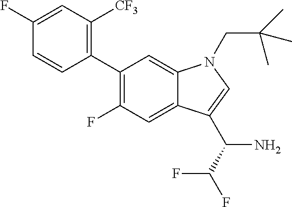 [SMILES: CC(C)(C)Cn1cc([C@H](N)C(F)F)c2cc(F)c(-c3ccc(F)cc3C(F)(F)F)cc21]

The title compound was prepared following the same general protocol as described for Step 3, Example 2, using (S)—N—((S)-2,2-difluoro-1-(5-fluoro-6-(4-fluoro-2-(trifluoromethyl)phenyl)-1-neopentyl-1H-indol-3-yl)ethyl)-2-methylpropane-2-sulfinamide. ESI-MS (m/z): 430.2 [M-NH₂]⁺.

Step 3: The title compound was prepared following the same general protocol as described for Step 4, Example 2, using (S)-2,2-difluoro-1-(5-fluoro-6-(4-fluoro-2-(trifluoromethyl)phenyl)-1-neopentyl-1H-indol-3-yl)ethan-1-amine and cyclobutanesulfonyl chloride. ESI-MS (m/z): 564.47 [M+1]⁺.

### Example 171: (S)—N-(2,2,2-trifluoro-1-(5-fluoro-1-neopentyl-6-(2-(trifluoromethyl)pyridin-3-yl)-1H-indol-3-yl)ethyl)cyclopropanesulfonamide

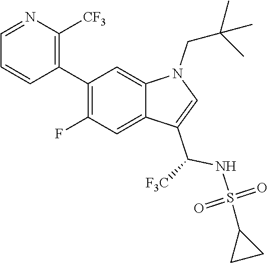 [SMILES: CC(C)(C)Cn1cc([C@H](NS(=O)(=O)C2CC2)C(F)(F)F)c2cc(F)c(-c3cccnc3C(F)(F)F)cc21]

### Step 1: (S)-2-methyl-N—((S)-2,2,2-trifluoro-1-(5-fluoro-1-neopentyl-6-(2-(trifluoromethyl)pyridin-3-yl)-1H-indol-3-yl)ethyl)propane-2-sulfinamide

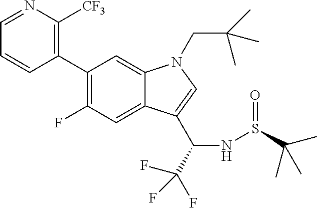 [SMILES: CC(C)(C)Cn1cc([C@H](N[S@@](=O)C(C)(C)C)C(F)(F)F)c2cc(F)c(-c3cccnc3C(F)(F)F)cc21]

The title compound was prepared following the same general protocol as described for Step 6, Example 2, using (S)—N—((S)-1-(6-bromo-5-fluoro-1-neopentyl-1H-indol-3-yl)-2,2,2-trifluoroethyl)-2-methylpropane-2-sulfinamide and (2-(trifluoromethyl)pyridin-3-yl)boronic acid. ESI-MS (m/z): 551.6 [M+1]⁺.

### Step 2: (S)-2,2,2-trifluoro-1-(5-fluoro-1-neopentyl-6-(2-(trifluoromethyl)pyridin-3-yl)-1H-indol-3-yl)ethan-1-amine

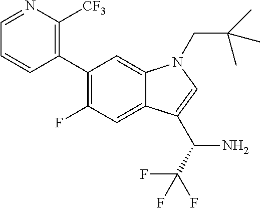 [SMILES: CC(C)(C)Cn1cc([C@H](N)C(F)(F)F)c2cc(F)c(-c3cccnc3C(F)(F)F)cc21]

The title compound was prepared following the same general protocol as described for Step 3, Example 2, using (S)-2-methyl-N—((S)-2,2,2-trifluoro-1-(5-fluoro-1-neopentyl-6-(2-(trifluoromethyl)pyridin-3-yl)-1H-indol-3-yl)ethyl)propane-2-sulfinamide. ESI-MS (m/z): 431.2 [M-NH₂]⁺.

Step 3: The title compound was prepared following the same general protocol as described for Step 4, Example 2, using (S)-2,2-difluoro-1-(5-fluoro-1-neopentyl-6-(2-(trifluoromethyl)pyridin-3-yl)-1H-indol-3-yl)ethan-1-amine and cyclopropanesulfonyl chloride. ESI-MS (m/z): 551.99 [M+1]⁺.

### Example 172: (S)—N-(2,2-difluoro-1-(5-fluoro-1-neopentyl-6-(tetrahydro-2H-pyran-4-yl)-1H-indol-3-yl)ethyl)cyclopropanesulfonamide

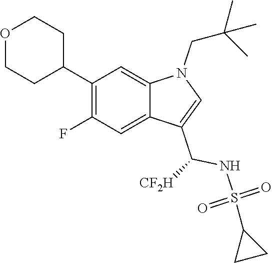 [SMILES: CC(C)(C)Cn1cc([C@H](NS(=O)(=O)C2CC2)C(F)F)c2cc(F)c(C3CCOCC3)cc21]

A mixture of(S)—N-(1-(6-(3,6-dihydro-2H-pyran-4-yl)-5-fluoro-1-neopentyl-1H-indol-3-yl)-2,2-difluoroethyl)cyclopropanesulfonamide and 10% Pd/C in methanol was stirred at room temperature overnight under H₂. The mixture was filtered, the filtrate was concentrated to give the title compound. ESI-MS (m/z): 472.55 [M+1]⁺.

### Example 173: (S)—N-(1-(6-(3,6-dihydro-2H-pyran-4-yl)-1-neopentyl-1H-indol-3-yl)-2,2-difluoroethyl)cyclopropanesulfonamide

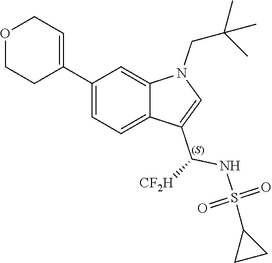

The title compound was prepared following the same general protocol as described for Step 6, Example 2, using (S)—N-(1-(6-bromo-1-neopentyl-1H-indol-3-yl)-2,2-difluoroethyl)cyclopropanesulfonamide and 2-(3,6-dihydro-2H-pyran-4-yl)-4,4,5,5-tetramethyl-1,3,2-dioxaborolane. ESI-MS (m/z): 452.61 [M+1]⁺.

### Example 174: (S)—N-(2,2-difluoro-1-(1-neopentyl-6-(tetrahydro-2H-pyran-4-yl)-1H-indol-3-yl)ethyl)cyclopropanesulfonamide

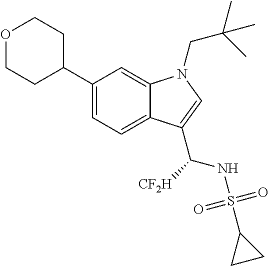 [SMILES: CC(C)(C)Cn1cc([C@H](NS(=O)(=O)C2CC2)C(F)F)c2ccc(C3CCOCC3)cc21]

A mixture of(S)—N-(1-(6-(3,6-dihydro-2H-pyran-4-yl)-1-neopentyl-1H-indol-3-yl)-2,2-difluoroethyl)cyclopropanesulfonamide and 10% Pd/C in methanol was stirred at room temperature overnight under H₂. The mixture was filtered and the filtrate was concentrated to give the title compound. ESI-MS (m/z): 454.62 [M+1]⁺.

### Example 175: (S)—N-(1-(1-cyclobutyl-5-fluoro-6-(6-oxo-2-(trifluoromethyl)-1,6-dihydropyridin-3-yl)-1H-pyrrolo[2,3-b]pyridin-3-yl)-2,2-difluoroethyl)cyclopropanesulfonamide

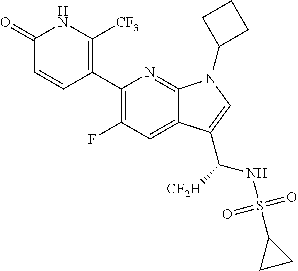 [SMILES: O=c1ccc(-c2nc3c(cc2F)c([C@H](NS(=O)(=O)C2CC2)C(F)F)cn3C2CCC2)c(C(F)(F)F)[nH]1]

The title compound was prepared following the same general protocol as described for Step 2, Example 158, using (S)—N-(1-(1-cyclobutyl-5-fluoro-6-(tributylstannyl)-1H-pyrrolo[2,3-b]pyridin-3-yl)-2,2-difluoroethyl)cyclopropanesulfonamide and 5-bromo-6-(trifluoromethyl)pyridin-2 (1H)-one. ¹H NMR (CDCl₃, 400 MHz) δ 7.84 (d, J=9.7 Hz, 1H), 7.67 (d, J=9.0 Hz, 1H), 7.60 (s, 1H), 6.93 (d, J=9.0 Hz, 1H), 6.17 (td, J=55.8 Hz, 2.2 Hz, 1H), 5.29-5.06 (m, 3H), 2.57-2.40 (m, 5H), 1.93-1.82 (m, 2H), 1.21-1.06 (m, 2H), 0.92-0.83 (m, 2H)

### Example 176: (S)—N-(2,2-difluoro-1-(5-fluoro-6-(4-fluoro-2-(trifluoromethyl)phenyl)-1-(oxetan-3-yl)-1H-indol-3-yl)ethyl)cyclopropanesulfonamide

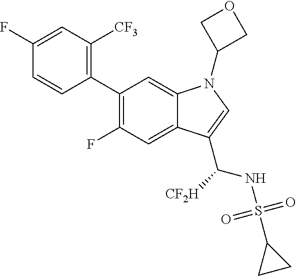 [SMILES: O=S(=O)(N[C@@H](c1cn(C2COC2)c2cc(-c3ccc(F)cc3C(F)(F)F)c(F)cc12)C(F)F)C1CC1]

### Step 1:1-(6-bromo-5-fluoro-1-(oxetan-3-yl)-1H-indol-3-yl)-2,2-difluoroethan-1-one

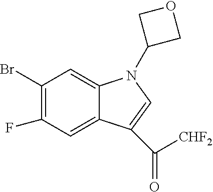 [SMILES: O=C(c1cn(C2COC2)c2cc(Br)c(F)cc12)C(F)F]

The title compound was prepared following the same general protocol as described for Step 1, Example 2, using 1-(6-bromo-5-fluoro-1H-indol-3-yl)-2,2-difluoroethan-1-one and 3-bromooxetane. ¹H NMR (CDCl₃, 400 MHz) δ 8.29 (t, J=1.6 Hz, 1H), 8.19 (d, J=8.9 Hz, 1H), 7.81 (d, J=5.4 Hz, 1H), 6.10 (t, J=54.0 Hz, 1H), 5.56-5.49 (m, 1H), 5.26 (t, J=7.2 Hz, 2H), 5.06 (dd, J=6.0 Hz, 6.0 Hz, 2H)

### Step 2: (S,E)-N-(1-(6-bromo-5-fluoro-1-(oxetan-3-yl)-1H-indol-3-yl)-2,2-difluoroethylidene)-2-methylpropane-2-sulfinamide

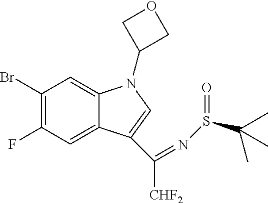 [SMILES: CC(C)(C)[S@](=O)/N=C(\c1cn(C2COC2)c2cc(Br)c(F)cc12)C(F)F]

The title compound was prepared following the same general protocol as described for Step 2, Example 11, using 1-(6-bromo-5-fluoro-1-(oxetan-3-yl)-1H-indol-3-yl)-2,2-difluoroethan-1-one and (S)-2-methylpropane-2-sulfinamide, and the reaction was stirred for 2 h at 90° C. 1H NMR (CDCl₃, 400 MHz) δ 8.12 (t, J=2.0 Hz, 1H), 8.02 (d, J=9.5 Hz, 1H), 7.79 (d, J=5.6 Hz, 1H), 5.52-5.47 (m, 1H), 5.22 (t, J=7.2 Hz, 2H), 5.09-5.04 (m, 2H), 1.36 (s, 9H)

### Step 3: (S)—N—((S)-1-(6-bromo-5-fluoro-1-(oxetan-3-yl)-1H-indol-3-yl)-2,2-difluoroethyl)-2-methylpropane-2-sulfinamide

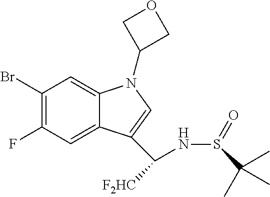 [SMILES: CC(C)(C)[S@](=O)N[C@@H](c1cn(C2COC2)c2cc(Br)c(F)cc12)C(F)F]

The title compound was prepared following the same general protocol as described for Step 3, Example 31, using (S,E)-N-(1-(6-bromo-5-fluoro-1-(oxetan-3-yl)-1H-indol-3-yl)-2,2-difluoroethylidene)-2-methylpropane-2-sulfinamide. 1H NMR (CDCl₃, 400 MHz) δ 7.72 (d, J=5.6 Hz, 1H), 7.56 (s, 1H), 7.39 (d, J=9.1 Hz, 1H), 6.07 (td, J=55.8 Hz, 2.2 Hz, 1H), 5.49-5.42 (m, 1H), 5.18 (t, J=7.2 Hz, 2H), 5.07 (t, J=6.1 Hz, 1H), 5.00 (t, J=6.1 Hz, 1H), 4.88-4.82 (m, 1H), 1.23 (s, 9H)

### Step 4: (S)—N-(1-(6-bromo-5-fluoro-1-(oxetan-3-yl)-1H-indol-3-yl)-2,2-difluoroethyl)formamide

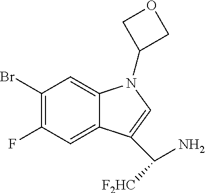 [SMILES: N[C@@H](c1cn(C2COC2)c2cc(Br)c(F)cc12)C(F)F]

The title compound was prepared following the same general protocol as described for Step 3, Example 2, using (S)—N—((S)-1-(6-bromo-5-fluoro-1-(oxetan-3-yl)-1H-indol-3-yl)-2,2-difluoroethyl)-2-methylpropane-2-sulfinamide

### Step 5: (S)—N-(1-(6-bromo-5-fluoro-1-(oxetan-3-yl)-1H-indol-3-yl)-2,2-difluoroethyl)cyclopropanesulfonamide

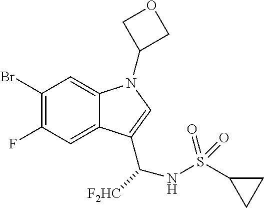 [SMILES: O=S(=O)(N[C@@H](c1cn(C2COC2)c2cc(Br)c(F)cc12)C(F)F)C1CC1]

The title compound was prepared following the same general protocol as described for Step 4, Example 2, using (S)—N-(1-(6-bromo-5-fluoro-1-(oxetan-3-yl)-1H-indol-3-yl)-2,2-difluoroethyl)formamide and cyclopropanesulfonyl chloride. 1H NMR (CDCl₃, 400 MHz) δ 7.56-7.53 (m, 1H), 7.48-7.44 (m, 2H), 6.07 (td, J=55.8 Hz, 2.2 Hz, 1H), 5.55 (d, J=8.7 Hz, 1H), 5.06-4.98 (m, 1H), 4.61-4.57 (m, 1H), 4.08-3.80 (m, 4H), 2.37-2.27 (m, 1H), 1.19-1.09 (m, 2H), 0.93-0.83 (m, 2H)

### Step 6: (S)—N-(2,2-difluoro-1-(5-fluoro-6-(4-fluoro-2-(trifluoromethyl)phenyl)-1-(oxetan-3-yl)-1H-indol-3-yl)ethyl)cyclopropanesulfonamide

The title compound was prepared following the same general protocol as described for Step 6, Example 2, using (S)—N-(1-(6-bromo-5-fluoro-1-(oxetan-3-yl)-1H-indol-3-yl)-2,2-difluoroethyl)cyclopropanesulfonamide and (4-fluoro-2-(trifluoromethyl)phenyl)boronic acid with anhydrous dioxane as solvent, and the reaction temperature of 140° C. ¹H NMR (CDCl₃, 400 MHz) δ 7.51-7.48 (m, 3H), 7.39-7.28 (m, 2H), 7.21 (d, 5.7 Hz, 1H), 6.14 (t, J=56.0 Hz, 1H), 5.15-5.01 (m, 2H), 4.73-4.63 (m, 1H), 4.14-3.89 (m, 4H), 2.47-2.44 (m, 1H), 1.19-1.09 (m, 2H), 0.93-0.83 (m, 2H)

### Example 177: (S)—N-(2,2-difluoro-1-(5-fluoro-6-(4-fluoro-2-(trifluoromethyl)phenyl)-1-(oxetan-3-yl)-1H-pyrrolo[2,3-b]pyridin-3-yl)ethyl)cyclopropanesulfonamide

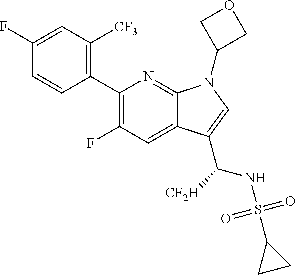 [SMILES: O=S(=O)(N[C@@H](c1cn(C2COC2)c2nc(-c3ccc(F)cc3C(F)(F)F)c(F)cc12)C(F)F)C1CC1]

### Step 1:1-(6-chloro-5-fluoro-1H-pyrrolo[2,3-b]pyridin-3-yl)-2,2-difluoroethan-1-one

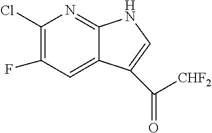 [SMILES: O=C(c1c[nH]c2nc(Cl)c(F)cc12)C(F)F]

The title compound was prepared following the same general protocol as described for Step 1, Example 11, using 6-chloro-3-(2,2-difluoroethyl)-5-fluoro-1H-pyrrolo[2,3-b]pyridine. 1H NMR (methanol-d4, 400 MHz) δ 7.81 (d, J=9.1 Hz, 1H), 7.43 (d, J=3.5 Hz, 1H), 6.47 (s, 1H), 6.03 (t, J=54.0 Hz, 1H)

### Step 2:1-(6-chloro-5-fluoro-1-(oxetan-3-yl)-1H-pyrrolo[2,3-b]pyridin-3-yl)-2,2-difluoroethan-1-one

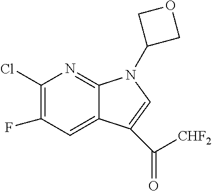 [SMILES: O=C(c1cn(C2COC2)c2nc(Cl)c(F)cc12)C(F)F]

The title compound was prepared following the same general protocol as described for Step 1, Example 2, using 1-(6-chloro-5-fluoro-1H-pyrrolo[2,3-b]pyridin-3-yl)-2,2-difluoroethan-1-one and 3-bromooxetane. ¹H NMR (methanol-d₄, 400 MHz) δ 8.85 (s, 1H), 8.43 (d, J=8.6 Hz, 1H), 6.54 (t, J=54.0 Hz, 1H), 6.00-5.93 (m, 1H), 5.20-5.08 (m, 4H)

### Step 3: (S,E)-N-(1-(6-chloro-5-fluoro-1-(oxetan-3-yl)-1H-pyrrolo[2,3-b]pyridin-3-yl)-2,2-difluoroethylidene)-2-methylpropane-2-sulfinamide

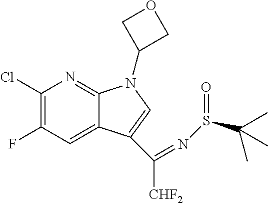 [SMILES: CC(C)(C)[S@](=O)/N=C(\c1cn(C2COC2)c2nc(Cl)c(F)cc12)C(F)F]

The title compound was prepared following the same general protocol as described for Step 2, Example 11, using 1-(6-chloro-5-fluoro-1-(oxetan-3-yl)-1H-pyrrolo[2,3-b]pyridin-3-yl)-2,2-difluoroethan-1-one, and the reaction was stirred for 2 h at 90° C. ¹H NMR (CDCl₃, 400 MHz) δ 8.41 (t, J=1.8 Hz, 1H), 8.20 (d, J=8.7 Hz, 1H), 7.8 (t, J=54.0 Hz, 1H), 6.13-5.98 (m, 1H), 5.23-5.18 (m, 2H), 5.02-4.97 (m, 2H), 1.35 (s, 9H)

### Step 4: (S)—N—((S)-1-(6-chloro-5-fluoro-1-(oxetan-3-yl)-1H-pyrrolo[2,3-b]pyridin-3-yl)-2,2-difluoroethyl)-2-methylpropane-2-sulfinamide

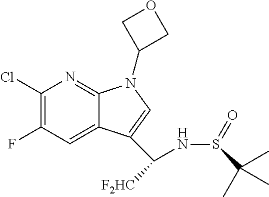 [SMILES: CC(C)(C)[S@](=O)N[C@@H](c1cn(C2COC2)c2nc(Cl)c(F)cc12)C(F)F]

The title compound was prepared following the same general protocol as described for Step 3, Example 31, using (S,E)-N-(1-(6-chloro-5-fluoro-1-(oxetan-3-yl)-1H-pyrrolo[2,3-b]pyridin-3-yl)-2,2-difluoroethylidene)-2-methylpropane-2-sulfinamide. ¹H NMR (CDCl₃, 400 MHz) δ 7.86 (s, 1H), 7.72 (d, J=8.6 Hz, 1H), 6.19 (td, J=55.8 Hz, 2.2 Hz, 1H), 5.98 (q, J=8.0 Hz, 1H), 5.17 (t, J=7.2 Hz, 2H), 4.97 (t, J=6.1 Hz, 1H), 4.91-4.85 (m, 2H), 4.01 (d, J=3.4 Hz, 1H), 1.21 (s, 9H)

### Step 5: (S)-1-(6-chloro-5-fluoro-1-(oxetan-3-yl)-1H-pyrrolo[2,3-b]pyridin-3-yl)-2,2-difluoroethan-1-amine

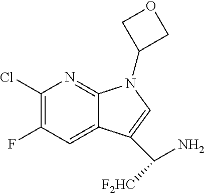 [SMILES: N[C@@H](c1cn(C2COC2)c2nc(Cl)c(F)cc12)C(F)F]

The title compound was prepared following the same general protocol as described for Step 3, Example 2, using (S)—N—((S)-1-(6-chloro-5-fluoro-1-(oxetan-3-yl)-1H-pyrrolo[2,3-b]pyridin-3-yl)-2,2-difluoroethyl)-2-methylpropane-2-sulfinamide

### Step 6: (S)—N-(1-(6-chloro-5-fluoro-1-(oxetan-3-yl)-1H-pyrrolo[2,3-b]pyridin-3-yl)-2,2-difluoroethyl)cyclopropanesulfonamide

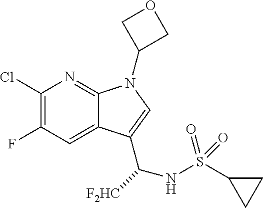 [SMILES: O=S(=O)(N[C@@H](c1cn(C2COC2)c2nc(Cl)c(F)cc12)C(F)F)C1CC1]

The title compound was prepared following the same general protocol as described for Step 4, Example 2, using (S)-1-(6-chloro-5-fluoro-1-(oxetan-3-yl)-1H-pyrrolo[2,3-b]pyridin-3-yl)-2,2-difluoroethan-1-amine and cyclopropanesulfonyl chloride. ¹H NMR (CDCl₃, 400 MHz) δ 7.89 (dd, J=8.5 Hz, 2.2 Hz, 1H), 7.53 (d, J=2.5 Hz, 1H), 6.09 (td, J=55.8 Hz, 2.2 Hz, 1H), 5.47-5.41 (m, 1H), 5.07-4.89 (m, 2H), 4.13-3.96 (m, 4H), 2.38-2.31 (m, 1H), 1.19-1.09 (m, 2H), 0.93-0.83 (m, 2H)

### Step 7: (S)—N-(2,2-difluoro-1-(5-fluoro-6-(4-fluoro-2-(trifluoromethyl)phenyl)-1-(oxetan-3-yl)-1H-pyrrolo[2,3-b]pyridin-3-yl)ethyl)cyclopropanesulfonamide

The title compound was prepared following the same general protocol as described for Step 6, Example 2, using (S)—N-(1-(6-chloro-5-fluoro-1-(oxetan-3-yl)-1H-pyrrolo[2,3-b]pyridin-3-yl)-2,2-difluoroethyl)cyclopropanesulfonamide and (4-fluoro-2-(trifluoromethyl)phenyl)boronic acid with anhydrous dioxane as solvent, and the reaction temperature of 140° C. ¹H NMR (CDCl₃, 400 MHz) δ 7.96-7.92 (m, 1H), 7.55-7.47 (m, 3H), 7.34 (td, J=10.6 Hz, 2.6 Hz, 1H), 6.09 (td, J=55.8 Hz, 2.2 Hz, 1H), 5.19-5.11 (m, 2H), 4.95-4.75 (m, 1H), 4.11-3.97 (m, 4H), 2.45-2.36 (m, 1H), 1.19-1.09 (m, 2H), 0.93-0.83 (m, 2H)

### Example 178: (S)—N-(2,2-difluoro-1-(5-fluoro-6-(5-fluoro-2-(trifluoromethyl)pyridin-3-yl)-1-(oxetan-3-yl)-1H-pyrrolo[2,3-b]pyridin-3-yl)ethyl)cyclopropanesulfonamide

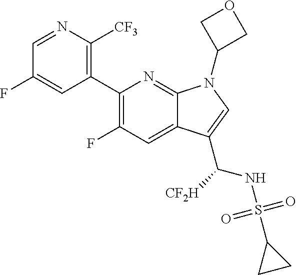 [SMILES: O=S(=O)(N[C@@H](c1cn(C2COC2)c2nc(-c3cc(F)cnc3C(F)(F)F)c(F)cc12)C(F)F)C1CC1]

The title compound was prepared following the same general protocol as described for Step 6, Example 2, using (S)—N-(1-(6-chloro-5-fluoro-1-(oxetan-3-yl)-1H-pyrrolo[2,3-b]pyridin-3-yl)-2,2-difluoroethyl)cyclopropanesulfonamide and 5-fluoro-3-(4,4,5,5-tetramethyl-1,3,2-dioxaborolan-2-yl)-2-(trifluoromethyl)pyridine with anhydrous dioxane as solvent, and the reaction temperature of 140° C. ¹H NMR (CDCl₃, 400 MHz) δ 8.66 (d, J=2.6 Hz, 1H), 7.98 (dd, J=9.5 Hz, 3.7 Hz, 1H), 7.62-7.60 (m, 2H), 6.18 (td, J=55.8 Hz, 2.2 Hz, 1H), 5.26-5.05 (m, 2H), 4.95-4.89 (m, 1H), 422-3.98 (m, 4H), 2.44-2.38 (m, 1H), 1.27-1.13 (m, 2H), 0.99-0.88 (m, 2H).

### Example 179: (S)—N-(1-(1-(3-cyanocyclobutyl)-5-fluoro-6-(4-fluoro-2-(trifluoromethyl)phenyl)-1H-indol-3-yl)-2,2-difluoroethyl)cyclopropanesulfonamide

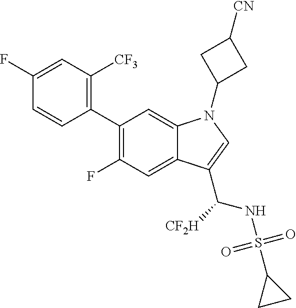 [SMILES: N#CC1CC(n2cc([C@H](NS(=O)(=O)C3CC3)C(F)F)c3cc(F)c(-c4ccc(F)cc4C(F)(F)F)cc32)C1]

The title compound was prepared following the same general protocol as described for Step 6, Example 2, using (S)—N-(1-(6-bromo-1-(3-cyanocyclobutyl)-5-fluoro-1H-indol-3-yl)-2,2-difluoroethyl)cyclopropanesulfonamide and (4-fluoro-2-(trifluoromethyl)phenyl)boronic acid with anhydrous dioxane as solvent, and the reaction temperature of 140° C. ¹H NMR (CDCl₃, 400 MHz) δ 7.51-7.48 (m, 3H), 7.36-7.28 (m, 2H), 7.12 (d, 5.7 Hz, 1H), 6.14 (t, J=56.0 Hz, 1H), 5.45-5.03 (m, 2H), 4.77-4.73 (m, 1H), 3.02-2.75 (m, 5H), 2.47-2.44 (m, 1H), 1.19-1.09 (m, 2H), 0.93-0.83 (m, 2H).

### Example 180: (S)—N-(1-(6-(6-amino-2-(trifluoromethyl)pyridin-3-yl)-1-cyclobutyl-5-fluoro-1H-pyrrolo[2,3-b]pyridin-3-yl)-2,2-difluoroethyl)cyclopropanesulfonamide

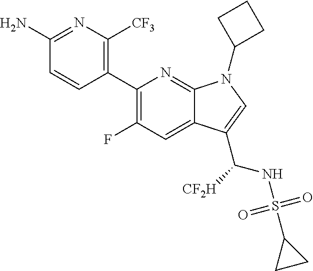 [SMILES: Nc1ccc(-c2nc3c(cc2F)c([C@H](NS(=O)(=O)C2CC2)C(F)F)cn3C2CCC2)c(C(F)(F)F)n1]

The title compound was prepared following the same general protocol as described for Step 2, Example 158, using (S)—N-(1-(1-cyclobutyl-5-fluoro-6-(tributylstannyl)-1H-pyrrolo[2,3-b]pyridin-3-yl)-2,2-difluoroethyl)cyclopropanesulfonamide and 5-bromo-6-(trifluoromethyl)pyridin-2-amine. ¹H NMR (CDCl₃, 400 MHz) δ 7.79 (d, J=9.6 Hz, 1H), 7.59-7.57 (m, 2H), 6.71 (d, J=8.5 Hz, 1H), 6.15 (td, J=55.8 Hz, 2.2 Hz, 1H), 5.28-5.22 (m, 2H), 5.08-5.05 (m, 1H), 2.56-2.39 (m, 5H), 1.88-1.81 (m, 2H), 1.21-1.06 (m, 2H), 0.92-0.83 (m, 2H).

### Example 181: (S)—N-(2,2-difluoro-1-(5-fluoro-6-(4-fluoro-2-(trifluoromethyl)phenyl)-1-(3-fluorocyclobutyl)-1H-indol-3-yl)ethyl)cyclopropanesulfonamide

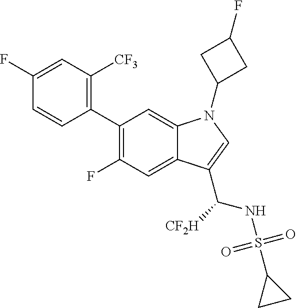 [SMILES: O=S(=O)(N[C@@H](c1cn(C2CC(F)C2)c2cc(-c3ccc(F)cc3C(F)(F)F)c(F)cc12)C(F)F)C1CC1]

### Step 1:3-(benzyloxy)cyclobutan-1-ol

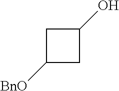 [SMILES: OC1CC(OCc2ccccc2)C1]

To an ice-cooled solution of 3-(benzyloxy)cyclobutanone (3 g, 17 mmol, 1 eq) in methanol (50 mL) was added sodium borohydride (1.28 g, 34 mmol, 2 eq) in portions. The mixture was stirred at 0° for 2 hours by which time the analytical HPLC showed complete conversion of starting material. The mixture was quenched with water and methanol was evaporated under reduced pressure. The mixture was diluted with ethyl acetate (100 ml). Phases were separated and the aqueous phase was extracted with ethyl acetate (2×50 mL). The combined organic phase was washed with brine and dried over sodium sulfate. The crude product was used for the next step without any further purifications (2.9 g, 92%). ¹H NMR (400 MHz, CDCl₃) δ (ppm): 7.28-7.39 (m, 5H), 4.43 (s, 2H), 3.88 (quin, J=7.08 Hz, 1H), 3.61 (quin, J=7.08 Hz, 1H), 2.76-2.69 (m, 2H), 2.07 (s, 1H), 1.99-1.91 (m, 2H).

### Step 2:3-(benzyloxy)cyclobutyl 4-methylbenzenesulfonate

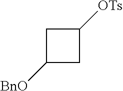 [SMILES: Cc1ccc(S(=O)(=O)OC2CC(OCc3ccccc3)C2)cc1]

To an ice-cooled solution of 3-(benzyloxy)cyclobutan-1-ol (2.9 g, 16.2 mmol, 1 eq) in dichloromethane (50 mL) was added triethylamine (10 mL) followed by the dropwise addition of p-toluenesulfonyl chloride (6.1 g, 32 mmol, 1.98 eq) in dichloromethane (20 mL). The resulting solution was stirred overnight at room temperature. The mixture was diluted with dichloromethane (50 ml) and washed with water (2×50 mL) and the aqueous phase was back extracted with dichloromethane (2×25 mL). The combined organic phase was washed with brine and dried over sodium sulfate and concentrated in vacuo to give crude product which was further purified by flash chromatography affording a pure light yellowish compound (5 g, 90%). ¹H NMR (400 MHz, CDCl₃) δ (ppm): 7.78 (dd, J=6.72 Hz, J=1.64 Hz, 2H), 7.36-7.36 (m, 7H), 4.44 (quin, J=7.28 Hz, 1H), 4.3 (s, 2H) 3.60 (quin, J=7.2 Hz, 1H), 2.65-2.59 (m, 2H), 2.46 (s, 3H), 2.21-2.14 (m, 2H).

### Step 3:1-(3-(benzyloxy)cyclobutyl)-6-bromo-5-fluoro-1H-indole

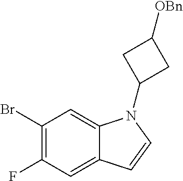 [SMILES: Fc1cc2ccn(C3CC(OCc4ccccc4)C3)c2cc1Br]

To a mixture of 6-bromo-5-fluoro-1H-indole (1.3 g, 6.07 mmol, 1 eq) and 3-(benzyloxy)cyclobutyl 4-methylbenzenesulfonate (2.3 g, 6.67 mmol, 1.1 eq) in N,N-Dimethylformamide (30 ml) was added cesium carbonate (4.5 g, 12.14 mmol, 2 eq) at room temperature. The resulting mixture was stirred at 100° C. The progress of the reaction was monitored by analytical HPLC until the consumption of the starting materials. The reaction was allowed to cool down and water (200 ml) was added to the mixture and extracted twice with ethyl acetate (100 ml). The organic phase was washed with brine, dried over sodium sulfate, mixed with celite and concentrated in vacuo. The desired alkylated indole was purified by column chromatography and obtained as transparent oil (1.7 g, 82%). ¹H NMR (400 MHz, CDCl₃) δ (ppm): 7.49 (dd, J=5.68 Hz, J=0.32 Hz, 1H), 7.42-7.40 (m, 4H), 7.38-7.34 (m, 2H), 7.29 (d, J=3.2 Hz, 1H), 6.49 (dd, J=3.24 Hz, J=0.72 Hz, 1H), 5.03 (quin, J=8 Hz, 1H), 4.54 (s, 2H) 3.60 (m, 1H), 2.80-2.74 (m, 2H), 2.69-2.62 (m, 2H).

### Step 4:3-(6-bromo-5-fluoro-1H-indol-1-yl)cyclobutan-1-ol

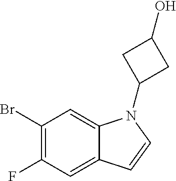 [SMILES: OC1CC(n2ccc3cc(F)c(Br)cc32)C1]

3-(benzyloxy)cyclobutyl 4-methylbenzenesulfonate (1.7 g, 4.55 mmol, 1 eq) was dissolved in dry dichloromethane under argon and the mixture was cooled at −78° C. Boron trichloride (9.1 ml, 1M in dichloromethane, 2 eq) was added dropwise and the reaction mixture was stirred at −78° C. for 2 hours. The total conversion of the starting material was indicated by analytical HPLC. The mixture was diluted with dichloromethane (100 ml) and cold water was added slowly to quench the unreacted boron trichloride. The organic phase was separated and washed with brine and dried over sodium sulfate. The crude product was purified by column chromatography and the title compound was isolated as a white solid (600 mg, 47%). ¹H NMR (600 MHz, CD₃OD) δ (ppm): 7.43 (d, J=5.7 Hz, 1H), 7.35 (d, J=3.24 Hz, 1H), 7.19 (d, J=9.42 Hz, 1H), 6.34 (dd, J=3.24 Hz, J=0.72 Hz, 1H), 4.93 (quin, J=5.58 Hz, 1H), 4.4 (m, 1H), 2.58-2.53 (m, 2H), 2.48-2.43 (m, 2H).

### Step 5:6-bromo-5-fluoro-1-(3-fluorocyclobutyl)-1H-indole

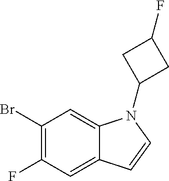 [SMILES: Fc1cc2ccn(C3CC(F)C3)c2cc1Br]

To an ice-cooled solution of 3-(6-bromo-5-fluoro-1H-indol-1-yl)cyclobutan-1-ol (600 mg, 2.12 mmol) in dry dichloromethane (40 ml) under argon was added DAST (420 μl, 3.18 mmol). The ice bath was removed and the reaction mixture was stirred at room temperature overnight. The solution was diluted with dichloromethane (100 ml) and quenched with sodium bicarbonate. The organic phase was washed with brine, dried over sodium sulfate and concentrated in vacuo. The crude product was purified by column chromatography yielding 200 mg of white solid. ¹H NMR (600 MHz, CD₃OD) δ (ppm): 7.39 (d, J=5.58 Hz, 1H), 7.27 (d, J=9.12 Hz, 1H), 7.21 (d, J=4.74 Hz, 1H), 6.42 (d, J=3.24 Hz, 1H), 5.04-4.99 (m, 0.5H, CH—F), 4.97-4.83 (m, 0.5H, CH—F), 4.29-4.22 (m, 1H), 3.10-3.01 (m, 2H), 2.65-2.52 (m, 2H).

### Step 6:1-(6-bromo-5-fluoro-1-(3-fluorocyclobutyl)-1H-indol-3-yl)-2,2-difluoroethan-1-one

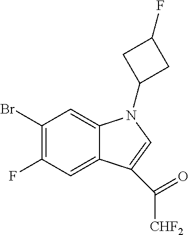 [SMILES: O=C(c1cn(C2CC(F)C2)c2cc(Br)c(F)cc12)C(F)F]

The title compound was prepared following the same general protocol as described for Step 1, Example 11, using 6-bromo-5-fluoro-1-(3-fluorocyclobutyl)-1H-indole-3-carbaldehyde and 2,2-difluoroacetic anhydride. ¹H NMR (CDCl₃, 400 MHz) δ 8.13-8.10 (m, 2H), 7.51 (d, J=5.4 Hz, 1H), 6.10 (t, J=54.1 Hz, 1H), 5.18-4.98 (m, 1H), 4.40 (q, J=7.7 Hz, 1H), 3.39-3.20 (m, 2H), 2.80-2.65 (m, 2H)

### Step 7: (S,E)-N-(1-(6-bromo-5-fluoro-1-(3-fluorocyclobutyl)-1H-indol-3-yl)-2,2-difluoroethylidene)-2-methylpropane-2-sulfinamide

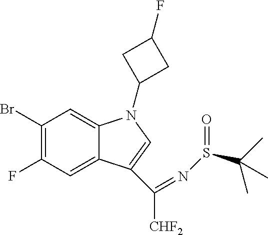 [SMILES: CC(C)(C)[S@](=O)/N=C(\c1cn(C2CC(F)C2)c2cc(Br)c(F)cc12)C(F)F]

The title compound was prepared following the same general protocol as described for Step 2, Example 11, using 1-(6-bromo-5-fluoro-1-(3-fluorocyclobutyl)-1H-indol-3-yl)-2,2-difluoroethan-1-one and (S)-2-methylpropane-2-sulfinamide, and the reaction was stirred for 2 h at 90° C. ¹H NMR (CDCl3, 400 MHz) δ 8.13-8.10 (m, 2H), 7.51 (d, J=5.4 Hz, 1H), 6.10 (t, J=54.1 Hz, 1H), 5.18-4.98 (m, 1H), 4.40 (q, J=7.7 Hz, 1H), 3.39-3.20 (m, 2H), 2.80-2.65 (m, 2H)

### Step 8: (S)—N—((S)-1-(6-bromo-5-fluoro-1-(3-fluorocyclobutyl)-1H-indol-3-yl)-2,2-difluoroethyl)-2-methylpropane-2-sulfinamide

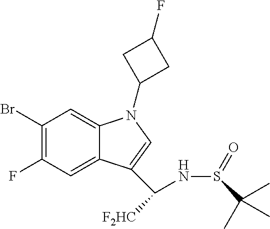 [SMILES: CC(C)(C)[S@](=O)N[C@@H](c1cn(C2CC(F)C2)c2cc(Br)c(F)cc12)C(F)F]

The title compound was prepared following the same general protocol as described for Step 3, Example 31, using (S,E)-N-(1-(6-bromo-5-fluoro-1-(3-fluorocyclobutyl)-1H-indol-3-yl)-2,2-difluoroethylidene)-2-methylpropane-2-sulfinamide. ¹H NMR (CDCl₃, 400 MHz) δ 7.47 (d, J=5.6 Hz, 1H), 7.41 (s, 1H), 7.36 (d, J=9.2 Hz, 1H), 6.06 (td, J=55.8 Hz, 4.2 Hz, 1H), 5.11-4.88 (m, 1H), 4.83-4.80 (m, 1H), 4.37-4.28 (m, 1H), 3.90 (broad s, 1H), 3.18-3.10 (m, 2H), 2.76-2.57 (m, 2H), 1.21 (s, 9H)

### Step 9: (S)-1-(6-bromo-1-cyclobutyl-5-fluoro-1H-indol-3-yl)-2,2-difluoroethan-1-amine

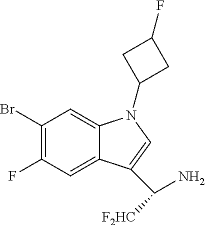 [SMILES: N[C@@H](c1cn(C2CC(F)C2)c2cc(Br)c(F)cc12)C(F)F]

The title compound was prepared following the same general protocol as described for Step 4, Example 2, using (S)—N—((S)-1-(6-bromo-5-fluoro-1-(3-fluorocyclobutyl)-1H-indol-3-yl)-2,2-difluoroethyl)-2-methylpropane-2-sulfinamide.

### Step 10: (S)—N-(1-(6-bromo-5-fluoro-1-(3-fluorocyclobutyl)-1H-indol-3-yl)-2,2-difluoroethyl)cyclopropanesulfonamide

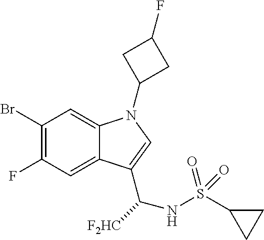 [SMILES: O=S(=O)(N[C@@H](c1cn(C2CC(F)C2)c2cc(Br)c(F)cc12)C(F)F)C1CC1]

The title compound was prepared following the same general protocol as described for Step 4, Example 2, using (S)-1-(6-bromo-1-cyclobutyl-5-fluoro-1H-indol-3-yl)-2,2-difluoroethan-1-amine and cyclopropanesulfonyl chloride. ¹H NMR (CDCl₃, 400 MHz) δ 7.49 (d, J=2.3 Hz, 1H), 7.47 (s, 1H), 7.36 (d, J=3.4 Hz, 1H), 6.12 (td, J=55.8 Hz, 4.2 Hz, 1H), 5.13-5.03 (m, 1H), 4.97-4.92 (m, 1H), 4.37-4.29 (m, 1H), 3.82-3.63 (m, 1H), 3.19-3.11 (m, 2H), 2.72-2.58 (m, 2H), 2.41-2.34 (m, 1H), 1.19-1.08 (m, 2H), 0.95-0.86 (m, 2H)

### Step 11: (S)—N-(2,2-difluoro-1-(5-fluoro-6-(4-fluoro-2-(trifluoromethyl)phenyl)-1-(3-fluorocyclobutyl)-1H-indol-3-yl)ethyl)cyclopropanesulfonamide

The title compound was prepared following the same general protocol as described for Step 6, Example 2, using (S)—N-(1-(6-bromo-5-fluoro-1-(3-fluorocyclobutyl)-1H-indol-3-yl)-2,2-difluoroethyl)cyclopropanesulfonamide and (4-fluoro-2-(trifluoromethyl)phenyl)boronic acid with anhydrous dioxane as solvent, and the reaction temperature of 140° C. ¹H NMR (CDCl₃, 400 MHz) δ 7.51-7.46 (m, 2H), 7.41 (s, 1H), 7.39-7.27 (m, 2H), 7.15 (d, J=5.9 Hz, 1H), 6.17 (td, J=55.8 Hz, 4.2 Hz, 1H), 5.30-5.02 (m, 2H), 4.95-4.88 (m, 1H), 4.38-4.31 (m, 1H), 3.16-3.02 (m, 2H), 2.75-2.60 (m, 2H), 2.41-2.34 (m, 1H), 1.23-1.14 (m, 2H), 0.95-0.83 (m, 2H).

### Example 182: (S)—N-(2,2-difluoro-1-(5-fluoro-6-(5-fluoro-2-(trifluoromethyl)pyridin-3-yl)-1-(3-fluorocyclobutyl)-1H-indol-3-yl)ethyl)cyclopropanesulfonamide

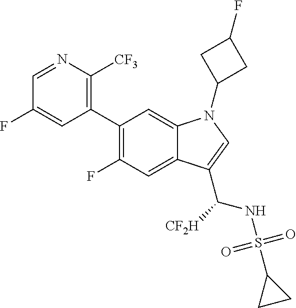 [SMILES: O=S(=O)(N[C@@H](c1cn(C2CC(F)C2)c2cc(-c3cc(F)cnc3C(F)(F)F)c(F)cc12)C(F)F)C1CC1]

The title compound was prepared following the same general protocol as described for Step 6, Example 2, using (S)—N-(1-(6-bromo-5-fluoro-1-(3-fluorocyclobutyl)-1H-indol-3-yl)-2,2-difluoroethyl)cyclopropanesulfonamide and 6-fluoro-3-(4,4,5,5-tetramethyl-1,3,2-dioxaborolan-2-yl)-2-(trifluoromethyl)pyridine with anhydrous dioxane as solvent, and the reaction temperature of 140° C. ¹H NMR (CDCl₃, 400 MHz) δ 8.61 (d, J=2.6 Hz, 1H), 7.54-7.50 (m, 2H), 7.43 (s, 1H), 7.18 (d, J=5.8 Hz, 1H), 6.18 (td, J=55.8 Hz, 4.2 Hz, 1H), 5.17-4.92 (m, 2H), 4.89-4.87 (m, 1H), 4.40-4.33 (m, 1H), 3.19-3.08 (m, 2H), 2.74-2.61 (m, 2H), 2.47-2.40 (m, 1H), 1.23-1.14 (m, 2H), 0.95-0.83 (m, 2H)

### Biological Examples

Cell culture. HEK293 were purchased from American Type Culture Collection (ATCC) and cultured in in DMEM supplemented with 10% FBS, 2 mM L-glutamine, and 1% penicillin/streptomycin at 37° C., 5% CO₂ under standard culture conditions.

Luciferase reporter assays. HEK293 cells were plated 24 hours prior to transfection in 10 cm dishes at a density of 350,000 cells/10 ml/plate. Transfections were performed using Lipofectamine 3000 (Invitrogen) according to manufacturer's protocol. Cells were co-transfected with pG5-UAS, pCMV-Gal4-REV-ERBα (residues 206-614, containing the hinge and the ligand binding domain), along with pACT (Promega) fused with the mouse NCOR receptor interaction domain (RID, residues 1828-2471) or pACT empty vector expressing the VP-16 fusion protein only as a control. Sixteen hours post-transfection, cells were trypsinized, counted and plated at 10,000 cells/20 ul/well in 384 well plates. 4 h post cell plating, cells were treated with vehicle or compound. Ligand stocks were prepared via serial dilution in DMSO, added to 8 wells per concentration, and plates were incubated at 37° C. Twenty-four hours post-treatment, luciferase activity was measured using BriteLite (PerkinElmer Life and Analytical Sciences) and read using an Envision multilabel plate reader (PerkinElmer Life and Analytical Sciences). All values were normalized to DMSO to produce fold induction values. Data were plotted using GraphPad Prism.

TR-FRET corepressor interaction assay. The time-resolved fluorescence resonance energy transfer (TR-FRET) assay was performed in black low-volume 384-well plates (Greiner). Each well contained 4 nM 6×Histag-REV-ERBα LBD (human; residues 281-614 A324-422) or 6×Histag-REV-ERBα LBD (human; residues 381-579) protein expressed in and purified from Escherichia coli using nickel affinity and size exclusion chromatography; 1 nM LanthaScreen Elite Tb-anti-HIS Antibody (Thermo Fisher Scientific); and 400 nM FITC-labeled peptide derived from the SMRT corepressor (TNMGLEAIIRKALMGKYDQWEE) containing a N-terminal FITC label with a six-carbon linker (Ahx) and an amidated C-terminus for stability (synthesized by LifeTein) in TR-FRET buffer (20 mM potassium phosphate, pH 7.4, 50 mM potassium chloride, 1 mM dithiothreitol, and 0.005% Tween-20). Ligand stocks were prepared via serial dilution in DMSO, added to wells in triplicate, and plates were incubated at 4° C. for 2 h and read using a BioTek Synergy Neo multimode plate reader. The Tb donor was excited at 340 nm; its fluorescence emission was monitored at 495 nm, and the acceptor FITC emission was measured at 520 nm; and the TR-FRET ratio was calculated as the signal at 520 nm divided by the signal at 495 nm. Data were plotted using GraphPad Prism as TR-FRET ratio vs. ligand concentration and fit to sigmoidal dose response curve equation.

Exemplary compounds of the present disclosure were assayed as described above to assess agonism of REV-ERBα. Results of the FRET assay are shown in Table 2 below (A: EC₅₀<100 nM; B: EC₅₀ 100 nM-1000 nM; C: EC₅₀>1000 nM; NT: not tested).

TABLE 2
Compounds and Agonism of REV-ERBα

| Com- | | RE- |
|---|---|---|
| pound | Structure | VERBα |
| 1 | 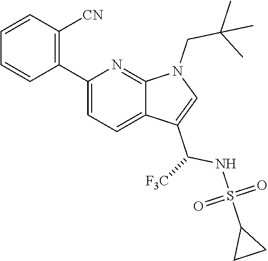 [SMILES: CC(C)(C)Cn1cc([C@H](NS(=O)(=O)C2CC2)C(F)(F)F)c2ccc(-c3ccccc3C#N)nc21] | B |
| 2 | 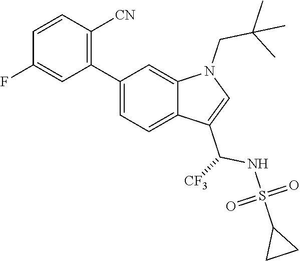 [SMILES: CC(C)(C)Cn1cc([C@H](NS(=O)(=O)C2CC2)C(F)(F)F)c2ccc(-c3cc(F)ccc3C#N)cc21] | B |
| 3 | 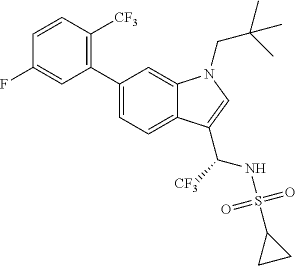 [SMILES: CC(C)(C)Cn1cc([C@H](NS(=O)(=O)C2CC2)C(F)(F)F)c2ccc(-c3cc(F)ccc3C(F)(F)F)cc21] | A |
| 4 | 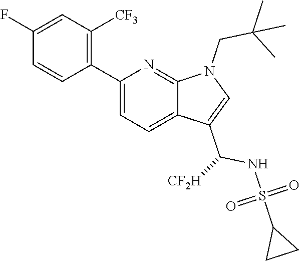 [SMILES: CC(C)(C)Cn1cc([C@H](NS(=O)(=O)C2CC2)C(F)F)c2ccc(-c3ccc(F)cc3C(F)(F)F)nc21] | A |
| 5 | 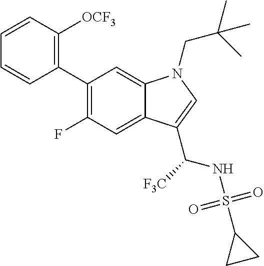 [SMILES: CC(C)(C)Cn1cc([C@H](NS(=O)(=O)C2CC2)C(F)(F)F)c2cc(F)c(-c3ccccc3OC(F)(F)F)cc21] | A |
| 6 | 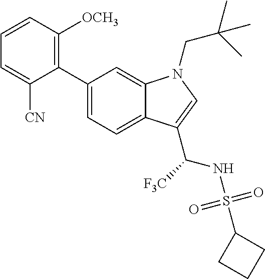 [SMILES: COc1cccc(C#N)c1-c1ccc2c([C@H](NS(=O)(=O)C3CCC3)C(F)(F)F)cn(CC(C)(C)C)c2c1] | B |
| 7 | 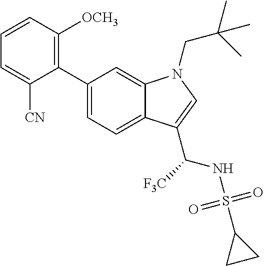 [SMILES: COc1cccc(C#N)c1-c1ccc2c([C@H](NS(=O)(=O)C3CC3)C(F)(F)F)cn(CC(C)(C)C)c2c1] | B |
| 8 | 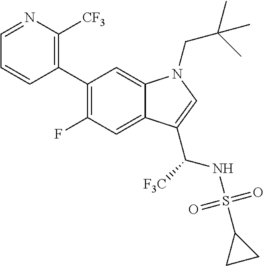 [SMILES: CC(C)(C)Cn1cc([C@H](NS(=O)(=O)C2CC2)C(F)(F)F)c2cc(F)c(-c3cccnc3C(F)(F)F)cc21] | B |
| 9 | 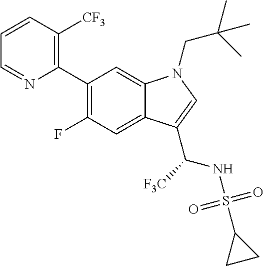 [SMILES: CC(C)(C)Cn1cc([C@H](NS(=O)(=O)C2CC2)C(F)(F)F)c2cc(F)c(-c3ncccc3C(F)(F)F)cc21] | B |
| 10 | 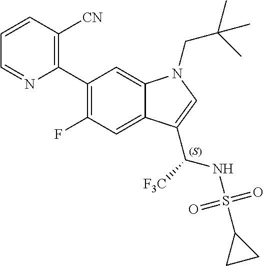 | B |
| 11 | 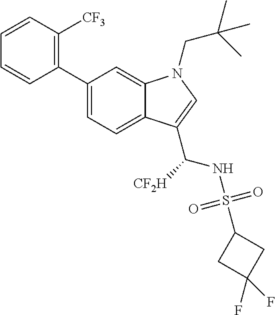 [SMILES: CC(C)(C)Cn1cc([C@H](NS(=O)(=O)C2CC(F)(F)C2)C(F)F)c2ccc(-c3ccccc3C(F)(F)F)cc21] | A |
| 12 | 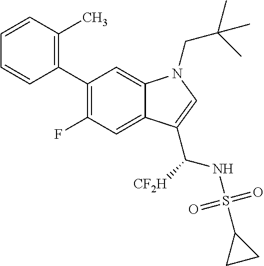 [SMILES: Cc1ccccc1-c1cc2c(cc1F)c([C@H](NS(=O)(=O)C1CC1)C(F)F)cn2CC(C)(C)C] | A |
| 13 | 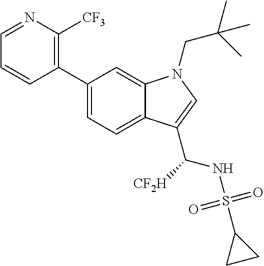 [SMILES: CC(C)(C)Cn1cc([C@H](NS(=O)(=O)C2CC2)C(F)F)c2ccc(-c3cccnc3C(F)(F)F)cc21] | A |
| 14 | 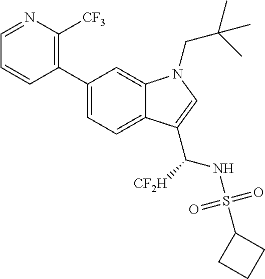 [SMILES: CC(C)(C)Cn1cc([C@H](NS(=O)(=O)C2CCC2)C(F)F)c2ccc(-c3cccnc3C(F)(F)F)cc21] | A |
| 15 | 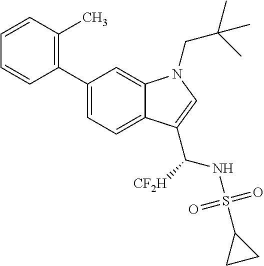 [SMILES: Cc1ccccc1-c1ccc2c([C@H](NS(=O)(=O)C3CC3)C(F)F)cn(CC(C)(C)C)c2c1] | A |
| 16 | 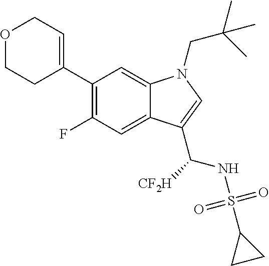 [SMILES: CC(C)(C)Cn1cc([C@H](NS(=O)(=O)C2CC2)C(F)F)c2cc(F)c(C3=CCOCC3)cc21] | A |
| 17 | 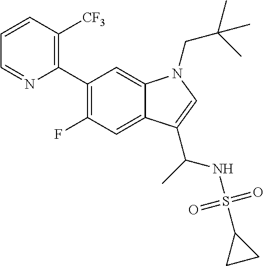 [SMILES: CC(NS(=O)(=O)C1CC1)c1cn(CC(C)(C)C)c2cc(-c3ncccc3C(F)(F)F)c(F)cc12] | B |
| 18 | 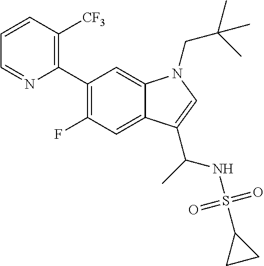 [SMILES: CC(NS(=O)(=O)C1CC1)c1cn(CC(C)(C)C)c2cc(-c3ncccc3C(F)(F)F)c(F)cc12] | A |
| 19 | 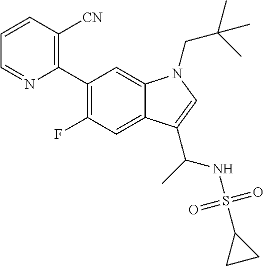 [SMILES: CC(NS(=O)(=O)C1CC1)c1cn(CC(C)(C)C)c2cc(-c3ncccc3C#N)c(F)cc12] | A |
| 20 | 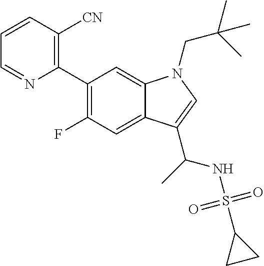 [SMILES: CC(NS(=O)(=O)C1CC1)c1cn(CC(C)(C)C)c2cc(-c3ncccc3C#N)c(F)cc12] | B |
| 21 | 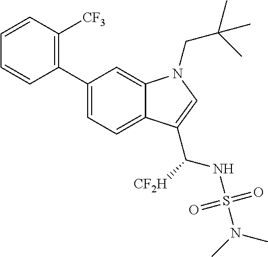 [SMILES: CN(C)S(=O)(=O)N[C@@H](c1cn(CC(C)(C)C)c2cc(-c3ccccc3C(F)(F)F)ccc12)C(F)F] | A |
| 22 | 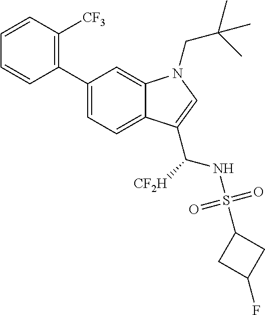 [SMILES: CC(C)(C)Cn1cc([C@H](NS(=O)(=O)C2CC(F)C2)C(F)F)c2ccc(-c3ccccc3C(F)(F)F)cc21] | A |
| 23 | 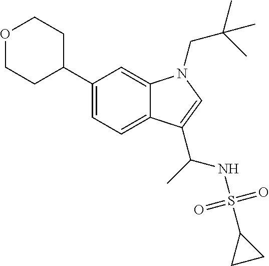 [SMILES: CC(NS(=O)(=O)C1CC1)c1cn(CC(C)(C)C)c2cc(C3CCOCC3)ccc12] | A |
| 24 | 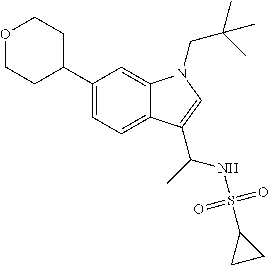 [SMILES: CC(NS(=O)(=O)C1CC1)c1cn(CC(C)(C)C)c2cc(C3CCOCC3)ccc12] | B |
| 26 | 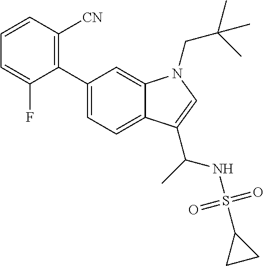 [SMILES: CC(NS(=O)(=O)C1CC1)c1cn(CC(C)(C)C)c2cc(-c3c(F)cccc3C#N)ccc12] | B |
| 27 | 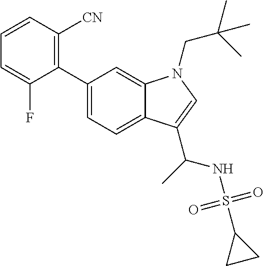 [SMILES: CC(NS(=O)(=O)C1CC1)c1cn(CC(C)(C)C)c2cc(-c3c(F)cccc3C#N)ccc12] | A |
| 28 | 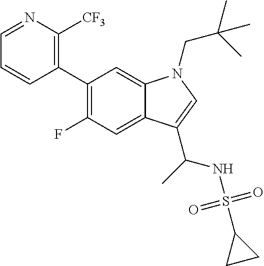 [SMILES: CC(NS(=O)(=O)C1CC1)c1cn(CC(C)(C)C)c2cc(-c3cccnc3C(F)(F)F)c(F)cc12] | A |
| 29 | 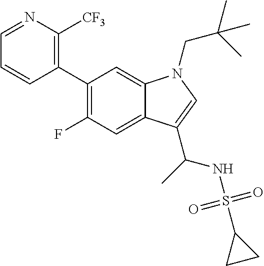 [SMILES: CC(NS(=O)(=O)C1CC1)c1cn(CC(C)(C)C)c2cc(-c3cccnc3C(F)(F)F)c(F)cc12] | B |
| 30 | 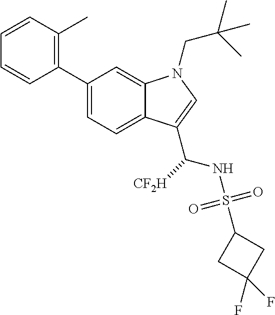 [SMILES: Cc1ccccc1-c1ccc2c([C@H](NS(=O)(=O)C3CC(F)(F)C3)C(F)F)cn(CC(C)(C)C)c2c1] | B |
| 31 | 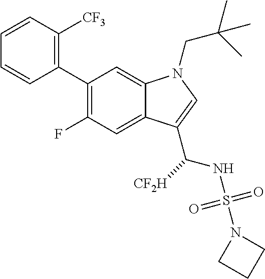 [SMILES: CC(C)(C)Cn1cc([C@H](NS(=O)(=O)N2CCC2)C(F)F)c2cc(F)c(-c3ccccc3C(F)(F)F)cc21] | A |
| 32 | 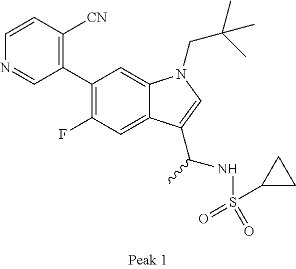 [SMILES: CC(NS(=O)(=O)C1CC1)c1cn(CC(C)(C)C)c2cc(-c3cnccc3C#N)c(F)cc12] | A |
| 33 | 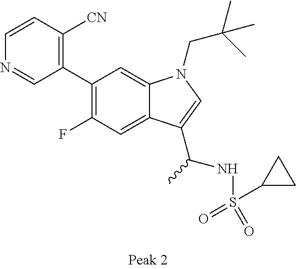 [SMILES: CC(NS(=O)(=O)C1CC1)c1cn(CC(C)(C)C)c2cc(-c3cnccc3C#N)c(F)cc12] | B |
| 34 | 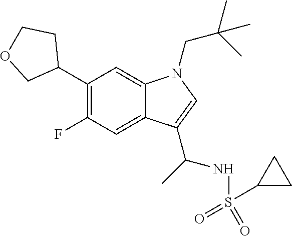 [SMILES: CC(NS(=O)(=O)C1CC1)c1cn(CC(C)(C)C)c2cc(C3CCOC3)c(F)cc12] | A |
| 35 | 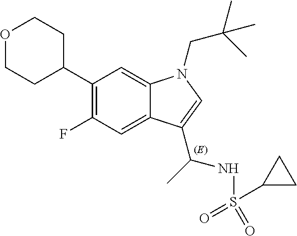 [SMILES: CC(NS(=O)(=O)C1CC1)c1cn(CC(C)(C)C)c2cc(C3CCOCC3)c(F)cc12] | A |
| 36 | 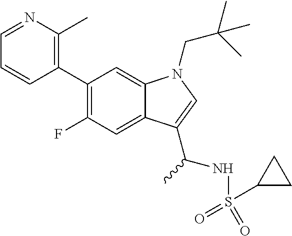 [SMILES: Cc1ncccc1-c1cc2c(cc1F)c(C(C)NS(=O)(=O)C1CC1)cn2CC(C)(C)C] | A |
| 37 | 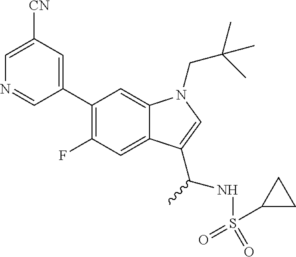 [SMILES: CC(NS(=O)(=O)C1CC1)c1cn(CC(C)(C)C)c2cc(-c3cncc(C#N)c3)c(F)cc12] | B |
| 38 | 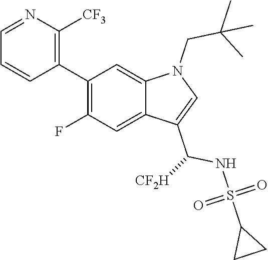 [SMILES: CC(C)(C)Cn1cc([C@H](NS(=O)(=O)C2CC2)C(F)F)c2cc(F)c(-c3cccnc3C(F)(F)F)cc21] | AA |
| 39 | 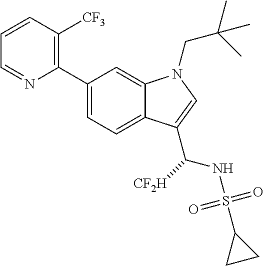 [SMILES: CC(C)(C)Cn1cc([C@H](NS(=O)(=O)C2CC2)C(F)F)c2ccc(-c3ncccc3C(F)(F)F)cc21] | A |
| 40 | 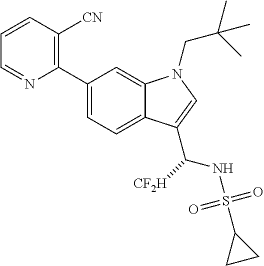 [SMILES: CC(C)(C)Cn1cc([C@H](NS(=O)(=O)C2CC2)C(F)F)c2ccc(-c3ncccc3C#N)cc21] | A |
| 41 | 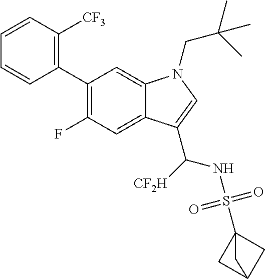 [SMILES: CC(C)(C)Cn1cc(C(NS(=O)(=O)C23CC(C2)C3)C(F)F)c2cc(F)c(-c3ccccc3C(F)(F)F)cc21] | A |
| 42 | 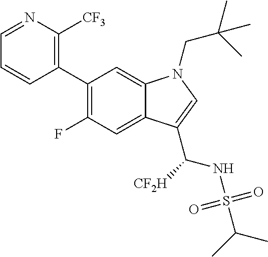 [SMILES: CC(C)S(=O)(=O)N[C@@H](c1cn(CC(C)(C)C)c2cc(-c3cccnc3C(F)(F)F)c(F)cc12)C(F)F] | A |
| 43 | 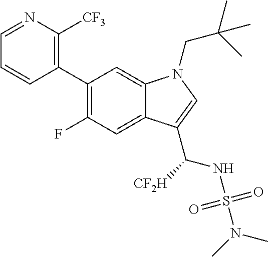 [SMILES: CN(C)S(=O)(=O)N[C@@H](c1cn(CC(C)(C)C)c2cc(-c3cccnc3C(F)(F)F)c(F)cc12)C(F)F] | A |
| 44 | 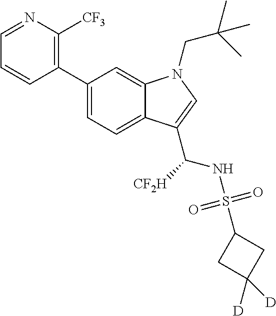 [SMILES: [2H]C1([2H])CC(S(=O)(=O)N[C@@H](c2cn(CC(C)(C)C)c3cc(-c4cccnc4C(F)(F)F)ccc23)C(F)F)C1] | NT |
| 45 | 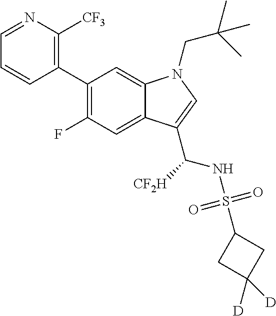 [SMILES: [2H]C1([2H])CC(S(=O)(=O)N[C@@H](c2cn(CC(C)(C)C)c3cc(-c4cccnc4C(F)(F)F)c(F)cc23)C(F)F)C1] | A |
| 46 | 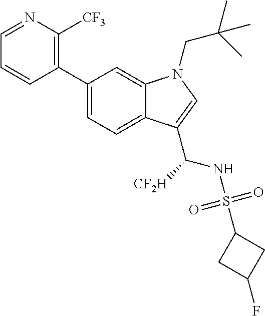 [SMILES: CC(C)(C)Cn1cc([C@H](NS(=O)(=O)C2CC(F)C2)C(F)F)c2ccc(-c3cccnc3C(F)(F)F)cc21] | A |
| 47 | 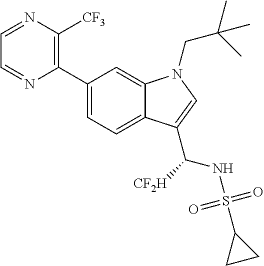 [SMILES: CC(C)(C)Cn1cc([C@H](NS(=O)(=O)C2CC2)C(F)F)c2ccc(-c3nccnc3C(F)(F)F)cc21] | A |
| 48 | 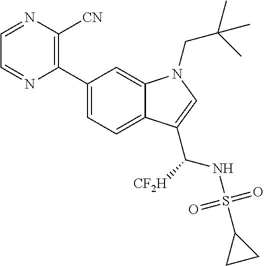 [SMILES: CC(C)(C)Cn1cc([C@H](NS(=O)(=O)C2CC2)C(F)F)c2ccc(-c3nccnc3C#N)cc21] | B |
| 49 | 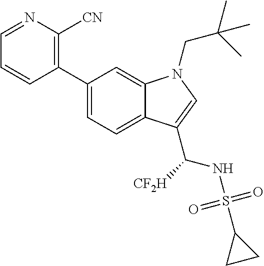 [SMILES: CC(C)(C)Cn1cc([C@H](NS(=O)(=O)C2CC2)C(F)F)c2ccc(-c3cccnc3C#N)cc21] | A |
| 51 | 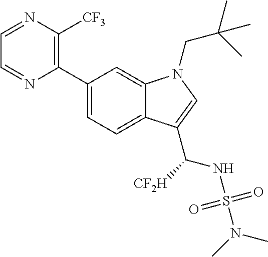 [SMILES: CN(C)S(=O)(=O)N[C@@H](c1cn(CC(C)(C)C)c2cc(-c3nccnc3C(F)(F)F)ccc12)C(F)F] | A |
| 52 | 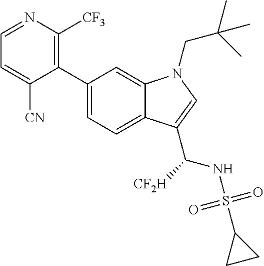 [SMILES: CC(C)(C)Cn1cc([C@H](NS(=O)(=O)C2CC2)C(F)F)c2ccc(-c3c(C#N)ccnc3C(F)(F)F)cc21] | B |
| 53 | 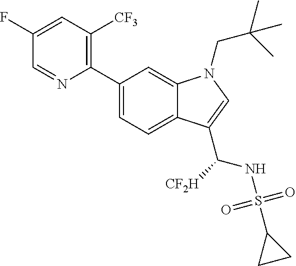 [SMILES: CC(C)(C)Cn1cc([C@H](NS(=O)(=O)C2CC2)C(F)F)c2ccc(-c3ncc(F)cc3C(F)(F)F)cc21] | A |
| 54 | 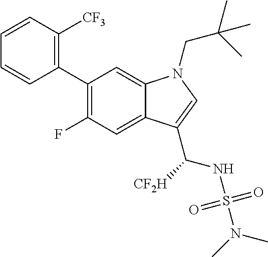 [SMILES: CN(C)S(=O)(=O)N[C@@H](c1cn(CC(C)(C)C)c2cc(-c3ccccc3C(F)(F)F)c(F)cc12)C(F)F] | A |
| 55 | 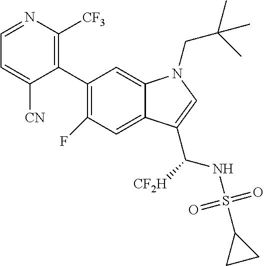 [SMILES: CC(C)(C)Cn1cc([C@H](NS(=O)(=O)C2CC2)C(F)F)c2cc(F)c(-c3c(C#N)ccnc3C(F)(F)F)cc21] | A |
| 56 | 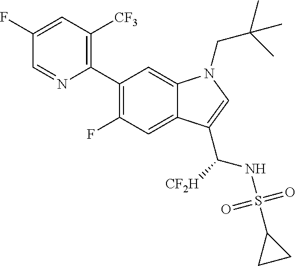 [SMILES: CC(C)(C)Cn1cc([C@H](NS(=O)(=O)C2CC2)C(F)F)c2cc(F)c(-c3ncc(F)cc3C(F)(F)F)cc21] | A |
| 57 | 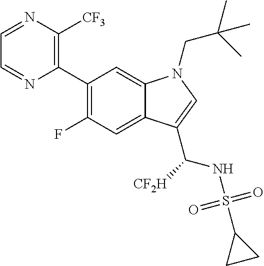 [SMILES: CC(C)(C)Cn1cc([C@H](NS(=O)(=O)C2CC2)C(F)F)c2cc(F)c(-c3nccnc3C(F)(F)F)cc21] | A |
| 58 | 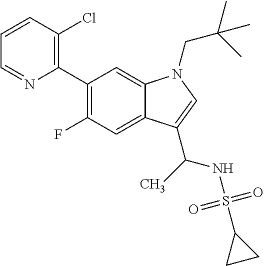 [SMILES: CC(NS(=O)(=O)C1CC1)c1cn(CC(C)(C)C)c2cc(-c3ncccc3Cl)c(F)cc12] | A |
| 59 | 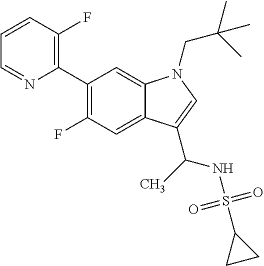 [SMILES: CC(NS(=O)(=O)C1CC1)c1cn(CC(C)(C)C)c2cc(-c3ncccc3F)c(F)cc12] | A |
| 60 | 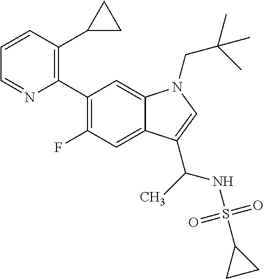 [SMILES: CC(NS(=O)(=O)C1CC1)c1cn(CC(C)(C)C)c2cc(-c3ncccc3C3CC3)c(F)cc12] | A |
| 61 | 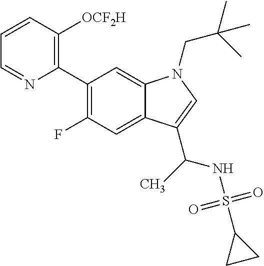 | A |
| 62 | 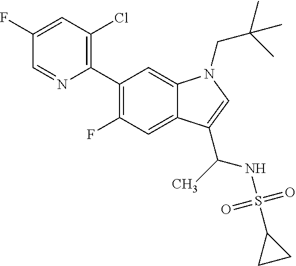 [SMILES: CC(NS(=O)(=O)C1CC1)c1cn(CC(C)(C)C)c2cc(-c3ncc(F)cc3Cl)c(F)cc12] | A |
| 63 | 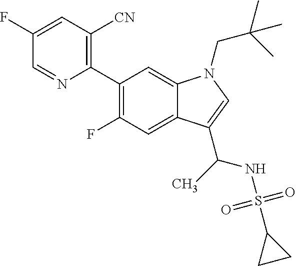 [SMILES: CC(NS(=O)(=O)C1CC1)c1cn(CC(C)(C)C)c2cc(-c3ncc(F)cc3C#N)c(F)cc12] | A |
| 64 | 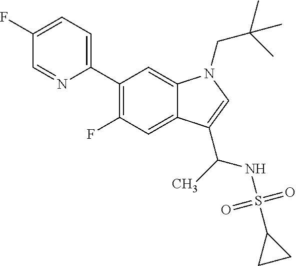 [SMILES: CC(NS(=O)(=O)C1CC1)c1cn(CC(C)(C)C)c2cc(-c3ccc(F)cn3)c(F)cc12] | B |
| 65 | 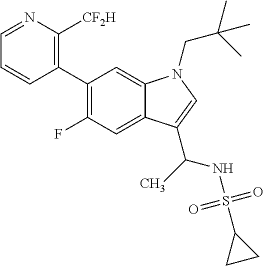 [SMILES: CC(NS(=O)(=O)C1CC1)c1cn(CC(C)(C)C)c2cc(-c3cccnc3C(F)F)c(F)cc12] | A |
| 66 | 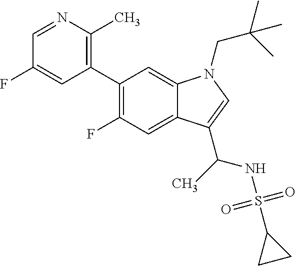 [SMILES: Cc1ncc(F)cc1-c1cc2c(cc1F)c(C(C)NS(=O)(=O)C1CC1)cn2CC(C)(C)C] | A |
| 67 | 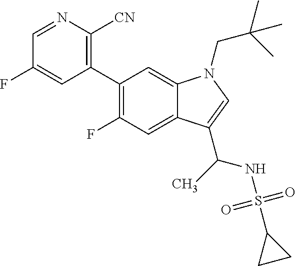 [SMILES: CC(NS(=O)(=O)C1CC1)c1cn(CC(C)(C)C)c2cc(-c3cc(F)cnc3C#N)c(F)cc12] | A |
| 68 | 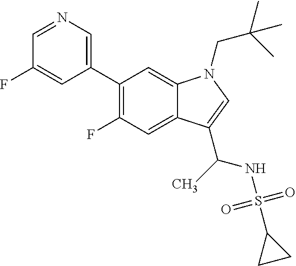 [SMILES: CC(NS(=O)(=O)C1CC1)c1cn(CC(C)(C)C)c2cc(-c3cncc(F)c3)c(F)cc12] | B |
| 69 | 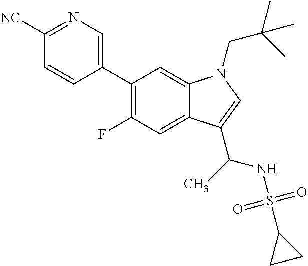 [SMILES: CC(NS(=O)(=O)C1CC1)c1cn(CC(C)(C)C)c2cc(-c3ccc(C#N)nc3)c(F)cc12] | B |
| 70 | 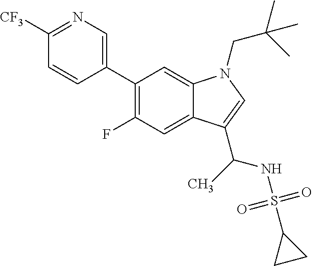 [SMILES: CC(NS(=O)(=O)C1CC1)c1cn(CC(C)(C)C)c2cc(-c3ccc(C(F)(F)F)nc3)c(F)cc12] | B |
| 71 | 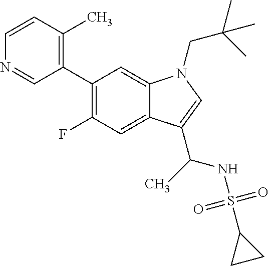 [SMILES: Cc1ccncc1-c1cc2c(cc1F)c(C(C)NS(=O)(=O)C1CC1)cn2CC(C)(C)C] | A |
| 72 | 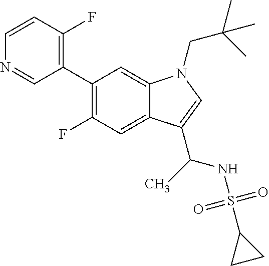 [SMILES: CC(NS(=O)(=O)C1CC1)c1cn(CC(C)(C)C)c2cc(-c3cnccc3F)c(F)cc12] | B |
| 73 | 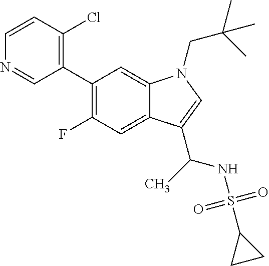 [SMILES: CC(NS(=O)(=O)C1CC1)c1cn(CC(C)(C)C)c2cc(-c3cnccc3Cl)c(F)cc12] | A |
| 74 | 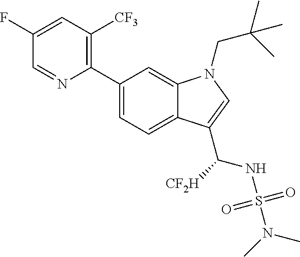 [SMILES: CN(C)S(=O)(=O)N[C@@H](c1cn(CC(C)(C)C)c2cc(-c3ncc(F)cc3C(F)(F)F)ccc12)C(F)F] | A |
| 75 | 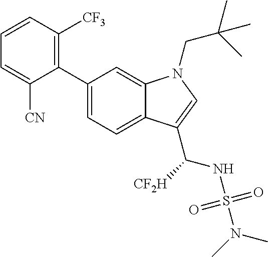 [SMILES: CN(C)S(=O)(=O)N[C@@H](c1cn(CC(C)(C)C)c2cc(-c3c(C#N)cccc3C(F)(F)F)ccc12)C(F)F] | A |
| 76 | 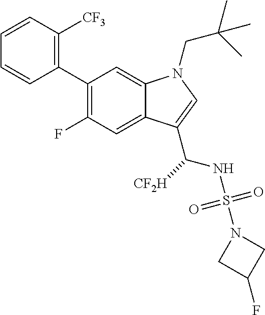 [SMILES: CC(C)(C)Cn1cc([C@H](NS(=O)(=O)N2CC(F)C2)C(F)F)c2cc(F)c(-c3ccccc3C(F)(F)F)cc21] | A |
| 77 | 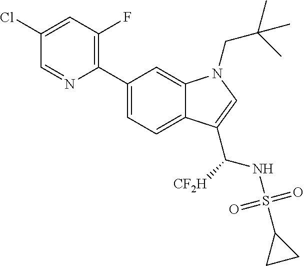 [SMILES: CC(C)(C)Cn1cc([C@H](NS(=O)(=O)C2CC2)C(F)F)c2ccc(-c3ncc(Cl)cc3F)cc21] | A |
| 78 | 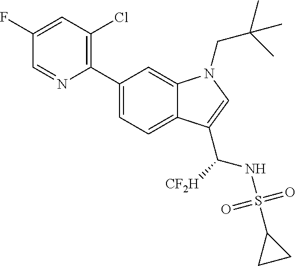 [SMILES: CC(C)(C)Cn1cc([C@H](NS(=O)(=O)C2CC2)C(F)F)c2ccc(-c3ncc(F)cc3Cl)cc21] | A |
| 79 | 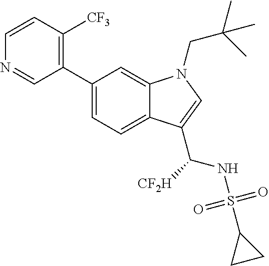 [SMILES: CC(C)(C)Cn1cc([C@H](NS(=O)(=O)C2CC2)C(F)F)c2ccc(-c3cnccc3C(F)(F)F)cc21] | A |
| 80 | 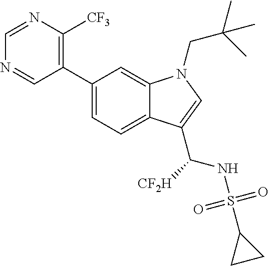 [SMILES: CC(C)(C)Cn1cc([C@H](NS(=O)(=O)C2CC2)C(F)F)c2ccc(-c3cncnc3C(F)(F)F)cc21] | A |
| 81 | 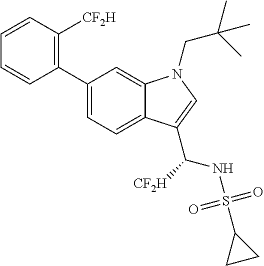 [SMILES: CC(C)(C)Cn1cc([C@H](NS(=O)(=O)C2CC2)C(F)F)c2ccc(-c3ccccc3C(F)F)cc21] | A |
| 82 | 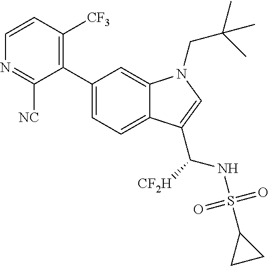 [SMILES: CC(C)(C)Cn1cc([C@H](NS(=O)(=O)C2CC2)C(F)F)c2ccc(-c3c(C(F)(F)F)ccnc3C#N)cc21] | A |
| 83 | 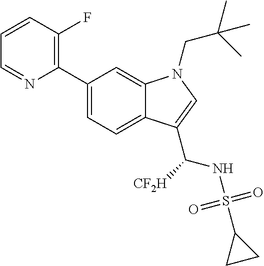 [SMILES: CC(C)(C)Cn1cc([C@H](NS(=O)(=O)C2CC2)C(F)F)c2ccc(-c3ncccc3F)cc21] | A |
| 84 | 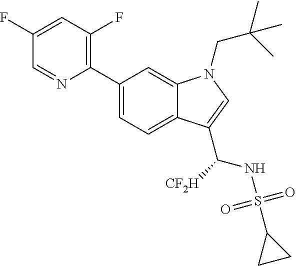 [SMILES: CC(C)(C)Cn1cc([C@H](NS(=O)(=O)C2CC2)C(F)F)c2ccc(-c3ncc(F)cc3F)cc21] | A |
| 85 | 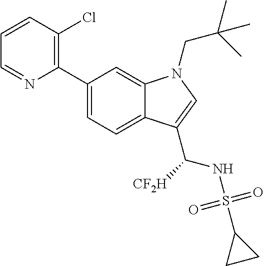 [SMILES: CC(C)(C)Cn1cc([C@H](NS(=O)(=O)C2CC2)C(F)F)c2ccc(-c3ncccc3Cl)cc21] | A |
| 86 | 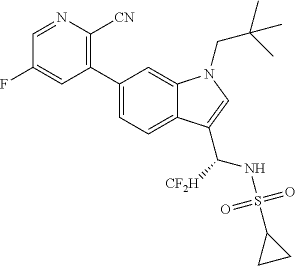 [SMILES: CC(C)(C)Cn1cc([C@H](NS(=O)(=O)C2CC2)C(F)F)c2ccc(-c3cc(F)cnc3C#N)cc21] | A |
| 87 | 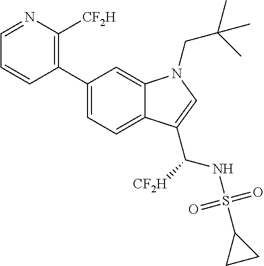 [SMILES: CC(C)(C)Cn1cc([C@H](NS(=O)(=O)C2CC2)C(F)F)c2ccc(-c3cccnc3C(F)F)cc21] | A |
| 88 | 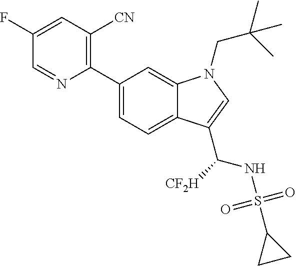 [SMILES: CC(C)(C)Cn1cc([C@H](NS(=O)(=O)C2CC2)C(F)F)c2ccc(-c3ncc(F)cc3C#N)cc21] | A |
| 89 | 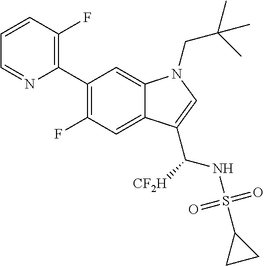 [SMILES: CC(C)(C)Cn1cc([C@H](NS(=O)(=O)C2CC2)C(F)F)c2cc(F)c(-c3ncccc3F)cc21] | A |
| 90 | 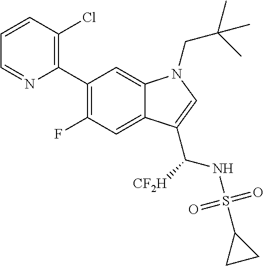 [SMILES: CC(C)(C)Cn1cc([C@H](NS(=O)(=O)C2CC2)C(F)F)c2cc(F)c(-c3ncccc3Cl)cc21] | A |
| 91 | 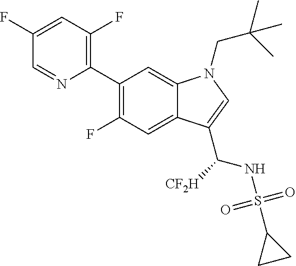 [SMILES: CC(C)(C)Cn1cc([C@H](NS(=O)(=O)C2CC2)C(F)F)c2cc(F)c(-c3ncc(F)cc3F)cc21] | A |
| 92 | 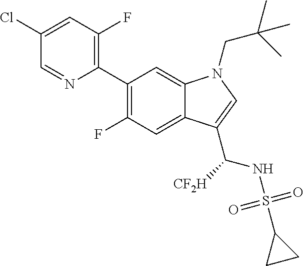 [SMILES: CC(C)(C)Cn1cc([C@H](NS(=O)(=O)C2CC2)C(F)F)c2cc(F)c(-c3ncc(Cl)cc3F)cc21] | A |
| 93 | 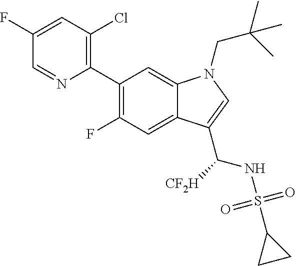 [SMILES: CC(C)(C)Cn1cc([C@H](NS(=O)(=O)C2CC2)C(F)F)c2cc(F)c(-c3ncc(F)cc3Cl)cc21] | A |
| 94 | 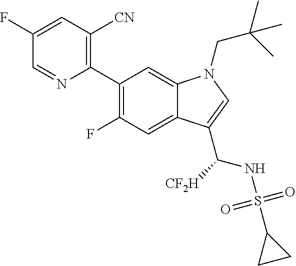 [SMILES: CC(C)(C)Cn1cc([C@H](NS(=O)(=O)C2CC2)C(F)F)c2cc(F)c(-c3ncc(F)cc3C#N)cc21] | A |
| 95 | 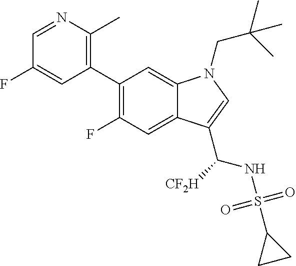 [SMILES: Cc1ncc(F)cc1-c1cc2c(cc1F)c([C@H](NS(=O)(=O)C1CC1)C(F)F)cn2CC(C)(C)C] | A |
| 96 | 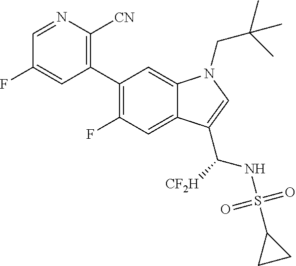 [SMILES: CC(C)(C)Cn1cc([C@H](NS(=O)(=O)C2CC2)C(F)F)c2cc(F)c(-c3cc(F)cnc3C#N)cc21] | A |
| 97 | 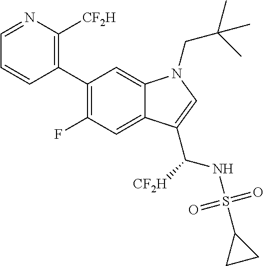 [SMILES: CC(C)(C)Cn1cc([C@H](NS(=O)(=O)C2CC2)C(F)F)c2cc(F)c(-c3cccnc3C(F)F)cc21] | A |
| 98 | 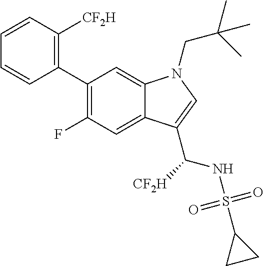 [SMILES: CC(C)(C)Cn1cc([C@H](NS(=O)(=O)C2CC2)C(F)F)c2cc(F)c(-c3ccccc3C(F)F)cc21] | |
| 99 | 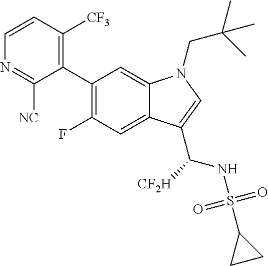 [SMILES: CC(C)(C)Cn1cc([C@H](NS(=O)(=O)C2CC2)C(F)F)c2cc(F)c(-c3c(C(F)(F)F)ccnc3C#N)cc21] | A |
| 100 | 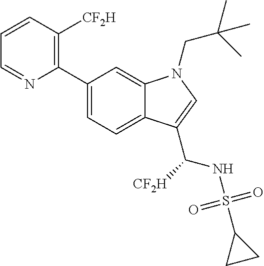 [SMILES: CC(C)(C)Cn1cc([C@H](NS(=O)(=O)C2CC2)C(F)F)c2ccc(-c3ncccc3C(F)F)cc21] | A |
| 101 | 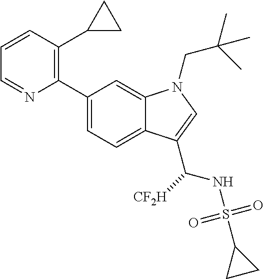 [SMILES: CC(C)(C)Cn1cc([C@H](NS(=O)(=O)C2CC2)C(F)F)c2ccc(-c3ncccc3C3CC3)cc21] | A |
| 102 | 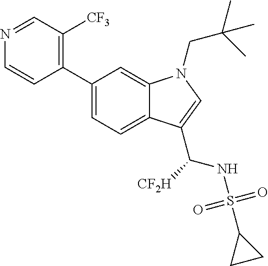 [SMILES: CC(C)(C)Cn1cc([C@H](NS(=O)(=O)C2CC2)C(F)F)c2ccc(-c3ccncc3C(F)(F)F)cc21] | A |
| 103 | 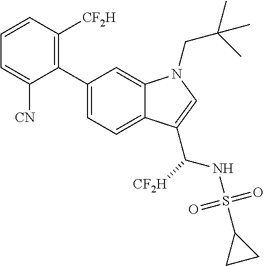 | A |
| 104 | 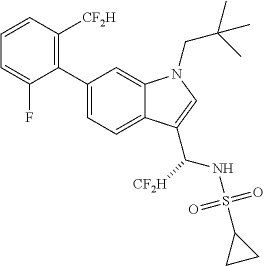 [SMILES: CC(C)(C)Cn1cc([C@H](NS(=O)(=O)C2CC2)C(F)F)c2ccc(-c3c(F)cccc3C(F)F)cc21] | A |
| 105 | 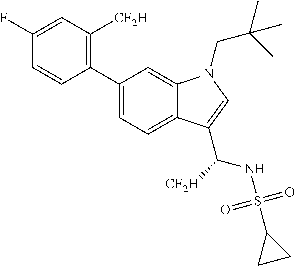 [SMILES: CC(C)(C)Cn1cc([C@H](NS(=O)(=O)C2CC2)C(F)F)c2ccc(-c3ccc(F)cc3C(F)F)cc21] | A |
| 106 | 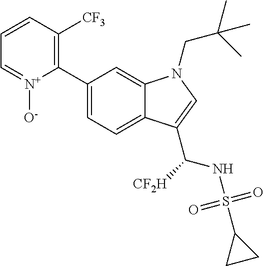 [SMILES: CC(C)(C)Cn1cc([C@H](NS(=O)(=O)C2CC2)C(F)F)c2ccc(-c3c(C(F)(F)F)ccc[n+]3[O-])cc21] | B |
| 107 | 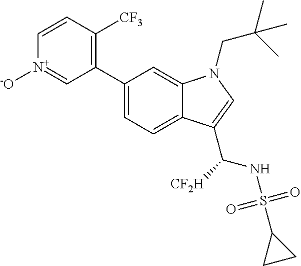 | A |
| 108 | 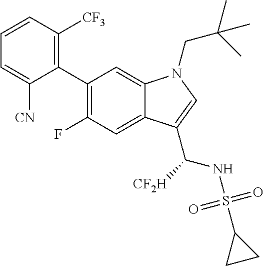 | A |
| 109 | 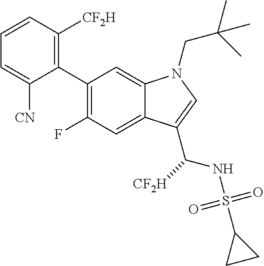 | A |
| 110 | 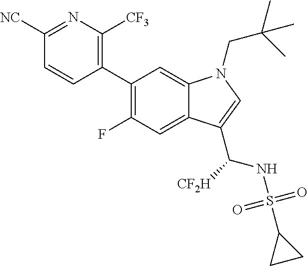 [SMILES: CC(C)(C)Cn1cc([C@H](NS(=O)(=O)C2CC2)C(F)F)c2cc(F)c(-c3ccc(C#N)nc3C(F)(F)F)cc21] | A |
| 111 | 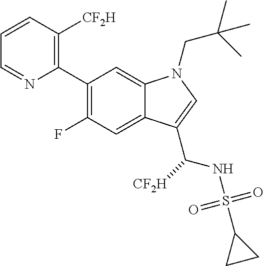 [SMILES: CC(C)(C)Cn1cc([C@H](NS(=O)(=O)C2CC2)C(F)F)c2cc(F)c(-c3ncccc3C(F)F)cc21] | A |
| 112 | 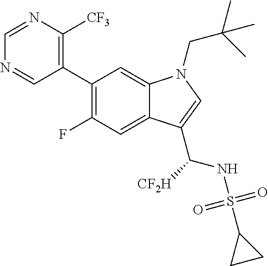 [SMILES: CC(C)(C)Cn1cc([C@H](NS(=O)(=O)C2CC2)C(F)F)c2cc(F)c(-c3cncnc3C(F)(F)F)cc21] | A |
| 113 | 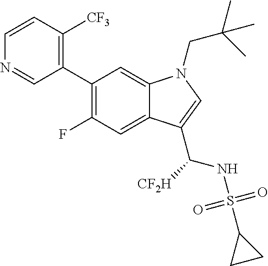 [SMILES: CC(C)(C)Cn1cc([C@H](NS(=O)(=O)C2CC2)C(F)F)c2cc(F)c(-c3cnccc3C(F)(F)F)cc21] | A |
| 114 | 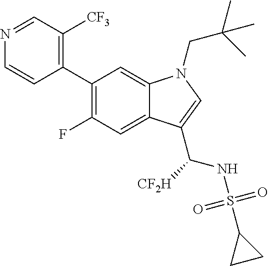 [SMILES: CC(C)(C)Cn1cc([C@H](NS(=O)(=O)C2CC2)C(F)F)c2cc(F)c(-c3ccncc3C(F)(F)F)cc21] | A |
| 115 | 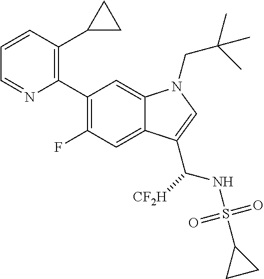 [SMILES: CC(C)(C)Cn1cc([C@H](NS(=O)(=O)C2CC2)C(F)F)c2cc(F)c(-c3ncccc3C3CC3)cc21] | A |
| 116 | 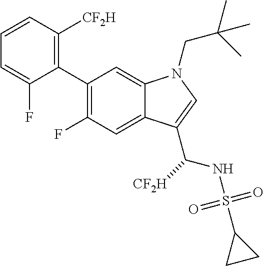 [SMILES: CC(C)(C)Cn1cc([C@H](NS(=O)(=O)C2CC2)C(F)F)c2cc(F)c(-c3c(F)cccc3C(F)F)cc21] | A |
| 117 | 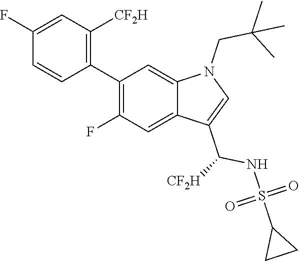 [SMILES: CC(C)(C)Cn1cc([C@H](NS(=O)(=O)C2CC2)C(F)F)c2cc(F)c(-c3ccc(F)cc3C(F)F)cc21] | A |
| 118 | 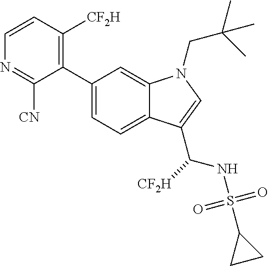 | A |
| 119 | 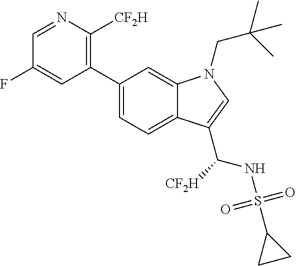 [SMILES: CC(C)(C)Cn1cc([C@H](NS(=O)(=O)C2CC2)C(F)F)c2ccc(-c3cc(F)cnc3C(F)F)cc21] | A |
| 120 | 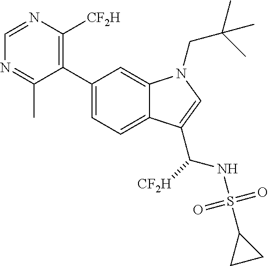 [SMILES: Cc1ncnc(C(F)F)c1-c1ccc2c([C@H](NS(=O)(=O)C3CC3)C(F)F)cn(CC(C)(C)C)c2c1] | A |
| 121 | 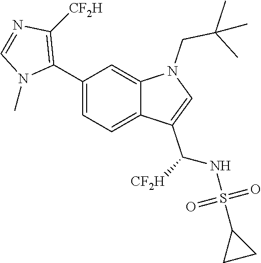 [SMILES: Cn1cnc(C(F)F)c1-c1ccc2c([C@H](NS(=O)(=O)C3CC3)C(F)F)cn(CC(C)(C)C)c2c1] | A |
| 122 | 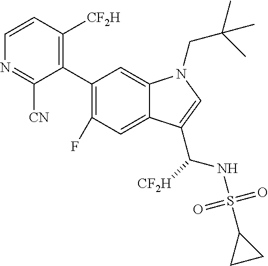 [SMILES: CC(C)(C)Cn1cc([C@H](NS(=O)(=O)C2CC2)C(F)F)c2cc(F)c(-c3c(C(F)F)ccnc3C#N)cc21] | A |
| 123 | 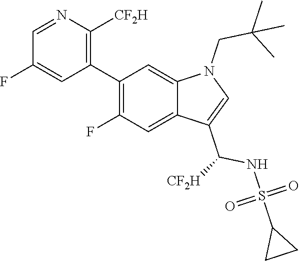 [SMILES: CC(C)(C)Cn1cc([C@H](NS(=O)(=O)C2CC2)C(F)F)c2cc(F)c(-c3cc(F)cnc3C(F)F)cc21] | A |
| 124 | 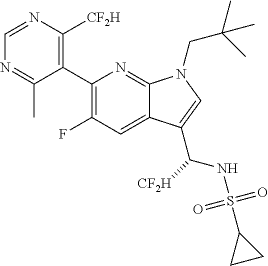 [SMILES: Cc1ncnc(C(F)F)c1-c1nc2c(cc1F)c([C@H](NS(=O)(=O)C1CC1)C(F)F)cn2CC(C)(C)C] | A |
| 125 | 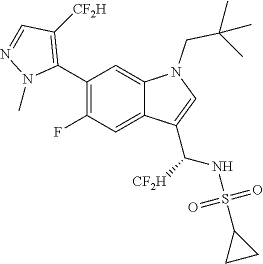 [SMILES: Cn1ncc(C(F)F)c1-c1cc2c(cc1F)c([C@H](NS(=O)(=O)C1CC1)C(F)F)cn2CC(C)(C)C] | A |
| 126 | 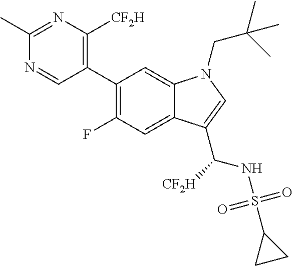 [SMILES: Cc1ncc(-c2cc3c(cc2F)c([C@H](NS(=O)(=O)C2CC2)C(F)F)cn3CC(C)(C)C)c(C(F)F)n1] | A |
| 127 | 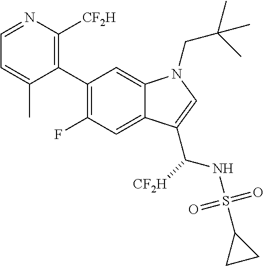 [SMILES: Cc1ccnc(C(F)F)c1-c1cc2c(cc1F)c([C@H](NS(=O)(=O)C1CC1)C(F)F)cn2CC(C)(C)C] | A |
| 128 | 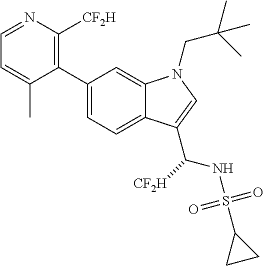 [SMILES: Cc1ccnc(C(F)F)c1-c1ccc2c([C@H](NS(=O)(=O)C3CC3)C(F)F)cn(CC(C)(C)C)c2c1] | A |
| 129 | 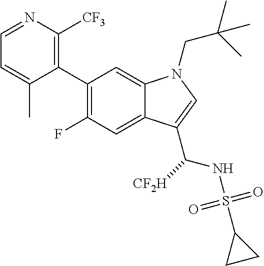 [SMILES: Cc1ccnc(C(F)(F)F)c1-c1cc2c(cc1F)c([C@H](NS(=O)(=O)C1CC1)C(F)F)cn2CC(C)(C)C] | A |
| 130 | 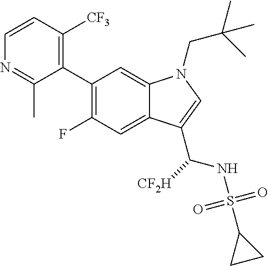 [SMILES: Cc1nccc(C(F)(F)F)c1-c1cc2c(cc1F)c([C@H](NS(=O)(=O)C1CC1)C(F)F)cn2CC(C)(C)C] | A |
| 131 | 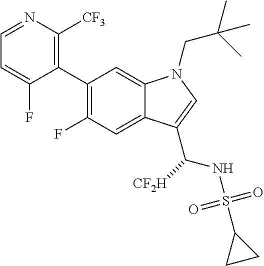 [SMILES: CC(C)(C)Cn1cc([C@H](NS(=O)(=O)C2CC2)C(F)F)c2cc(F)c(-c3c(F)ccnc3C(F)(F)F)cc21] | A |
| 132 | 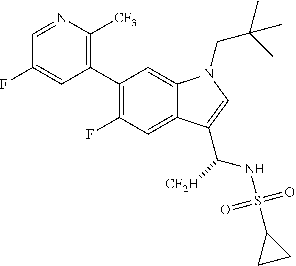 [SMILES: CC(C)(C)Cn1cc([C@H](NS(=O)(=O)C2CC2)C(F)F)c2cc(F)c(-c3cc(F)cnc3C(F)(F)F)cc21] | A |
| 133 | 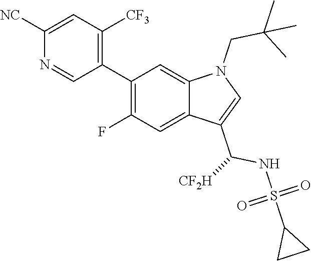 [SMILES: CC(C)(C)Cn1cc([C@H](NS(=O)(=O)C2CC2)C(F)F)c2cc(F)c(-c3cnc(C#N)cc3C(F)(F)F)cc21] | A |
| 134 | 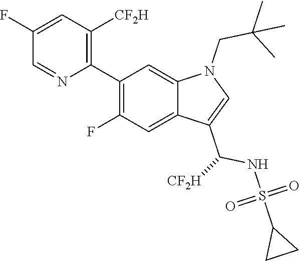 [SMILES: CC(C)(C)Cn1cc([C@H](NS(=O)(=O)C2CC2)C(F)F)c2cc(F)c(-c3ncc(F)cc3C(F)F)cc21] | A |
| 135 | 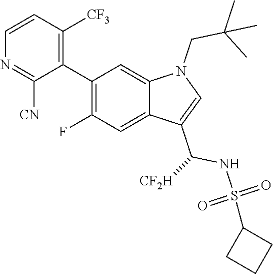 | A |
| 136 | 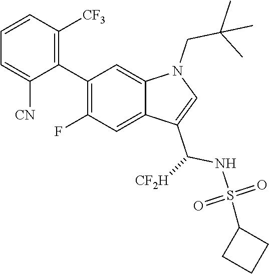 | A |
| 137 | 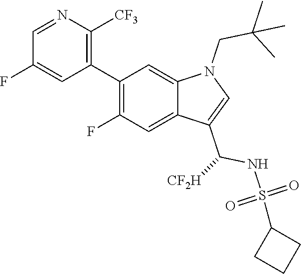 [SMILES: CC(C)(C)Cn1cc([C@H](NS(=O)(=O)C2CCC2)C(F)F)c2cc(F)c(-c3cc(F)cnc3C(F)(F)F)cc21] | A |
| 138 | 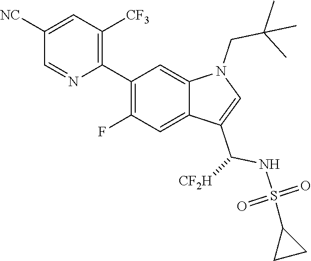 [SMILES: CC(C)(C)Cn1cc([C@H](NS(=O)(=O)C2CC2)C(F)F)c2cc(F)c(-c3ncc(C#N)cc3C(F)(F)F)cc21] | A |
| 139 | 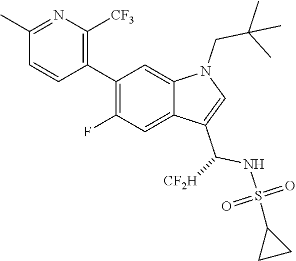 [SMILES: Cc1ccc(-c2cc3c(cc2F)c([C@H](NS(=O)(=O)C2CC2)C(F)F)cn3CC(C)(C)C)c(C(F)(F)F)n1] | A |
| 140 | 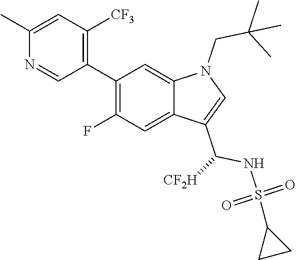 [SMILES: Cc1cc(C(F)(F)F)c(-c2cc3c(cc2F)c([C@H](NS(=O)(=O)C2CC2)C(F)F)cn3CC(C)(C)C)cn1] | A |
| 141 | 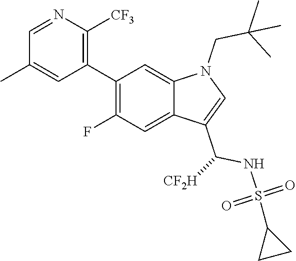 [SMILES: Cc1cnc(C(F)(F)F)c(-c2cc3c(cc2F)c([C@H](NS(=O)(=O)C2CC2)C(F)F)cn3CC(C)(C)C)c1] | A |
| 142 | 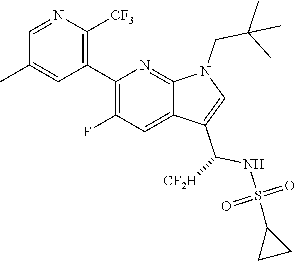 [SMILES: Cc1cnc(C(F)(F)F)c(-c2nc3c(cc2F)c([C@H](NS(=O)(=O)C2CC2)C(F)F)cn3CC(C)(C)C)c1] | A |
| 143 | 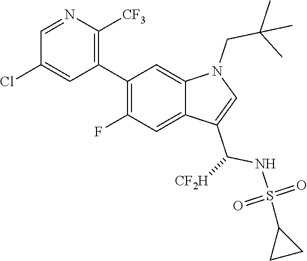 [SMILES: CC(C)(C)Cn1cc([C@H](NS(=O)(=O)C2CC2)C(F)F)c2cc(F)c(-c3cc(Cl)cnc3C(F)(F)F)cc21] | A |
| 144 | 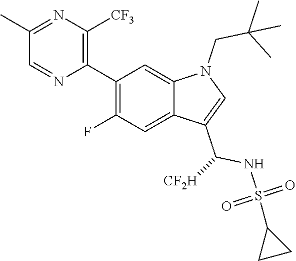 [SMILES: Cc1cnc(-c2cc3c(cc2F)c([C@H](NS(=O)(=O)C2CC2)C(F)F)cn3CC(C)(C)C)c(C(F)(F)F)n1] | A |
| 145 | 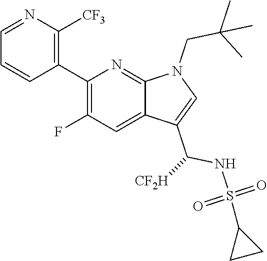 [SMILES: CC(C)(C)Cn1cc([C@H](NS(=O)(=O)C2CC2)C(F)F)c2cc(F)c(-c3cccnc3C(F)(F)F)nc21] | A |
| 146 | 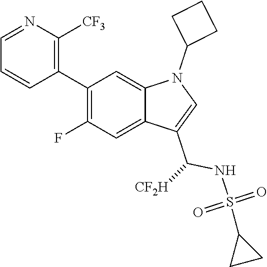 [SMILES: O=S(=O)(N[C@@H](c1cn(C2CCC2)c2cc(-c3cccnc3C(F)(F)F)c(F)cc12)C(F)F)C1CC1] | A |
| 147 | 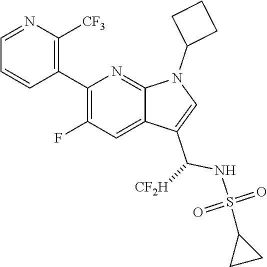 [SMILES: O=S(=O)(N[C@@H](c1cn(C2CCC2)c2nc(-c3cccnc3C(F)(F)F)c(F)cc12)C(F)F)C1CC1] | A |
| 148 | 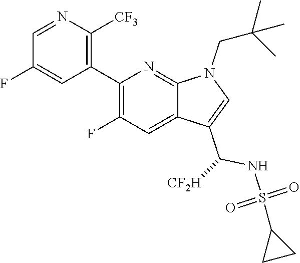 [SMILES: CC(C)(C)Cn1cc([C@H](NS(=O)(=O)C2CC2)C(F)F)c2cc(F)c(-c3cc(F)cnc3C(F)(F)F)nc21] | A |
| 149 | 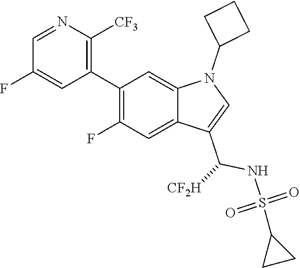 [SMILES: O=S(=O)(N[C@@H](c1cn(C2CCC2)c2cc(-c3cc(F)cnc3C(F)(F)F)c(F)cc12)C(F)F)C1CC1] | A |
| 150 | 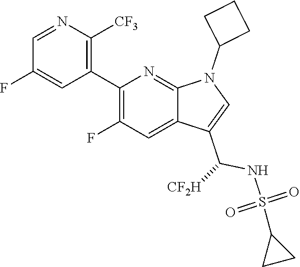 [SMILES: O=S(=O)(N[C@@H](c1cn(C2CCC2)c2nc(-c3cc(F)cnc3C(F)(F)F)c(F)cc12)C(F)F)C1CC1] | A |
| 151 | 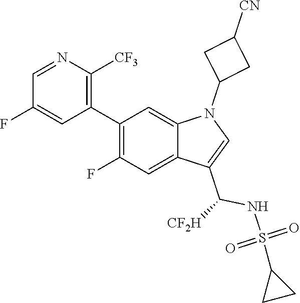 [SMILES: N#CC1CC(n2cc([C@H](NS(=O)(=O)C3CC3)C(F)F)c3cc(F)c(-c4cc(F)cnc4C(F)(F)F)cc32)C1] | A |
| 152 | 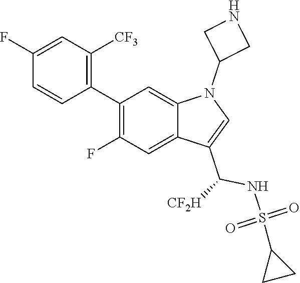 [SMILES: O=S(=O)(N[C@@H](c1cn(C2CNC2)c2cc(-c3ccc(F)cc3C(F)(F)F)c(F)cc12)C(F)F)C1CC1] | C |
| 153 | 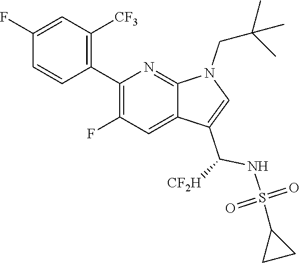 [SMILES: CC(C)(C)Cn1cc([C@H](NS(=O)(=O)C2CC2)C(F)F)c2cc(F)c(-c3ccc(F)cc3C(F)(F)F)nc21] | A |
| 154 | 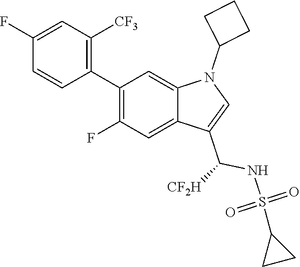 [SMILES: O=S(=O)(N[C@@H](c1cn(C2CCC2)c2cc(-c3ccc(F)cc3C(F)(F)F)c(F)cc12)C(F)F)C1CC1] | A |
| 155 | 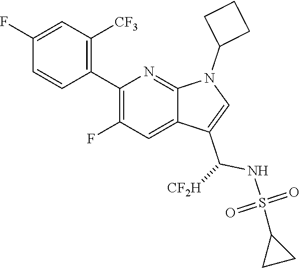 [SMILES: O=S(=O)(N[C@@H](c1cn(C2CCC2)c2nc(-c3ccc(F)cc3C(F)(F)F)c(F)cc12)C(F)F)C1CC1] | A |
| 156 | 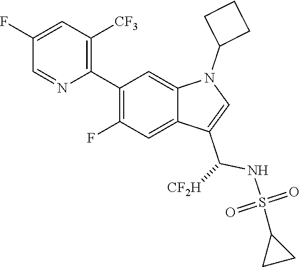 [SMILES: O=S(=O)(N[C@@H](c1cn(C2CCC2)c2cc(-c3ncc(F)cc3C(F)(F)F)c(F)cc12)C(F)F)C1CC1] | A |
| 157 | 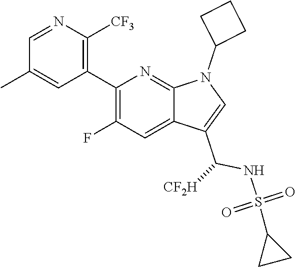 [SMILES: Cc1cnc(C(F)(F)F)c(-c2nc3c(cc2F)c([C@H](NS(=O)(=O)C2CC2)C(F)F)cn3C2CCC2)c1] | A |
| 158 | 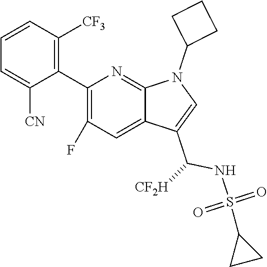 [SMILES: N#Cc1cccc(C(F)(F)F)c1-c1nc2c(cc1F)c([C@H](NS(=O)(=O)C1CC1)C(F)F)cn2C1CCC1] | A |
| 159 | 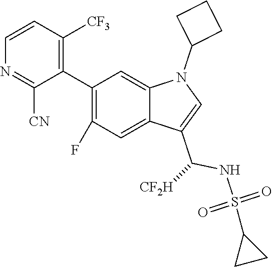 [SMILES: N#Cc1nccc(C(F)(F)F)c1-c1cc2c(cc1F)c([C@H](NS(=O)(=O)C1CC1)C(F)F)cn2C1CCC1] | A |
| 160 | 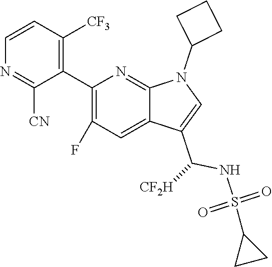 [SMILES: N#Cc1nccc(C(F)(F)F)c1-c1nc2c(cc1F)c([C@H](NS(=O)(=O)C1CC1)C(F)F)cn2C1CCC1] | A |
| 161 | 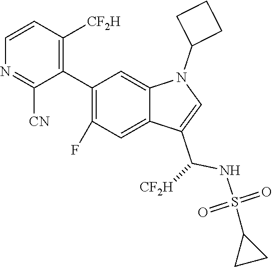 [SMILES: N#Cc1nccc(C(F)F)c1-c1cc2c(cc1F)c([C@H](NS(=O)(=O)C1CC1)C(F)F)cn2C1CCC1] | A |
| 162 | 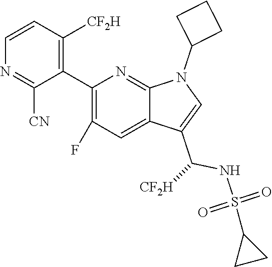 [SMILES: N#Cc1nccc(C(F)F)c1-c1nc2c(cc1F)c([C@H](NS(=O)(=O)C1CC1)C(F)F)cn2C1CCC1] | A |
| 163 | 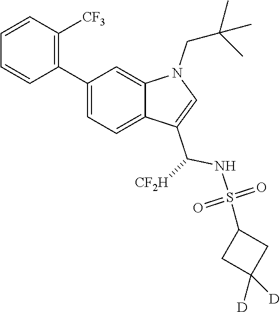 [SMILES: [2H]C1([2H])CC(S(=O)(=O)N[C@@H](c2cn(CC(C)(C)C)c3cc(-c4ccccc4C(F)(F)F)ccc23)C(F)F)C1] | A |
| 164 | 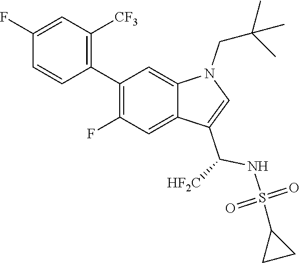 | A |
| 165 | 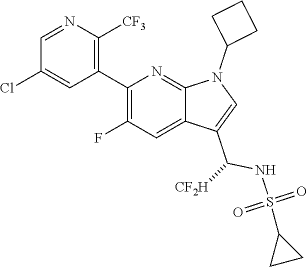 [SMILES: O=S(=O)(N[C@@H](c1cn(C2CCC2)c2nc(-c3cc(Cl)cnc3C(F)(F)F)c(F)cc12)C(F)F)C1CC1] | A |
| 166 | 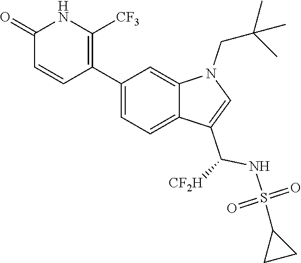 [SMILES: CC(C)(C)Cn1cc([C@H](NS(=O)(=O)C2CC2)C(F)F)c2ccc(-c3ccc(=O)[nH]c3C(F)(F)F)cc21] | A |
| 167 | 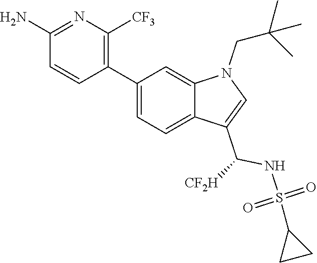 [SMILES: CC(C)(C)Cn1cc([C@H](NS(=O)(=O)C2CC2)C(F)F)c2ccc(-c3ccc(N)nc3C(F)(F)F)cc21] | A |
| 168 | 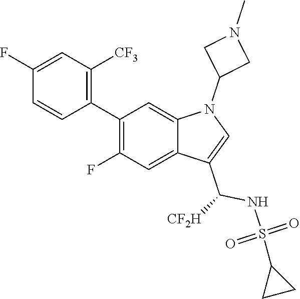 [SMILES: CN1CC(n2cc([C@H](NS(=O)(=O)C3CC3)C(F)F)c3cc(F)c(-c4ccc(F)cc4C(F)(F)F)cc32)C1] | B |
| 169 | 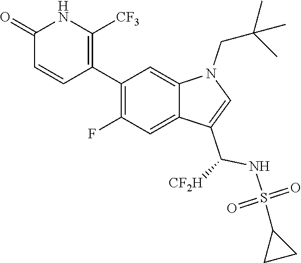 [SMILES: CC(C)(C)Cn1cc([C@H](NS(=O)(=O)C2CC2)C(F)F)c2cc(F)c(-c3ccc(=O)[nH]c3C(F)(F)F)cc21] | A |
| 170 | 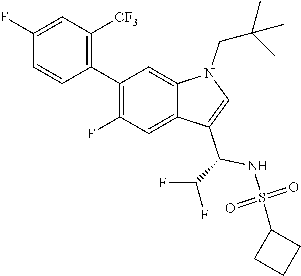 [SMILES: CC(C)(C)Cn1cc([C@H](NS(=O)(=O)C2CCC2)C(F)F)c2cc(F)c(-c3ccc(F)cc3C(F)(F)F)cc21] | A |
| 171 | 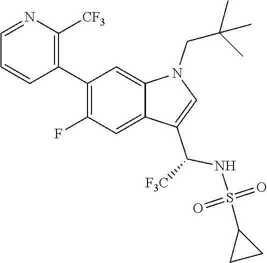 [SMILES: CC(C)(C)Cn1cc([C@H](NS(=O)(=O)C2CC2)C(F)(F)F)c2cc(F)c(-c3cccnc3C(F)(F)F)cc21] | A |
| 172 | 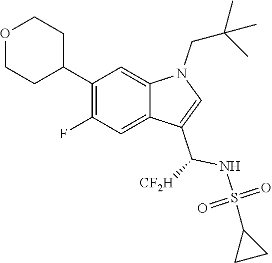 [SMILES: CC(C)(C)Cn1cc([C@H](NS(=O)(=O)C2CC2)C(F)F)c2cc(F)c(C3CCOCC3)cc21] | A |
| 173 | 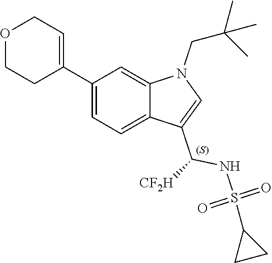 | |
| 174 | 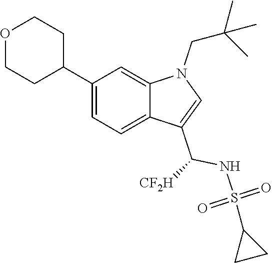 [SMILES: CC(C)(C)Cn1cc([C@H](NS(=O)(=O)C2CC2)C(F)F)c2ccc(C3CCOCC3)cc21] | A |
| 175 | 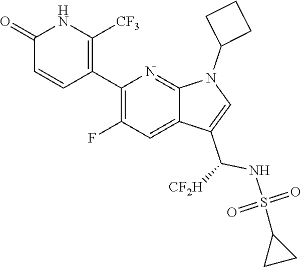 [SMILES: O=c1ccc(-c2nc3c(cc2F)c([C@H](NS(=O)(=O)C2CC2)C(F)F)cn3C2CCC2)c(C(F)(F)F)[nH]1] | A |
| 176 | 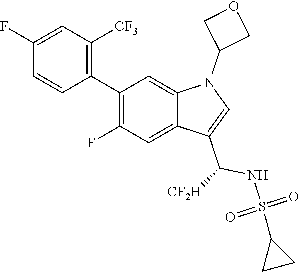 [SMILES: O=S(=O)(N[C@@H](c1cn(C2COC2)c2cc(-c3ccc(F)cc3C(F)(F)F)c(F)cc12)C(F)F)C1CC1] | B |
| 177 | 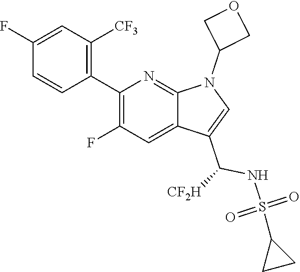 [SMILES: O=S(=O)(N[C@@H](c1cn(C2COC2)c2nc(-c3ccc(F)cc3C(F)(F)F)c(F)cc12)C(F)F)C1CC1] | B |
| 178 | 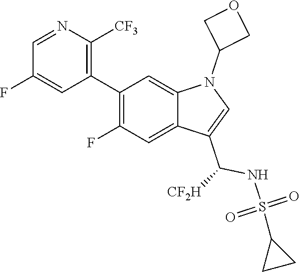 [SMILES: O=S(=O)(N[C@@H](c1cn(C2COC2)c2cc(-c3cc(F)cnc3C(F)(F)F)c(F)cc12)C(F)F)C1CC1] | B |
| 179 | 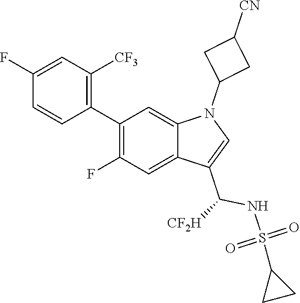 [SMILES: N#CC1CC(n2cc([C@H](NS(=O)(=O)C3CC3)C(F)F)c3cc(F)c(-c4ccc(F)cc4C(F)(F)F)cc32)C1] | A |
| 180 | 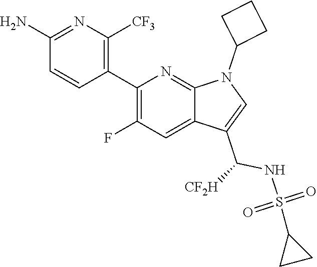 [SMILES: Nc1ccc(-c2nc3c(cc2F)c([C@H](NS(=O)(=O)C2CC2)C(F)F)cn3C2CCC2)c(C(F)(F)F)n1] | A |
| 181 | 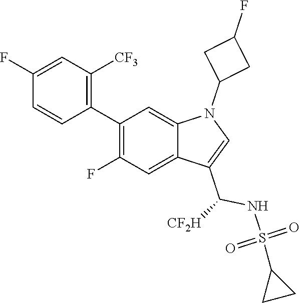 [SMILES: O=S(=O)(N[C@@H](c1cn(C2CC(F)C2)c2cc(-c3ccc(F)cc3C(F)(F)F)c(F)cc12)C(F)F)C1CC1] | A |
| 182 | 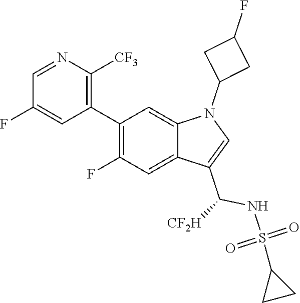 [SMILES: O=S(=O)(N[C@@H](c1cn(C2CC(F)C2)c2cc(-c3cc(F)cnc3C(F)(F)F)c(F)cc12)C(F)F)C1CC1] | A |

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A compound of pharmaceutically acceptable salt thereof, wherein the compound is one selected from the following table:

| Compound | Structure |
|---|---|
| 1 | 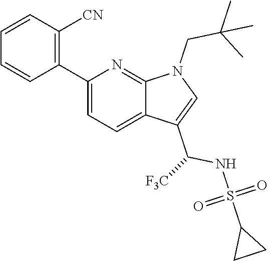 [SMILES: CC(C)(C)Cn1cc([C@H](NS(=O)(=O)C2CC2)C(F)(F)F)c2ccc(-c3ccccc3C#N)nc21] |
| 2 | 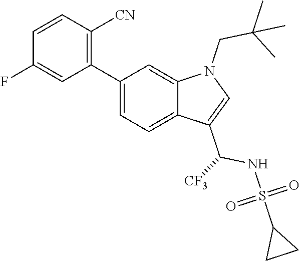 [SMILES: CC(C)(C)Cn1cc([C@H](NS(=O)(=O)C2CC2)C(F)(F)F)c2ccc(-c3cc(F)ccc3C#N)cc21] |
| 3 | 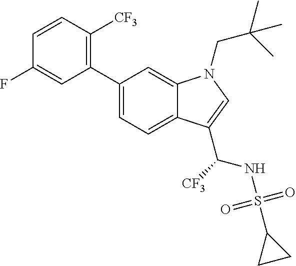 [SMILES: CC(C)(C)Cn1cc([C@H](NS(=O)(=O)C2CC2)C(F)(F)F)c2ccc(-c3cc(F)ccc3C(F)(F)F)cc21] |
| 4 | 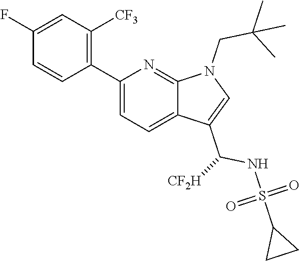 [SMILES: CC(C)(C)Cn1cc([C@H](NS(=O)(=O)C2CC2)C(F)F)c2ccc(-c3ccc(F)cc3C(F)(F)F)nc21] |
| 5 | 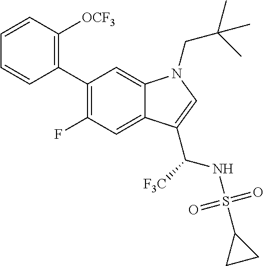 [SMILES: CC(C)(C)Cn1cc([C@H](NS(=O)(=O)C2CC2)C(F)(F)F)c2cc(F)c(-c3ccccc3OC(F)(F)F)cc21] |
| 6 | 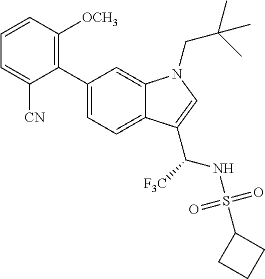 [SMILES: COc1cccc(C#N)c1-c1ccc2c([C@H](NS(=O)(=O)C3CCC3)C(F)(F)F)cn(CC(C)(C)C)c2c1] |
| 7 | 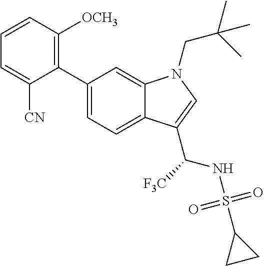 [SMILES: COc1cccc(C#N)c1-c1ccc2c([C@H](NS(=O)(=O)C3CC3)C(F)(F)F)cn(CC(C)(C)C)c2c1] |
| 8 | 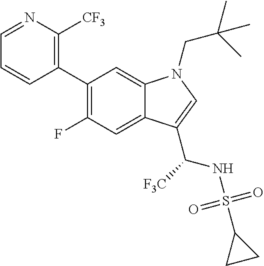 [SMILES: CC(C)(C)Cn1cc([C@H](NS(=O)(=O)C2CC2)C(F)(F)F)c2cc(F)c(-c3cccnc3C(F)(F)F)cc21] |
| 9 | 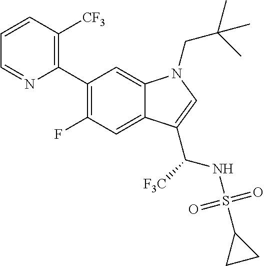 [SMILES: CC(C)(C)Cn1cc([C@H](NS(=O)(=O)C2CC2)C(F)(F)F)c2cc(F)c(-c3ncccc3C(F)(F)F)cc21] |
| 10 | 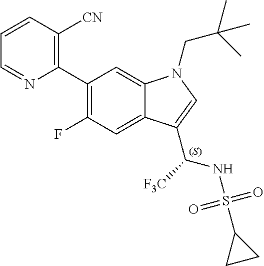 |
| 11 | 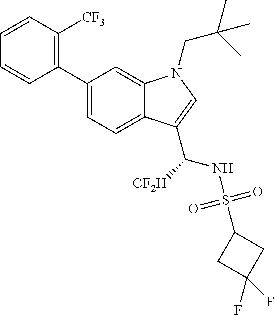 [SMILES: CC(C)(C)Cn1cc([C@H](NS(=O)(=O)C2CC(F)(F)C2)C(F)F)c2ccc(-c3ccccc3C(F)(F)F)cc21] |
| 12 | 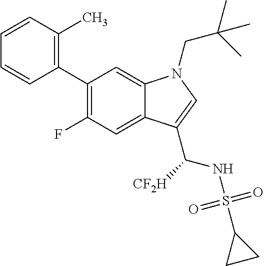 [SMILES: Cc1ccccc1-c1cc2c(cc1F)c([C@H](NS(=O)(=O)C1CC1)C(F)F)cn2CC(C)(C)C] |
| 13 | 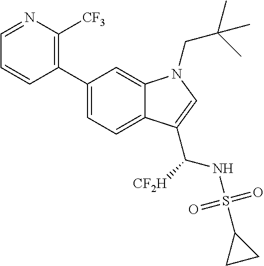 [SMILES: CC(C)(C)Cn1cc([C@H](NS(=O)(=O)C2CC2)C(F)F)c2ccc(-c3cccnc3C(F)(F)F)cc21] |
| 14 | 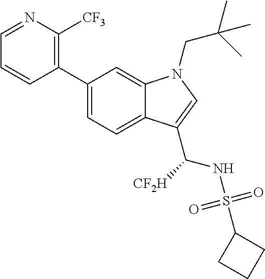 [SMILES: CC(C)(C)Cn1cc([C@H](NS(=O)(=O)C2CCC2)C(F)F)c2ccc(-c3cccnc3C(F)(F)F)cc21] |
| 15 | 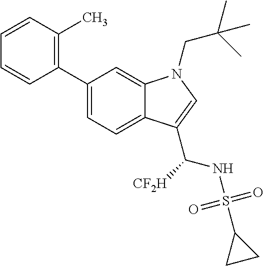 [SMILES: Cc1ccccc1-c1ccc2c([C@H](NS(=O)(=O)C3CC3)C(F)F)cn(CC(C)(C)C)c2c1] |
| 16 | 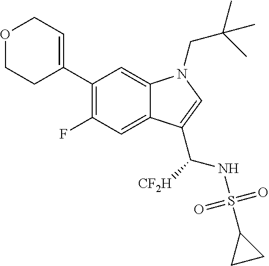 [SMILES: CC(C)(C)Cn1cc([C@H](NS(=O)(=O)C2CC2)C(F)F)c2cc(F)c(C3=CCOCC3)cc21] |
| 17 | 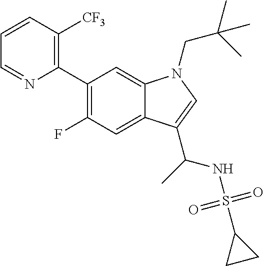 [SMILES: CC(NS(=O)(=O)C1CC1)c1cn(CC(C)(C)C)c2cc(-c3ncccc3C(F)(F)F)c(F)cc12] |
| 18 | 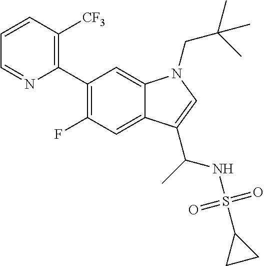 [SMILES: CC(NS(=O)(=O)C1CC1)c1cn(CC(C)(C)C)c2cc(-c3ncccc3C(F)(F)F)c(F)cc12] |
| 19 | 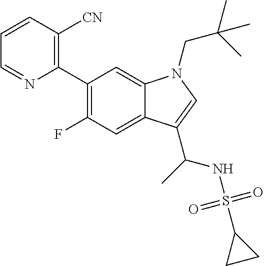 [SMILES: CC(NS(=O)(=O)C1CC1)c1cn(CC(C)(C)C)c2cc(-c3ncccc3C#N)c(F)cc12] |
| 20 | 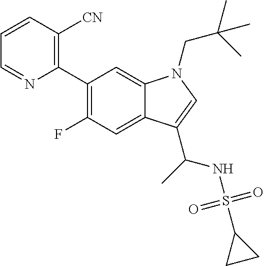 [SMILES: CC(NS(=O)(=O)C1CC1)c1cn(CC(C)(C)C)c2cc(-c3ncccc3C#N)c(F)cc12] |
| 21 | 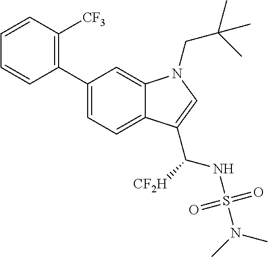 [SMILES: CN(C)S(=O)(=O)N[C@@H](c1cn(CC(C)(C)C)c2cc(-c3ccccc3C(F)(F)F)ccc12)C(F)F] |
| 22 | 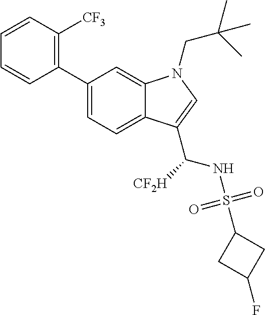 [SMILES: CC(C)(C)Cn1cc([C@H](NS(=O)(=O)C2CC(F)C2)C(F)F)c2ccc(-c3ccccc3C(F)(F)F)cc21] |
| 23 | 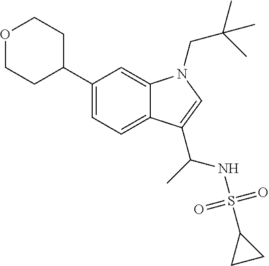 [SMILES: CC(NS(=O)(=O)C1CC1)c1cn(CC(C)(C)C)c2cc(C3CCOCC3)ccc12] |
| 24 | 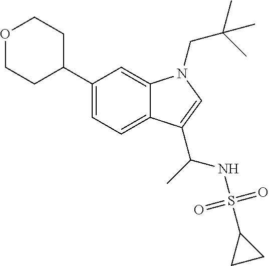 [SMILES: CC(NS(=O)(=O)C1CC1)c1cn(CC(C)(C)C)c2cc(C3CCOCC3)ccc12] |
| 26 | 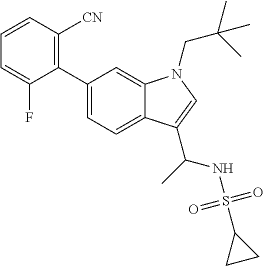 [SMILES: CC(NS(=O)(=O)C1CC1)c1cn(CC(C)(C)C)c2cc(-c3c(F)cccc3C#N)ccc12] |
| 27 | 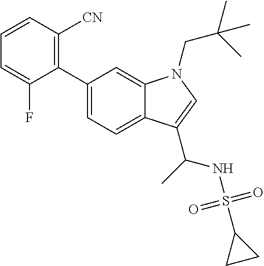 [SMILES: CC(NS(=O)(=O)C1CC1)c1cn(CC(C)(C)C)c2cc(-c3c(F)cccc3C#N)ccc12] |
| 28 | 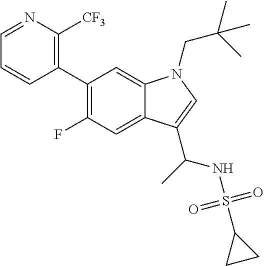 [SMILES: CC(NS(=O)(=O)C1CC1)c1cn(CC(C)(C)C)c2cc(-c3cccnc3C(F)(F)F)c(F)cc12] |
| 29 | 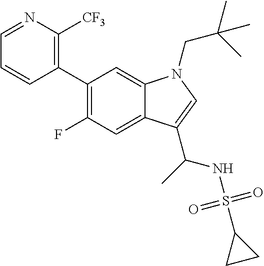 [SMILES: CC(NS(=O)(=O)C1CC1)c1cn(CC(C)(C)C)c2cc(-c3cccnc3C(F)(F)F)c(F)cc12] |
| 30 | 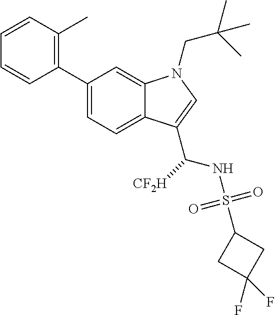 [SMILES: Cc1ccccc1-c1ccc2c([C@H](NS(=O)(=O)C3CC(F)(F)C3)C(F)F)cn(CC(C)(C)C)c2c1] |
| 31 | 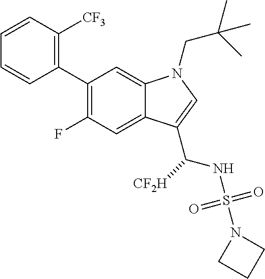 [SMILES: CC(C)(C)Cn1cc([C@H](NS(=O)(=O)N2CCC2)C(F)F)c2cc(F)c(-c3ccccc3C(F)(F)F)cc21] |
| 32 | 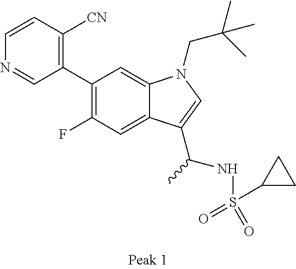 [SMILES: CC(NS(=O)(=O)C1CC1)c1cn(CC(C)(C)C)c2cc(-c3cnccc3C#N)c(F)cc12] |
| 33 | 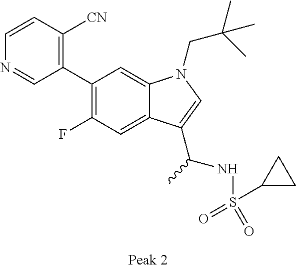 [SMILES: CC(NS(=O)(=O)C1CC1)c1cn(CC(C)(C)C)c2cc(-c3cnccc3C#N)c(F)cc12] |
| 34 | 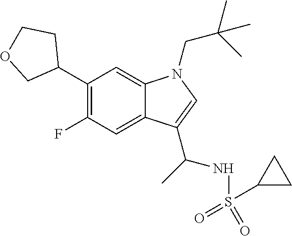 [SMILES: CC(NS(=O)(=O)C1CC1)c1cn(CC(C)(C)C)c2cc(C3CCOC3)c(F)cc12] |
| 35 | 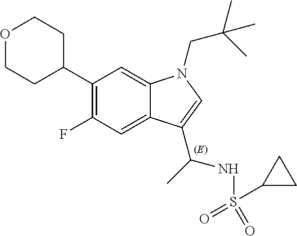 [SMILES: CC(NS(=O)(=O)C1CC1)c1cn(CC(C)(C)C)c2cc(C3CCOCC3)c(F)cc12] |
| 36 | 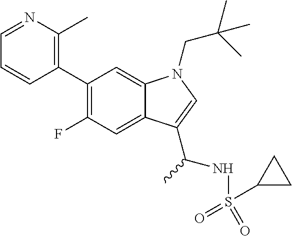 [SMILES: Cc1ncccc1-c1cc2c(cc1F)c(C(C)NS(=O)(=O)C1CC1)cn2CC(C)(C)C] |
| 37 | 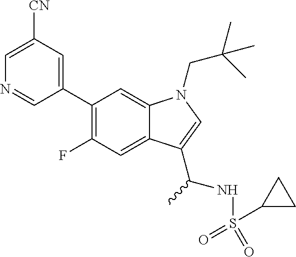 [SMILES: CC(NS(=O)(=O)C1CC1)c1cn(CC(C)(C)C)c2cc(-c3cncc(C#N)c3)c(F)cc12] |
| 38 | 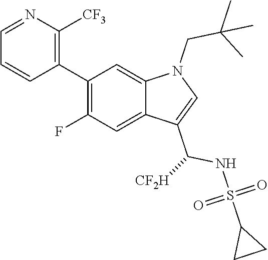 [SMILES: CC(C)(C)Cn1cc([C@H](NS(=O)(=O)C2CC2)C(F)F)c2cc(F)c(-c3cccnc3C(F)(F)F)cc21] |
| 39 | 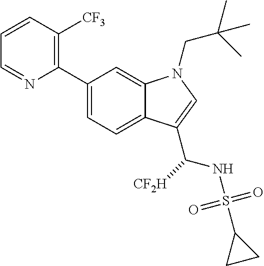 [SMILES: CC(C)(C)Cn1cc([C@H](NS(=O)(=O)C2CC2)C(F)F)c2ccc(-c3ncccc3C(F)(F)F)cc21] |
| 40 | 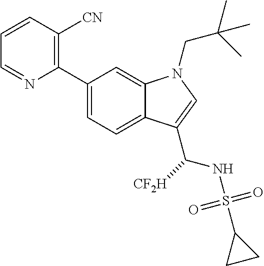 [SMILES: CC(C)(C)Cn1cc([C@H](NS(=O)(=O)C2CC2)C(F)F)c2ccc(-c3ncccc3C#N)cc21] |
| 41 | 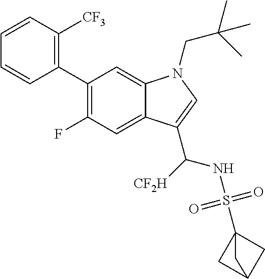 [SMILES: CC(C)(C)Cn1cc(C(NS(=O)(=O)C23CC(C2)C3)C(F)F)c2cc(F)c(-c3ccccc3C(F)(F)F)cc21] |
| 42 | 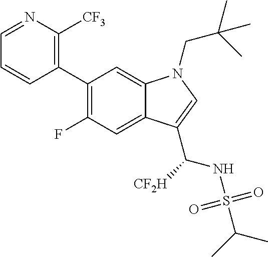 [SMILES: CC(C)S(=O)(=O)N[C@@H](c1cn(CC(C)(C)C)c2cc(-c3cccnc3C(F)(F)F)c(F)cc12)C(F)F] |
| 43 | 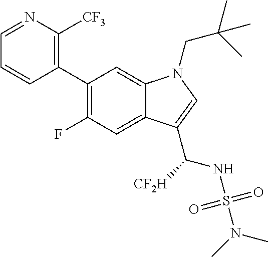 [SMILES: CN(C)S(=O)(=O)N[C@@H](c1cn(CC(C)(C)C)c2cc(-c3cccnc3C(F)(F)F)c(F)cc12)C(F)F] |
| 44 | 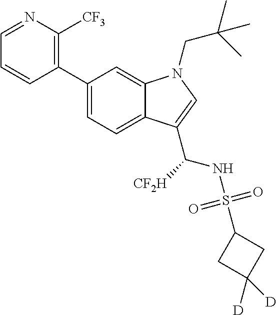 [SMILES: [2H]C1([2H])CC(S(=O)(=O)N[C@@H](c2cn(CC(C)(C)C)c3cc(-c4cccnc4C(F)(F)F)ccc23)C(F)F)C1] |
| 45 | 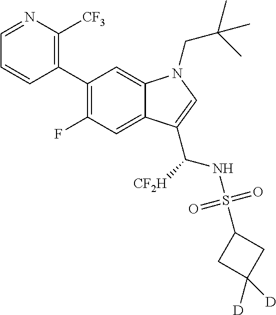 [SMILES: [2H]C1([2H])CC(S(=O)(=O)N[C@@H](c2cn(CC(C)(C)C)c3cc(-c4cccnc4C(F)(F)F)c(F)cc23)C(F)F)C1] |
| 46 | 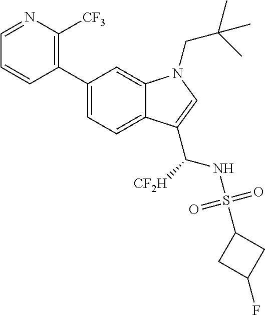 [SMILES: CC(C)(C)Cn1cc([C@H](NS(=O)(=O)C2CC(F)C2)C(F)F)c2ccc(-c3cccnc3C(F)(F)F)cc21] |
| 47 | 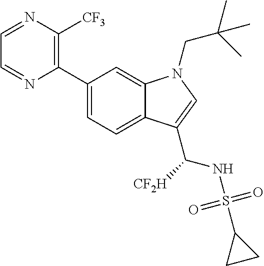 [SMILES: CC(C)(C)Cn1cc([C@H](NS(=O)(=O)C2CC2)C(F)F)c2ccc(-c3nccnc3C(F)(F)F)cc21] |
| 48 | 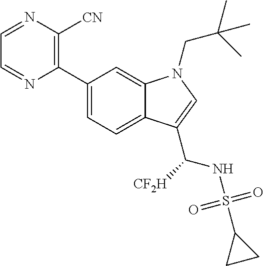 [SMILES: CC(C)(C)Cn1cc([C@H](NS(=O)(=O)C2CC2)C(F)F)c2ccc(-c3nccnc3C#N)cc21] |
| 49 | 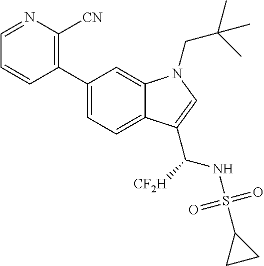 [SMILES: CC(C)(C)Cn1cc([C@H](NS(=O)(=O)C2CC2)C(F)F)c2ccc(-c3cccnc3C#N)cc21] |
| 51 | 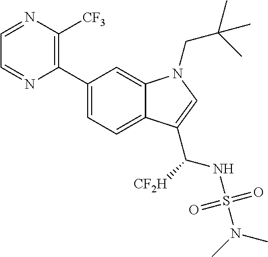 [SMILES: CN(C)S(=O)(=O)N[C@@H](c1cn(CC(C)(C)C)c2cc(-c3nccnc3C(F)(F)F)ccc12)C(F)F] |
| 52 | 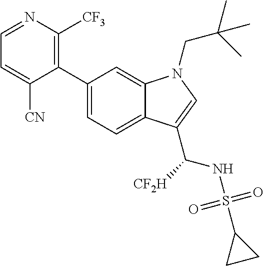 [SMILES: CC(C)(C)Cn1cc([C@H](NS(=O)(=O)C2CC2)C(F)F)c2ccc(-c3c(C#N)ccnc3C(F)(F)F)cc21] |
| 53 | 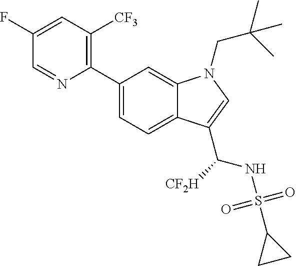 [SMILES: CC(C)(C)Cn1cc([C@H](NS(=O)(=O)C2CC2)C(F)F)c2ccc(-c3ncc(F)cc3C(F)(F)F)cc21] |
| 54 | 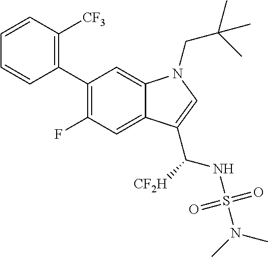 [SMILES: CN(C)S(=O)(=O)N[C@@H](c1cn(CC(C)(C)C)c2cc(-c3ccccc3C(F)(F)F)c(F)cc12)C(F)F] |
| 55 | 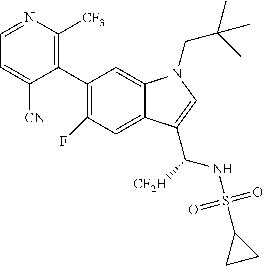 [SMILES: CC(C)(C)Cn1cc([C@H](NS(=O)(=O)C2CC2)C(F)F)c2cc(F)c(-c3c(C#N)ccnc3C(F)(F)F)cc21] |
| 56 | 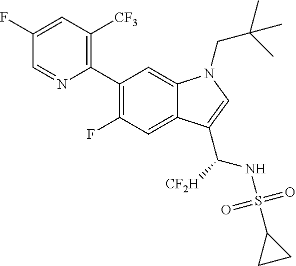 [SMILES: CC(C)(C)Cn1cc([C@H](NS(=O)(=O)C2CC2)C(F)F)c2cc(F)c(-c3ncc(F)cc3C(F)(F)F)cc21] |
| 57 | 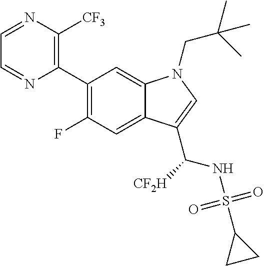 [SMILES: CC(C)(C)Cn1cc([C@H](NS(=O)(=O)C2CC2)C(F)F)c2cc(F)c(-c3nccnc3C(F)(F)F)cc21] |
| 58 | 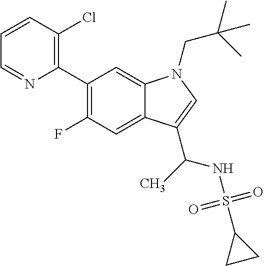 [SMILES: CC(NS(=O)(=O)C1CC1)c1cn(CC(C)(C)C)c2cc(-c3ncccc3Cl)c(F)cc12] |
| 59 | 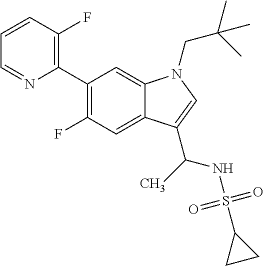 [SMILES: CC(NS(=O)(=O)C1CC1)c1cn(CC(C)(C)C)c2cc(-c3ncccc3F)c(F)cc12] |
| 60 | 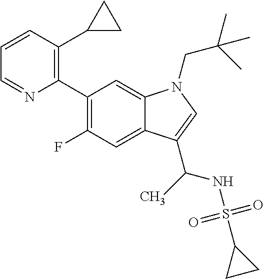 [SMILES: CC(NS(=O)(=O)C1CC1)c1cn(CC(C)(C)C)c2cc(-c3ncccc3C3CC3)c(F)cc12] |
| 61 | 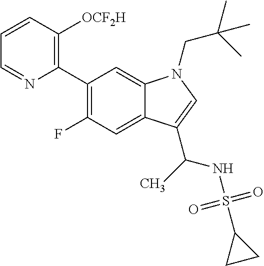 |
| 62 | 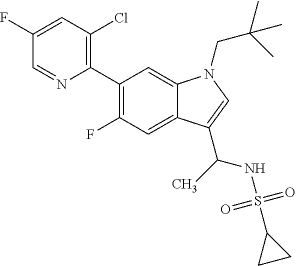 [SMILES: CC(NS(=O)(=O)C1CC1)c1cn(CC(C)(C)C)c2cc(-c3ncc(F)cc3Cl)c(F)cc12] |
| 63 | 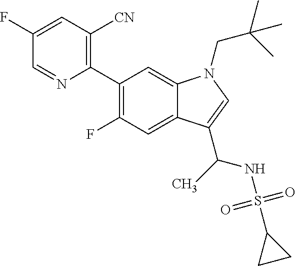 [SMILES: CC(NS(=O)(=O)C1CC1)c1cn(CC(C)(C)C)c2cc(-c3ncc(F)cc3C#N)c(F)cc12] |
| 64 | 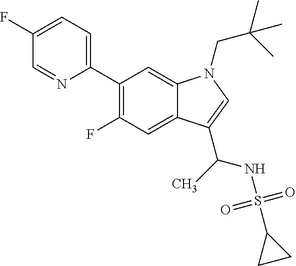 [SMILES: CC(NS(=O)(=O)C1CC1)c1cn(CC(C)(C)C)c2cc(-c3ccc(F)cn3)c(F)cc12] |
| 65 | 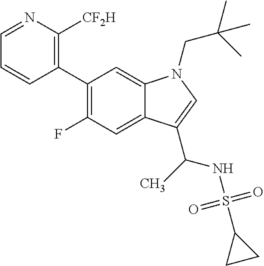 [SMILES: CC(NS(=O)(=O)C1CC1)c1cn(CC(C)(C)C)c2cc(-c3cccnc3C(F)F)c(F)cc12] |
| 66 | 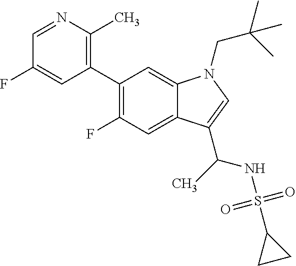 [SMILES: Cc1ncc(F)cc1-c1cc2c(cc1F)c(C(C)NS(=O)(=O)C1CC1)cn2CC(C)(C)C] |
| 67 | 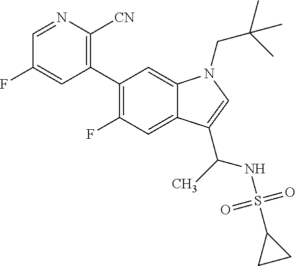 [SMILES: CC(NS(=O)(=O)C1CC1)c1cn(CC(C)(C)C)c2cc(-c3cc(F)cnc3C#N)c(F)cc12] |
| 68 | 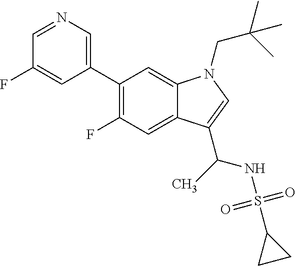 [SMILES: CC(NS(=O)(=O)C1CC1)c1cn(CC(C)(C)C)c2cc(-c3cncc(F)c3)c(F)cc12] |
| 69 | 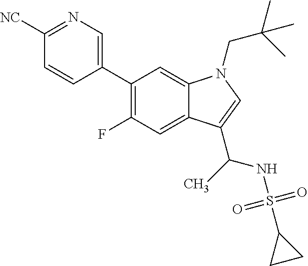 [SMILES: CC(NS(=O)(=O)C1CC1)c1cn(CC(C)(C)C)c2cc(-c3ccc(C#N)nc3)c(F)cc12] |
| 70 | 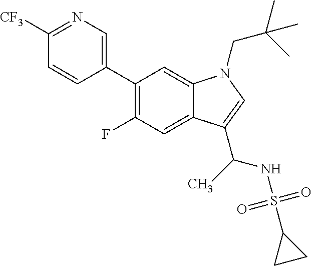 [SMILES: CC(NS(=O)(=O)C1CC1)c1cn(CC(C)(C)C)c2cc(-c3ccc(C(F)(F)F)nc3)c(F)cc12] |
| 71 | 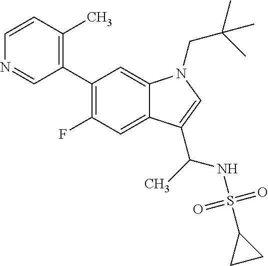 [SMILES: Cc1ccncc1-c1cc2c(cc1F)c(C(C)NS(=O)(=O)C1CC1)cn2CC(C)(C)C] |
| 72 | 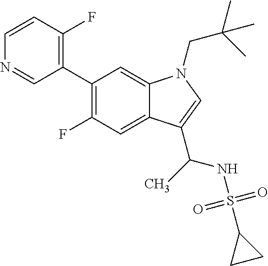 [SMILES: CC(NS(=O)(=O)C1CC1)c1cn(CC(C)(C)C)c2cc(-c3cnccc3F)c(F)cc12] |
| 73 | 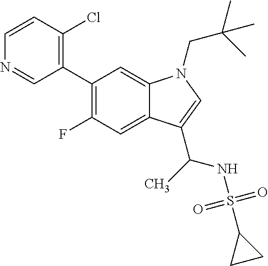 [SMILES: CC(NS(=O)(=O)C1CC1)c1cn(CC(C)(C)C)c2cc(-c3cnccc3Cl)c(F)cc12] |
| 74 | 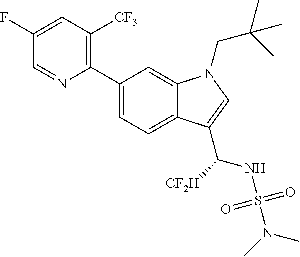 [SMILES: CN(C)S(=O)(=O)N[C@@H](c1cn(CC(C)(C)C)c2cc(-c3ncc(F)cc3C(F)(F)F)ccc12)C(F)F] |
| 75 | 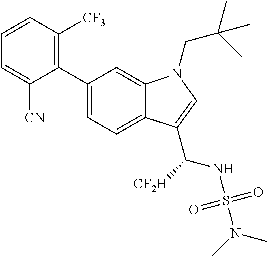 [SMILES: CN(C)S(=O)(=O)N[C@@H](c1cn(CC(C)(C)C)c2cc(-c3c(C#N)cccc3C(F)(F)F)ccc12)C(F)F] |
| 76 | 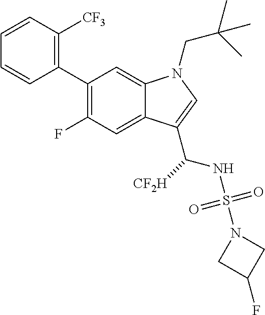 [SMILES: CC(C)(C)Cn1cc([C@H](NS(=O)(=O)N2CC(F)C2)C(F)F)c2cc(F)c(-c3ccccc3C(F)(F)F)cc21] |
| 77 | 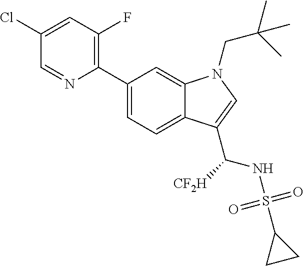 [SMILES: CC(C)(C)Cn1cc([C@H](NS(=O)(=O)C2CC2)C(F)F)c2ccc(-c3ncc(Cl)cc3F)cc21] |
| 78 | 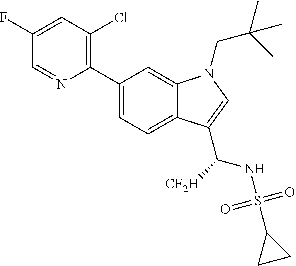 [SMILES: CC(C)(C)Cn1cc([C@H](NS(=O)(=O)C2CC2)C(F)F)c2ccc(-c3ncc(F)cc3Cl)cc21] |
| 79 | 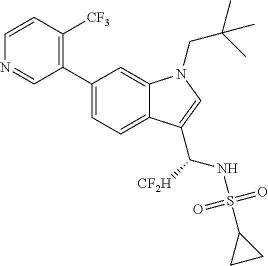 [SMILES: CC(C)(C)Cn1cc([C@H](NS(=O)(=O)C2CC2)C(F)F)c2ccc(-c3cnccc3C(F)(F)F)cc21] |
| 80 | 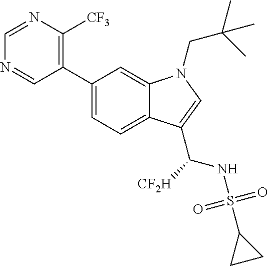 [SMILES: CC(C)(C)Cn1cc([C@H](NS(=O)(=O)C2CC2)C(F)F)c2ccc(-c3cncnc3C(F)(F)F)cc21] |
| 81 | 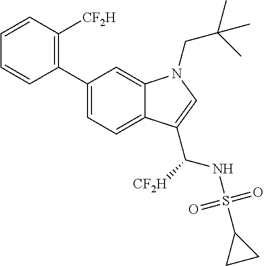 [SMILES: CC(C)(C)Cn1cc([C@H](NS(=O)(=O)C2CC2)C(F)F)c2ccc(-c3ccccc3C(F)F)cc21] |
| 82 | 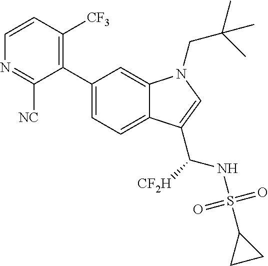 [SMILES: CC(C)(C)Cn1cc([C@H](NS(=O)(=O)C2CC2)C(F)F)c2ccc(-c3c(C(F)(F)F)ccnc3C#N)cc21] |
| 83 | 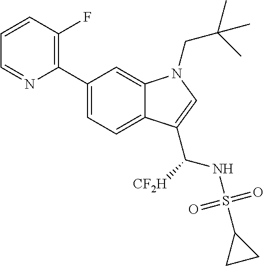 [SMILES: CC(C)(C)Cn1cc([C@H](NS(=O)(=O)C2CC2)C(F)F)c2ccc(-c3ncccc3F)cc21] |
| 84 | 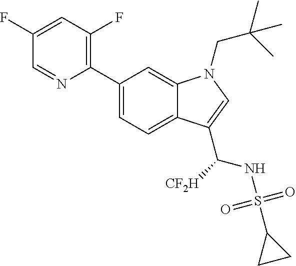 [SMILES: CC(C)(C)Cn1cc([C@H](NS(=O)(=O)C2CC2)C(F)F)c2ccc(-c3ncc(F)cc3F)cc21] |
| 85 | 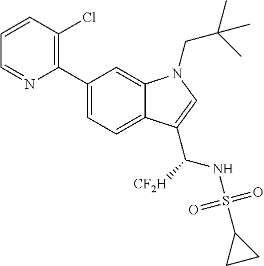 [SMILES: CC(C)(C)Cn1cc([C@H](NS(=O)(=O)C2CC2)C(F)F)c2ccc(-c3ncccc3Cl)cc21] |
| 86 | 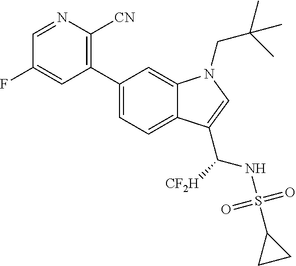 [SMILES: CC(C)(C)Cn1cc([C@H](NS(=O)(=O)C2CC2)C(F)F)c2ccc(-c3cc(F)cnc3C#N)cc21] |
| 87 | 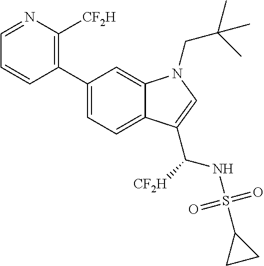 [SMILES: CC(C)(C)Cn1cc([C@H](NS(=O)(=O)C2CC2)C(F)F)c2ccc(-c3cccnc3C(F)F)cc21] |
| 88 | 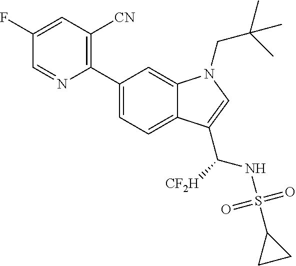 [SMILES: CC(C)(C)Cn1cc([C@H](NS(=O)(=O)C2CC2)C(F)F)c2ccc(-c3ncc(F)cc3C#N)cc21] |
| 89 | 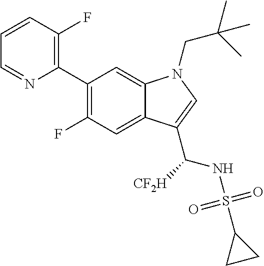 [SMILES: CC(C)(C)Cn1cc([C@H](NS(=O)(=O)C2CC2)C(F)F)c2cc(F)c(-c3ncccc3F)cc21] |
| 90 | 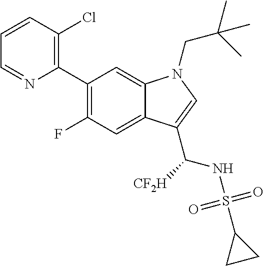 [SMILES: CC(C)(C)Cn1cc([C@H](NS(=O)(=O)C2CC2)C(F)F)c2cc(F)c(-c3ncccc3Cl)cc21] |
| 91 | 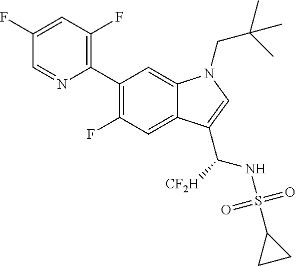 [SMILES: CC(C)(C)Cn1cc([C@H](NS(=O)(=O)C2CC2)C(F)F)c2cc(F)c(-c3ncc(F)cc3F)cc21] |
| 92 | 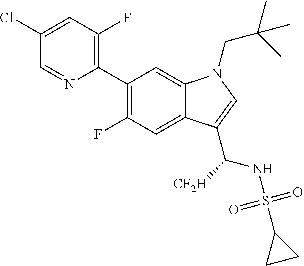 [SMILES: CC(C)(C)Cn1cc([C@H](NS(=O)(=O)C2CC2)C(F)F)c2cc(F)c(-c3ncc(Cl)cc3F)cc21] |
| 93 | 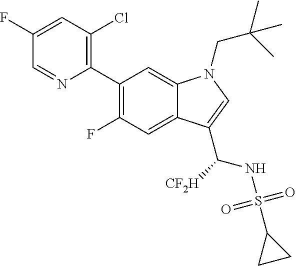 [SMILES: CC(C)(C)Cn1cc([C@H](NS(=O)(=O)C2CC2)C(F)F)c2cc(F)c(-c3ncc(F)cc3Cl)cc21] |
| 94 | 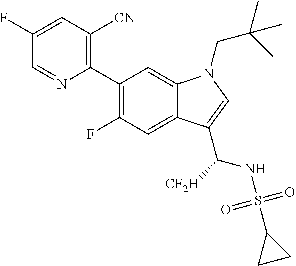 [SMILES: CC(C)(C)Cn1cc([C@H](NS(=O)(=O)C2CC2)C(F)F)c2cc(F)c(-c3ncc(F)cc3C#N)cc21] |
| 95 | 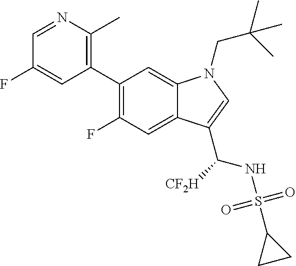 [SMILES: Cc1ncc(F)cc1-c1cc2c(cc1F)c([C@H](NS(=O)(=O)C1CC1)C(F)F)cn2CC(C)(C)C] |
| 96 | 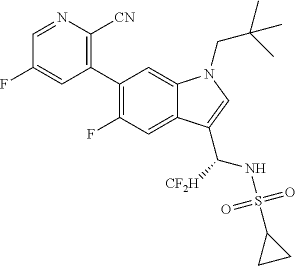 [SMILES: CC(C)(C)Cn1cc([C@H](NS(=O)(=O)C2CC2)C(F)F)c2cc(F)c(-c3cc(F)cnc3C#N)cc21] |
| 97 | 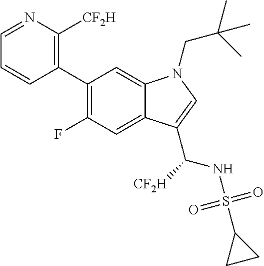 [SMILES: CC(C)(C)Cn1cc([C@H](NS(=O)(=O)C2CC2)C(F)F)c2cc(F)c(-c3cccnc3C(F)F)cc21] |
| 98 | 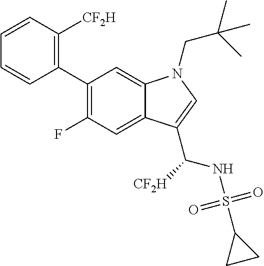 [SMILES: CC(C)(C)Cn1cc([C@H](NS(=O)(=O)C2CC2)C(F)F)c2cc(F)c(-c3ccccc3C(F)F)cc21] |
| 99 | 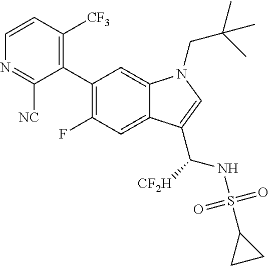 [SMILES: CC(C)(C)Cn1cc([C@H](NS(=O)(=O)C2CC2)C(F)F)c2cc(F)c(-c3c(C(F)(F)F)ccnc3C#N)cc21] |
| 100 | 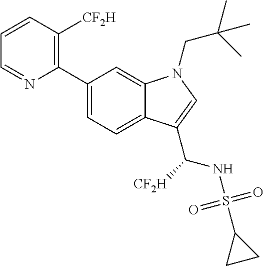 [SMILES: CC(C)(C)Cn1cc([C@H](NS(=O)(=O)C2CC2)C(F)F)c2ccc(-c3ncccc3C(F)F)cc21] |
| 101 | 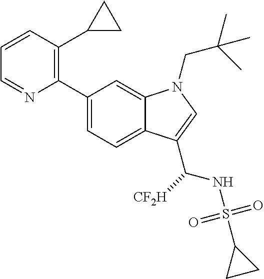 [SMILES: CC(C)(C)Cn1cc([C@H](NS(=O)(=O)C2CC2)C(F)F)c2ccc(-c3ncccc3C3CC3)cc21] |
| 102 | 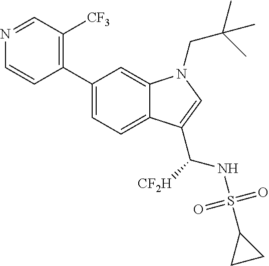 [SMILES: CC(C)(C)Cn1cc([C@H](NS(=O)(=O)C2CC2)C(F)F)c2ccc(-c3ccncc3C(F)(F)F)cc21] |
| 103 | 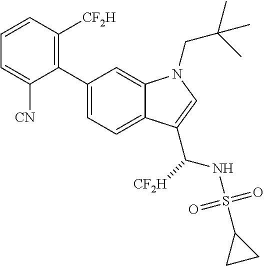 |
| 104 | 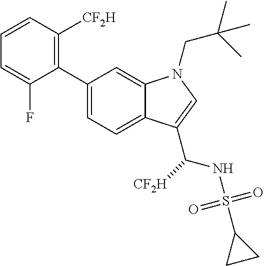 [SMILES: CC(C)(C)Cn1cc([C@H](NS(=O)(=O)C2CC2)C(F)F)c2ccc(-c3c(F)cccc3C(F)F)cc21] |
| 105 | 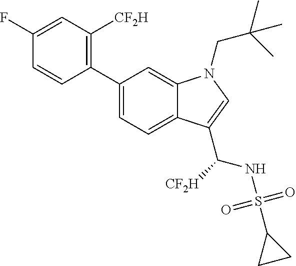 [SMILES: CC(C)(C)Cn1cc([C@H](NS(=O)(=O)C2CC2)C(F)F)c2ccc(-c3ccc(F)cc3C(F)F)cc21] |
| 106 | 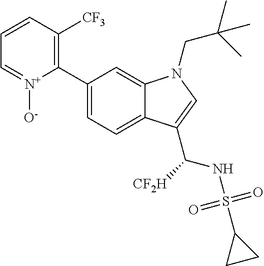 [SMILES: CC(C)(C)Cn1cc([C@H](NS(=O)(=O)C2CC2)C(F)F)c2ccc(-c3c(C(F)(F)F)ccc[n+]3[O-])cc21] |
| 107 | 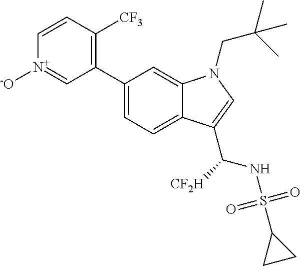 |
| 108 | 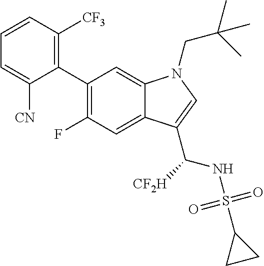 |
| 109 | 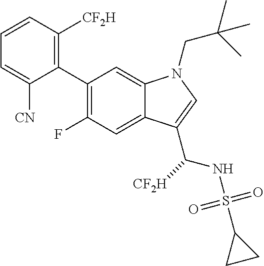 |
| 110 | 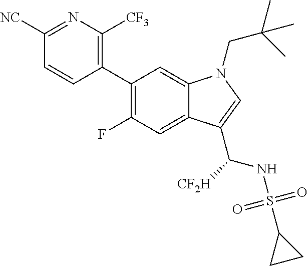 [SMILES: CC(C)(C)Cn1cc([C@H](NS(=O)(=O)C2CC2)C(F)F)c2cc(F)c(-c3ccc(C#N)nc3C(F)(F)F)cc21] |
| 111 | 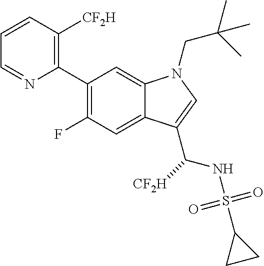 [SMILES: CC(C)(C)Cn1cc([C@H](NS(=O)(=O)C2CC2)C(F)F)c2cc(F)c(-c3ncccc3C(F)F)cc21] |
| 112 | 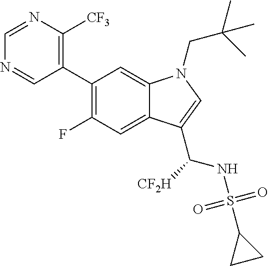 [SMILES: CC(C)(C)Cn1cc([C@H](NS(=O)(=O)C2CC2)C(F)F)c2cc(F)c(-c3cncnc3C(F)(F)F)cc21] |
| 113 | 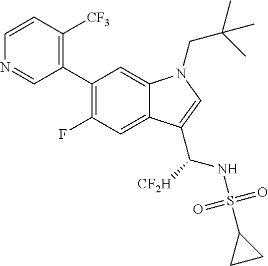 [SMILES: CC(C)(C)Cn1cc([C@H](NS(=O)(=O)C2CC2)C(F)F)c2cc(F)c(-c3cnccc3C(F)(F)F)cc21] |
| 114 | 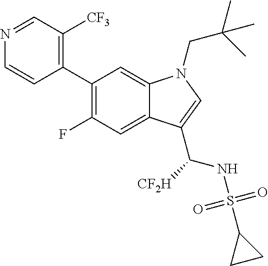 [SMILES: CC(C)(C)Cn1cc([C@H](NS(=O)(=O)C2CC2)C(F)F)c2cc(F)c(-c3ccncc3C(F)(F)F)cc21] |
| 115 | 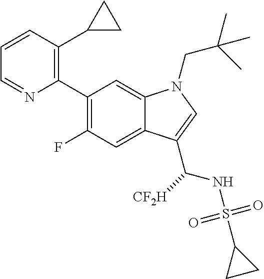 [SMILES: CC(C)(C)Cn1cc([C@H](NS(=O)(=O)C2CC2)C(F)F)c2cc(F)c(-c3ncccc3C3CC3)cc21] |
| 116 | 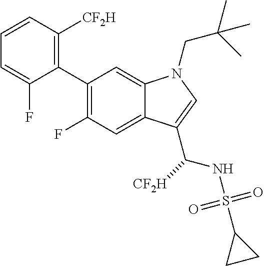 [SMILES: CC(C)(C)Cn1cc([C@H](NS(=O)(=O)C2CC2)C(F)F)c2cc(F)c(-c3c(F)cccc3C(F)F)cc21] |
| 117 | 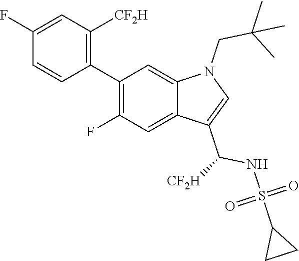 [SMILES: CC(C)(C)Cn1cc([C@H](NS(=O)(=O)C2CC2)C(F)F)c2cc(F)c(-c3ccc(F)cc3C(F)F)cc21] |
| 118 | 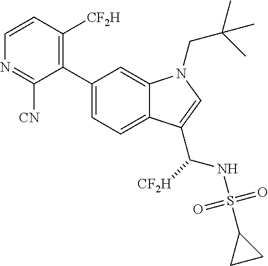 |
| 119 | 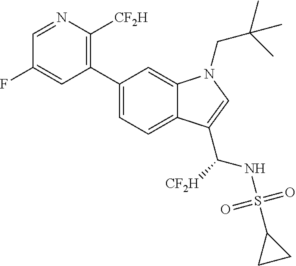 [SMILES: CC(C)(C)Cn1cc([C@H](NS(=O)(=O)C2CC2)C(F)F)c2ccc(-c3cc(F)cnc3C(F)F)cc21] |
| 120 | 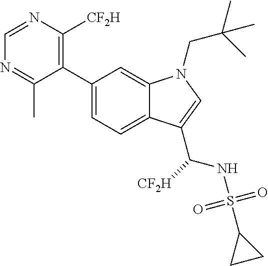 [SMILES: Cc1ncnc(C(F)F)c1-c1ccc2c([C@H](NS(=O)(=O)C3CC3)C(F)F)cn(CC(C)(C)C)c2c1] |
| 121 | 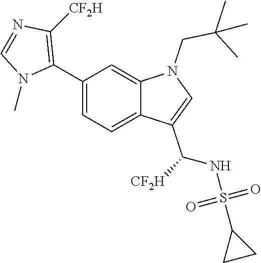 [SMILES: Cn1cnc(C(F)F)c1-c1ccc2c([C@H](NS(=O)(=O)C3CC3)C(F)F)cn(CC(C)(C)C)c2c1] |
| 122 | 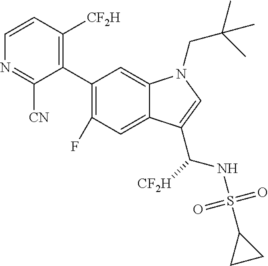 [SMILES: CC(C)(C)Cn1cc([C@H](NS(=O)(=O)C2CC2)C(F)F)c2cc(F)c(-c3c(C(F)F)ccnc3C#N)cc21] |
| 123 | 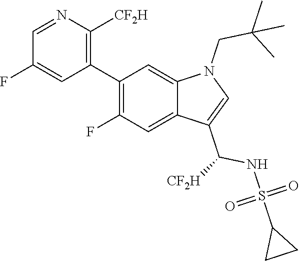 [SMILES: CC(C)(C)Cn1cc([C@H](NS(=O)(=O)C2CC2)C(F)F)c2cc(F)c(-c3cc(F)cnc3C(F)F)cc21] |
| 124 | 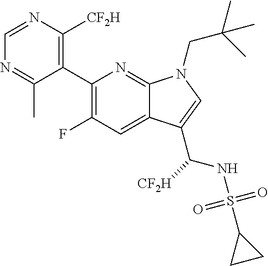 [SMILES: Cc1ncnc(C(F)F)c1-c1nc2c(cc1F)c([C@H](NS(=O)(=O)C1CC1)C(F)F)cn2CC(C)(C)C] |
| 125 | 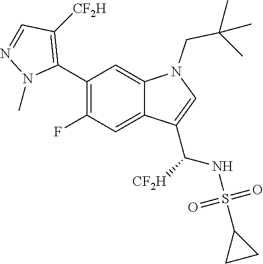 [SMILES: Cn1ncc(C(F)F)c1-c1cc2c(cc1F)c([C@H](NS(=O)(=O)C1CC1)C(F)F)cn2CC(C)(C)C] |
| 126 | 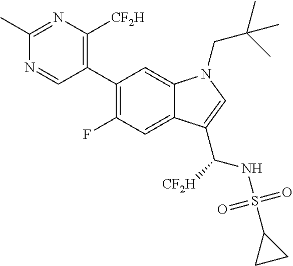 [SMILES: Cc1ncc(-c2cc3c(cc2F)c([C@H](NS(=O)(=O)C2CC2)C(F)F)cn3CC(C)(C)C)c(C(F)F)n1] |
| 127 | 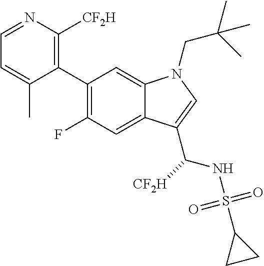 [SMILES: Cc1ccnc(C(F)F)c1-c1cc2c(cc1F)c([C@H](NS(=O)(=O)C1CC1)C(F)F)cn2CC(C)(C)C] |
| 128 | 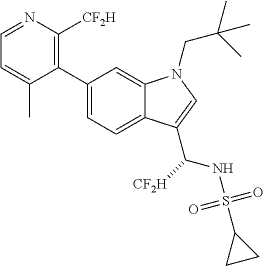 [SMILES: Cc1ccnc(C(F)F)c1-c1ccc2c([C@H](NS(=O)(=O)C3CC3)C(F)F)cn(CC(C)(C)C)c2c1] |
| 129 | 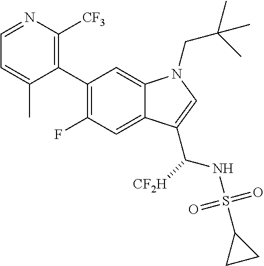 [SMILES: Cc1ccnc(C(F)(F)F)c1-c1cc2c(cc1F)c([C@H](NS(=O)(=O)C1CC1)C(F)F)cn2CC(C)(C)C] |
| 130 | 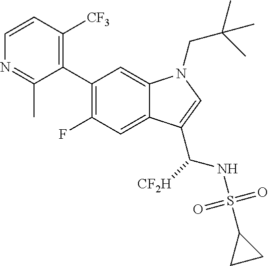 [SMILES: Cc1nccc(C(F)(F)F)c1-c1cc2c(cc1F)c([C@H](NS(=O)(=O)C1CC1)C(F)F)cn2CC(C)(C)C] |
| 131 | 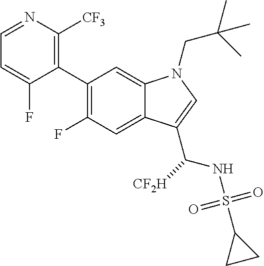 [SMILES: CC(C)(C)Cn1cc([C@H](NS(=O)(=O)C2CC2)C(F)F)c2cc(F)c(-c3c(F)ccnc3C(F)(F)F)cc21] |
| 132 | 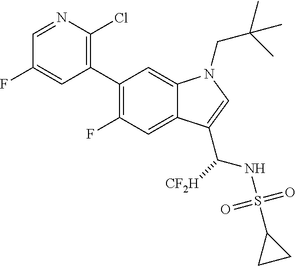 [SMILES: CC(C)(C)Cn1cc([C@H](NS(=O)(=O)C2CC2)C(F)F)c2cc(F)c(-c3cc(F)cnc3Cl)cc21] |
| 133 | 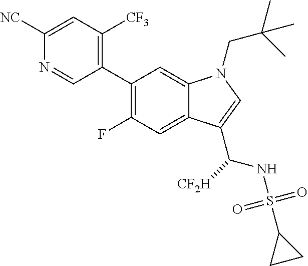 [SMILES: CC(C)(C)Cn1cc([C@H](NS(=O)(=O)C2CC2)C(F)F)c2cc(F)c(-c3cnc(C#N)cc3C(F)(F)F)cc21] |
| 134 | 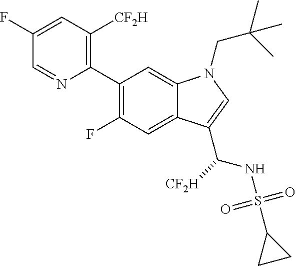 [SMILES: CC(C)(C)Cn1cc([C@H](NS(=O)(=O)C2CC2)C(F)F)c2cc(F)c(-c3ncc(F)cc3C(F)F)cc21] |
| 135 | 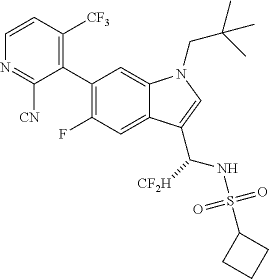 |
| 136 | 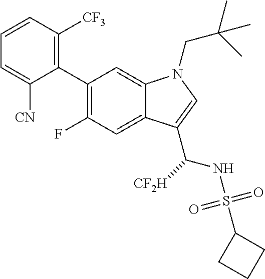 |
| 137 | 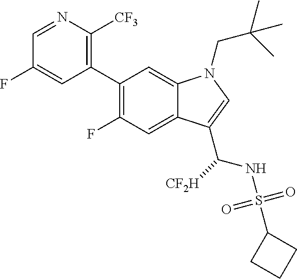 [SMILES: CC(C)(C)Cn1cc([C@H](NS(=O)(=O)C2CCC2)C(F)F)c2cc(F)c(-c3cc(F)cnc3C(F)(F)F)cc21] |
| 138 | 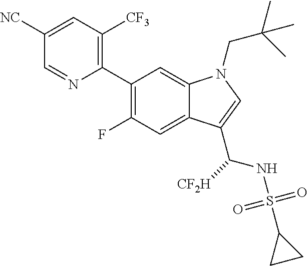 [SMILES: CC(C)(C)Cn1cc([C@H](NS(=O)(=O)C2CC2)C(F)F)c2cc(F)c(-c3ncc(C#N)cc3C(F)(F)F)cc21] |
| 139 | 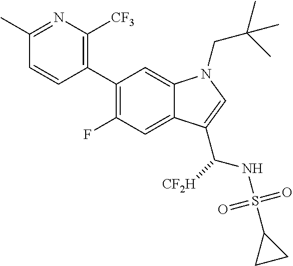 [SMILES: Cc1ccc(-c2cc3c(cc2F)c([C@H](NS(=O)(=O)C2CC2)C(F)F)cn3CC(C)(C)C)c(C(F)(F)F)n1] |
| 140 | 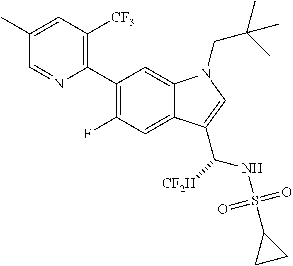 [SMILES: Cc1cnc(-c2cc3c(cc2F)c([C@H](NS(=O)(=O)C2CC2)C(F)F)cn3CC(C)(C)C)c(C(F)(F)F)c1] |
| 141 | 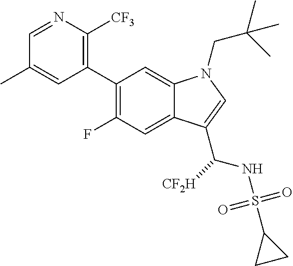 [SMILES: Cc1cnc(C(F)(F)F)c(-c2cc3c(cc2F)c([C@H](NS(=O)(=O)C2CC2)C(F)F)cn3CC(C)(C)C)c1] |
| 142 | 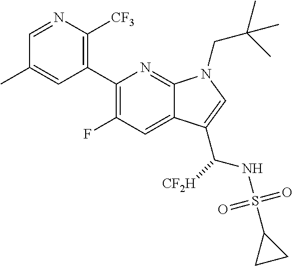 [SMILES: Cc1cnc(C(F)(F)F)c(-c2nc3c(cc2F)c([C@H](NS(=O)(=O)C2CC2)C(F)F)cn3CC(C)(C)C)c1] |
| 143 | 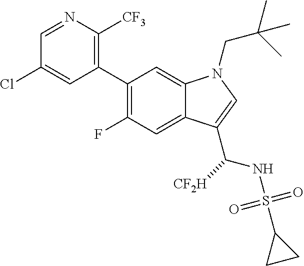 [SMILES: CC(C)(C)Cn1cc([C@H](NS(=O)(=O)C2CC2)C(F)F)c2cc(F)c(-c3cc(Cl)cnc3C(F)(F)F)cc21] |
| 144 | 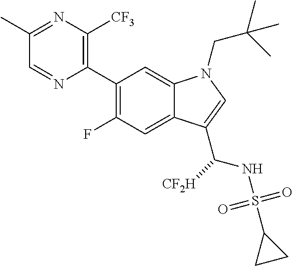 [SMILES: Cc1cnc(-c2cc3c(cc2F)c([C@H](NS(=O)(=O)C2CC2)C(F)F)cn3CC(C)(C)C)c(C(F)(F)F)n1] |
| 145 | 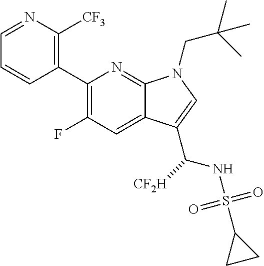 [SMILES: CC(C)(C)Cn1cc([C@H](NS(=O)(=O)C2CC2)C(F)F)c2cc(F)c(-c3cccnc3C(F)(F)F)nc21] |
| 146 | 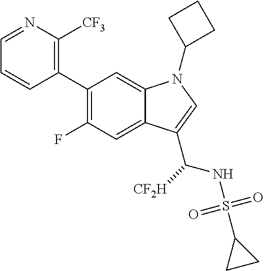 [SMILES: O=S(=O)(N[C@@H](c1cn(C2CCC2)c2cc(-c3cccnc3C(F)(F)F)c(F)cc12)C(F)F)C1CC1] |
| 147 | 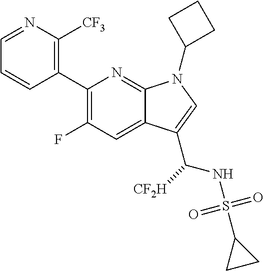 [SMILES: O=S(=O)(N[C@@H](c1cn(C2CCC2)c2nc(-c3cccnc3C(F)(F)F)c(F)cc12)C(F)F)C1CC1] |
| 148 | 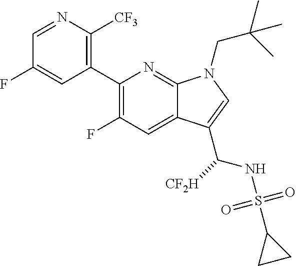 [SMILES: CC(C)(C)Cn1cc([C@H](NS(=O)(=O)C2CC2)C(F)F)c2cc(F)c(-c3cc(F)cnc3C(F)(F)F)nc21] |
| 149 | 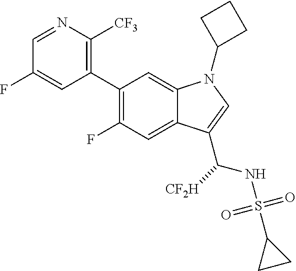 [SMILES: O=S(=O)(N[C@@H](c1cn(C2CCC2)c2cc(-c3cc(F)cnc3C(F)(F)F)c(F)cc12)C(F)F)C1CC1] |
| 150 | 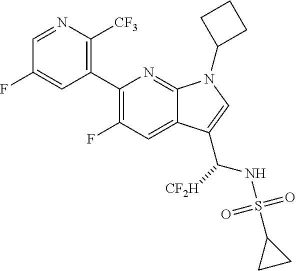 [SMILES: O=S(=O)(N[C@@H](c1cn(C2CCC2)c2nc(-c3cc(F)cnc3C(F)(F)F)c(F)cc12)C(F)F)C1CC1] |
| 151 | 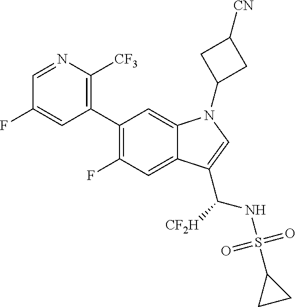 [SMILES: N#CC1CC(n2cc([C@H](NS(=O)(=O)C3CC3)C(F)F)c3cc(F)c(-c4cc(F)cnc4C(F)(F)F)cc32)C1] |
| 152 | 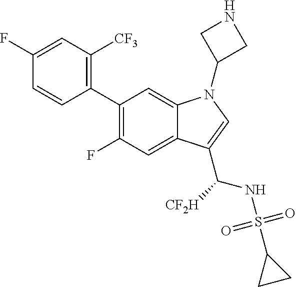 [SMILES: O=S(=O)(N[C@@H](c1cn(C2CNC2)c2cc(-c3ccc(F)cc3C(F)(F)F)c(F)cc12)C(F)F)C1CC1] |
| 153 | 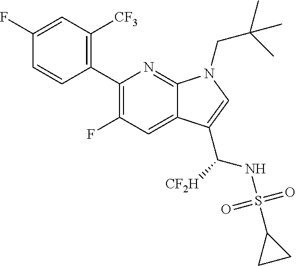 [SMILES: CC(C)(C)Cn1cc([C@H](NS(=O)(=O)C2CC2)C(F)F)c2cc(F)c(-c3ccc(F)cc3C(F)(F)F)nc21] |
| 154 | 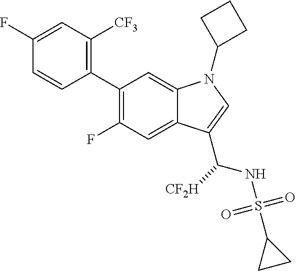 [SMILES: O=S(=O)(N[C@@H](c1cn(C2CCC2)c2cc(-c3ccc(F)cc3C(F)(F)F)c(F)cc12)C(F)F)C1CC1] |
| 155 | 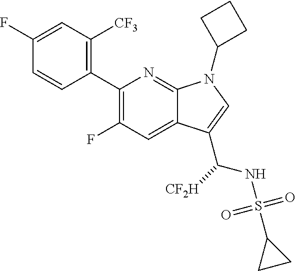 [SMILES: O=S(=O)(N[C@@H](c1cn(C2CCC2)c2nc(-c3ccc(F)cc3C(F)(F)F)c(F)cc12)C(F)F)C1CC1] |
| 156 | 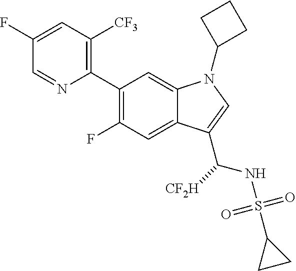 [SMILES: O=S(=O)(N[C@@H](c1cn(C2CCC2)c2cc(-c3ncc(F)cc3C(F)(F)F)c(F)cc12)C(F)F)C1CC1] |
| 157 | 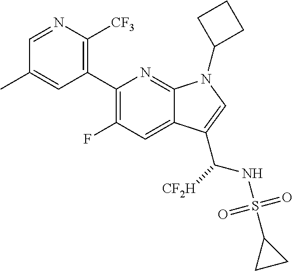 [SMILES: Cc1cnc(C(F)(F)F)c(-c2nc3c(cc2F)c([C@H](NS(=O)(=O)C2CC2)C(F)F)cn3C2CCC2)c1] |
| 158 | 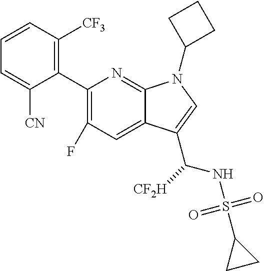 [SMILES: N#Cc1cccc(C(F)(F)F)c1-c1nc2c(cc1F)c([C@H](NS(=O)(=O)C1CC1)C(F)F)cn2C1CCC1] |
| 159 | 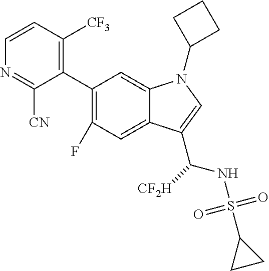 [SMILES: N#Cc1nccc(C(F)(F)F)c1-c1cc2c(cc1F)c([C@H](NS(=O)(=O)C1CC1)C(F)F)cn2C1CCC1] |
| 160 | 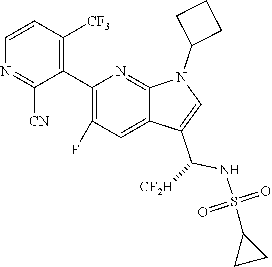 [SMILES: N#Cc1nccc(C(F)(F)F)c1-c1nc2c(cc1F)c([C@H](NS(=O)(=O)C1CC1)C(F)F)cn2C1CCC1] |
| 161 | 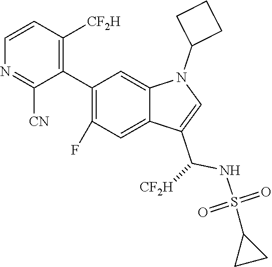 [SMILES: N#Cc1nccc(C(F)F)c1-c1cc2c(cc1F)c([C@H](NS(=O)(=O)C1CC1)C(F)F)cn2C1CCC1] |
| 162 | 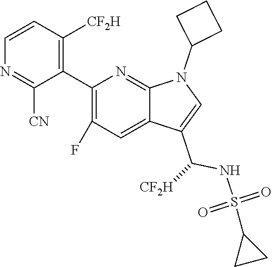 [SMILES: N#Cc1nccc(C(F)F)c1-c1nc2c(cc1F)c([C@H](NS(=O)(=O)C1CC1)C(F)F)cn2C1CCC1] |
| 163 | 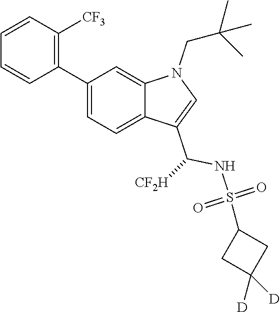 [SMILES: [2H]C1([2H])CC(S(=O)(=O)N[C@@H](c2cn(CC(C)(C)C)c3cc(-c4ccccc4C(F)(F)F)ccc23)C(F)F)C1] |
| 164 | 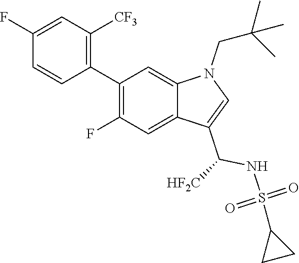 |
| 165 | 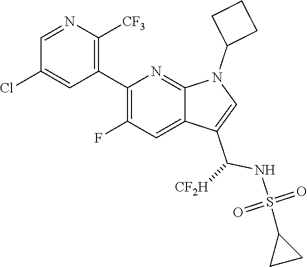 [SMILES: O=S(=O)(N[C@@H](c1cn(C2CCC2)c2nc(-c3cc(Cl)cnc3C(F)(F)F)c(F)cc12)C(F)F)C1CC1] |
| 166 | 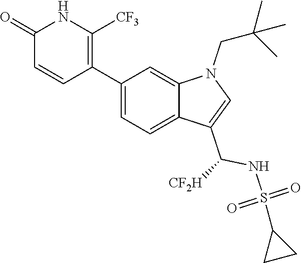 [SMILES: CC(C)(C)Cn1cc([C@H](NS(=O)(=O)C2CC2)C(F)F)c2ccc(-c3ccc(=O)[nH]c3C(F)(F)F)cc21] |
| 167 | 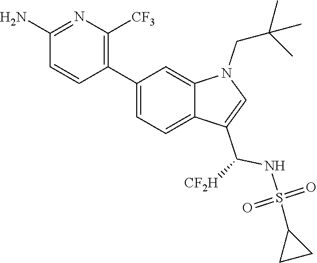 [SMILES: CC(C)(C)Cn1cc([C@H](NS(=O)(=O)C2CC2)C(F)F)c2ccc(-c3ccc(N)nc3C(F)(F)F)cc21] |
| 168 | 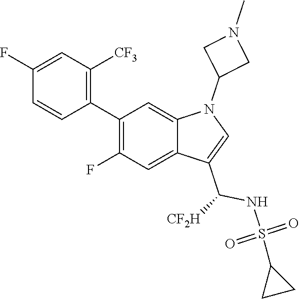 [SMILES: CN1CC(n2cc([C@H](NS(=O)(=O)C3CC3)C(F)F)c3cc(F)c(-c4ccc(F)cc4C(F)(F)F)cc32)C1] |
| 169 | 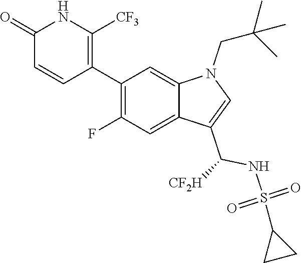 [SMILES: CC(C)(C)Cn1cc([C@H](NS(=O)(=O)C2CC2)C(F)F)c2cc(F)c(-c3ccc(=O)[nH]c3C(F)(F)F)cc21] |
| 170 | 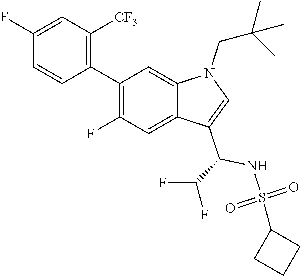 [SMILES: CC(C)(C)Cn1cc([C@H](NS(=O)(=O)C2CCC2)C(F)F)c2cc(F)c(-c3ccc(F)cc3C(F)(F)F)cc21] |
| 171 | 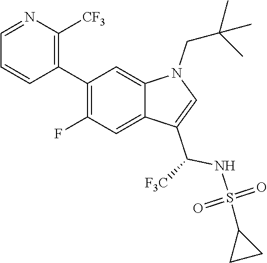 [SMILES: CC(C)(C)Cn1cc([C@H](NS(=O)(=O)C2CC2)C(F)(F)F)c2cc(F)c(-c3cccnc3C(F)(F)F)cc21] |
| 172 | 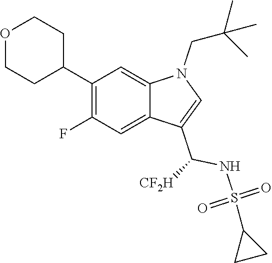 [SMILES: CC(C)(C)Cn1cc([C@H](NS(=O)(=O)C2CC2)C(F)F)c2cc(F)c(C3CCOCC3)cc21] |
| 173 | 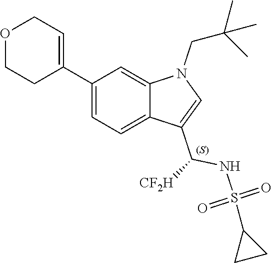 |
| 174 | 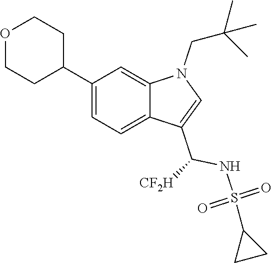 [SMILES: CC(C)(C)Cn1cc([C@H](NS(=O)(=O)C2CC2)C(F)F)c2ccc(C3CCOCC3)cc21] |
| 175 | 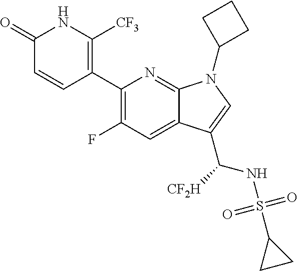 [SMILES: O=c1ccc(-c2nc3c(cc2F)c([C@H](NS(=O)(=O)C2CC2)C(F)F)cn3C2CCC2)c(C(F)(F)F)[nH]1] |
| 176 | 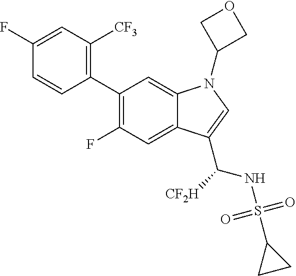 [SMILES: O=S(=O)(N[C@@H](c1cn(C2COC2)c2cc(-c3ccc(F)cc3C(F)(F)F)c(F)cc12)C(F)F)C1CC1] |
| 177 | 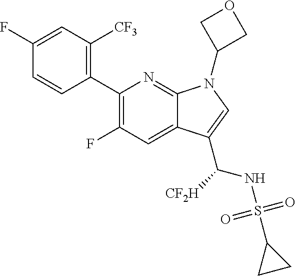 [SMILES: O=S(=O)(N[C@@H](c1cn(C2COC2)c2nc(-c3ccc(F)cc3C(F)(F)F)c(F)cc12)C(F)F)C1CC1] |
| 178 | 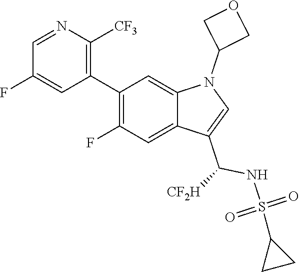 [SMILES: O=S(=O)(N[C@@H](c1cn(C2COC2)c2cc(-c3cc(F)cnc3C(F)(F)F)c(F)cc12)C(F)F)C1CC1] |
| 179 | 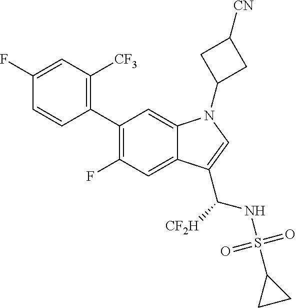 [SMILES: N#CC1CC(n2cc([C@H](NS(=O)(=O)C3CC3)C(F)F)c3cc(F)c(-c4ccc(F)cc4C(F)(F)F)cc32)C1] |
| 180 | 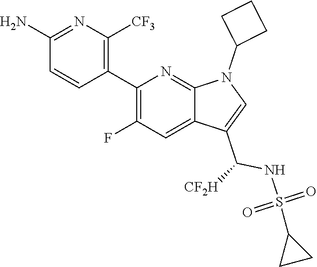 [SMILES: Nc1ccc(-c2nc3c(cc2F)c([C@H](NS(=O)(=O)C2CC2)C(F)F)cn3C2CCC2)c(C(F)(F)F)n1] |
| 181 | 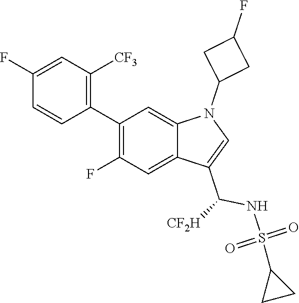 [SMILES: O=S(=O)(N[C@@H](c1cn(C2CC(F)C2)c2cc(-c3ccc(F)cc3C(F)(F)F)c(F)cc12)C(F)F)C1CC1] |
| 182 | 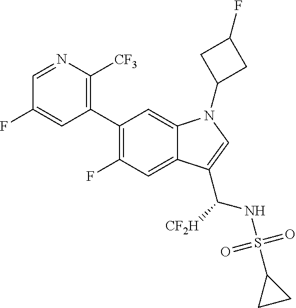 [SMILES: O=S(=O)(N[C@@H](c1cn(C2CC(F)C2)c2cc(-c3cc(F)cnc3C(F)(F)F)c(F)cc12)C(F)F)C1CC1] |
| 184 | 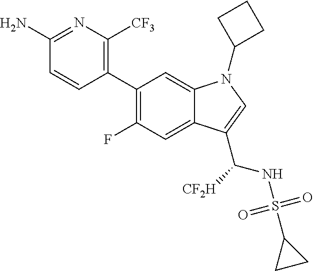 [SMILES: Nc1ccc(-c2cc3c(cc2F)c([C@H](NS(=O)(=O)C2CC2)C(F)F)cn3C2CCC2)c(C(F)(F)F)n1] |
| 185 | 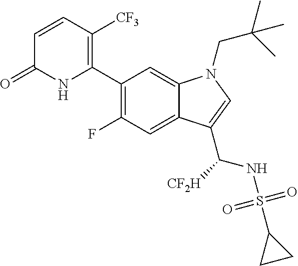 [SMILES: CC(C)(C)Cn1cc([C@H](NS(=O)(=O)C2CC2)C(F)F)c2cc(F)c(-c3[nH]c(=O)ccc3C(F)(F)F)cc21] |
| 186 | 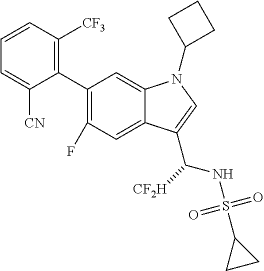 [SMILES: N#Cc1cccc(C(F)(F)F)c1-c1cc2c(cc1F)c([C@H](NS(=O)(=O)C1CC1)C(F)F)cn2C1CCC1] |
| 187 | 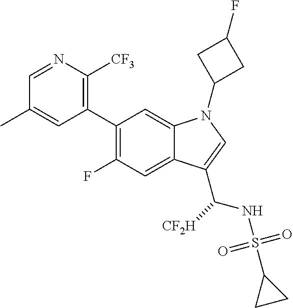 [SMILES: Cc1cnc(C(F)(F)F)c(-c2cc3c(cc2F)c([C@H](NS(=O)(=O)C2CC2)C(F)F)cn3C2CC(F)C2)c1] |
| 188 | 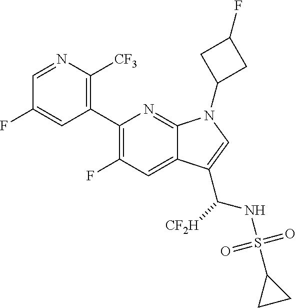 [SMILES: O=S(=O)(N[C@@H](c1cn(C2CC(F)C2)c2nc(-c3cc(F)cnc3C(F)(F)F)c(F)cc12)C(F)F)C1CC1] |
| 189 | 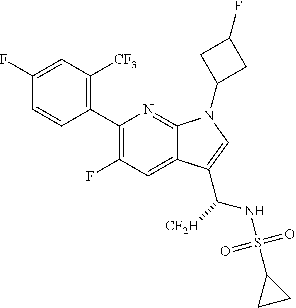 [SMILES: O=S(=O)(N[C@@H](c1cn(C2CC(F)C2)c2nc(-c3ccc(F)cc3C(F)(F)F)c(F)cc12)C(F)F)C1CC1] |
| 190 | 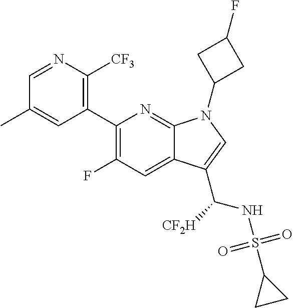 [SMILES: Cc1cnc(C(F)(F)F)c(-c2nc3c(cc2F)c([C@H](NS(=O)(=O)C2CC2)C(F)F)cn3C2CC(F)C2)c1] |
| 191 | 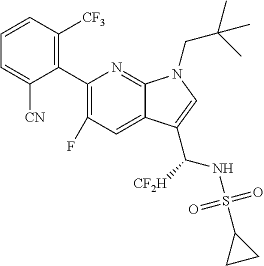 [SMILES: CC(C)(C)Cn1cc([C@H](NS(=O)(=O)C2CC2)C(F)F)c2cc(F)c(-c3c(C#N)cccc3C(F)(F)F)nc21] |
| 192 | 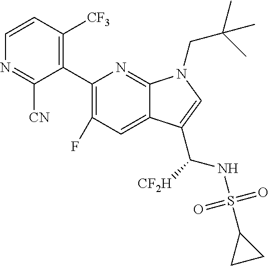 [SMILES: CC(C)(C)Cn1cc([C@H](NS(=O)(=O)C2CC2)C(F)F)c2cc(F)c(-c3c(C(F)(F)F)ccnc3C#N)nc21] |
| 193 | 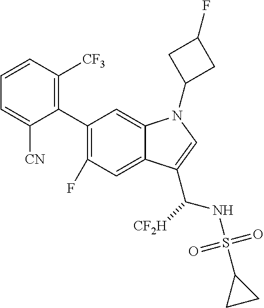 [SMILES: N#Cc1cccc(C(F)(F)F)c1-c1cc2c(cc1F)c([C@H](NS(=O)(=O)C1CC1)C(F)F)cn2C1CC(F)C1] |
| 194 | 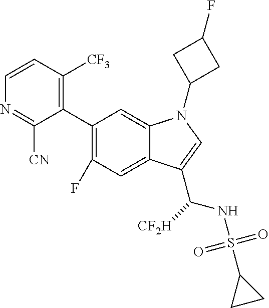 [SMILES: N#Cc1nccc(C(F)(F)F)c1-c1cc2c(cc1F)c([C@H](NS(=O)(=O)C1CC1)C(F)F)cn2C1CC(F)C1] |
| 195 | 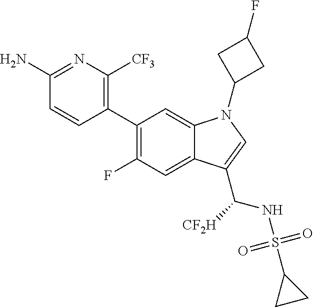 [SMILES: Nc1ccc(-c2cc3c(cc2F)c([C@H](NS(=O)(=O)C2CC2)C(F)F)cn3C2CC(F)C2)c(C(F)(F)F)n1] |
| 196 | 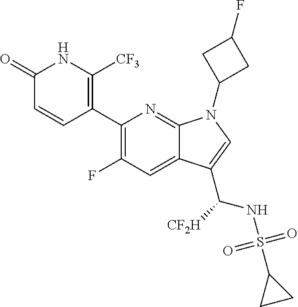 [SMILES: O=c1ccc(-c2nc3c(cc2F)c([C@H](NS(=O)(=O)C2CC2)C(F)F)cn3C2CC(F)C2)c(C(F)(F)F)[nH]1] |
| 197 | 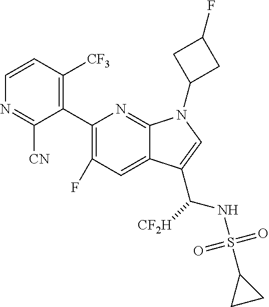 [SMILES: N#Cc1nccc(C(F)(F)F)c1-c1nc2c(cc1F)c([C@H](NS(=O)(=O)C1CC1)C(F)F)cn2C1CC(F)C1] |
| 198 | 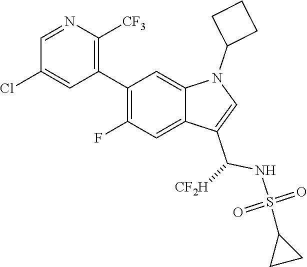 [SMILES: O=S(=O)(N[C@@H](c1cn(C2CCC2)c2cc(-c3cc(Cl)cnc3C(F)(F)F)c(F)cc12)C(F)F)C1CC1] |
| 199 | 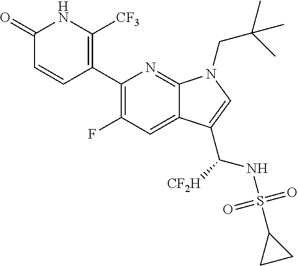 [SMILES: CC(C)(C)Cn1cc([C@H](NS(=O)(=O)C2CC2)C(F)F)c2cc(F)c(-c3ccc(=O)[nH]c3C(F)(F)F)nc21] |
| 200 | 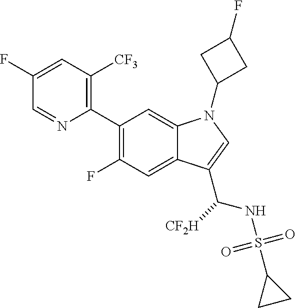 [SMILES: O=S(=O)(N[C@@H](c1cn(C2CC(F)C2)c2cc(-c3ncc(F)cc3C(F)(F)F)c(F)cc12)C(F)F)C1CC1] |
| 201 | 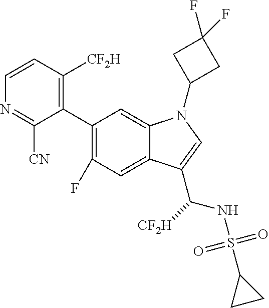 [SMILES: N#Cc1nccc(C(F)F)c1-c1cc2c(cc1F)c([C@H](NS(=O)(=O)C1CC1)C(F)F)cn2C1CC(F)(F)C1] |
| 202 | 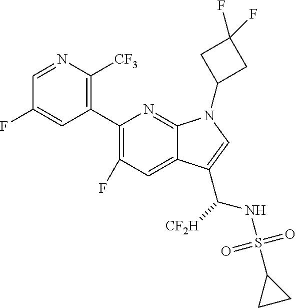 [SMILES: O=S(=O)(N[C@@H](c1cn(C2CC(F)(F)C2)c2nc(-c3cc(F)cnc3C(F)(F)F)c(F)cc12)C(F)F)C1CC1] |
| 203 | 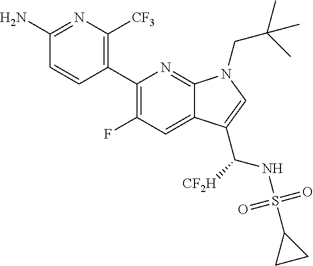 [SMILES: CC(C)(C)Cn1cc([C@H](NS(=O)(=O)C2CC2)C(F)F)c2cc(F)c(-c3ccc(N)nc3C(F)(F)F)nc21] |
| 204 | 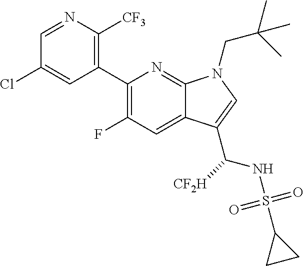 [SMILES: CC(C)(C)Cn1cc([C@H](NS(=O)(=O)C2CC2)C(F)F)c2cc(F)c(-c3cc(Cl)cnc3C(F)(F)F)nc21] |
| 205 | 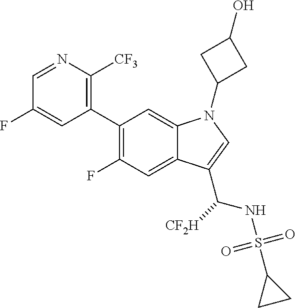 [SMILES: O=S(=O)(N[C@@H](c1cn(C2CC(O)C2)c2cc(-c3cc(F)cnc3C(F)(F)F)c(F)cc12)C(F)F)C1CC1] |
| 206 | 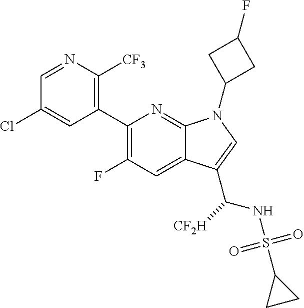 [SMILES: O=S(=O)(N[C@@H](c1cn(C2CC(F)C2)c2nc(-c3cc(Cl)cnc3C(F)(F)F)c(F)cc12)C(F)F)C1CC1] |
| 207 | 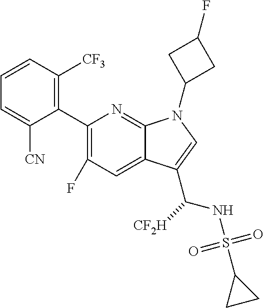 [SMILES: N#Cc1cccc(C(F)(F)F)c1-c1nc2c(cc1F)c([C@H](NS(=O)(=O)C1CC1)C(F)F)cn2C1CC(F)C1] |
| 208 | 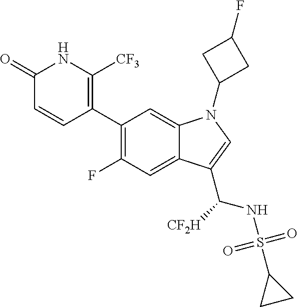 [SMILES: O=c1ccc(-c2cc3c(cc2F)c([C@H](NS(=O)(=O)C2CC2)C(F)F)cn3C2CC(F)C2)c(C(F)(F)F)[nH]1] |
| 209 | 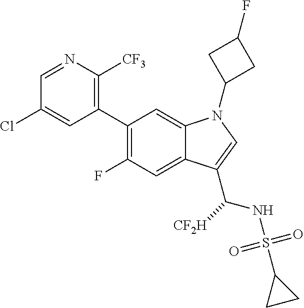 [SMILES: O=S(=O)(N[C@@H](c1cn(C2CC(F)C2)c2cc(-c3cc(Cl)cnc3C(F)(F)F)c(F)cc12)C(F)F)C1CC1] |
| 210 | 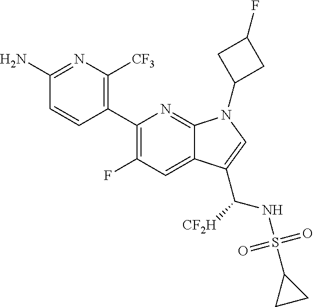 [SMILES: Nc1ccc(-c2nc3c(cc2F)c([C@H](NS(=O)(=O)C2CC2)C(F)F)cn3C2CC(F)C2)c(C(F)(F)F)n1] |
| 211 | 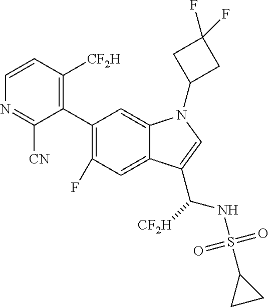 [SMILES: N#Cc1nccc(C(F)F)c1-c1cc2c(cc1F)c([C@H](NS(=O)(=O)C1CC1)C(F)F)cn2C1CC(F)(F)C1] |
| 212 | 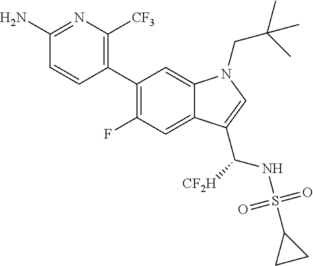 [SMILES: CC(C)(C)Cn1cc([C@H](NS(=O)(=O)C2CC2)C(F)F)c2cc(F)c(-c3ccc(N)nc3C(F)(F)F)cc21] |

2. A pharmaceutical composition comprising a compound according to claim 1 and a pharmaceutically acceptable carrier.

3. A method for treating a subject suffering from hyperglycemia, dyslipidemia, atherosclerosis, an autoimmune or inflammatory disorder or disease, or a cancer, comprising administering to the subject a therapeutically effective amount of a compound or pharmaceutically acceptable salt thereof according to claim 1.

4. The method according to claim 3, wherein the cancer is one selected from the group consisting of glioblastoma, colorectal cancer, and hepatocellular carcinoma.

5. A method for repressing T_{H}17 cell development in a subject, comprising administering to the subject a compound or pharmaceutically acceptable salt thereof according to claim 1.

6. A method for selectively agonizing REV-ERBα over REV-ERBβ in a subject, comprising administering to the subject a compound or pharmaceutically acceptable salt thereof according to claim 1.

7. Use of a compound or pharmaceutically acceptable salt thereof according to claim 1 for treating a subject suffering from hyperglycemia, dyslipidemia, atherosclerosis, an autoimmune or inflammatory disorder or disease, or a cancer.

8. The use according to claim 7, wherein the cancer is one selected from glioblastoma, colorectal cancer, and hepatocellular carcinoma.

9. Use of a compound or pharmaceutically acceptable salt thereof according to claim 1 for repressing T_{H}17 cell development in a subject.

10. Use of a compound or pharmaceutically acceptable salt thereof according to claim 1 for selectively agonizing REV-ERBα over REV-ERBβ in a subject.
